(19) United States
(12) Patent Application Publication
(10) Pub. No.: US 2023/0092431 A1
(43) Pub. Date: Mar. 23, 2023

(54) BACTERIA ENGINEERED TO TREAT DISORDERS IN WHICH OXALATE IS DETRIMENTAL

(71) Applicant: Synlogic Operating Company, Inc., Cambridge, MA (US)

(72) Inventors: Vincent M. Isabella, Medford, MA (US); David Lubkowicz, Somerville, MA (US); Michael James, Melrose, MA (US)

(21) Appl. No.: 17/792,030

(22) PCT Filed: Jan. 14, 2021
(86) PCT No.: PCT/US21/13401

§ 371 Date: Jul. 11, 2022

Related U.S. Application Data
(60) Provisional application No. 62/960,950, filed on Jan. 14, 2020.
(60) Provisional application No. 62/993,301, filed on Mar. 23, 2020.
(60) Provisional application No. 63/028,902, filed on May 22, 2020.
(60) Provisional application No. 63/065,752, filed on Aug. 14, 2020.
(60) Provisional application No. 63/089,758, filed on Oct. 9, 2020.
(60) Provisional application No. 63/091,620, filed on Oct. 14, 2020.
(60) Provisional application No. 63/111,376, filed on Nov. 9, 2020.

(51) Int. Cl. A61K 35/74; C12N 9/00; C12N 9/88; C12N 9/10; C12N 15/70; A61K 31/4439; A61P 13/12; A61P 3/00

(52) U.S. Cl. CPC A61K 35/74; C12N 9/93; C12N 9/88; C12N 9/13; C12Y 602/01008; C12Y 401/01008; C12Y 208/03016; C12N 15/70; A61K 31/4439; A61P 13/12; A61P 3/00

(57) ABSTRACT

The present invention provides recombinant bacterial cells comprising at least one heterologous gene encoding at least one oxalate catabolism enzyme. In another aspect, the recombinant bacterial cells further comprise at least one heterologous gene encoding an importer of oxalate. The invention further provides pharmaceutical compositions comprising the recombinant bacteria, and methods for treating disorders in which oxalate is detrimental, such as hyperoxaluria, using the pharmaceutical compositions of the invention.

### RELATED APPLICATIONS

The instant application claims priority to U.S. Provisional Application No. 63/111,376 filed on Nov. 9, 2020; U.S. Provisional Application No. 63/091,620 filed on Oct. 14, 2020; U.S. Provisional Application No. 63/089,758 filed on Oct. 9, 2020; U.S. Provisional Application No. 63/065,752 filed on Aug. 14, 2020; U.S. Provisional Application No. 63/028,902 filed on May 22, 2020; U.S. Provisional Application No. 62/993,301 filed on Mar. 23, 2020; and U.S. Provisional Application No. 62/960,950 filed on Jan. 14, 2020; the entire contents of each of which are expressly incorporated by reference herein in their entireties.

### SEQUENCE LISTING

The instant application contains a Sequence Listing which has been submitted electronically in ASCII format and is hereby incorporated by reference in its entirety. Said ASCII copy, created on Jan. 8, 2021, is named 126046-05120_SL.txt and is 153,109 bytes in size.

### BACKGROUND

Oxalate, the ionic form of oxalic acid, arises in the human body from dietary intake or from endogenous synthesis. Oxalate is ubiquitous in plants and plant-derived foods, and as such, is inevitably part of the human diet. Endogenously-synthesized oxalate is primarily derived from glyoxylate in the liver where excess glyoxylate is converted to oxalate by glycolate oxidase or lactate dehydrogenase (Robijn et al., Kidney Int. 80: 1146-58 (2011)). Healthy individuals normally excrete urinary oxalate in ranges between 20-40 mg of oxalate per 24 hours. However, urinary oxalate excretion at concentrations exceeding 40-45 mg per 24 hours is clinically considered hyperoxaluria (Robijn et al. (2011)). Hyperoxaluria is characterized by increased urinary excretion of and elevated systemic levels of oxalate, and urinary oxalate levels are typically about 90-500 mg per 24 hours in primary hyperoxaluria and about 45-130 mg per 24 hours in enteric hyperpxaluria. If left untreated, hyperoxaluria can cause significant morbidity and mortality, including the development of renal stones (kidney stones), nephrocalcinosis (increased calcium in the kidney), crystallopathy and most significantly, End Stage Renal Disease (Tasian et al., J. Am. Soc. Nephrol., 2018; 29(6):1731-1740 and Siener et al., Kidney International, 2013:83:1144-1149).

Hyperoxalurias can generally be divided into two clinical categories: primary and secondary hyperoxalurias. Primary hyperoxalurias are autosomal-recessive inherited diseases resulting from mutations in one of several genes involved in oxalate metabolism (Hoppe et al., Nephr. Dial. Transplant. 26: 3609-15 (2011)). The primary hyperoxalurias are characterized by elevated urinary oxalate excretion which ultimately may result in recurrent urolithiasis, crystallopathy, progressive nephrocalcinosis and early end-stage renal disease. In addition, when chronic renal insufficiency occurs in patients with primary hyperoxalurias, systemic deposition of calcium oxalate (also known as oxalosis) may occur in various organ systems which can lead to bone disease, erythropoietin refractory anemia, skin ulcers, digital gangrene, cardiac arrhythmias, and cardiomyopathy (Hoppe et al. (2011)).

Primary hyperoxaluria type I (PHI) is the most common and severe form of hyperoxaluria, and is caused by a defect in the vitamin B6-dependent hepatic peroxisomal enzyme, alanine glyoxalate aminotransferase (AGT, encoded by the AGXT gene), which catalyzes the transamination of glyoxalate to glycine (Purdue et al., J. Cell Biol. 111: 2341-51 (1990); Hoppe et al., Kidney Int 75: 1264-71 (2009)). AGT deficiency allows glyoxylate to be reduced to glycolate which is then oxidized to produce oxalate. Over 140 mutations of the human AGXT gene have been identified (Williams et al., Hum. Mut. 30: 910-7 (2009)). Primary hyperoxaluria type II (PHII) is caused by mutations of the enzyme glyoxylate/hydroxypyruvate reductase (GRHPR), an enzyme having glyoxylate reductase (GR), hydroxypyruvate reductase (HPR), and D-glycerate dehydrogenase (DGDH) activities (see, e.g., Cramer et al., Hum. Mol. Gen. 8:2063-9 (1999)). More than a dozen mutation of the human GRHPR gene have been identified (Cregeen et al., Hum. Mut. 22: 497 (2003)). Both PHI and PHII result in severe hyperoxaluria (Robijn et al. (2011)). Primary hyperoxaluria type III (PHIII) is caused by a mutation in the HOGA1 gene, which encodes a 4-hydroxy 2-oxoglutarate aldolase, a mitochondrial enzyme that breaks down 4-hydroxy 2-oxoglutarate into pyruvate and glyoxalate (Pitt et al., JIMD Reports 15: 1-6 (2015)). 15 mutations in the human HOGA1 gene have been identified (Bhasin et al., World J Nephrol. 4: 235-44 (2015)).

Secondary hyperoxaluria typically results from conditions underlying increased absorption of oxalate, including increased dietary intake of oxalate, increased intestinal absorption of oxalate, excessive intake of oxalate precursors, gut microflora imbalances, and genetic variations of intestinal oxalate transporters (Bhasin et al., 2015; Robijn et al. (2011)). Increased oxalate absorption with consequent hyperoxaluria, often referred to as enteric hyperoxaluria, is observed in patients with a variety of intestinal disorders, including the syndrome of bacterial overgrowth, Crohn's disease, inflammatory bowel disease, as well as other malabsorptive states, such as, after jejunoileal bypass for obesity, after gastric ulcer surgery, and chronic mesenteric ischemia (Pardi et al., Am. J. Gastroenterol. 93: 500-14 (1998); Hylander et al., Scand. J. Gastroent. 15: 349-52 (1980); Canos et al., Can. Med. Assoc. J. 124: 729-33 (1981); Drenick et al., Ann. Intern. Med. 89: 594-9 (1978)). In addition, hyperoxaluria may also occur following renal transplantation (Robijn et al. (2011)). Patients with secondary hyperoxalurias and enteric hyperoxalurias are predisposed to developing calcium oxalate stones, which may lead to significant renal damage and ultimately result in End Stage Renal Disease.

Currently available treatments for hyperoxalurias are inadequate. Strategies for the treatment of primary hyperoxalurias include reducing urinary oxalate with pyridoxine, which is only effective in less than half of patients with PHI, and ineffective in patients with PHII and PHIII (Hoppe et al. (2011)). Further, treatments with citrate, orthophosphate, and magnesium to increase the urinary solubility of calcium oxalate, and thus preserve renal function, are not well characterized (Hoppe et al. (2011)). Other strategies for the treatment of secondary and enteric hyperoxalurias, which are quite arduous and often ineffective, include reducing the dietary intake of oxalate, oral calcium supplementation, and the use of bile acid sequestrants (Parivar et al, J. Urol. 155: 432-40 (1996); Hylander et al. (1980); McLeod and Churchhill, J. Urol. 148: 974-8 (1992)). Generally, dietary restrictions are not entirely effective because patients cannot readily identify the food products to avoid (Parivar et al. (1996)). Therefore, there is significant unmet need for effective, reliable, and/or long-term treatment of hyperoxalurias.

### SUMMARY

The present disclosure provides engineered bacterial cells, pharmaceutical compositions thereof, and methods of modulating and treating disorders in which oxalate is detrimental. Specifically, the engineered bacteria disclosed herein have been constructed to comprise genetic circuits composed of, for example, one or more oxalate catabolism genes to treat the disease, as well as other optional circuitry designed to ensure the safety and non-colonization of a subject that is administered the engineered bacteria, such as, for example, auxotrophies, kill switches, and combinations thereof. These engineered bacteria are safe and well tolerated and augment the innate activities of the subject's microbiome to achieve a therapeutic effect.

In one embodiment, disclosed herein is a method for reducing the levels of oxalate in a subject, the method comprising administering to the subject a pharmaceutical composition comprising a recombinant bacterium comprising one or more gene sequences encoding one or more oxalate catabolism enzymes operably linked to a directly or indirectly first promoter that is not associated with the oxalate catabolism enzyme gene in nature, thereby reducing the levels of oxalate in the subject.

In one embodiment, the recombinant bacterium has an oxalate consumption activity of 1 μmol/1×10⁹ cell. In one embodiment, the recombinant bacterium has an oxalate consumption activity of about 50-600 mg/day, about 100-550 mg/day, about 100-500 mg/day, about 100-400 mg/day, about 100-300 mg/day. In one embodiment, the recombinant bacterium has an oxalate consumption activity of about 150-300 mg/day. In one embodiment, the recombinant bacterium has an oxalate consumption activity of about 50 mg/day, about 100 mg/day, about 150 mg/day, about 200 mg/day, about 210 mg/day, about 211 mg/day, about 225 mg/day, about 250 mg/day, about 275 mg/day, about 300 mg/day, about 325 mg/day, about 350 mg/day, about 350 mg/day, about 375 mg/day, about 400 mg/day, about 425 mg/day, about 450 mg/day, about 475 mg/day, about 500 mg/day, about 525 mg/day, a bout 550 mg/day, about 575 mg/day, or about 600 mg/day. In one embodiment, the recombinant bacterium has an oxalate consumption activity of about 50 mg/day, about 100 mg/day, about 150 mg/day, about 200 mg/day, about 211 mg/day, about 225 mg/day, about 250 mg/day, about 275 mg/day, about 300 mg/day, about 325 mg/day, about 350 mg/day, about 350 mg/day, about 375 mg/day, about 400 mg/day, about 425 mg/day, about 450 mg/day, about 475 mg/day, about 500 mg/day, about 525 mg/day, a bout 550 mg/day, about 575 mg/day, or about 600 mg/day under anaerobic conditions. In one embodiment, the recombinant bacterium has an oxalate consumption activity of about 50 mg/day, about 100 mg/day, about 150 mg/day, about 200 mg/day, about 211 mg/day, about 225 mg/day, about 250 mg/day, about 275 mg/day, about 300 mg/day, about 325 mg/day, about 350 mg/day, about 350 mg/day, about 375 mg/day, about 400 mg/day, about 425 mg/day, about 450 mg/day, about 475 mg/day, about 500 mg/day, about 525 mg/day, a bout 550 mg/day, about 575 mg/day, or about 600 mg/day under anaerobic conditions when administered to the subject three times per day. In one embodiment, the anaerobic conditions are conditions in the intestine and/or colon of the subject.

In one embodiment, the method reduces acute levels of oxalate in the subject by about two fold. In one embodiment, the method reduces acute levels of oxalate in the subject by about three fold. In one embodiment, the method reduces chronic levels of oxalate in the subject by about two fold. In one embodiment, the method reduces chronic levels of oxalate in the subject by about three fold.

In one embodiment, the method reduces acute levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day. In one embodiment, the method reduces chronic levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day.

In one embodiment, the method reduces acute levels of oxalate in the subject by at least about 40% by day 5 after administration. In one embodiment, the method reduces acute levels of oxalate in the subject by at least about 50% by day 5 after administration. In one embodiment, the method reduces acute levels of oxalate in the subject by at least about 60% by day 5 after administration. In one embodiment, the method reduces acute levels of oxalate in the subject by at least about 70% by day 5 after administration. In one embodiment, the method reduces acute levels of oxalate in the subject by at least about 80% by day 5 after administration.

In one embodiment, the method reduces acute levels of oxalate in the subject by at least about 10% by about 24 hours after administration. In one embodiment, the method reduces acute levels of oxalate in the subject by at least about 15% by about 24 hours after administration. In one embodiment, the method reduces acute levels of oxalate in the subject by at least about 20% by about 24 hours after administration.

In one embodiment, the recombinant bacterium is of the genus Escherichia. In one embodiment, the recombinant bacterium is of the species Escherichia coli strain Nissle.

In one embodiment, the pharmaceutical composition is administered orally. In one embodiment, the subject is fed a meal within one hour of administering the pharmaceutical composition. In one embodiment, the subject is fed a meal concurrently with administering the pharmaceutical composition. In one embodiment, the subject is a human subject.

In one embodiment, disclosed herein is a recombinant bacterium comprising one or more gene sequences encoding one or more oxalate catabolism enzymes operably linked to a directly or indirectly first inducible promoter that is not associated with the oxalate catabolism enzyme gene in nature.

In one embodiment, the recombinant bacterium has an oxalate consumption activity of 1 μmol/1×10⁹ cell. In one embodiment, the recombinant bacterium has an oxalate consumption activity of about 150-300 mg/day under anaerobic conditions. In one embodiment, the recombinant bacterium has an oxalate consumption activity of about 200 mg/day under anaerobic conditions. In one embodiment, the recombinant bacterium has an oxalate consumption activity of about 200 mg/day under anaerobic conditions when administered to the subject three times per day. In one embodiment, the anaerobic conditions are conditions in the intestine and/or colon of the subject.

In one embodiment, the one or more gene sequences comprise a scaaE3 gene, an frc gene, and an oxdC gene. In one embodiment, the scaaE3 gene comprises a sequence having at least 90%, 91%, 92%, 93%, 94%, 95%, 96%, 97%, 98%, or 99% identity to, comprises, or consists of SEQ ID NO:3. In one embodiment, the frc gene comprises a sequence having at least 90%, 91%, 92%, 93%, 94%, 95%, 96%, 97%, 98%, or 99% identity to, comprises, or consists of SEQ ID NO:1. In one embodiment, the scaaE3 gene comprises SEQ ID NO:3. In one embodiment, the oxdC gene comprises a sequence having at least 90%, 91%, 92%, 93%, 94%, 95%, 96%, 97%, 98%, or 99% identity to, comprises, or consists of SEQ ID NO:2. In one embodiment, the frc gene comprises SEQ ID NO:2.

In one embodiment, the recombinant bacterium further comprises a gene encoding an oxalate importer. In one embodiment, the gene encoding the oxalate importer is an oxlT gene. In one embodiment, the oxlT gene comprises a sequence having at least 90%, 91%, 92%, 93%, 94%, 95%, 96%, 97%, 98%, or 99% identity to, comprises, or consists of SEQ ID NO: 11. In one embodiment, the oxlT gene comprises SEQ ID NO: 11.

In one embodiment, the recombinant bacterium further comprises an auxotrophy. In one embodiment, the auxotrophy is a thyA auxotrophy. In one embodiment, thyA has at least 90%, 91%, 92%, 93%, 94%, 95%, 96%, 97%, 98%, or 99% identity to, comprises, or consists of SEQ ID NO: 62.

In one embodiment, the recombinant bacterium further comprises a deletion in an endogenous phage. In one embodiment, the endogenous phage comprises a sequence having at least 90%, 91%, 92%, 93%, 94%, 95%, 96%, 97%, 98%, or 99% identity to, comprises, or consists of SEQ ID NO:63. In one embodiment, the endogenous phase comprises a sequence of SEQ ID NO:63.

In one embodiment, the recombinant bacterium does not comprise a gene encoding for antibiotic resistance. In one embodiment, the first promoter is an inducible promoter. In one embodiment, the inducible promoter is induced by low oxygen or anaerobic conditions, temperature, or the hypoxic environment of a tumor. In one embodiment, the inducible promoter is an FNR promoter. In one embodiment, the FNR promoter is a promoter selected from the group consisting of any one of SEQ ID NOs:13-29.

In one embodiment, the recombinant bacterium comprises an oxlT gene under the control of an inducible promoter, optionally an FNR promoter; an scaaE3 gene, an oxcd gene, and an frc gene under the control of an inducible promoter, optionally an FRN promoter, a thyA deletion (or auxotrophy) and a deletion of endogenous phage 3. In one embodiment, the recombinant bacterium comprises HA910::FNR_oxlT, HA12::FNR_scaaE3-oxcd-frc, ΔthyA, Δphage 3.

In one embodiment, the recombinant bacterium is SYN5752, SYN7169, or SYNB8802. In one embodiment, the recombinant bacterium is SYNB8802.

In one embodiment, the subject has hyperoxaluria. In one embodiment, the hyperoxaluria is primary hyperoxaluria, dietary hyperoxaluria, or enteric hyperoxaluria. In one embodiment, the subject has short bowel syndrome, chronic pancreatitis, inflammatory bowel disease (IBD), cystic fibrosis, kidney disease, and/or Roux-en-Y gastric bypass.

In one embodiment, the subject has urinary oxalate (Uox) levels of at least 70 mg/day prior to the administering. In one embodiment, the subject exhibits a decrease in Uox levels of at least 10%, 15%, 20%, 25%, 30%, 35%, 40%, 45%, 50%, 55%, 60%, 65%, 70%, 75%, or 80% after the administering. In one embodiment, the subject has eGFR<30 mL/min/1.73 m², requires hemodialysis, or has systemic oxalosis prior to the administering.

In one embodiment, the recombinant bacteria is administered at a dose of about 1×10¹¹ live recombinant bacteria, about 2×10¹¹ live recombinant bacteria, about 3×10¹¹ live recombinant bacteria, about 4×10¹¹ live recombinant bacteria, about 5×10¹¹ live recombinant bacteria, about 6×10¹¹ live recombinant bacteria, about 1×10¹² live recombinant bacteria, or about 2×10¹² live recombinant bacteria. In one embodiment, the recombinant bacteria is administered at a dose of about 6×10¹¹ live recombinant bacteria. In one embodiment, the recombinant bacteria is administered at a dose of about 3×10¹¹ live recombinant bacteria. In one embodiment, the recombinant bacteria is administered at a dose of about 1×10¹¹ live recombinant bacteria. In one embodiment, the administering is about 5×10¹¹ live recombinant bacteria. In one embodiment, the recombinant bacteria is administered at a dose of about 1×10¹² live recombinant bacteria. In one embodiment, the recombinant bacteria is administered at a dose of about 2×10¹² live recombinant bacteria. In one embodiment, the administering is about 5×10¹¹ live recombinant bacteria with meals three times per day. In one embodiment, the recombinant bacteria is administered at a dose of about 1×10¹¹ live recombinant bacteria to about 2×10¹² live recombinant bacteria. In one embodiment, the recombinant bacteria is administered at a dose of about 1×10¹² live recombinant bacteria to about 2×10¹² live recombinant bacteria. In one embodiment, the recombinant bacteria is administered at a dose of about 5×10¹¹ live recombinant bacteria to about 2×10¹² live recombinant bacteria.

In another embodiment, the administering is oral, with meals, twice per day. In another embodiment, the administering is oral, with meals, three times per day.

In another embodiment, a proton pump inhibitor (PPI) is administered to the subject. In another embodiment, the the PPI is esomeprazole. In another embodiment, the administering of the PPI is once a day.

In some embodiments, the disclosure provides a bacterial cell that has been genetically engineered to comprise one or more genes, gene cassettes, and/or synthetic circuits encoding one or more oxalate catabolism enzyme(s) or oxalate catabolism pathway, and is capable of metabolizing oxalate and/or other metabolites, such as oxalyl-CoA. Thus, the genetically engineered bacterial cells and pharmaceutical compositions comprising the bacterial cells may be used to treat and/or prevent diseases associated with disorders in which oxalate is detrimental, such as primary hyperoxalurias and secondary hyperoxalurias.

In some embodiments, the disclosure provides a bacterial cell that has been engineered to comprise gene sequence(s) encoding one or more oxalate catabolism enzyme(s). In some embodiments, the disclosure provides a bacterial cell has been engineered to comprise gene sequence(s) encoding one or more oxalate catabolism enzyme(s) and is capable of reducing the level of oxalate and/or other metabolites, for example, oxalyl-CoA. In some embodiments, the bacterial cell has been engineered to comprise gene sequence(s) encoding one or more transporter(s) (importer(s)) of oxalate. In some embodiments, the bacterial cell has been engineered to comprise gene sequence(s) encoding one or more exporter(s) of formate. In some embodiments, the engineered bacteria comprise gene sequence(s) encoding one or more polypeptide(s) which mediate both the transport (import) of oxalate and the export of formate (e.g., oxalate:formate antiporter(s)). In some embodiments, the engineered bacteria comprise gene sequence(s) encoding one or more of the following: (i) one or more transporter(s) of oxalate; (ii) one or more exporter(s) of formate; (iii) one or more polypeptide(s) which mediate both the transport (import) of oxalate and the export of formate (e.g., oxalate:formate antiporter(s)); and (iv) any combination thereof. In some embodiments, the bacterial cell has been engineered to comprise gene sequence(s) encoding one or more oxalate catabolism enzyme(s) and one or more transporter(s) (importer(s)) of oxalate. In some embodiments, the bacterial cell of the disclosure has been genetically engineered to comprise gene sequence(s) encoding one or more oxalate catabolism enzyme(s) and one or more exporter(s) of formate. In some embodiments, genetically engineered bacteria comprise gene sequence(s) encoding one or more oxalate catabolism enzyme(s) and one or more polypeptide(s), which mediate both the transport (import) of oxalate and the export of formate (e.g., oxalate:formate antiporter(s)). In some embodiments, the bacterial cell has been engineered to comprise gene sequence(s) encoding one or more oxalate catabolism enzyme(s) and gene sequence(s) encoding one or more of the following: (i) one or more transporter(s) of oxalate; (ii) one or more exporter(s) of formate; (iii) one or more polypeptide(s) which mediate both the transport (import) of oxalate and the export of formate (e.g., oxalate:formate antiporter(s)); and (iv) any combination thereof.

In some embodiments, the gene sequence(s) encoding one or more oxalate catabolism enzyme(s) is operably linked to an inducible promoter. In some embodiments, the gene sequence(s) encoding one or more oxalate transporter(s) (importer(s)) is operably linked to an inducible promoter. In some embodiments, the gene sequence(s) encoding one or more exporter(s) of formate is operably linked to an inducible promoter. In some embodiments, the gene sequence(s) encoding one or more polypeptide(s) which mediate both the transport (import) of oxalate and the export of formate (e.g., oxalate:formate antiporter(s)) is operably linked to an inducible promoter. In some embodiments, the gene sequence(s) encoding one or more oxalate catabolism enzyme(s) and the gene sequence(s) encoding one or more oxalate transporter(s) (importer(s)) are operably linked to an inducible promoter. In some embodiments, the gene sequence(s) encoding one or more oxalate catabolism enzyme(s) and the gene sequence(s) encoding one or more exporter(s) of formate are operably linked to an inducible promoter. In some embodiments, the gene sequence(s) encoding one or more oxalate catabolism enzyme(s) and the gene sequence(s) encoding one or more polypeptide(s) which mediate both the transport (import) of oxalate and the export of formate (e.g., oxalate:formate antiporter(s)) are operably linked to an inducible promoter. In some embodiments, any one or more of the following gene sequences, if present in the bacterial cell, are operably linked to an inducible promoter: (i) gene sequence(s) encoding one or more oxalate catabolism enzyme(s); (ii) gene sequence(s) encoding one or more oxalate transporter(s); (iii) gene sequence(s) encoding one or more exporter(s) of formate; and (iv) gene sequence(s) encoding one or more polypeptide(s) which mediate both the transport (import) of oxalate and the export of formate (e.g., oxalate:formate antiporter(s)).

In some embodiments, the disclosure provides a bacterial cell which has been engineered to comprise gene sequence(s) encoding one or more oxalate catabolism enzyme(s) operably linked to an inducible promoter that is induced under low oxygen and/or anaerobic conditions, e.g., such as those conditions found in the mammalian gut. In some embodiments, the disclosure provides a bacterial cell which has been engineered to comprise gene sequence(s) encoding one or more oxalate transporter(s) (importer(s)) operably linked to an inducible promoter that is induced under low oxygen and/or anaerobic conditions, e.g., such as those conditions found in the mammalian gut. In some embodiments, the disclosure provides a bacterial cell which has been engineered to comprise gene sequence(s) encoding one or more exporter(s) of formate operably linked to an inducible promoter that is induced under low oxygen and/or anaerobic conditions, e.g., such as those conditions found in the mammalian gut. In some embodiments, the disclosure provides a bacterial cell which has been engineered to comprise gene sequence(s) encoding one or more polypeptide(s) which mediate both the transport (import) of oxalate and the export of formate (e.g., oxalate:formate antiporter(s)) operably linked to an inducible promoter that is induced under low oxygen and/or anaerobic conditions, e.g., such as those conditions found in the mammalian gut. In some embodiments, the gene sequence(s) encoding one or more oxalate catabolism enzyme(s) and the gene sequence(s) encoding one or more oxalate transporter(s) (importer(s)) are operably linked to an inducible promoter that is induced under low oxygen and/or anaerobic conditions. In some embodiments, the gene sequence(s) encoding one or more oxalate catabolism enzyme(s) and the gene sequence(s) encoding one or more exporter(s) of formate are operably linked to an inducible promoter that is induced under low oxygen and/or anaerobic conditions. In some embodiments, the gene sequence(s) encoding one or more oxalate catabolism enzyme(s) and the gene sequence(s) encoding one or more polypeptide(s) which mediate both the transport (import) of oxalate and the export of formate (e.g., oxalate:formate antiporter(s)) are operably linked to an inducible promoter that is induced under low oxygen and/or anaerobic conditions. In some embodiments, any one or more of the following gene sequences, if present in the bacterial cell, are operably linked to an inducible promoter that is induced under low oxygen and/or anaerobic conditions: (i) gene sequence(s) encoding one or more oxalate catabolism enzyme(s); (ii) gene sequence(s) encoding one or more oxalate transporter(s); (iii) gene sequence(s) encoding one or more exporter(s) of formate; and (iv) gene sequence(s) encoding one or more polypeptide(s) which mediate both the transport (import) of oxalate and the export of formate (e.g., oxalate:formate antiporter(s)).

In one embodiment, the inducible promoter is a lad promoter which can be induced with IPTG. In another embodiment, the inducible promoter is a pBAD promoter which can be induced with arabinose.

In some embodiments, the disclosure provides a bacterial cell that has been engineered to comprise gene sequence(s) encoding one or more oxalate catabolism enzyme(s) that is operably linked to an inducible promoter that is induced by environmental signals and/or conditions found in the mammalian gut (e.g., induced by metabolites (e.g., oxalate metabolites) or other biomolecules found in the mammalian gut, and/or induced by inflammatory conditions (e.g., reactive nitrogen species and/or reactive oxygen species)). The environmental signals and/or conditions found in the mammalian gut may be signals and conditions found in a healthy mammalian gut or signals and conditions found in a diseased mammalian gut, such as the gut of a subject having hyperoxaluria or other condition in which the level of oxalate and/or an oxalate metabolite is elevated, and/or the gut of a subject having an inflammatory condition, such as irritable bowel disease, an autoimmune disease, and any other condition that results in inflammation in the gut. In some embodiments, the disclosure provides a bacterial cell which has been engineered to comprise gene sequence(s) encoding one or more oxalate catabolism enzyme(s) operably linked to an inducible promoter that is induced under inflammatory conditions, e.g., such as inflammatory conditions found in a mammalian gut. In some embodiments, the disclosure provides a bacterial cell which has been engineered to comprise gene sequence(s) encoding one or more oxalate transporter(s) (importer(s)) operably linked to an inducible promoter that is induced under inflammatory conditions, e.g., such as inflammatory conditions found in a mammalian gut. In some embodiments, the disclosure provides a bacterial cell which has been engineered to comprise gene sequence(s) encoding one or more exporter(s) of formate operably linked to an inducible promoter that is induced under inflammatory conditions, e.g., such as inflammatory conditions found in a mammalian gut. In some embodiments, the disclosure provides a bacterial cell which has been engineered to comprise gene sequence(s) encoding one or more polypeptide(s) which mediate both the transport (import) of oxalate and the export of formate (e.g., oxalate:formate antiporter(s)) operably linked to an inducible promoter that is induced under inflammatory conditions, e.g., such as inflammatory conditions found in a mammalian gut. In some embodiments, the gene sequence(s) encoding one or more oxalate catabolism enzyme(s) and the gene sequence(s) encoding one or more oxalate transporter(s) (importer(s)) are operably linked to an inducible promoter that is induced under inflammatory conditions. In some embodiments, the gene sequence(s) encoding one or more oxalate catabolism enzyme(s) and the gene sequence(s) encoding one or more exporter(s) of formate are operably linked to an inducible promoter that is induced under inflammatory conditions. In some embodiments, the gene sequence(s) encoding one or more oxalate catabolism enzyme(s) and the gene sequence(s) encoding one or more polypeptide(s) which mediate both the transport (import) of oxalate and the export of formate (e.g., oxalate:formate antiporter(s)) are operably linked to an inducible promoter that is induced under inflammatory conditions. In some embodiments, any one or more of the following gene sequences, if present in the bacterial cell, are operably linked to an inducible promoter that is induced under inflammatory conditions: (i) gene sequence(s) encoding one or more oxalate catabolism enzyme(s); (ii) gene sequence(s) encoding one or more oxalate transporter(s); (iii) gene sequence(s) encoding one or more exporter(s) of formate; and (iv) gene sequence(s) encoding one or more polypeptide(s) which mediate both the transport (import) of oxalate and the export of formate (e.g., oxalate:formate antiporter(s)).

In some embodiments, the disclosure provides a bacterial cell that has been engineered to comprise gene sequence(s) encoding one or more polypeptide(s) capable of reducing the level of oxalate and/or other metabolites, for example, oxalyl-CoA, in low-oxygen environments, e.g., the gut. In some embodiments, the bacterial cell that has been engineered to comprise gene sequence(s) encoding one or more of the following: (i) one or more oxalate catabolism enzyme(s); (ii) one or more oxalate transporter(s); (ii) one or more formate exporter(s); and (iv) one or more oxalate:formate antiporter(s). In some embodiments, the bacterial cell has been genetically engineered to comprise one or more circuits encoding one or more oxalate catabolism enzyme(s) and is capable of processing and reducing levels of oxalate, and/or oxalyl-CoA e.g., in low-oxygen environments, e.g., the gut. Thus, in some embodiments, the genetically engineered bacterial cells and pharmaceutical compositions comprising the bacterial cells of the disclosure may be used to import excess oxalate and/or oxalyl-CoA into the bacterial cell in order to treat and/or prevent conditions associated with disorders in which oxalate is detrimental, such as primary hyperoxalurias and secondary hyperoxalurias. In some embodiments, the genetically engineered bacterial cells and pharmaceutical compositions comprising the bacterial cells of the disclosure may be used to convert excess oxalate and/or oxalyl-CoA into non-toxic molecules in order to treat and/or prevent conditions associated with disorders in which oxalate is detrimental, such as primary hyperoxalurias and secondary hyperoxalurias.

The present invention provides recombinant bacterial cells, pharmaceutical compositions thereof, and methods of modulating and treating disorders in which oxalate is detrimental. The genetically engineered bacterial cells and pharmaceutical compositions comprising the bacterial cells of the invention may be used to convert excess oxalate and/or oxalic acid into non-toxic molecules in order to treat and/or prevent conditions associated with disorders in which oxalate is detrimental, such as primary hyperoxalurias and secondary hyperoxalurias. In some embodiments, a bacterial cell of the invention has been engineered to comprise at least one heterologous gene encoding at least one oxalate catabolism enzyme and is capable of processing and reducing levels of oxalate, in low-oxygen environments, e.g., the gut. In some embodiments, a bacterial cell of the invention has been engineered to comprise at least one heterologous gene encoding an importer of oxalate and is capable of reducing levels of oxalate, in low-oxygen environments, e.g., the gut. In some embodiments, a bacterial cell of the invention has been engineered to comprise at least one heterologous gene encoding an exporter of formate and is capable of reducing levels of oxalate, in low-oxygen environments, e.g., the gut. In some embodiments, a bacterial cell of the invention has been engineered to comprise at least one heterologous gene encoding an oxalate:formate antiporter and is capable of reducing levels of oxalate, in low-oxygen environments, e.g., the gut. In some embodiments, a bacterial cell of the invention has been engineered to comprise at least one heterologous gene encoding at least one oxalate catabolism enzyme and is capable of processing and reducing levels of oxalate, in inflammatory environments, such as may be present in the gut. In some embodiments, a bacterial cell of the invention has been engineered to comprise at least one heterologous gene encoding an importer of oxalate and is capable of reducing levels of oxalate, in inflammatory environments, e.g., such as may be present in the gut. In some embodiments, a bacterial cell of the invention has been engineered to comprise at least one heterologous gene encoding an exporter of formate and is capable of reducing levels of oxalate, in inflammatory environments, e.g., such as may be present in the gut. In some embodiments, a bacterial cell of the invention has been engineered to comprise at least one heterologous gene encoding an oxalate:formate antiporter and is capable of reducing levels of oxalate, in inflammatory environments, e.g., such as may be present in the gut.

In some embodiments, the at least one oxalate catabolism enzyme converts oxalate to formate or formyl CoA. In some embodiments, the at least one oxalate catabolism enzyme is selected from an oxalate-CoA ligase, (e.g., ScAAE3 from S. cerevisiae), an oxalyl-CoA decarboxylase (Oxc, e.g., from O. formigenes), and a formyl-CoA transferase (e.g., Frc, e.g., from O. formigenes). In some embodiments, the at least one heterologous gene encoding at least one oxalate catabolism enzyme is selected from afrc gene and an oxc gene. In one embodiment, the at least one heterologous gene encoding an oxalate transporter is an oxlT gene. In some embodiments, the at least one heterologous gene encoding at least one oxalate catabolism enzyme is located on a plasmid in the bacterial cell. In some embodiments, the at least one heterologous gene encoding at least one oxalate catabolism enzyme is located on a chromosome in the bacterial cell. In some embodiments, the at least one heterologous gene encoding an oxalate transporter is located on a plasmid in the bacterial cell. In some embodiments, the at least one heterologous gene encoding the oxalate transporter is located on a chromosome in the bacterial cell. In some embodiments, the at least one heterologous gene encoding a formate exporter is located on a plasmid in the bacterial cell. In some embodiments, the at least one heterologous gene encoding a formate exporter is located on a chromosome in the bacterial cell. In some embodiments, the at least one heterologous gene encoding an oxalate:formate antiporter is located on a plasmid in the bacterial cell. In some embodiments, the at least one heterologous gene encoding an oxalate:formate antiporter is located on a chromosome in the bacterial cell.

In some embodiments, the engineered bacterial cell is a probiotic bacterial cell. In some embodiments, the engineered bacterial cell is a member of a genus selected from the group consisting of Bacteroides, Bifidobacterium, Clostridium, Escherichia, Lactobacillus and Lactococcus. In some embodiments, the engineered bacterial cell is of the genus Escherichia. In some embodiments, the recombinant bacterial cell is of the species Escherichia coli strain Nissle.

In some embodiments, the engineered bacterial cell is an auxotroph in a gene that is complemented when the engineered bacterial cell is present in a mammalian gut. In some embodiments, the mammalian gut is a human gut. In some embodiments, the engineered bacterial cell is an auxotroph in diaminopimelic acid or an enzyme in the thymine biosynthetic pathway. In some embodiments, the engineered bacterial cell further comprises a heterologous gene encoding a substance that is toxic to the bacterial cell that is operably linked to an inducible promoter, wherein the inducible promoter is directly or indirectly induced by an environmental condition not naturally present in the mammalian gut.

In another aspect, the invention provides a pharmaceutical composition comprising a recombinant bacterial cell comprising at least one heterologous gene encoding at least one oxalate catabolism enzyme operably linked to a first inducible promoter and a pharmaceutically acceptable carrier. In another aspect, the invention provides a pharmaceutical composition comprising a recombinant bacterial cell comprising at least one heterologous gene encoding at least one oxalate catabolism enzyme operably linked to a first inducible promoter, at least one heterologous gene encoding an oxalate transporter operably linked to a second inducible promoter, which may be the same or different promoter from the first inducible promoter, and a pharmaceutically acceptable carrier. In another aspect, the invention provides a pharmaceutical composition comprising a recombinant bacterial cell comprising at least one heterologous gene encoding at least one oxalate catabolism enzyme operably linked to a first inducible promoter, at least one heterologous gene encoding a formate exporter operably linked to a second inducible promoter, which may be the same or different promoter from the first inducible promoter, and a pharmaceutically acceptable carrier. In another aspect, the invention provides a pharmaceutical composition comprising a recombinant bacterial cell comprising at least one heterologous gene encoding at least one oxalate catabolism enzyme operably linked to a first inducible promoter, at least one heterologous gene encoding an oxalate:formate antiporter operably linked to a second inducible promoter, which may be the same or different promoter from the first inducible promoter, and a pharmaceutically acceptable carrier. In any of these embodiments, the first promoter and the second promoter may be separate copies of the same promoter. In some embodiments, the first inducible promoter, the second inducible promoter, or the first inducible promoter and the second inducible promoter, are each directly induced by environmental conditions. In some embodiments, the first inducible promoter, the second inducible promoter, or the first inducible promoter and the second inducible promoter, are each indirectly induced by environmental conditions. In some embodiments, the first inducible promoter, the second inducible promoter, or the first inducible promoter and the second inducible promoter, are each directly or indirectly induced by environmental conditions found in the gut of a mammal. In some embodiments, the first inducible promoter, the second inducible promoter, or the first inducible promoter and the second inducible promoter, are each directly or indirectly induced by low-oxygen or anaerobic conditions. In some embodiments, the first inducible promoter, the second inducible promoter, or the first inducible promoter and the second inducible promoter, are each directly or indirectly induced by inflammatory conditions. In some embodiments, the first inducible promoter, the second inducible promoter, or the first inducible promoter and the second inducible promoter, are each an FNR responsive promoter. In some embodiments, the first inducible promoter, the second inducible promoter, or the first inducible promoter and the second inducible promoter, are each an RNSresponsive promoter. In some embodiments, the first inducible promoter, the second inducible promoter, or the first inducible promoter and the second inducible promoter, are each an ROS responsive promoter. In another aspect, the invention provides a method for treating a disease or disorder in which oxalate is detrimental in a subject, the method comprising administering a an engineered bacterial cell or a pharmaceutical composition comprising an engineered bacterial cell to the subject, wherein the engineered bacterial cell comprises gene sequence encoding one or more oxalate catabolism enzyme(s). In another aspect, the invention provides a method for treating a disease or disorder in which oxalate is detrimental in a subject, the method comprising administering an engineered bacterial cell or a pharmaceutical composition comprising an engineered bacterial cell to the subject, wherein the engineered bacterial cell comprises gene sequence encoding one or more oxalate transporter(s). In another aspect, the invention provides a method for treating a disease or disorder in which oxalate is detrimental in a subject, the method comprising administering an engineered bacterial cell or a pharmaceutical composition comprising an engineered bacterial cell to the subject, wherein the engineered bacterial cell comprises gene sequence encoding one or more formate exporter(s). In another aspect, the invention provides a method for treating a disease or disorder in which oxalate is detrimental in a subject, the method comprising administering an engineered bacterial cell or a pharmaceutical composition comprising an engineered bacterial cell to the subject, wherein the engineered bacterial cell comprises gene sequence encoding one or more oxalate:formate antiporter(s). In another aspect, the invention provides a method for treating a disease or disorder in which oxalate is detrimental in a subject, the method comprising administering an engineered bacterial cell or a pharmaceutical composition comprising an engineered bacterial cell to the subject, wherein the engineered bacterial cell comprises gene sequence encoding one or more of the following: (i) oxalate catabolism enzyme(s); (ii) one or more oxalate transporter(s); (iii) one or more formate exporter(s); and (iv) one or more oxalate:formate antiporter(s).

In another aspect, the invention provides a method for treating a disease or disorder in which oxalate is detrimental in a subject, the method comprising administering an engineered bacterial cell or a pharmaceutical composition comprising an engineered bacterial cell to the subject, wherein the engineered bacterial cell expresses at least one heterologous gene encoding at least one oxalate catabolism enzyme in response to an exogenous environmental condition in the subject, thereby treating the disease or disorder in which oxalate is detrimental in the subject. In some embodiments, the engineered bacterial cell further expresses one or more of the following: (i) at least one heterologous gene encoding an importer of oxalate; (ii) at least one heterologous gene encoding an exporter of formate; and/or (iii) at least one heterologous gene encoding an oxalate:formate antiporter. In one aspect, the invention provides a method for treating a disorder in which oxalate is detrimental in a subject, the method comprising administering an engineered bacterial cell or a pharmaceutical composition of the invention to the subject, thereby treating the disorder in which oxalate is detrimental in the subject. In another aspect, the invention provides a method for decreasing a level of oxalate in plasma of a subject, the method comprising administering an engineered bacterial cell or a pharmaceutical composition of the invention to the subject, thereby decreasing the level of oxalate in the plasma of the subject. In another aspect, the invention provides a method for decreasing a level of oxalate in urine of a subject, the method comprising administering an engineered bacterial cell or a pharmaceutical composition of the invention to the subject, thereby decreasing the level of oxalate in the urine of the subject. In one embodiment, the level of oxalate is decreased in plasma of the subject after administering the engineered bacterial cell or pharmaceutical composition to the subject. In another embodiment, the level of oxalate is reduced in urine of the subject after administering the engineered bacterial cell or pharmaceutical composition to the subject. In one embodiment, the engineered bacterial cell or pharmaceutical composition is administered orally. In another embodiment, the method further comprises isolating a plasma sample from the subject or a urine sample from the subject after administering the engineered bacterial cell or pharmaceutical composition to the subject, and determining the level of oxalate in the plasma sample from the subject or the urine sample from the subject. In another embodiment, the method further comprises comparing the level of oxalate in the plasma sample from the subject or the urine sample from the subject to a control level of oxalate. In one embodiment, the control level of oxalate is the level of oxalate in the plasma of the subject or in the urine of the subject before administration of the engineered bacterial cell or pharmaceutical composition.

In one embodiment, the disorder in which oxalate is detrimental is a hyperoxaluria. In one embodiment, the hyperoxaluria is primary hyperoxaluria type I. In another embodiment, the hyperoxaluria is primary hyperoxaluria type II. In another embodiment, the hyperoxaluria is primary hyperoxaluria type III. In one embodiment, the hyperoxaluria is enteric hyperoxaluria. In another embodiment, the hyperoxaluria is dietary hyperoxaluria. In another embodiment, the hyperoxaluria is idiopathic hyperoxaluria.

In one embodiment, the subject is fed a meal within one hour of administering the pharmaceutical composition. In another embodiment, the subject is fed a meal concurrently with administering the pharmaceutical composition.

### BRIEF DESCRIPTION OF THE DRAWINGS

FIG. 1 depicts a graph showing the results of an in vitro oxalate degradation assay using an engineered E. coli. Nissle strain as compared to a wild type E. coli Nissle strain.

FIG. 2A depicts a bar graph showing the results of an in vivo oxalate consumption experiment by measuring acute ¹³C-oxalate urinary recovery when using an engineered E. coli Nissle strain (Engineered EcN) as compared to a wild type E. coli Nissle strain (EcN). FIG. 2B depicts a bar graph showing the results of an in vivo oxalate consumption experiment by measuring chronic urinary oxalate recovery when using an engineered E. coli Nissle strain (Engineered EcN) as compared to a wild type E. coli Nissle strain (EcN).

FIG. 3 is a figure summarizing the disease pathogenesis of enteric hyperoxaluria.

FIG. 4A depicts the components of strain SYNB8802, and FIG. 4B depicts a graph showing the results of an in vitro oxalate degradation assay using SYNB8802 as compared to a wild type E. coli Nissle strain. FIG. 4C depicts as graph showing the results of an in vitro oxalate degradation and formate production assay using SYNB8802 as compared to a wild type E. coli Nissle strain.

FIG. 5A depicts oxalate consumption with SYN-HOX (SYN5752) when SYN-HOX was activated in simulated stomach and colon fluid. FIG. 5B depicts that an engineered E. coli Nissle strain (Engineered EcN), SYN5752 consumed oxalate in mice in the gut. SYN5752 is an integrated strain with antibiotic resistance. SYN7169 is an integrated strain with antibiotic resistance, auxotrophy, and phage 3 deletion. ¹³C-oxalate consumption was measured in multiple acute mouse studies, and the efficacy of the strain ranged between 50-75%. SYN7169 behaved similarly to SYN5752 in this mouse model. FIG. 5C depicts oxalate consumption with SYNB8802 in the gastrointestinal (GI) tract of healthy mice. Data presented as mean urinary ¹³C-oxalate recovery normalized by creatinine±standard error of the mean. Statistical analysis was performed using one-way analysis of variance followed by Dunnett's multiple comparison test. ****p<0.0001.

FIG. 6 depicts an attenuation of urinary oxalate increase in healthy monkeys.

FIG. 7A depicts a bar graph showing dose-dependent recovery of urinary oxalate in healthy monkeys (NHP) after treatment with SYN7169. FIG. 7B depicts a bar graph showing dose-dependent recovery of urinary ¹³C-oxalate in healthy monkeys after treatment with SYN7169. FIG. 7C depicts oxalate and ¹³C-oxalate consumption in the GI tract of cynomolgus monkeys with acute hyperoxaluria. Data presented as mean urinary oxalate or ¹³C-oxalate recovery normalized by creatinine±standard error of the mean. Statistical analysis was performed using paired t-test. **p<0.01.

FIG. 8A depicts that SYNB8802 is viable in vivo and cleared from feces of mice by 24 hours. FIG. 8B depicts recovered SYN-HOX (SYN8802) from feces of cynomolgus monkeys over 6 and 24 hours.

FIG. 9 depicts oxalate consumption with SYNB8802 lyophilized (Lyo) and frozen liquid (FL) in non-human primates (NHP).

FIG. 10 depicts oxalate consumption in mice with SYN-HOX (SYN7169) lyophilized (Lyo) and frozen liquid based on CFU and live cell.

FIG. 11A depicts a graph modeling dose-dependent recovery of urinary oxalate in human patients after treatment with SYNB8802. FIG. 11B depicts a schematic of enteric hyperoxaluria in silico simulation (ISS) model. FIG. 11C depicts in silico simulation (ISS), urinary oxalate percent change from baseline after dosing with SYNB8802. This modeling suggests that SYNB8802 has the potential to achieve >20% urinary oxalate lowering at target dose range.

FIG. 12 is a schematic summarizing the organization of the clinical trial.

### DETAILED DESCRIPTION

The disclosure includes engineered and programmed microorganisms, e.g., bacteria, yeast, viruses etc., pharmaceutical compositions thereof, and methods of modulating and treating disorders in which oxalate is detrimental. In some embodiments, the microorganism, e.g., bacterium, yeast, or virus, has been genetically engineered to comprise heterologous gene sequence(s) encoding one or more oxalate catabolism enzyme(s). In some embodiments, the microorganism, e.g., bacterium, yeast, or virus, has been genetically engineered to comprise heterologous gene sequence(s) encoding one or more oxalate catabolism enzyme(s) and is capable of processing and reducing oxalate and/or oxalic acid in low-oxygen environments, e.g., the gut. In some embodiments, the engineered microorganism comprises heterologous gene sequence(s) encoding one or more oxalate catabolism enzyme(s) and is capable of transporting oxalic acid and/or oxalate and/or another related metabolite(s) into the bacterium. Thus, the recombinant microorganism and pharmaceutical compositions comprising the microorganism of the invention may be used to catabolize oxalate or oxalic acid to treat and/or prevent conditions associated with disorders in which oxalate is detrimental. In one embodiment, the disorder in which oxalate is detrimental is a disorder involving the abnormal levels of oxalate, such as primary hyperoxalurias (i.e., PHI, PHII, and PHIII), secondary hyperoxaluria, enteric hyperoxaluria, dietary hyperoxaluria, or idiopathic hyperoxaluria.

In some embodiments, the engineered microorganism comprise gene sequence(s) encoding one or more of the following: (i) one or more transporter(s) of oxalate; (ii) one or more exporter(s) of formate; (iii) one or more polypeptide(s) which mediate both the transport (import) of oxalate and the export of formate (e.g., oxalate:formate antiporter(s)); and (iv) any combination thereof. In some embodiments, the microorganism has been engineered to comprise gene sequence(s) encoding one or more oxalate catabolism enzyme(s) and one or more of the following: (i) one or more transporter(s) of oxalate; (ii) one or more exporter(s) of formate; (iii) one or more polypeptide(s) which mediate both the transport (import) of oxalate and the export of formate (e.g., oxalate:formate antiporter(s)); and (iv) any combination thereof.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “microorganism” or “recombinant microorganism” refers to a microorganism, e.g., bacterial or viral cell, or bacteria or virus, that has been genetically modified from its native state. Thus, a “recombinant bacterial cell” or “recombinant bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells disclosed herein may comprise exogenous nucleotide sequences on plasmids. Alternatively, recombinant bacterial cells may comprise exogenous nucleotide sequences stably incorporated into their chromosome.

A “programmed or engineered microorganism” refers to a microorganism, e.g., bacterial or viral cell, or bacteria or virus, that has been genetically modified from its native state to perform a specific function. Thus, a “programmed or engineered bacterial cell” or “programmed or engineered bacteria” or “genetically engineered bacterial cell or bacteria” refers to a bacterial cell or bacteria that has been genetically modified from its native state to perform a specific function, e.g., to metabolize a metabolite, e.g., oxalate. In certain embodiments, the programmed or engineered bacterial cell has been modified to express one or more proteins, for example, one or more proteins that have a therapeutic activity or serve a therapeutic purpose. The programmed or engineered bacterial cell may additionally have the ability to stop growing or to destroy itself once the protein(s) of interest have been expressed.

As used herein, the term “gene” refers to a nucleic acid fragment that encodes a protein or fragment thereof, optionally including regulatory sequences preceding (5′ non-coding sequences) and following (3′ non-coding sequences) the coding sequence. In one embodiment, a “gene” does not include regulatory sequences preceding and following the coding sequence. A “native gene” refers to a gene as found in nature, optionally with its own regulatory sequences preceding and following the coding sequence. A “chimeric gene” refers to any gene that is not a native gene, optionally comprising regulatory sequences preceding and following the coding sequence, wherein the coding sequences and/or the regulatory sequences, in whole or in part, are not found together in nature. Thus, a chimeric gene may comprise regulatory sequences and coding sequences that are derived from different sources, or regulatory and coding sequences that are derived from the same source, but arranged differently than is found in nature.

As used herein, the term “gene sequence” is meant to refer to a genetic sequence, e.g., a nucleic acid sequence. The gene sequence or genetic sequence is meant to include a complete gene sequence or a partial gene sequence. The gene sequence or genetic sequence is meant to include sequence that encodes a protein or polypeptide and is also meant to include genetic sequence that does not encode a protein or polypeptide, e.g., a regulatory sequence, leader sequence, signal sequence, or other non-protein coding sequence.

As used herein, a “heterologous” gene or “heterologous sequence” refers to a nucleotide sequence that is not normally found in a given cell in nature. As used herein, a heterologous sequence encompasses a nucleic acid sequence that is exogenously introduced into a given cell and can be a native sequence (naturally found or expressed in the cell) or non-native sequence (not naturally found or expressed in the cell) and can be a natural or wild-type sequence or a variant, non-natural, or synthetic sequence. “Heterologous gene” includes a native gene, or fragment thereof, that has been introduced into the host cell in a form that is different from the corresponding native gene. For example, a heterologous gene may include a native coding sequence that is a portion of a chimeric gene to include non-native regulatory regions that is reintroduced into the host cell. A heterologous gene may also include a native gene, or fragment thereof, introduced into a non-native host cell. Thus, a heterologous gene may be foreign or native to the recipient cell; a nucleic acid sequence that is naturally found in a given cell but expresses an unnatural amount of the nucleic acid and/or the polypeptide which it encodes; and/or two or more nucleic acid sequences that are not found in the same relationship to each other in nature. As used herein, the term “endogenous gene” refers to a native gene in its natural location in the genome of an organism. As used herein, the term “transgene” refers to a gene that has been introduced into the host organism, e.g., host bacterial cell, genome.

As used herein, a “non-native” nucleic acid sequence refers to a nucleic acid sequence not normally present in a microorganism, e.g., an extra copy of an endogenous sequence, or a heterologous sequence such as a sequence from a different species, strain, or substrain of bacteria or virus, or a sequence that is modified and/or mutated as compared to the unmodified sequence from bacteria or virus of the same subtype. In some embodiments, the non-native nucleic acid sequence is a synthetic, non-naturally occurring sequence (see, e.g., Purcell et al., 2013). The non-native nucleic acid sequence may be a regulatory region, a promoter, a gene, and/or one or more genes in gene cassette. In some embodiments, “non-native” refers to two or more nucleic acid sequences that are not found in the same relationship to each other in nature. The non-native nucleic acid sequence may be present on a plasmid or chromosome. In some embodiments, the genetically engineered microorganism of the disclosure comprises a gene and/or gene cassette that is operably linked to a promoter that is not associated with said gene in nature. For example, in some embodiments, the genetically engineered bacteria disclosed herein comprise a gene or gene cassette encoding one or more oxalate-metabolizing enzyme(s) described herein and/or one or more oxalate transporter(s), one or more exporter(s) (e.g., of formate) and/or one or more antiporter(s)(e.g., oxalate:formate antiporter(s)) that is operably linked to a directly or indirectly inducible promoter that is not associated with said gene in nature, e.g., an FNR responsive one or more oxalate-metabolizing enzyme(s) described herein and/or one or more oxalate transporter(s), one or more exporter(s) (e.g., of formate) and/or one or more antiporter(s)(e.g., oxalate:formate antiporter(s)) (or other promoter disclosed herein) operably linked to a gene encoding a one or more oxalate-metabolizing enzyme(s) described herein and/or one or more oxalate transporter(s), one or more exporter(s) (e.g., of formate) and/or one or more antiporter(s)(e.g., oxalate:formate antiporter(s)). In some embodiments, the genetically engineered virus of the disclosure comprises a gene or gene cassette that is operably linked to a directly or indirectly inducible promoter that is not associated with said gene or gene cassette in nature, e.g., a promoter operably linked to a gene and/or gene cassette encoding one or more oxalate-metabolizing enzyme(s) and/or one or more oxalate transporter(s) and/or one or more exporter(s) (e.g., of formate) and/or one or more antiporter(s)(e.g., oxalate:formate antiporter(s)).

As used herein, the term “coding region” refers to a nucleotide sequence that codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing, RNA stability, or translation of the associated coding sequence. Examples of regulatory sequences include, but are not limited to, promoters, translation leader sequences, effector binding sites, signal sequences, and stem-loop structures. In one embodiment, the regulatory sequence comprises a promoter, e.g., an FNR responsive promoter or other promoter disclosed herein.

“Operably linked” refers to the association of nucleic acid sequences on a single nucleic acid fragment so that the function of one is affected by the other. A regulatory element is operably linked with a coding sequence when it is capable of affecting the expression of the gene coding sequence, regardless of the distance between the regulatory element and the coding sequence. More specifically, operably linked refers to a nucleic acid sequence, e.g., a gene or gene cassette encoding one or more an oxalate catabolism enzyme, that is joined to a regulatory sequence in a manner which allows expression of the nucleic acid sequence, e.g., the gene(s) or gene cassettes encoding one or more oxalate catabolism enzyme(s) and/or one or more oxalate transporter(s), one or more exporter(s) (e.g., of formate) and/or one or more antiporter(s)(e.g., oxalate:formate antiporter(s)). In other words, the regulatory sequence acts in cis. In one embodiment, a gene may be “directly linked” to a regulatory sequence in a manner which allows expression of the gene. In another embodiment, a gene may be “indirectly linked” to a regulatory sequence in a manner which allows expression of the gene. In one embodiment, two or more genes may be directly or indirectly linked to a regulatory sequence in a manner which allows expression of the two or more genes. A regulatory region or sequence is a nucleic acid that can direct transcription of a gene of interest and may comprise promoter sequences, enhancer sequences, response elements, protein recognition sites, inducible elements, promoter control elements, protein binding sequences, 5′ and 3′ untranslated regions, transcriptional start sites, termination sequences, polyadenylation sequences, and introns.

A “promoter” as used herein, refers to a nucleotide sequence that is capable of controlling the expression of a coding sequence or gene. Promoters are generally located 5′ of the sequence that they regulate. Promoters may be derived in their entirety from a native gene, or be composed of different elements derived from promoters found in nature, and/or comprise synthetic nucleotide segments. Those skilled in the art will readily ascertain that different promoters may regulate expression of a coding sequence or gene in response to a particular stimulus, e.g., in a cell- or tissue-specific manner, in response to different environmental or physiological conditions, or in response to specific compounds. Prokaryotic promoters are typically classified into two classes: inducible and constitutive. A “constitutive promoter” refers to a promoter that allows for continual transcription of the coding sequence or gene under its control.

“Constitutive promoter” refers to a promoter that is capable of facilitating continuous transcription of a coding sequence or gene under its control and/or to which it is operably linked. Constitutive promoters and variants are well known in the art and include, but are not limited to, Ptac promoter, BBa_J23100, a constitutive Escherichia coli σ^{s} promoter (e.g., an osmY promoter (International Genetically Engineered Machine (iGEM) Registry of Standard Biological Parts Name BBa_J45992; BBa_J45993)), a constitutive Escherichia coli σ³² promoter (e.g., htpG heat shock promoter (BBa_J45504)), a constitutive Escherichia coli σ⁷⁰ promoter (e.g., lacq promoter (BBa_J54200; BBa_J56015), E. coli CreABCD phosphate sensing operon promoter (BBa_J64951), GlnRS promoter (BBa_K088007), lacZ promoter (BBa_K119000; BBa_K119001); M13K07 gene I promoter (BBa_M13101); M13K07 gene II promoter (BBa_M13102), M13K07 gene III promoter (BBa_M13103), M13K07 gene IV promoter (BBa_M13104), M13K07 gene V promoter (BBa_M13105), M13K07 gene VI promoter (BBa_M13106), M13K07 gene VIII promoter (BBa_M13108), M13110 (BBa_M13110)), a constitutive Bacillus subtilis σ^{A} promoter (e.g., promoter veg (BBa_K143013), promoter 43 (BBa_K143013), P_{liaG} (BBa_K823000), P_{lepA} (BBa_K823002), P_{veg} (BBa_K823003)), a constitutive Bacillus subtilis σ^{B} promoter (e.g., promoter ctc (BBa_K143010), promoter gsiB (BBa_K143011)), a Salmonella promoter (e.g., Pspv2 from Salmonella (BBa_K112706), Pspv from Salmonella (BBa_K112707)), a bacteriophage T7 promoter (e.g., T7 promoter (BBa_I712074; BBa_I719005; BBa_J34814; BBa_J64997; BBa_K113010; BBa_K113011; BBa_K113012; BBa_R0085; BBa_R0180; BBa_R0181; BBa_R0182; BBa_R0183; BBa_Z0251; BBa_Z0252; BBa_Z0253)), and a bacteriophage SP6 promoter (e.g., SP6 promoter (BBa_J64998)).

An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition. A “directly inducible promoter” refers to a regulatory region, wherein the regulatory region is operably linked to a gene and/or gene cassette encoding one or more oxalate-metabolizing enzyme(s) and/or one or more oxalate transporter(s) and/or one or more exporter(s) (e.g., of formate) and/or one or more antiporter(s) (e.g., oxalate:formate antiporter(s)), where, in the presence of an inducer of said regulatory region, the protein or polypeptide is expressed. An “indirectly inducible promoter” refers to a regulatory system comprising two or more regulatory regions, for example, a first regulatory region that is operably linked to a first gene encoding a first protein, polypeptide, or factor, e.g., a transcriptional regulator, which is capable of regulating a second regulatory region that is operably linked to a second gene, the second regulatory region may be activated or repressed, thereby activating or repressing expression of the second gene. Both a directly inducible promoter and an indirectly inducible promoter are encompassed by “inducible promoter.” Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, a P_{TetR} promoter, and a P_{LacI} promoter, each of which are described in more detail herein. Examples of other inducible promoters are provided herein below.

As used herein, “stably maintained” or “stable” bacterium is used to refer to a bacterial host cell carrying non-native genetic material, e.g., a gene and/or gene cassette encoding one or more oxalate-metabolizing enzyme(s) and/or one or more oxalate transporter(s) and/or one or more exporter(s) (e.g., of formate) and/or one or more antiporter(s)(e.g., oxalate:formate antiporter(s)), which is incorporated into the host genome or propagated on a self-replicating extra-chromosomal plasmid, such that the non-native genetic material is retained, expressed, and propagated. The stable bacterium is capable of survival and/or growth in vitro, e.g., in medium, and/or in vivo, e.g., in the gut. For example, the stable bacterium may be a genetically engineered bacterium comprising a gene and/or gene cassette encoding one or more oxalate-metabolizing enzyme(s) and/or one or more oxalate transporter(s) and/or one or more exporter(s) (e.g., of formate) and/or one or more antiporter(s)(e.g., oxalate:formate antiporter(s)), in which the plasmid or chromosome carrying the gene is stably maintained in the bacterium, such that the one or more oxalate-metabolizing enzyme(s) and/or one or more oxalate transporter(s) and/or one or more exporter(s) (e.g., of formate) and/or one or more antiporter(s)(e.g., oxalate:formate antiporter(s)) can be expressed in the bacterium, and the bacterium is capable of survival and/or growth in vitro and/or in vivo. In some embodiments, copy number affects the stability of expression of the non-native genetic material. In some embodiments, copy number affects the level of expression of the non-native genetic material.

As used herein, the term “expression” refers to the transcription and stable accumulation of sense (mRNA) or anti-sense RNA derived from a nucleic acid, and/or to translation of an mRNA into a polypeptide.

As used herein, the term “plasmid” or “vector” refers to an extrachromosomal nucleic acid, e.g., DNA, construct that is not integrated into a bacterial cell's genome. Plasmids are usually circular and capable of autonomous replication. Plasmids may be low-copy, medium-copy, or high-copy, as is well known in the art. Plasmids may optionally comprise a selectable marker, such as an antibiotic resistance gene, which helps select for bacterial cells containing the plasmid and which ensures that the plasmid is retained in the bacterial cell. A plasmid disclosed herein may comprise a nucleic acid sequence encoding a heterologous gene, e.g., a gene and/or gene cassette encoding one or more oxalate-metabolizing enzyme(s) and/or one or more oxalate transporter(s) and/or one or more exporter(s) (e.g., of formate) and/or one or more antiporter(s) (e.g., oxalate:formate antiporter(s)).

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The term “genetic modification,” as used herein, refers to any genetic change. Exemplary genetic modifications include those that increase, decrease, or abolish the expression of a gene, including, for example, modifications of native chromosomal or extrachromosomal genetic material. Exemplary genetic modifications also include the introduction of at least one plasmid, modification, mutation, base deletion, base addition, base substitution, and/or codon modification of chromosomal or extrachromosomal genetic sequence(s), gene over-expression, gene amplification, gene suppression, promoter modification or substitution, gene addition (either single or multi-copy), antisense expression or suppression, or any other change to the genetic elements of a host cell, whether the change produces a change in phenotype or not. Genetic modification can include the introduction of a plasmid, e.g., a plasmid comprising a gene and/or gene cassette encoding one or more oxalate-metabolizing enzyme(s) and/or one or more oxalate transporter(s) and/or one or more exporter(s) (e.g., of formate) and/or one or more antiporter(s) (e.g., oxalate:formate antiporter(s)) operably linked to a promoter, into a bacterial cell. Genetic modification can also involve a targeted replacement in the chromosome, e.g., to replace a native gene promoter with an inducible promoter, regulated promoter, strong promoter, or constitutive promoter. Genetic modification can also involve gene amplification, e.g., introduction of at least one additional copy of a native gene into the chromosome of the cell. Alternatively, chromosomal genetic modification can involve a genetic mutation.

As used herein, the term “genetic mutation” refers to a change or changes in a nucleotide sequence of a gene or related regulatory region that alters the nucleotide sequence as compared to its native or wild-type sequence. Mutations include, for example, substitutions, additions, and deletions, in whole or in part, within the wild-type sequence. Such substitutions, additions, or deletions can be single nucleotide changes (e.g., one or more point mutations), or can be two or more nucleotide changes, which may result in substantial changes to the sequence. Mutations can occur within the coding region of the gene as well as within the non-coding and regulatory sequence of the gene. The term “genetic mutation” is intended to include silent and conservative mutations within a coding region as well as changes which alter the amino acid sequence of the polypeptide encoded by the gene. A genetic mutation in a gene coding sequence may, for example, increase, decrease, or otherwise alter the activity (e.g., enzymatic activity) of the gene's polypeptide product. A genetic mutation in a regulatory sequence may increase, decrease, or otherwise alter the expression of sequences operably linked to the altered regulatory sequence.

Specifically, the term “genetic modification that reduces export of oxalate from the bacterial cell” refers to a genetic modification that reduces the rate of export or quantity of export of an oxalate from the bacterial cell, as compared to the rate of export or quantity of export of oxalate from a bacterial cell not having said modification, e.g., a wild-type bacterial cell. In one embodiment, a recombinant bacterial cell having a genetic modification that reduces export of oxalate from the bacterial cell comprises a genetic mutation in a native gene. In another embodiment, a recombinant bacterial cell having a genetic modification that reduces export of oxalate from the bacterial cell comprises a genetic mutation in a native promoter, which reduces or inhibits transcription of a gene encoding an oxalate exporter. In another embodiment, a recombinant bacterial cell having a genetic modification that reduces export of oxalate from the bacterial cell comprises a genetic mutation leading to overexpression of a repressor of an exporter of oxalate. In another embodiment, a recombinant bacterial cell having a genetic modification that reduces export of oxalate from the bacterial cell comprises a genetic mutation which reduces or inhibits translation of the gene encoding the oxalate exporter.

Moreover, the term “genetic modification that increases import of oxalate into the bacterial cell” refers to a genetic modification that increases the uptake rate or increases the uptake quantity of oxalate into the cytosol of the bacterial cell, as compared to the uptake rate or uptake quantity of the oxalate into the cytosol of a bacterial cell not having said modification, e.g., a wild-type bacterial cell. In some embodiments, an engineered bacterial cell having a genetic modification that increases import of oxalate into the bacterial cell refers to a bacterial cell comprising a heterologous gene sequence (native or non-native) encoding one or more importer/transporter(s) of oxalate. In some embodiments, the genetically engineered bacteria comprising genetic modification that increases import of oxalate into the bacterial cell comprise gene sequence(s) encoding an oxalate transporter or other metabolite transporter or an antiporter, e.g. an oxalate:formate antiporter, that transports oxalate into the bacterial cell. The transporter can be any transporter that assists or allows import of oxalate into the cell. In certain embodiments, the oxalate transporter is antiporter, e.g. an oxalate:formate antiporter, e.g., OxlT, e.g. from O. formigenes. In certain embodiments, the engineered bacterial cell contains gene sequence encoding OxlT, e.g. from O. formigenes. In some embodiments, the engineered bacteria comprise more than one copy of gene sequence encoding an oxalate transporter, e.g., an oxalate:formate antiporter, e.g., OxlT, e.g. from O. formigenes. In some embodiments, the engineered bacteria comprise gene sequence(s) encoding more than one oxalate transporter, e.g., two or more different oxalate transporters.

As used herein, the term “transporter” is meant to refer to a mechanism, e.g., protein, proteins, or protein complex, for importing a molecule, e.g., amino acid, peptide (di-peptide, tri-peptide, polypeptide, etc.), toxin, metabolite, substrate, as well as other biomolecules into the microorganism from the extracellular milieu. As used herein, the term “transporter” also includes antiporters, which can import and export metabolites, e.g. such as oxalate:formate antiporters described herein. As used herein, the terms “transporter” and “importer” are used equivalently.

The term “oxalate” as used herein, refers to the dianion of the formula C₂O₄²⁻. Oxalate is the conjugate base of oxalic acid. The term “oxalic acid,” as used herein, refers to a dicarboxylic acid with the chemical formula H₂C₂O₄.

As used herein, the phrase “exogenous environmental condition” or “exogenous environment signal” refers to settings, circumstances, stimuli, or biological molecules under which a promoter described herein is directly or indirectly induced. The phrase “exogenous environmental conditions” is meant to refer to the environmental conditions external to the engineered microorganism, but endogenous or native to the host subject environment. Thus, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal. In some embodiments, the exogenous environmental conditions are specific to the upper gastrointestinal tract of a mammal. In some embodiments, the exogenous environmental conditions are specific to the lower gastrointestinal tract of a mammal. In some embodiments, the exogenous environmental conditions are specific to the small intestine of a mammal. In some embodiments, the exogenous environmental conditions are low-oxygen, microaerobic, or anaerobic conditions, such as the environment of the mammalian gut. In some embodiments, exogenous environmental conditions are molecules or metabolites that are specific to the mammalian gut, e.g., propionate. In some embodiments, the exogenous environmental condition is a tissue-specific or disease-specific metabolite or molecule(s). In some embodiments, the exogenous environmental condition is specific to a disease, e.g., hyperoxaluria. In some embodiments, the exogenous environmental condition is a low-pH environment. In some embodiments, the genetically engineered microorganism of the disclosure comprises a pH-dependent promoter. In some embodiments, the genetically engineered microorganism of the disclosure comprise an oxygen level-dependent promoter. In some aspects, bacteria have evolved transcription factors that are capable of sensing oxygen levels. Different signaling pathways may be triggered by different oxygen levels and occur with different kinetics. An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR (fumarate and nitrate reductase), ANR, and DNR. Corresponding FNR-responsive promoters, ANR (anaerobic nitrate respiration)-responsive promoters, and DNR (dissimilatory nitrate respiration regulator)-responsive promoters are known in the art (see, e.g., Castiglione et al., 2009; Eiglmeier et al., 1989; Galimand et al., 1991; Hasegawa et al., 1998; Hoeren et al., 1993; Salmon et al., 2003), and non-limiting examples are shown in Table 1.

In a non-limiting example, a promoter (PfnrS) was derived from the E. coli Nissle fumarate and nitrate reductase gene S (fnrS) that is known to be highly expressed under conditions of low or no environmental oxygen (Durand and Storz, 2010; Boysen et al, 2010). The PfnrS promoter is activated under anaerobic conditions by the global transcriptional regulator FNR that is naturally found in Nissle. Under anaerobic conditions, FNR forms a dimer and binds to specific sequences in the promoters of specific genes under its control, thereby activating their expression. However, under aerobic conditions, oxygen reacts with iron-sulfur clusters in FNR dimers and converts them to an inactive form. In this way, the PfnrS inducible promoter is adopted to modulate the expression of proteins or RNA. PfnrS is used interchangeably in this application as FNRS, fnrs, FNR, P-FNRS promoter and other such related designations to indicate the promoter PfnrS.

TABLE 1
Examples of transcription factors and
responsive genes and regulatory regions

| | Examples of responsive genes, promoters, |
|---|---|
| Transcription Factor | and/or regulatory regions: |
| FNR | nirB, ydfZ, pdhR, focA, ndH, hlyE, narK, |
| | narX, narG, yfiD, tdcD |
| ANR | arcDABC |
| DNR | norb, norC |

presence or absence of reactive oxygen species (ROS). In other embodiments, the exogenous environmental conditions are the presence or absence of reactive nitrogen species (RNS). In some embodiments, exogenous environmental conditions are biological molecules that are involved in the inflammatory response, for example, molecules present in an inflammatory disorder of the gut. In some embodiments, the exogenous environmental conditions or signals exist naturally or are naturally absent in the environment in which the recombinant bacterial cell resides. In some embodiments, the exogenous environmental conditions or signals are artificially created, for example, by the creation or removal of biological conditions and/or the administration or removal of biological molecules.

In some embodiments, the exogenous environmental condition(s) and/or signal(s) stimulates the activity of an inducible promoter. In some embodiments, the exogenous environmental condition(s) and/or signal(s) that serves to activate the inducible promoter is not naturally present within the gut of a mammal. In some embodiments, the inducible promoter is stimulated by a molecule or metabolite that is administered in combination with the pharmaceutical composition of the disclosure, for example, tetracycline, arabinose, or any biological molecule that serves to activate an inducible promoter. In some embodiments, the exogenous environmental condition(s) and/or signal(s) is added to culture media comprising a recombinant bacterial cell of the disclosure. In some embodiments, the exogenous environmental condition that serves to activate the inducible promoter is naturally present within the gut of a mammal (for example, low oxygen or anaerobic conditions, or biological molecules involved in an inflammatory response). In some embodiments, the loss of exposure to an exogenous environmental condition (for example, in vivo) inhibits the activity of an inducible promoter, as the exogenous environmental condition is not present to induce the promoter (for example, an aerobic environment outside the gut). “Gut” refers to the organs, glands, tracts, and systems that are responsible for the transfer and digestion of food, absorption of nutrients, and excretion of waste. In humans, the gut comprises the gastrointestinal (GI) tract, which starts at the mouth and ends at the anus, and additionally comprises the esophagus, stomach, small intestine, and large intestine. The gut also comprises accessory organs and glands, such as the spleen, liver, gallbladder, and pancreas. The upper gastrointestinal tract comprises the esophagus, stomach, and duodenum of the small intestine. The lower gastrointestinal tract comprises the remainder of the small intestine, i.e., the jejunum and ileum, and all of the large intestine, i.e., the cecum, colon, rectum, and anal canal. Bacteria can be found throughout the gut, e.g., in the gastrointestinal tract, and particularly in the intestines.

“Microorganism” refers to an organism or microbe of microscopic, submicroscopic, or ultramicroscopic size that typically consists of a single cell. Examples of microorganisms include bacteria, viruses, parasites, fungi, certain algae, and protozoa. In some aspects, the microorganism is engineered (“engineered microorganism”) to produce one or more therapeutic molecules, e.g., oxalate catabolism enzyme(s). In certain embodiments, the engineered microorganism is an engineered bacterium. In certain embodiments, the engineered microorganism is an engineered virus.

“Non-pathogenic bacteria” refer to bacteria that are not capable of causing disease or harmful responses in a host. In some embodiments, non-pathogenic bacteria are Gram-negative bacteria. In some embodiments, non-pathogenic bacteria are Gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis and Saccharomyces boulardii (Sonnenborn et al., 2009; Dinleyici et al., 2014; U.S. Pat. Nos. 6,835,376; 6,203,797; 5,589,168; 7,731,976). Non-pathogenic bacteria also include commensal bacteria, which are present in the indigenous microbiota of the gut. In one embodiment, the disclosure further includes non-pathogenic Saccharomyces, such as Saccharomyces boulardii. Naturally pathogenic bacteria may be genetically engineered to reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are Gram-negative bacteria. In some embodiments, the probiotic bacteria are Gram-positive bacteria. Some species, strains, and/or subtypes of non-pathogenic bacteria are currently recognized as probiotic bacteria. Examples of probiotic bacteria include, but are not limited to, certain strains belonging to the genus Bifidobacteria, Escherichia coli, Lactobacillus, and Saccharomyces e.g., Bifidobacterium bifidum, Enterococcus faecium, Escherichia coli strain Nissle, Lactobacillus acidophilus, Lactobacillus bulgaricus, Lactobacillus paracasei, and Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, the term “auxotroph” or “auxotrophic” refers to an organism that requires a specific factor, e.g., an amino acid, a sugar, or other nutrient) to support its growth. An “auxotrophic modification” is a genetic modification that causes the organism to die in the absence of an exogenously added nutrient essential for survival or growth because it is unable to produce said nutrient. As used herein, the term “essential gene” refers to a gene which is necessary to for cell growth and/or survival. Essential genes are described in more detail infra and include, but are not limited to, DNA synthesis genes (such as thyA), cell wall synthesis genes (such as dapA), and amino acid genes (such as serA and metA).

As used herein, the terms “modulate” and “treat” and their cognates refer to an amelioration of a disease, disorder, and/or condition, or at least one discernible symptom thereof. In another embodiment, “modulate” and “treat” refer to an amelioration of at least one measurable physical parameter, not necessarily discernible by the patient. In another embodiment, “modulate” and “treat” refer to inhibiting the progression of a disease, disorder, and/or condition, either physically (e.g., stabilization of a discernible symptom), physiologically (e.g., stabilization of a physical parameter), or both. In another embodiment, “modulate” and “treat” refer to slowing the progression or reversing the progression of a disease, disorder, and/or condition. As used herein, “prevent” and its cognates refer to delaying the onset or reducing the risk of acquiring a given disease, disorder and/or condition or a symptom associated with such disease, disorder, and/or condition.

Those in need of treatment may include individuals already having a particular medical disease, as well as those at risk of having, or who may ultimately acquire the disease. The need for treatment is assessed, for example, by the presence of one or more risk factors associated with the development of a disease, the presence or progression of a disease, or likely receptiveness to treatment of a subject having the disease. Disorders in which oxalate is detrimental, e.g., a hyperoxaluria, may be caused by inborn genetic mutations for which there are no known cures. Diseases can also be secondary to other conditions, e.g., an intestinal disorder. Treating diseases in which oxalate is detrimental, such as a primary hyperoxaluria or secondary hyperoxaluria, may encompass reducing normal levels of oxalate and/or oxalic acid, reducing excess levels of oxalate and/or oxalic acid, or eliminating oxalate, and/or oxalic acid, and does not necessarily encompass the elimination of the underlying disease.

As used herein, the term “catabolism” refers to the cellular uptake of oxalate, and/or degradation of oxalate into its corresponding oxalyl CoA, and/or the degradation of oxalyl CoA formate and carbon dioxide. In one embodiment, the cellular uptake of oxalate occurs in the kidney. In one embodiment, the cellular uptake occurs in the liver. In one embodiment, the cellular uptake of oxalate occurs in the intestinal tract. In one embodiment, the cellular uptake of oxalate occurs in the stomach. In one embodiment, the cellular uptake is mediated by a SLC26 transporting protein (see Robijn et al. (2011)). In one embodiment, the cellular uptake is mediated by the transport protein SLC26A1. In one embodiment, the cellular uptake is mediated by the transport protein SLC26A6. In one embodiment, the cellular uptake of oxalate is mediated by a paracellular transport system. In one embodiment, the cellular uptake of oxalate is mediated by a transcellular transport system.

In one embodiment, “abnormal catabolism” refers to a decrease in the rate of cellular uptake of oxalate. In one embodiment, “abnormal catabolism” refers to any condition(s), disorder(s), disease(s), predisposition(s), and/or genetic mutations(s) that result in daily urinary oxalate excretion over 40 mg per 24 hours. In one embodiment, “abnormal catabolism” refers to an inability and/or decreased capacity of an organ and/or system to process and/or mediate the cellular uptake of oxalate. In one embodiment, said inability or decreased capacity of an organ and/or system to process and/or mediate the cellular uptake of oxalate is caused by the increased endogenous production of oxalate. In one embodiment, increased endogenous production of oxalate results from the absence of, or a deficiency in, the peroxisomal liver enzyme AGT. In one embodiment, increased endogenous production of oxalate results from the absence of, or a deficiency in the enzyme GRHPR. In one embodiment, increased endogenous production of oxalate results from the absence of, or a deficiency in the enzyme 4-hydroxy-2-oxoglutarate aldolase. In one embodiment, said inability or decreased capacity of an organ and/or system to process and/or mediate the cellular uptake of oxalate is caused by increased absorption of oxalate. In one embodiment, said increased absorption of oxalate results from an increased dietary intake of oxalate. In one embodiment, said increased absorption of oxalate results from increased intestinal absorption of oxalate. In one embodiment, said increased absorption of oxalate results from excessive intake of oxalate precursors. In one embodiment, said increased absorption of oxalate results from a decrease in intestinal oxalate-degrading microorganisms. In one embodiment, said increased absorption of oxalate results from genetic variations of intestinal oxalate transporters.

In one embodiment, a “disorder in which oxalate is detrimental” is a disease or disorder involving the abnormal, e.g., increased, levels of oxalate and/or oxalic acid or molecules directly upstream, such as glyoxylate. In one embodiment, the disorder in which oxalate is detrimental is a disorder or disease in which hyperoxaluria is observed in the subject. In one embodiment the disorder in which oxalate is detrimental refers to any condition(s), disorder(s), disease(s), predisposition(s), and/or genetic mutations(s) that result in daily urinary oxalate excretion over 40 mg per 24 hours. In one embodiment the disorder in which oxalate is detrimental is a disorder or disease selected from the group consisting of: PHI, PHII, PHII, secondary hyperoxaluria, enteric hyperoxaluria, syndrome of bacterial overgrowth, Crohn's disease, inflammatory bowel disease, hyperoxaluria following renal transplantation, hyperoxaluria after a jejunoileal bypass for obesity, hyperoxaluria after gastric ulcer surgery, chronic mesenteric ischemia, gastric bypass, cystic fibrosis, short bowel syndrome, biliary/pancreatic diseases (e.g., chronic pancreatitis).

As used herein a “pharmaceutical composition” refers to a preparation of genetically engineered microorganism of the disclosure, e.g., genetically engineered bacteria or virus, with other components such as a physiologically suitable carrier and/or excipient. In one embodiment, the pharmaceutical composition is a frozen liquid composition. In another embodiment, the pharmaceutical composition is a lyophilized composition.

The phrases “physiologically acceptable carrier” and “pharmaceutically acceptable carrier” which may be used interchangeably refer to a carrier or a diluent that does not cause significant irritation to an organism and does not abrogate the biological activity and properties of the administered bacterial or viral compound. An adjuvant is included under these phrases.

The term “excipient” refers to an inert substance added to a pharmaceutical composition to further facilitate administration of an active ingredient. Examples include, but are not limited to, calcium bicarbonate, sodium bicarbonate calcium phosphate, various sugars and types of starch, cellulose derivatives, gelatin, vegetable oils, polyethylene glycols, and surfactants, including, for example, polysorbate 20.

The terms “therapeutically effective dose” and “therapeutically effective amount” are used to refer to an amount of a compound that results in prevention, delay of onset of symptoms, or amelioration of symptoms of a condition, e.g., a disorder in which oxalate is detrimental. A therapeutically effective amount may, for example, be sufficient to treat, prevent, reduce the severity, delay the onset, and/or reduce the risk of occurrence of one or more symptoms of a disease or condition associated with daily urinary oxalate excretion over 40 mg per 24 hours. A therapeutically effective amount, as well as a therapeutically effective frequency of administration, can be determined by methods known in the art and discussed below.

As used herein, the term “bacteriostatic” or “cytostatic” refers to a molecule or protein which is capable of arresting, retarding, or inhibiting the growth, division, multiplication or replication of recombinant bacterial cell of the disclosure.

As used herein, the term “bactericidal” refers to a molecule or protein which is capable of killing the recombinant bacterial cell of the disclosure.

As used herein, the term “toxin” refers to a protein, enzyme, or polypeptide fragment thereof, or other molecule which is capable of arresting, retarding, or inhibiting the growth, division, multiplication or replication of the recombinant bacterial cell of the disclosure, or which is capable of killing the recombinant bacterial cell of the disclosure. The term “toxin” is intended to include bacteriostatic proteins and bactericidal proteins. The term “toxin” is intended to include, but not limited to, lytic proteins, bacteriocins (e.g., microcins and colicins), gyrase inhibitors, polymerase inhibitors, transcription inhibitors, translation inhibitors, DNases, and RNases. The term “anti-toxin” or “antitoxin,” as used herein, refers to a protein or enzyme which is capable of inhibiting the activity of a toxin. The term anti-toxin is intended to include, but not limited to, immunity modulators, and inhibitors of toxin expression. Examples of toxins and antitoxins are known in the art and described in more detail infra.

As used herein, the term “oxalate catabolic or catabolism enzyme” or “oxalate catabolic or catabolism enzyme” or “oxalate metabolic enzyme” refers to any enzyme that is capable of metabolizing oxalate or capable of reducing accumulated oxalate or that can lessen, ameliorate, or prevent one or more diseases, or disease symptoms in which oxalate is detrimental. Examples of oxalate enzymes include, but are not limited to, formyl-CoA:oxalate CoA-transferase (also called formyl-CoA transferase), e.g., Frc from O. formigenes, oxalyl-CoA synthetase (also called oxalate-CoA ligase), e.g., Saccharomyces cerevisiae acyl-activating enzyme 3 (ScAAE3) from Saccharomyces cerevisiae, Oxalyl-CoA Decarboxylase, e.g., Oxc from O. formigenes (also referred to herein is oxdC or oxalate decarboxylase), acetyl-CoA:oxalate CoA transferase (ACOCT), e.g., YfdE from E. coli and any other enzymes that catabolizes oxalate, oxalyl-CoA or any other metabolite thereof. Catabolism enzymes also include alanine glyoxalate aminotransferase (AGT, encoded by the AGXT gene, e.g. the human form), glyoxylate/hydroxypyruvate reductase (GRHPR; an enzyme having glyoxylate reductase (GR), hydroxypyruvate reductase (HPR), and D-glycerate dehydrogenase (DGDH) activities, e.g., the human form), and 4-hydroxy 2-oxoglutarate aldolase (encoded by the HOGA1 gene, e.g. in humans, and which breaks down 4-hydroxy 2-oxoglutarate into pyruvate and glyoxalate). Functional deficiencies in these proteins result in the accumulation of oxalate or its corresponding α-keto acid in cells and tissues. Oxalate metabolic enzymes of the present disclosure include both wild-type or modified oxalate metabolic enzymes and can be produced using recombinant and synthetic methods or purified from nature sources. Oxalate metabolic enzymes include full-length polypeptides and functional fragments thereof, as well as homologs and variants thereof oxalate metabolic enzymes include polypeptides that have been modified from the wild-type sequence, including, for example, polypeptides having one or more amino acid deletions, insertions, and/or substitutions and may include, for example, fusion polypeptides and polypeptides having additional sequence, e.g., regulatory peptide sequence, linker peptide sequence, and other peptide sequence.

As used herein, “payload” refers to one or more molecules of interest to be produced by a genetically engineered microorganism, such as a bacteria or a virus. In some embodiments, the payload is a therapeutic payload, e.g., an oxalate catabolic enzyme or an oxalate transporter polypeptide. In some embodiments, the payload is a regulatory molecule, e.g., a transcriptional regulator such as FNR. In some embodiments, the payload comprises a regulatory element, such as a promoter or a repressor. In some embodiments, the payload comprises an inducible promoter, such as from FNRS. In some embodiments the payload comprises a repressor element, such as a kill switch. In some embodiments the payload comprises an antibiotic resistance gene or gene cassette. In some embodiments, the payload is encoded by a gene, multiple genes, gene cassette, or an operon. In alternate embodiments, the payload is produced by a biosynthetic or biochemical pathway, wherein the biosynthetic or biochemical pathway may optionally be endogenous to the microorganism. In alternate embodiments, the payload is produced by a biosynthetic or biochemical pathway, wherein the biosynthetic or biochemical pathway is not endogenous to the microorganism. In some embodiments, the genetically engineered microorganism comprises two or more payloads.

As used herein, the term “conventional hyperoxaluria treatment” or “conventional hyperoxaluria therapy” refers to treatment or therapy that is currently accepted, considered current standard of care, and/or used by most healthcare professionals for treating a disease or disorders in which oxalate is detrimental. It is different from alternative or complementary therapies, which are not as widely used.

As used herein, the term “polypeptide” includes “polypeptide” as well as “polypeptides,” and refers to a molecule composed of amino acid monomers linearly linked by amide bonds (i.e., peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including but not limited to glycosylation, acetylation, phosphorylation, amidation, derivatization, proteolytic cleavage, or modification by non-naturally occurring amino acids. A polypeptide may be derived from a natural biological source or produced by recombinant technology. In other embodiments, the polypeptide is produced by the genetically engineered bacteria or virus of the current invention. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides, which do not possess a defined three-dimensional structure, but rather can adopt a large number of different conformations, are referred to as unfolded. The term “peptide” or “polypeptide” may refer to an amino acid sequence that corresponds to a protein or a portion of a protein or may refer to an amino acid sequence that corresponds with non-protein sequence, e.g., a sequence selected from a regulatory peptide sequence, leader peptide sequence, signal peptide sequence, linker peptide sequence, and other peptide sequence.

An “isolated” polypeptide or a fragment, variant, or derivative thereof refers to a polypeptide that is not in its natural milieu. No particular level of purification is required. Recombinantly produced polypeptides and proteins expressed in host cells, including but not limited to bacterial or mammalian cells, are considered isolated for purposed of the invention, as are native or recombinant polypeptides which have been separated, fractionated, or partially or substantially purified by any suitable technique. Recombinant peptides, polypeptides or proteins refer to peptides, polypeptides or proteins produced by recombinant DNA techniques, i.e. produced from cells, microbial or mammalian, transformed by an exogenous recombinant DNA expression construct encoding the polypeptide. Proteins or peptides expressed in most bacterial cultures will typically be free of glycan. Fragments, derivatives, analogs or variants of the foregoing polypeptides, and any combination thereof are also included as polypeptides. The terms “fragment,” “variant,” “derivative” and “analog” include polypeptides having an amino acid sequence sufficiently similar to the amino acid sequence of the original peptide and include any polypeptides, which retain at least one or more properties of the corresponding original polypeptide. Fragments of polypeptides of the present invention include proteolytic fragments, as well as deletion fragments. Fragments also include specific antibody or bioactive fragments or immunologically active fragments derived from any polypeptides described herein. Variants may occur naturally or be non-naturally occurring. Non-naturally occurring variants may be produced using mutagenesis methods known in the art. Variant polypeptides may comprise conservative or non-conservative amino acid substitutions, deletions or additions.

Polypeptides also include fusion proteins. As used herein, the term “variant” includes a fusion protein, which comprises a sequence of the original peptide or sufficiently similar to the original peptide. As used herein, the term “fusion protein” refers to a chimeric protein comprising amino acid sequences of two or more different proteins. Typically, fusion proteins result from well known in vitro recombination techniques. Fusion proteins may have a similar structural function (but not necessarily to the same extent), and/or similar regulatory function (but not necessarily to the same extent), and/or similar biochemical function (but not necessarily to the same extent) and/or immunological activity (but not necessarily to the same extent) as the individual original proteins which are the components of the fusion proteins. “Derivatives” include but are not limited to peptides, which contain one or more naturally occurring amino acid derivatives of the twenty standard amino acids. “Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, EMBO J. 8 (1989), 779-785. For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu.

As used herein, the term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the peptides of the invention. Such variants generally retain the functional activity of the peptides of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

As used herein the term “linker”, “linker peptide” or “peptide linkers” or “linker” refers to synthetic or non-native or non-naturally-occurring amino acid sequences that connect or link two polypeptide sequences, e.g., that link two polypeptide domains. As used herein the term “synthetic” refers to amino acid sequences that are not naturally occurring. Exemplary linkers are described herein. Additional exemplary linkers are provided in US 20140079701, the contents of which are herein incorporated by reference in its entirety.

As used herein the term “codon-optimized” refers to the modification of codons in the gene or coding regions of a nucleic acid molecule to reflect the typical codon usage of the host organism without altering the polypeptide encoded by the nucleic acid molecule. Such optimization includes replacing at least one, or more than one, or a significant number, of codons with one or more codons that are more frequently used in the genes of the host organism. A “codon-optimized sequence” refers to a sequence, which was modified from an existing coding sequence, or designed, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. Many organisms display a bias or preference for use of particular codons to code for insertion of a particular amino acid in a growing polypeptide chain. Codon preference or codon bias, differences in codon usage between organisms, is allowed by the degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

As used herein, the terms “secretion system” or “secretion protein” refers to a native or non-native secretion mechanism capable of secreting or exporting a biomolecule, e.g., polypeptide from the microbial, e.g., bacterial cytoplasm. The secretion system may comprise a single protein or may comprise two or more proteins assembled in a complex e.g. HlyBD. Non-limiting examples of secretion systems for gram negative bacteria include the modified type III flagellar, type I (e.g., hemolysin secretion system), type II, type W, type V, type VI, and type VII secretion systems, resistance-nodulation-division (RND) multi-drug efflux pumps, various single membrane secretion systems. Non-liming examples of secretion systems for gram positive bacteria include Sec and TAT secretion systems. In some embodiments, the polypeptide to be secreted include a “secretion tag” of either RNA or peptide origin to direct the polypeptide to specific secretion systems. In some embodiments, the secretion system is able to remove this tag before secreting the polypeptide from the engineered bacteria. For example, in Type V auto-secretion-mediated secretion the N-terminal peptide secretion tag is removed upon translocation of the “passenger” peptide from the cytoplasm into the periplasmic compartment by the native Sec system. Further, once the auto-secretor is translocated across the outer membrane the C-terminal secretion tag can be removed by either an autocatalytic or protease-catalyzed e.g., OmpT cleavage thereby releasing the therapeutic polypeptide into the extracellular milieu. In some embodiments, the secretion system involves the generation of a “leaky” or de-stabilized outer membrane, which may be accomplished by deleting or mutagenizing genes responsible for tethering the outer membrane to the rigid peptidoglycan skeleton, including for example, lpp, ompC, ompA, ompF, tolA, tolB, pal, degS, degP, and nlpl. Lpp functions as the primary ‘staple’ of the bacterial cell wall to the peptidoglycan. TolA-PAL and OmpA complexes function similarly to Lpp and are other deletion targets to generate a leaky phenotype. Additionally, leaky phenotypes have been observed when periplasmic proteases, such as degS, degP or nlpl, are deactivated. Thus, in some embodiments, the engineered bacteria have one or more deleted or mutated membrane genes, e.g., selected from lpp, ompA, ompA, ompF, tolA, tolB, and pal genes. In some embodiments, the engineered bacteria have one or more deleted or mutated periplasmic protease genes, e.g., selected from degS, degP, and nlpl. In some embodiments, the engineered bacteria have one or more deleted or mutated gene(s), selected from lpp, ompA, ompA, ompF, tolA, tolB, pal, degS, degP, and nlpl genes.

The articles “a” and “an,” as used herein, should be understood to mean “at least one,” unless clearly indicated to the contrary.

The phrase “and/or,” when used between elements in a list, is intended to mean either (1) that only a single listed element is present, or (2) that more than one element of the list is present. For example, “A, B, and/or C” indicates that the selection may be A alone; B alone; C alone; A and B; A and C; B and C; or A, B, and C. The phrase “and/or” may be used interchangeably with “at least one of” or “one or more of” the elements in a list.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### Bacteria

The genetically engineered microorganisms, or programmed microorganisms, such as genetically engineered bacteria of the disclosure are capable of producing one or more enzymes for metabolizing an oxalate and/or a metabolite thereof. In some aspects, the disclosure provides a bacterial cell that comprises one or more heterologous gene sequence(s) encoding an oxalate catabolism enzyme or other protein that results in a decrease in oxalate levels.

In certain embodiments, the genetically engineered bacteria are obligate anaerobic bacteria. In certain embodiments, the genetically engineered bacteria are facultative anaerobic bacteria. In certain embodiments, the genetically engineered bacteria are aerobic bacteria. In some embodiments, the genetically engineered bacteria are Gram-positive bacteria. In some embodiments, the genetically engineered bacteria are Gram-positive bacteria and lack LPS. In some embodiments, the genetically engineered bacteria are Gram-negative bacteria. In some embodiments, the genetically engineered bacteria are Gram-positive and obligate anaerobic bacteria. In some embodiments, the genetically engineered bacteria are Gram-positive and facultative anaerobic bacteria. In some embodiments, the genetically engineered bacteria are non-pathogenic bacteria. In some embodiments, the genetically engineered bacteria are commensal bacteria. In some embodiments, the genetically engineered bacteria are probiotic bacteria. In some embodiments, the genetically engineered bacteria are naturally pathogenic bacteria that are modified or mutated to reduce or eliminate pathogenicity. Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera. In certain embodiments, the genetically engineered bacteria are selected from the group consisting of Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Oxalobacter formigenes bacterial cell. Saccharomyces boulardii. In certain embodiments, the genetically engineered bacteria are selected from Bacteroides fragilis, Bacteroides thetaiotaomicron, Bacteroides subtilis, Bifidobacterium bifidum, Bifidobacterium infantis, Bifidobacterium lactis, Clostridium butyricum, Escherichia coli, Escherichia coli Nissle, Lactobacillus acidophilus, Lactobacillus plantarum, Lactobacillus reuteri, and Lactococcus lactis bacterial cell. In one embodiment, the bacterial cell is a Bacteroides fragilis bacterial cell. In one embodiment, the bacterial cell is a Bacteroides thetaiotaomicron bacterial cell. In one embodiment, the bacterial cell is a Bacteroides subtilis bacterial cell. In one embodiment, the bacterial cell is a Bifidobacterium bifidum bacterial cell. In one embodiment, the bacterial cell is a Bifidobacterium infantis bacterial cell. In one embodiment, the bacterial cell is a Bifidobacterium lactis or B. infantis bacterial cell. In one embodiment, the bacterial cell is a Clostridium butyricum bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment the bacterial cell is a Bifidobacterium lactis bacterial cell. In one embodiment, the bacterial cell is a Clostridium butyricum bacterial cell. In one embodiment, the bacterial cell is an Escherichia coli bacterial cell. In one embodiment, the bacterial cell is a Lactobacillus acidophilus bacterial cell. In one embodiment, the bacterial cell is a Lactobacillus plantarum bacterial cell. In one embodiment, the bacterial cell is a Lactobacillus reuteri bacterial cell. In one embodiment, the bacterial cell is a Lactococcus lactis bacterial cell. In one embodiment, the bacterial cell is a Oxalobacter formigenes bacterial cell. In another embodiment, the bacterial cell does not include Oxalobacter formigenes.

In some embodiments, the genetically engineered bacteria are Escherichia coli strain Nissle 1917 (E. coli Nissle), a Gram-negative bacterium of the Enterobacteriaceae family that has evolved into one of the best characterized probiotics (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007).

In one embodiment, the recombinant bacterial cell of the invention does not colonize the subject having the disorder in which oxalate is detrimental.

One of ordinary skill in the art would appreciate that the genetic modifications disclosed herein may be adapted for other species, strains, and subtypes of bacteria. Furthermore, genes from one or more different species can be introduced into one another, e.g., an oxalate catabolism gene from Lactococcus lactis can be expressed in Escherichia coli. Unmodified E. coli Nissle and the genetically engineered bacteria of the invention may be destroyed, e.g., by defense factors in the gut or blood serum (Sonnenborn et al., 2009). In some embodiments, the residence time is calculated for a human subject. In some embodiments, residence time in vivo is calculated for the genetically engineered bacteria of the invention.

In some embodiments, the bacterial cell is a genetically engineered bacterial cell. In another embodiment, the bacterial cell is a recombinant bacterial cell. In some embodiments, the disclosure comprises a colony of bacterial cells disclosed herein.

In another aspect, the disclosure provides a recombinant bacterial culture which comprises bacterial cells disclosed herein. In one aspect, the disclosure provides a recombinant bacterial culture which reduces levels of oxalate or oxalic acid in the media of the culture. In one embodiment, the levels of the oxalate or oxalic acid are reduced by about 50%, about 75%, or about 100% in the media of the cell culture. In another embodiment, the levels of the oxalate or oxalic acid are reduced by about two-fold, three-fold, four-fold, five-fold, six-fold, seven-fold, eight-fold, nine-fold, or ten-fold in the media of the cell culture. In one embodiment, the levels of the oxalate or oxalic acid are reduced below the limit of detection in the media of the cell culture.

In some embodiments of the above described genetically engineered bacteria, the gene and/or gene cassette encoding one or more oxalate catabolism enzyme(s) is present on a plasmid in the bacterium. In some embodiments of the above described genetically engineered bacteria, the gene and/or gene cassette encoding one or more oxalate catabolism enzyme(s) is present on a plasmid in the bacterium and operatively linked on the plasmid to a promoter that is induced under low-oxygen or anaerobic conditions, such as any of the promoters disclosed herein. In other embodiments, the gene and/or gene cassette encoding one or more oxalate catabolism enzyme(s) is present in the bacterial chromosome. In other embodiments, the gene and/or gene cassette encoding one or more oxalate catabolism enzyme(s) is present in the bacterial chromosome and is operatively linked in the chromosome to the promoter that is induced under low-oxygen or anaerobic conditions, such as any of the promoters disclosed herein. In some embodiments of the above described genetically engineered bacteria, the gene and/or gene cassette encoding one or more oxalate catabolism enzyme(s) is present on a plasmid in the bacterium and operatively linked on the plasmid to the promoter that is induced under inflammatory conditions, such as any of the promoters disclosed herein. In other embodiments, the gene and/or gene cassette encoding one or more oxalate catabolism enzyme(s) is present in the bacterial chromosome and is operatively linked in the chromosome to the promoter that is induced under inflammatory conditions, such as any of the promoters disclosed herein.

In some embodiments, the genetically engineered bacteria comprising the gene and/or gene cassette encoding one or more oxalate catabolism enzyme(s) further comprise gene sequence(s) encoding an oxalate transporter. In some embodiments, the genetically engineered bacteria comprising the gene and/or gene cassette encoding one or more oxalate catabolism enzyme(s) further comprise gene sequence(s) encoding a formate exporter. In some embodiments, the genetically engineered bacteria comprising the gene and/or gene cassette encoding one or more oxalate catabolism enzyme(s) further comprise gene sequence(s) encoding an oxalate:formate antiporter. In some embodiments, the genetically engineered bacteria comprising the gene and/or gene cassette encoding one or more oxalate catabolism enzyme(s) further comprise gene sequence(s) encoding one or more of the following: an oxalate transporter, a formate exporter, and/or an oxalate:formate antiporter.

In some embodiments, the genetically engineered bacteria comprising the gene and/or gene cassette encoding one or more oxalate catabolism enzyme(s) and/or oxalate transporter, and/or formate exporter, and/or oxalate:formate antiporter is an auxotroph. In one embodiment, the genetically engineered bacteria is an auxotroph selected from a cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1 auxotroph. In some embodiments, the engineered bacteria have more than one auxotrophy, for example, they may be a ΔthyA and ΔdapA auxotroph.

In some embodiments, the genetically engineered bacteria comprising the gene and/or gene cassette encoding one or more oxalate catabolism enzyme(s) and/or oxalate transporter, and/or formate exporter, and/or oxalate:formate antiporter further comprise gene sequence(s) encoding a secretion protein or protein complex for secreting a biomolecule, such as any of the secretion systems disclosed herein.

In some embodiments, the genetically engineered bacteria comprising the gene and/or gene cassette encoding one or more oxalate catabolism enzyme(s) and/or oxalate transporter, and/or formate exporter, and/or oxalate:formate antiporter further comprise gene sequence(s) encoding one or more antibiotic gene(s), such as any of the antibiotic genes disclosed herein.

In some embodiments, the genetically engineered bacteria comprising a gene and/or gene cassette encoding one or more oxalate catabolism enzyme(s) and/or oxalate transporter, and/or formate exporter, and/or oxalate:formate antiporter further comprise a kill-switch circuit, such as any of the kill-switch circuits provided herein. For example, in some embodiments, the genetically engineered bacteria further comprise one or more genes encoding one or more recombinase(s) under the control of an inducible promoter, and an inverted toxin sequence. In some embodiments, the genetically engineered bacteria further comprise one or more genes encoding an antitoxin. In some embodiments, the engineered bacteria further comprise one or more genes encoding one or more recombinase(s) under the control of an inducible promoter and one or more inverted excision genes, wherein the excision gene(s) encode an enzyme that deletes an essential gene. In some embodiments, the genetically engineered bacteria further comprise one or more genes encoding an antitoxin. In some embodiments, the engineered bacteria further comprise one or more genes encoding a toxin under the control of a promoter having a TetR repressor binding site and a gene encoding the TetR under the control of an inducible promoter that is induced by arabinose, such as P_{araBAD} In some embodiments, the genetically engineered bacteria further comprise one or more genes encoding an antitoxin.

In some embodiments, the genetically engineered bacteria is an auxotroph comprising the gene and/or gene cassette encoding one or more oxalate catabolism enzyme(s) and further comprises a kill-switch circuit, such as any of the kill-switch circuits described herein.

In some embodiments of the above described genetically engineered bacteria, the gene and/or gene cassette encoding one or more oxalate catabolism enzyme(s) is present on a plasmid in the bacterium. In some embodiments, the gene and/or gene cassette encoding one or more oxalate catabolism enzyme(s) is present in the bacterial chromosome. In some embodiments, the genetically engineered bacteria comprise one or more gene and/or gene cassette(s) encoding one or more oxalate transporter(s) that transports oxalate into the bacterial cell. In some embodiments, the gene sequence(s) encoding an oxalate transporter is present on a plasmid in the bacterium. In some embodiments, the gene sequence(s) encoding an oxalate transporter is present in the bacterial chromosome. In some embodiments, the gene sequence encoding a secretion protein or protein complex for secreting a biomolecule, such as any of the secretion systems disclosed herein, is present on a plasmid in the bacterium. In some embodiments, the gene sequence encoding a secretion protein or protein complex for secreting a biomolecule, such as any of the secretion systems disclosed herein, is present in the bacterial chromosome. In some embodiments, the gene sequence(s) encoding an antibiotic resistance gene is present on a plasmid in the bacterium. In some embodiments, the gene sequence(s) encoding an antibiotic resistance gene is present in the bacterial chromosome.

Oxalate Catabolism Enzymes

O. formigenes was the first oxalate-degrading obligate anaerobe to be described in humans and has served as the paradigm organism in which anaerobic oxalate degradation has been studied. O. formigenes has three enzymes involved in the catabolism of oxalic acid. First extracellular oxalate is taken up by the membrane-associated oxalate-formate antiporter, OxlT, encoded by the oxlT gene. The frc gene encodes formyl-CoA transferase, Frc, which activates the intracellular oxalate to form oxalyl-CoA. This is decarboxylated in a thiamine PPi-dependent reaction by the oxalyl-CoA decarboxylase, Oxc, enzyme, expressed from the oxc gene. Formate and carbon dioxide are the end products, and the oxalate-formate antiporter, OxlT, catalyzes the export of the intracellular formate out of the cells. In O. formigenes, the generation of energy is coupled to oxalate transport, mediated by the oxalate transport membrane protein OxlT, (as described in Abratt and Reid Oxalate-Degrading Bacteria of the Human Gut as Probiotics in the Management of Kidney Stone Disease, the contents of which is herein incorporated by reference in its entirety, and references therein).

As used herein, the term “oxalate catabolism enzyme” refers to an enzyme involved in the catabolism of oxalate to its corresponding oxalyl-CoA molecule, the catabolism of oxalyl-CoA to formate and carbon dioxide, or the catabolism of oxalate to another metabolite. Enzymes involved in the catabolism of oxalate are well known to those of skill in the art. For example, in the obligate anaerobe Oxalobacter formigenes, the formyl coenzyme A transferase FRC (encoded by the frc gene) transfers a coenzyme A moiety to oxalic acid, forming oxalyl-CoA (see, e.g., Sidhu et al., J. Bacteriol. 179: 3378-81 (1997), the entire contents of which are expressly incorporated herein by reference). Subsequently, the oxalyl-CoA is subject to a reaction mediated by the oxalyl-CoA decarboxylase OXC (encoded by the oxc gene), which leads to the formation of formate and carbon dioxide (see, e.g., Lung et al., J. Bacteriol. 176: 2468-72 (1994), the entire contents of which are expressly incorporated herein by reference). Further, the E. coli protein YfdW (Protein Data Bank Accession No. 1pt5) and YfdU (Protein Data Bank Accession No. E0SNC8) are a formyl-CoA transferase and an oxalyl-CoA decarboxylase that have been shown to be functional homologs of the O. formigenes FRC and OXC enzymes (see, e.g., Toyota et al., J. Bact. 190: 2256-64 (2008); Werther et al., FEBS J. 277: 2628-40 (2010); Fontenot et al., J. Bact. 195: 1446-55 (2013)).

Another oxalate catabolism enzyme, acetyl-CoA:oxalate CoA-transferase, converts acetyl-CoA and oxalate to oxalyl-CoA and acetate. In a non-limiting example, the acetyl-CoA:oxalate CoA-transferase is YfdE from E. coli (e.g., described in Function and X-ray crystal structure of Escherichia coli YfdE; PLoS One. 2013 Jul. 23; 8(7):e67901). Acetyl-CoA substrate a very ubiquitous metabolite in bacteria, such as E. coli, and acetate produced can for example diffuse into the extracellular space without the need of a transporter. In one example, acetyl-CoA:oxalate CoA-transferase reaction can be followed by oxalyl-CoA decarboxylase OXC (encoded by the oxc gene), which leads to the formation of formate and carbon dioxide. Formate can exit the cell, for example through a formate exporter, including but not limited to, OxlT from O. formigenes.

Another exemplary oxalate catabolism enzyme oxalyl-CoA synthetase (OCL; also called oxalate-CoA ligase), which converts oxalate and CoA and ATP to oxalyl-CoA and AMP and di-phosphate. In a non-limiting example, the oxalate-CoA ligase is Saccharomyces cerevisiae acyl-activating enzyme 3 (ScAAE3) (e.g., described in Foster and Nakata, An oxalyl-CoA synthetase is important for oxalate metabolism in Saccharomyces cerevisiae. FEBS Lett. 2014 Jan. 3; 588(1):160-6). In one example, oxalate-CoA ligase can be followed by oxalyl-CoA decarboxylase OXC (encoded by the oxc gene), which leads to the formation of formate and carbon dioxide. Formate can exit the cell, for example through a formate exporter, including but not limited to, OxlT from O. formigenes.

In some embodiments, the genetically engineered bacteria of the disclosure comprise one or more gene(s) and/or gene cassette(s) encoding at least one oxalate catabolism enzyme. In some embodiments, the engineered bacteria comprise one or more gene(s) and/or gene cassette(s) encoding at least one oxalate catabolism enzyme and are capable of converting oxalate into oxalyl-CoA. In some embodiments, the engineered bacteria comprise one or more gene(s) and/or gene cassette(s) encoding at least one oxalate catabolism enzyme and are capable of converting oxalyl-CoA into formate and carbon dioxide. In some embodiments, the engineered bacteria comprise one or more gene(s) and/or gene cassette(s) encoding at least one oxalate catabolism enzyme and are capable of converting oxalate into oxalyl-CoA, and oxalyl-CoA into formate and carbon dioxide. In some embodiments, the engineered bacteria of the disclosure comprise one or more gene(s) and or gene cassette encoding one or more oxalate catabolism enzyme(s) which convert oxalate and formyl CoA into oxalyl-CoA and formate. In some embodiments, the engineered bacteria of the disclosure comprise one or more gene(s) and/or gene cassette(s) encoding one or more oxalate catabolism enzyme(s) which convert oxalate and acetyl-coA into oxalyl-CoA and acetate. In some embodiments, the engineered bacteria of the disclosure comprise one or more gene(s) and/or gene cassette(s) encoding one or more oxalate catabolism enzyme(s) which convert oxalate and CoA into oxalyl-CoA (e.g., by converting one ATP to AMP plus diphosphate). In some embodiments, the engineered bacteria of the disclosure comprise one or more gene(s) and/or gene cassette(s) encoding one or more oxalate catabolism enzyme(s) which convert oxalyl-CoA to carbon dioxide and formyl-CoA. In some embodiments, the engineered bacteria produce formate as a result of oxalate catabolism. In some embodiments, the engineered bacteria produce formate and carbon dioxide as a result of oxalate catabolism. In some embodiments, the engineered bacteria produce acetate as a result of oxalate catabolism. In some embodiments, the engineered bacteria produce acetate and carbon dioxide as a result of oxalate catabolism. In some embodiments, the engineered bacteria produce formate, acetate, and carbon dioxide as a result of oxalate catabolism.

In some embodiments, the genetically engineered bacteria comprise gene sequence(s) encoding one or more oxalate catabolism enzyme (s). In some embodiments, the one or more oxalate catabolism enzyme(s) increases the rate of oxalate and/or oxalyl-CoA catabolism in the cell. In some embodiments, the one or more oxalate catabolism enzyme(s) decreases the level of oxalate in the cell. In some embodiments, the one or more oxalate catabolism enzyme(s) decreases the level of oxalyl-CoA in the cell. In some embodiments, the one or more oxalate catabolism enzyme(s) decreases the level of oxalic acid in the cell.

In some embodiments, the one or more oxalate catabolism enzyme(s) increases the level of oxalyl-CoA in the cell as compared to the level of its corresponding oxalate in the cell. In some embodiments, the one or more oxalate catabolism enzyme(s) increases the level of formate and carbon dioxide in the cell as compared to the level of its corresponding oxalyl-CoA in the cell. In some embodiments, the one or more oxalate catabolism enzyme(s) decreases the level of the oxalate and/or oxalyl CoA as compared to the level of oxalate in the cell.

Enzymes involved in the catabolism of oxalate may be expressed or modified in the bacteria of the invention in order to enhance catabolism of oxalate. Specifically, when at least one oxalate catabolism enzyme is expressed in the engineered bacterial cells of the invention, the engineered bacterial cells convert more oxalate into oxalyl-CoA, or convert more oxalyl-CoA into formate and carbon dioxide when the catabolism enzyme is expressed than unmodified bacteria of the same bacterial subtype under the same conditions. Thus, the genetically engineered bacteria comprising a heterologous gene encoding an oxalate catabolism enzyme can catabolize oxalate and/or oxalyl-CoA to treat disorders in which oxalate is detrimental, such as PHI, PHII, PHIII, and secondary hyperoxaluria, enteric hyperoxaluria, and idiopathic hyperoxaluria.

In one embodiment, the bacterial cell of the invention comprises at least one heterologous gene encoding at least one oxalate catabolism enzyme. In one embodiment, the bacterial cell of the invention comprises at least one heterologous gene encoding an importer of oxalate and at least one heterologous gene encoding at least one oxalate catabolism enzyme. In one embodiment, the bacterial cell of the invention comprises at least one heterologous gene encoding an exporter of formate and at least one heterologous gene encoding at least one oxalate catabolism enzyme. In one embodiment, the bacterial cell of the invention comprises at least one heterologous gene encoding an oxalate:formate antiporter and at least one heterologous gene encoding at least one oxalate catabolism enzyme.

In some embodiments, the invention provides a bacterial cell that comprises at least one heterologous gene encoding at least one oxalate catabolism enzyme operably linked to a first promoter. In one embodiment, the bacterial cell comprises at least one gene encoding at least one oxalate catabolism enzyme from a different organism, e.g., a different species of bacteria. In another embodiment, the bacterial cell comprises more than one copy of a native gene encoding an oxalate catabolism enzyme. In yet another embodiment, the bacterial cell comprises at least one native gene encoding at least one oxalate catabolism enzyme, as well as at least one copy of at least one gene encoding an oxalate catabolism enzyme from a different organism, e.g., a different species of bacteria. In one embodiment, the bacterial cell comprises at least one, two, three, four, five, or six copies of a gene encoding an oxalate catabolism enzyme. In one embodiment, the bacterial cell comprises multiple copies of a gene encoding an oxalate catabolism enzyme.

Oxalate catabolism enzymes are known in the art. In some embodiments, AN oxalate catabolism enzyme is encoded by at least one gene encoding at least one oxalate catabolism enzyme derived from a bacterial species. In some embodiments, an oxalate catabolism enzyme is encoded by a gene encoding an oxalate catabolism enzyme derived from a non-bacterial species. In some embodiments, an oxalate catabolism enzyme is encoded by a gene derived from a eukaryotic species, e.g., a yeast species or a plant species. In one embodiment, an oxalate catabolism enzyme is encoded by a gene derived from a human. In one embodiment, the gene sequence(s) encoding the one or more oxalate catabolism enzyme(s) is derived from an organism of the genus or species that includes, but is not limited to, Bifidobacterium, Bordetella, Bradyrhizobium, Burkholderia, Clostridium, Enterococcus, Escherichia, Eubacterium, Lactobacillus, Magnetospirillium, Mycobacterium, Neurospora, Oxalobacter, e.g., Oxalobacter formigenes, Ralstonia, Rhodopseudomonas, Shigella, Thermoplasma, and Thauera, e.g., Bifidobacterium animalis, Bifidobacterium bifidum, Bifidobacterium infantis, Bifidobacterium lactis, Bifidobacterium longum, Bordatella bronchiseptica, Bordatella parapertussis, Burkholderia fungorum, Burkholderia xenovorans, Bradyrhizobium japonicum, Clostridium acetobutylicum, Clostridium difficile, Clostridium scindens, Clostridium sporogenes, Clostridium tentani, Enterococcus faecalis, Escherichia coli, Eubacterium lentum, Lactobacillus acidophilus, Lactobacillus bulgaricus, Lactobacillus casei, Lactobacillus gasseri, Lactobacillus plantarum, Lactobacillus rhamnosus, Lactococcus lactis, Magnetospirillium magentotaticum, Mycobacterium avium, Mycobacterium intracellulare, Mycobacterium kansasii, Mycobacterium leprae, Mycobacterium smegmatis, Mycobacterium tuberculosis, Mycobacterium ulcerans, Neurospora crassa, Oxalobacter formigenes, Providencia rettgeri, Eubacterium lentum, Ralstonia eutropha, Ralstonia metallidurans, Rhodopseudomonas palustris, Shigella flexneri, Thermoplasma volcanium, and Thauera aromatica.

In one embodiment, one or more oxalate catabolism enzyme(s) encoded by the genetically engineered bacteria are derived from O. formigenes, e.g., oxc and frc described above.

In one embodiment, one or more oxalate catabolism enzyme(s) encoded by the engineered bacteria are derived from Enteroccoccus faecalis. An inducible oxalate catabolism system has been described in Enterococcus faecalis, which comprised homologs to O. formigenes Frc and Oxc (Hokama et al., Oxalate-degrading Enterococcus faecalis. Microbiol. Immunol. 44, 235-240).

In one embodiment, one or more oxalate catabolism enzyme(s) encoded by the engineered bacteria are derived from are from Eubacterium lentum. The oxalate-degrading proteins oxalyl-CoA decarboxylase and formyl-CoA transferase were reportedly isolated from this strain (Ito, H., Kotake, T., and Masai, M. (1996). In vitro degradation of oxalic acid by human feces. Int. J. Urol. 3, 207-211.).

In one embodiment, one or more oxalate catabolism enzyme(s) encoded by the engineered bacteria are derived from Providencia rettgeri, which have shown to have homologs to O. formigenes Frc and Oxc (e.g., as described in Abratt and Reid, Oxalate-degrading bacteria of the human gut as probiotics in the management of kidney stone disease; Adv Appl Microbiol. 2010; 72:63-87, and references therein).

In one embodiment, one or more oxalate catabolism enzyme(s) encoded by the engineered bacteria are derived from E. coli, e.g. from the yfdXWUVE operon. For example, the ydfU is thought to be a oxc homolog. In one embodiment, one or more oxalate catabolism enzyme(s) encoded by the engineered bacteria are derived from Lactobacillus and/or Bifidobacterium species. In a non-limiting example one or more oxalate catabolism enzyme(s) are derived from oxc and frc homologs Lactobacillus and/or Bifidobacterium species. Non-limiting examples of such Lactobacillus species include Lactobacillus, plantarum, Lactobacillus brevis, Lactobacillus acidophilus, Lactobacillus casei, Lactobacillus gasseri, Lactobacillus rhamnosus, and Lactobacillus salivarius. Non-limiting examples of such Bifidobacterium species include Bifidobacterium infantis, Bifidobacterium animalis, Bifidobacterium breve, Bifidobacterium longum, Bifidobacterium lactis, and Bifidobacterium adolescentis.

In one embodiment, the gene sequence(s) encoding the one or more oxalate catabolism enzyme(s) has been codon-optimized for use in the recombinant bacterial cell of the invention. In one embodiment, the gene sequence(s) encoding the one or more oxalate catabolism enzyme(s) has been codon-optimized for use in Escherichia coli. In one embodiment, the gene sequence(s) encoding the one or more oxalate catabolism enzyme(s) has not been codon-optimized for use in Escherichia coli. In another embodiment, the gene sequence(s) encoding the one or more oxalate catabolism enzyme(s) has been codon-optimized for use in Lactococcus. When the gene sequence(s) encoding the one or more oxalate catabolism enzyme(s) is expressed in the recombinant bacterial cells of the invention, the bacterial cells catabolize more oxalate or oxalyl-CoA than unmodified bacteria of the same bacterial subtype under the same conditions (e.g., culture or environmental conditions). Thus, the genetically engineered bacteria comprising at least one heterologous gene encoding at least one oxalate catabolism enzyme may be used to catabolize excess oxalate, oxalic acid, and/or oxalyl-CoA to treat a disorder in which oxalate is detrimental, such as PHI, PHII, PHIII, and secondary hyperoxaluria, enteric hyperoxaluria, and idiopathic hyperoxaluria.

The present invention further comprises genes encoding functional fragments of an oxalate catabolism enzyme or functional variants of an oxalate catabolism enzyme. As used herein, the term “functional fragment thereof” or “functional variant thereof” of an oxalate catabolism enzyme relates to an element having qualitative biological activity in common with the wild-type oxalate catabolism enzyme from which the fragment or variant was derived. For example, a functional fragment or a functional variant of a mutated oxalate catabolism enzyme is one which retains essentially the same ability to catabolize oxalyl-CoA as the oxalate catabolism enzyme from which the functional fragment or functional variant was derived. For example, a polypeptide having oxalate catabolism enzyme activity may be truncated at the N-terminus or C-terminus and the retention of oxalate catabolism enzyme activity assessed using assays known to those of skill in the art, including the exemplary assays provided herein. In one embodiment, the recombinant bacterial cell of the invention comprises a heterologous gene encoding an oxalate catabolism enzyme functional variant. In another embodiment, the recombinant bacterial cell of the invention comprises a heterologous gene encoding an oxalate catabolism enzyme functional fragment.

Assays for testing the activity of an oxalate catabolism enzyme, an oxalate catabolism enzyme functional variant, or an oxalate catabolism enzyme functional fragment are well known to one of ordinary skill in the art. For example, oxalate catabolism can be assessed by expressing the protein, functional variant, or fragment thereof, in a recombinant bacterial cell that lacks endogenous oxalate catabolism enzyme activity. Oxalate catabolism activity can be assessed by quantifying oxalate degradation in the culture media as described by Federici et al., Appl. Environ. Microbiol. 70: 5066-73 (2004), the entire contents of which are expressly incorporated herein by reference. Formyl-CoA transferase and oxalyl-CoA decarboxylase activities can be measured by capillary electrophoresis as described in Turroni et al., J. Appl. Microbiol. 103: 1600-9 (2007).

As used herein, the term “percent (%) sequence identity” or “percent (%) identity,” also including “homology,” is defined as the percentage of amino acid residues or nucleotides in a candidate sequence that are identical with the amino acid residues or nucleotides in the reference sequences after aligning the sequences and introducing gaps, if necessary, to achieve the maximum percent sequence identity, and not considering any conservative substitutions as part of the sequence identity. Optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl. Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.). In one embodiment, the gene or protein is at least 90%, 91%, 92%, 93%, 94$, 95%, 96%, 97%, 98%, 99% or 100% identical to a gene or protein disclosed herein.

The present invention encompasses genes encoding an oxalate catabolism enzyme comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T. Similarly, contemplated is replacing a basic amino acid with another basic amino acid (e.g., replacement among Lys, Arg, His), replacing an acidic amino acid with another acidic amino acid (e.g., replacement among Asp and Glu), replacing a neutral amino acid with another neutral amino acid (e.g., replacement among Ala, Gly, Ser, Met, Thr, Leu, Ile, Asn, Gln, Phe, Cys, Pro, Trp, Tyr, Val).

In some embodiments, the gene encoding an oxalate catabolism enzyme is mutagenized; mutants exhibiting increased activity are selected; and the mutagenized gene encoding the oxalate catabolism enzyme is isolated and inserted into the bacterial cell of the invention. In one embodiment, spontaneous mutants that arise that allow bacteria to grow on oxalate as the sole carbon source can be screened for and selected. The gene comprising the modifications described herein may be present on a plasmid or chromosome. Non-limiting examples of oxalate catabolism enzymes of the disclosure are listed in Table 2.

TABLE 2
Oxalate Catabolism Enzyme Polynucleotide Sequences

| Description | SEQ ID NO |
|---|---|
| frc (formyl-CoA transferase from O. formigenes) | SEQ ID NO: 1 |
| oxc (oxalylCoA decarboxylase from O. formigenes) also | SEQ ID NO: 2 |
| referred to as oxdc (oxalate decarboxylase) | |
| ScAAE3 (oxalate-CoA ligase from S. cerevisiae) | SEQ ID NO: 3 |
| yfdE (Acetyl-CoA: oxalate CoA-transferase from E. coli) | SEQ ID NO: 4 |

In one embodiment, the gene sequence(s) encoding the one or more oxalate catabolism enzyme(s) comprises a formyl-CoA:oxalate CoA-transferase sequence. In one embodiment, the formyl-CoA:oxalate CoA-transferase is frc, e.g., from O. formigenes. Accordingly, in one embodiment, the frc gene has at least about 80% identity with the entire sequence of SEQ ID NO: 1. Accordingly, in one embodiment, the frc gene has at least about 90% identity with the entire sequence of SEQ ID NO: 1. Accordingly, in one embodiment, the frc gene has at least about 95% identity with the entire sequence of SEQ ID NO: 1. Accordingly, in one embodiment, the frc gene has at least about 85%, 86%, 87%, 88%, 89%, 90%, 91%, 92%, 93%, 94%, 95%, 96%, 97%, 98%, or 99% identity with the entire sequence of SEQ ID NO: 1. In another embodiment, the frc gene comprises the sequence of SEQ ID NO: 1. In yet another embodiment the frc gene consists of the sequence of SEQ ID NO:1.

In one embodiment, the gene sequence(s) encoding the one or more oxalate catabolism enzyme(s) comprises a oxalyl-CoA decarboxylase sequence. In one embodiment, the oxalyl-CoA decarboxylase is oxc, e.g., from O. formigenes. Accordingly, in one embodiment, the oxc gene has at least about 80% identity with the entire sequence of SEQ ID NO: 2. Accordingly, in one embodiment, the oxc gene has at least about 90% identity with the entire sequence of SEQ ID NO: 2. Accordingly, in one embodiment, the oxc gene has at least about 95% identity with the entire sequence of SEQ ID NO: 2. Accordingly, in one embodiment, the oxc gene has at least about 85%, 86%, 87%, 88%, 89%, 90%, 91%, 92%, 93%, 94%, 95%, 96%, 97%, 98%, or 99% identity with the entire sequence of SEQ ID NO: 2. In another embodiment, the oxc gene comprises the sequence of SEQ ID NO: 2. In yet another embodiment the oxc gene consists of the sequence of SEQ ID NO: 2. In another embodiment, the oxc gene consists of the sequence of SEQ ID NO: 2.

In one embodiment, the at least one gene encoding the at least one oxalate catabolism enzyme comprises an oxalate-CoA ligase sequence. In one embodiment, the oxalate-CoA ligase is ScAAE3 from S. cerevisiae. Accordingly, in one embodiment, the ScAAE3 gene has at least about 80% identity with the entire sequence of SEQ ID NO: 3. Accordingly, in one embodiment, the ScAAE3 gene has at least about 90% identity with the entire sequence of SEQ ID NO: 3. Accordingly, in one embodiment, the ScAAE3 gene has at least about 95% identity with the entire sequence of SEQ ID NO: 3. Accordingly, in one embodiment, the ScAAE3 gene has at least about 85%, 86%, 87%, 88%, 89%, 90%, 91%, 92%, 93%, 94%, 95%, 96%, 97%, 98%, or 99% identity with the entire sequence of SEQ ID NO: 3. In another embodiment, the ScAAE3 gene comprises the sequence of SEQ ID NO: 3. In yet another embodiment the ScAAE3 gene consists of the sequence of SEQ ID NO: 3.

In one embodiment, the at least one gene encoding the at least one oxalate catabolism enzyme comprises an acetyl-CoA:oxalate CoA-transferase sequence. In one embodiment, the acetyl-CoA:oxalate CoA-transferase is YfdE from E. coli from S. cerevisiae. Accordingly, in one embodiment, the YfdE gene has at least about 80% identity with the entire sequence of SEQ ID NO: 4. Accordingly, in one embodiment, the YfdE gene has at least about 90% identity with the entire sequence of SEQ ID NO: 4. Accordingly, in one embodiment, the YfdE gene has at least about 95% identity with the entire sequence of SEQ ID NO: 4. Accordingly, in one embodiment, the YfdE gene has at least about 85%, 86%, 87%, 88%, 89%, 90%, 91%, 92%, 93%, 94%, 95%, 96%, 97%, 98%, or 99% identity with the entire sequence of SEQ ID NO: 4. In another embodiment, the YfdE gene comprises the sequence of SEQ ID NO: 4. In yet another embodiment the YfdE gene consists of the sequence of SEQ ID NO: 4.

Table 3 lists non-limiting examples of oxalate catabolism enzyme polypeptide sequences.

TABLE 3
Polypeptide Sequences of Oxalate Catabolism Enzymes

| Description | SEQ ID NO |
|---|---|
| Frc (Formyl-CoA transferase from O. formigenes) | SEQ ID NO: 5 |
| Oxc (oxalylCoA decarboxylase from O. formigenes); | SEQ ID NO: 6 |
| also referred to as oxcd herein | |
| ScAAE3 (Oxalate-CoA ligase from S. cerevisiae) | SEQ ID NO: 7 |
| yfdE (Acetyl-CoA: oxalate CoA-transferase from | SEQ ID NO: 8 |
| E. coli) | |
| yfdW (formyl CoA transferase from E. coli) | SEQ ID NO: 9 |
| yfdU (oxalyl-CoA decarboxylase E. coli) | SEQ ID NO: 10 |

In one embodiment, one or more polypeptide(s) encoded by the oxalate catabolism cassette(s) and expressed by the genetically engineered bacteria comprises a formyl-CoA transferase, e.g. frc from O. formigenes. In one embodiment the polypeptide(s) have at least about 80% identity with SEQ ID NO: 5. In another embodiment, one or more polypeptide(s) encoded by the oxalate catabolism gene(s) or gene cassette(s) and expressed by the genetically engineered bacteria have at least about 85% identity with SEQ ID NO: 5. In one embodiment, one or more polypeptide(s) encoded by the oxalate catabolism gene(s) or gene cassette(s) and expressed by the genetically engineered bacteria have at least about 90% identity with SEQ ID NO: 5. In one embodiment, one or more polypeptide(s) encoded by the oxalate catabolism gene(s) or gene cassette(s) and expressed by the genetically engineered bacteria have at least about 95% identity with SEQ ID NO: 5. In another embodiment, one or more polypeptide(s) encoded by the oxalate catabolism gene(s) or gene cassette(s) and expressed by the genetically engineered bacteria have at least about 96%, 97%, 98%, or 99% identity with SEQ ID NO: 5. Accordingly, in one embodiment, one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 5. In another embodiment, one or more polypeptide(s) encoded by the oxalate catabolism gene(s) or gene cassette(s) and expressed by the genetically engineered bacteria comprise the sequence of SEQ ID NO: 5. In yet another embodiment one or more polypeptide(s) encoded by the oxalate catabolism gene(s) or gene cassette(s) and expressed by the genetically engineered bacteria consist of the sequence of SEQ ID NO: 5.

In one embodiment, one or more polypeptide(s) encoded by the oxalate catabolism cassette(s) and expressed by the engineered bacteria comprises a oxalyl-CoA decarboxylase, e.g. oxc from O. formigenes. In one embodiment the polypeptide(s) have at least about 80% identity with SEQ ID NO: 6. In another embodiment, one or more polypeptide(s) encoded by the oxalate catabolism gene(s) or gene cassette(s and expressed by the engineered bacteria have at least about 85% identity with SEQ ID NO: 6. In one embodiment, one or more polypeptide(s) encoded by the oxalate catabolism gene(s) or gene cassette(s) and expressed by the engineered bacteria have at least about 90% identity with SEQ ID NO: 6. In one embodiment, one or more polypeptide(s) encoded by the oxalate catabolism gene(s) or gene cassette(s) and expressed by the engineered bacteria have at least about 95% identity with SEQ ID NO: 6. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 6. Accordingly, in one embodiment, one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 6. In another embodiment, one or more polypeptide(s) encoded by the oxalate catabolism gene(s) or gene cassette(s) and expressed by the engineered bacteria comprise the sequence of SEQ ID NO: 6. In yet another embodiment one or more polypeptide(s) encoded by the oxalate catabolism gene(s) or gene cassette(s) and expressed by the genetically engineered bacteria consist of the sequence of SEQ ID NO: 6.

In one embodiment, one or more polypeptide(s) encoded by the oxalate catabolism gene(s) or gene cassette(s) and expressed by the engineered bacteria comprises an oxalate-CoA ligase, e.g. ScAAE3 from S cerevisiae. In one embodiment the polypeptide(s) have at least about 80% identity with SEQ ID NO: 7. In another embodiment, one or more polypeptide(s) encoded by the oxalate catabolism gene(s) or gene cassette(s and expressed by the engineered bacteria have at least about 85% identity with SEQ ID NO: 7. In one embodiment, one or more polypeptide(s) encoded by the oxalate catabolism gene(s) or gene cassette(s) and expressed by the engineered bacteria have at least about 90% identity with SEQ ID NO: 7. In one embodiment, one or more polypeptide(s) encoded by the oxalate catabolism gene(s) or gene cassette(s) and expressed by the engineered bacteria have at least about 95% identity with SEQ ID NO: 7. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 7. Accordingly, in one embodiment, one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 7. In another embodiment, one or more polypeptide(s) encoded by the oxalate catabolism gene(s) or gene cassette(s) and expressed by the engineered bacteria comprise the sequence of SEQ ID NO: 7. In yet another embodiment one or more polypeptide(s) encoded by the oxalate catabolism cassette(s) and expressed by the genetically engineered bacteria consist of the sequence of SEQ ID NO: 7.

In one embodiment, one or more polypeptide(s) encoded by the oxalate catabolism cassette(s) and expressed by the engineered bacteria comprises an Acetyl-CoA:oxalate CoA-transferase from, e.g. YfdE from E. coli. In one embodiment the polypeptide(s) have at least about 80% identity with SEQ ID NO: 8. In another embodiment, one or more polypeptide(s) encoded by the oxalate catabolism gene(s) or gene cassette(s and expressed by the engineered bacteria have at least about 85% identity with SEQ ID NO: 8. In one embodiment, one or more polypeptide(s) encoded by the oxalate catabolism gene(s) or gene cassette(s) and expressed by the engineered bacteria have at least about 90% identity with SEQ ID NO: 8. In one embodiment, one or more polypeptide(s) encoded by the oxalate catabolism gene(s) or gene cassette(s) and expressed by the engineered bacteria have at least about 95% identity with SEQ ID NO: 8. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 8. Accordingly, in one embodiment, one or more polypeptide(s) encoded by the oxalate catabolism gene(s) or gene cassette(s) and expressed by the genetically engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 8. In another embodiment, one or more polypeptide(s) encoded by the oxalate catabolism gene(s) or gene cassette(s) and expressed by the engineered bacteria comprise the sequence of SEQ ID NO: 8. In yet another embodiment one or more polypeptide(s) encoded by the oxalate catabolism gene(s) or gene cassette(s) and expressed by the engineered bacteria consist of the sequence of with SEQ ID NO: 8.

In one embodiment, one or more polypeptide(s) encoded by the oxalate catabolism gene(s) or gene cassette(s) and expressed by the genetically engineered bacteria comprises a formyl CoA transferase, e.g., yfdW from E. coli. In one embodiment the polypeptide(s) have at least about 80% identity with SEQ ID NO: 9. In another embodiment, one or more polypeptide(s) encoded by the oxalate catabolism gene(s) or gene cassette(s and expressed by the engineered bacteria have at least about 85% identity with SEQ ID NO: 9. In one embodiment, one or more polypeptide(s) encoded by the oxalate catabolism gene(s) or gene cassette(s) and expressed by the engineered bacteria have at least about 90% identity with SEQ ID NO: 9. In one embodiment, one or more polypeptide(s) encoded by the oxalate catabolism gene(s) or gene cassette(s) and expressed by the engineered bacteria have at least about 95% identity with SEQ ID NO: 9. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 9. Accordingly, in one embodiment, one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 9. In another embodiment, one or more polypeptide(s) encoded by the oxalate catabolism gene(s) or gene cassette(s) and expressed by the engineered comprise the sequence of SEQ ID NO: 9. In yet another embodiment one or more polypeptide(s) encoded by the oxalate catabolism gene(s) or gene cassette(s) and expressed by the engineered bacteria consist of the sequence of SEQ ID NO: 9.

In one embodiment, one or more polypeptide(s) encoded by the oxalate catabolism gene(s) or gene cassette(s) and expressed by the engineered bacteria comprises a oxalyl-CoA decarboxylase, e.g., yfdU from E. coli. In one embodiment the polypeptide(s) have at least about 80% identity with SEQ ID NO: 10. In another embodiment, one or more polypeptide(s) encoded by the oxalate catabolism gene(s) or gene cassette(s and expressed by the engineered bacteria have at least about 85% identity with SEQ ID NO: 10. In one embodiment, one or more polypeptide(s) encoded by the oxalate catabolism gene(s) or gene cassette(s) and expressed by the engineered bacteria have at least about 90% identity with SEQ ID NO: 10. In one embodiment, one or more polypeptide(s) encoded by the oxalate catabolism gene(s) or gene cassette(s) and expressed by the engineered bacteria have at least about 95% identity with SEQ ID NO: 10. In another embodiment, one or more polypeptide(s) encoded by the oxalate catabolism gene(s) or gene cassette(s) and expressed by the engineered bacteria have at least about 96%, 97%, 98%, or 99% identity with SEQ ID NO: 10. Accordingly, in one embodiment, one or more polypeptide(s) encoded by the oxalate catabolism gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 10. In another embodiment, one or more polypeptide(s) encoded by the oxalate catabolism gene(s) or gene cassette(s) and expressed by the engineered bacteria comprise the sequence of SEQ ID NO: 10. In yet another embodiment one or more polypeptide(s) encoded by the oxalate catabolism gene(s) or gene cassette(s) and expressed by the engineered bacteria consist of the sequence of SEQ ID NO: 10.

In one embodiment, the gene sequence(s) encoding the one or more oxalate catabolism enzyme(s) is directly operably linked to a first promoter. In another embodiment, the gene sequence(s) encoding the one or more oxalate catabolism enzyme(s) is indirectly operably linked to a first promoter. In one embodiment, the promoter is not operably linked with the at least one gene encoding the oxalate catabolism enzyme in nature.

In some embodiments, the gene sequence(s) encoding the one or more oxalate catabolism enzyme(s) is expressed under the control of a constitutive promoter. In another embodiment, the gene sequence(s) encoding the one or more oxalate catabolism enzyme(s) is expressed under the control of an inducible promoter. In some embodiments, the gene sequence(s) encoding the one or more oxalate catabolism enzyme(s) is expressed under the control of a promoter that is directly or indirectly induced by exogenous environmental conditions. In one embodiment, the gene sequence(s) encoding the one or more oxalate catabolism enzyme(s) is expressed under the control of a promoter that is directly or indirectly induced by low-oxygen or anaerobic conditions, such as the environmental conditions of a mammalian gut, wherein expression of the gene sequence(s) encoding the one or more oxalate catabolism enzyme(s) is activated under low-oxygen or anaerobic environments, such as the environment of a mammalian gut. In some embodiments, the gene sequence(s) encoding the one or more oxalate catabolism enzyme(s) is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, a P_{TetR} promoter, and a P_{LacI} promoter, each of which are described in more detail herein. Inducible promoters are described in more detail infra.

The at least one gene encoding the at least one oxalate catabolism enzyme may be present on a plasmid or chromosome in the bacterial cell. In one embodiment, the gene sequence(s) encoding the one or more oxalate catabolism enzyme(s) is located on a plasmid in the bacterial cell. In another embodiment, the gene sequence(s) encoding the one or more oxalate catabolism enzyme(s) is located in the chromosome of the bacterial cell. In yet another embodiment, a native copy of the gene sequence(s) encoding the one or more oxalate catabolism enzyme(s) is located in the chromosome of the bacterial cell, and at least one gene encoding at least one oxalate catabolism enzyme from a different species of bacteria is located on a plasmid in the bacterial cell. In yet another embodiment, a native copy of the gene sequence(s) encoding the one or more oxalate catabolism enzyme(s) is located on a plasmid in the bacterial cell, and at least one gene encoding the at least one oxalate catabolism enzyme from a different species of bacteria is located on a plasmid in the bacterial cell. In yet another embodiment, a native copy of the gene sequence(s) encoding the one or more oxalate catabolism enzyme(s) is located in the chromosome of the bacterial cell, and at least one gene encoding the at least one oxalate catabolism enzyme from a different species of bacteria is located in the chromosome of the bacterial cell.

In some embodiments, the gene sequence(s) encoding the one or more oxalate catabolism enzyme(s) is expressed on a low-copy plasmid. In some embodiments, the gene sequence(s) encoding the one or more oxalate catabolism enzyme(s) is expressed on a high-copy plasmid. In some embodiments, the high-copy plasmid may be useful for increasing expression of the at least one oxalate catabolism enzyme, thereby increasing the catabolism of oxalate, oxalic acid, and/or oxalyl-CoA.

In some embodiments, a recombinant bacterial cell of the invention comprising at least one gene encoding at least one oxalate catabolism enzyme expressed on a high-copy plasmid does not increase oxalate catabolism or decrease oxalate and/or oxalic acid levels as compared to a recombinant bacterial cell comprising the same gene expressed on a low-copy plasmid in the absence of a heterologous importer of oxalate and additional copies of a native importer of oxalate. Furthermore, in some embodiments that incorporate an importer of oxalate into the recombinant bacterial cell, there may be additional advantages to using a low-copy plasmid comprising the gene sequence(s) encoding the one or more oxalate catabolism enzyme(s) in conjunction in order to enhance the stability of expression of the oxalate catabolism enzyme, while maintaining high oxalate catabolism and to reduce negative selection pressure on the transformed bacterium. In alternate embodiments, the importer of oxalate is used in conjunction with a high-copy plasmid.

Transporter (Importer) of Oxalate

The uptake of oxalate into the anaerobic bacterium, Oxalobacter formigenes, has been found to occur via the oxalate transporter OxlT (see, e.g., Ruan et al., J. Biol. Chem. 267: 10537-43 (1992), the entire contents of which are expressly incorporated herein by reference). OxlT catalyzes the exchange of extracellular oxalate, a divalent anion, for intracellular formate, a monovalent cation that is derived from the decarboxylation of oxalate, thus generating a proton-motive force. Other proteins that mediate the import of oxalate are well known to those of skill in the art.

Oxalate transporters, e.g., oxalate importers, may be expressed or modified in the bacteria of the invention in order to enhance oxalate transport into the cell. Specifically, when the importer of oxalate is expressed in the recombinant bacterial cells of the invention, the bacterial cells import more oxalate into the cell when the importer is expressed than unmodified bacteria of the same bacterial subtype under the same conditions. Thus, the genetically engineered bacteria comprising one or more heterologous gene sequence(s) encoding an importer of oxalate may be used to import oxalate into the bacteria so that any gene sequence(s) encoding an oxalate catabolism enzyme(s) expressed in the organism can be used to treat disorders in which oxalate is detrimental, such as PHI, PHII, PHIII, and secondary hyperoxaluria, enteric hyperoxaluria, and idiopathic hyperoxaluria. In one embodiment, the bacterial cell of the invention comprises a heterologous gene sequence(s) encoding a transporter (importer) of oxalate. In one embodiment, the bacterial cell of the invention comprises a heterologous gene sequence(s) encoding transporter of oxalate and one or more heterologous gene sequence(s) encoding one or more oxalate catabolism enzyme(s). In one embodiment, the bacterial cell of the invention comprises a heterologous gene sequence(s) encoding transporter of oxalate and one or more heterologous gene sequence(s) encoding one or more polypeptides selected from a formate exporter, an oxalate:formate antiporter, and combinations thereof. In one embodiment, the bacterial cell of the invention comprises a heterologous gene sequence(s) encoding a transporter of oxalate, one or more heterologous gene sequence(s) encoding one or more oxalate catabolism enzyme(s), and one or more heterologous gene sequence(s) encoding one or more polypeptides selected from a formate exporter, an oxalate:formate antiporter, and combinations thereof.

Thus, in some embodiments, the invention provides a bacterial cell that comprises one or more heterologous gene sequence(s) encoding an oxalate catabolism enzyme operably linked to a first promoter and one or more heterologous gene sequence(s) encoding an transporter (importer) of oxalate. In some embodiments, the invention provides a bacterial cell that comprises one or more heterologous gene sequence(s) encoding an transporter (importer) of oxalate operably linked to the first promoter. In another embodiment, the invention provides a bacterial cell that comprises one or more heterologous gene sequence(s) encoding one or more oxalate catabolism enzyme(s) operably linked to a first promoter and one or more heterologous gene sequence(s) encoding an transporter (importer) of oxalate operably linked to a second promoter. In one embodiment, the first promoter and the second promoter are separate copies of the same promoter. In another embodiment, the first promoter and the second promoter are different promoters.

In one embodiment, the bacterial cell comprises one or more gene sequence(s) encoding an transporter (importer) of oxalate from a different organism, e.g., a different species of bacteria. In one embodiment, the bacterial cell comprises one or more native gene sequence(s) encoding an transporter (importer) of oxalate. In some embodiments, the one or more native gene sequence(s) encoding an transporter (importer) of oxalate is not modified. In another embodiment, the bacterial cell comprises more than one copy of one or more native gene sequence(s) encoding an transporter (importer) of oxalate. In yet another embodiment, the bacterial cell comprises a copy of one or more gene sequence(s) encoding a native transporter (importer) of oxalate, as well as one or more copy of one or more heterologous gene sequence(s) encoding an transporter of oxalate from a different bacterial species. In one embodiment, the bacterial cell comprises one or more, two, three, four, five, or six copies of the one or more heterologous gene sequence(s) encoding an transporter of oxalate. In one embodiment, the bacterial cell comprises multiple copies of the one or more heterologous gene sequence(s) encoding an transporter of oxalate.

In some embodiments, the transporter of oxalate is encoded by an transporter of oxalate gene derived from a bacterial genus or species, including but not limited to, Oxalobacter. In some embodiments, the transporter of oxalate gene is derived from a bacteria of the species Oxalobacter formigenes. In some embodiments, the transporter is the OxlT Oxalate:Formate Antiporter from Oxalobacter formigenes

In other embodiments, transporter of oxalate is encoded by a gene selected from the oxalate:formate antiporter (OFA) family. The OFA family members belong to the major facilitator superfamily and are widely distributed in nature, being present in the bacterial, archaeal, and eukaryotic kingdoms (see, e.g., Pao et al., Major Facilitator Superfamily Microbiol. Mol. Biol. Rev. March 1998 vol 62 no. 1-34). In a non-limiting example, the transporter is a homolog and/or ortholog of the Oxalobacter formigenes oxalate:formate antiporter. In another non-limiting example, the transporter is a bacterially derived homolog and/or ortholog of the Oxalobacter formigenes oxalate:formate antiporter (OxlT). The present invention further comprises genes encoding functional fragments of an transporter of oxalate or functional variants of an transporter of oxalate. As used herein, the term “functional fragment thereof” or “functional variant thereof” of an transporter of oxalate relates to an element having qualitative biological activity in common with the wild-type transporter of oxalate from which the fragment or variant was derived. For example, a functional fragment or a functional variant of a mutated transporter of oxalate protein is one which retains essentially the same ability to import oxalate into the bacterial cell as does the transporter protein from which the functional fragment or functional variant was derived. In one embodiment, the recombinant bacterial cell of the invention comprises one or more heterologous gene sequence(s) encoding a functional fragment of an transporter of oxalate. In another embodiment, the recombinant bacterial cell of the invention comprises one or more heterologous gene sequence(s) encoding a functional variant of an transporter of oxalate.

Assays for testing the activity of an transporter of oxalate, an transporter of oxalate functional variant, or an transporter of oxalate functional fragment are well known to one of ordinary skill in the art. For example, oxalate import can be assessed by preparing detergent-extracted proteoliposomes from recombinant bacterial cells expressing the protein, functional variant, or fragment thereof, and determining [¹⁴C]oxalate uptake as described in Abe et al., J. Biol. Chem. 271: 6789-93 (1996), the entire contents of which are expressly incorporated herein by reference.

In one embodiment the genes encoding the transporter of oxalate have been codon-optimized for use in the host organism. In one embodiment, the genes encoding the transporter of oxalate have been codon-optimized for use in Escherichia coli.

The present invention also encompasses genes encoding an transporter of oxalate comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the one or more gene sequence(s) encoding an transporter of oxalate is mutagenized; mutants exhibiting increased oxalate transport are selected; and the mutagenized one or more gene sequence(s) encoding an transporter of oxalate is isolated and inserted into the bacterial cell of the invention. In some embodiments, the one or more gene sequence(s) encoding an transporter of oxalate is mutagenized; mutants exhibiting decreased oxalate transport are selected; and the mutagenized one or more gene sequence(s) encoding an transporter of oxalate is isolated and inserted into the bacterial cell of the invention. The transporter modifications described herein may be present on a plasmid or chromosome.

Table 4 lists polypeptide and polynucleotide sequences for a non-limiting example of an Oxalate:formate antiporter.

TABLE 4
OxlT sequences

| Description | SEQ ID NO |
|---|---|
| OxlT coding region (oxalate: formate | SEQ ID NO: 11 |
| antiporter from O. formigenes) | |
| OxlT (oxalate: formate antiporter from | SEQ ID NO: 12 |
| O. formigenes) | |

In one embodiment, the oxalate importer is the oxalate:formate antiporter OxlT. In one embodiment, the OxlT gene has at least about 80% identity to SEQ ID NO: 11. Accordingly, in one embodiment, the OxlT gene has at least about 90% identity to SEQ ID NO: 11. Accordingly, in one embodiment, the OxlT gene has at least about 95% identity to SEQ ID NO: 11. Accordingly, in one embodiment, the OxlT gene has at least about 85%, 86%, 87%, 88%, 89%, 90%, 91%, 92%, 93%, 94%, 95%, 96%, 97%, 98%, or 99% identity to SEQ ID NO: 11. In another embodiment, the OxlT gene comprises the sequence of SEQ ID NO: 11. In yet another embodiment the OxlT gene consists of the sequence of SEQ ID NO: 11.

In one embodiment, one or more polypeptide(s) encoded by one or more gene(s) or gene cassette(s) and expressed by the genetically engineered bacteria is the oxalate:formate antiporter OxlT. In one embodiment the polypeptide(s) have at least about 80% identity with SEQ ID NO: 12. In another embodiment, one or more polypeptide(s) encoded by one or more gene(s) or gene cassette(s) and expressed by the engineered bacteria have at least about 85% identity with SEQ ID NO: 12. In one embodiment, one or more polypeptide(s) encoded by one or more gene(s) or gene cassette(s) and expressed by the engineered bacteria have at least about 90% identity with SEQ ID NO: 12. In one embodiment, one or more polypeptide(s) encoded by one or more gene(s) or gene cassette(s) and expressed by the engineered bacteria have at least about 95% identity with SEQ ID NO: 12. In another embodiment, one or more polypeptide(s) encoded by one or more gene(s) or gene cassette(s) and expressed by the engineered bacteria have at least about 96%, 97%, 98%, or 99% identity with SEQ ID NO: 12. Accordingly, in one embodiment, one or more polypeptide(s) encoded by one or more gene(s) or gene cassette(s) and expressed by the engineered bacteria have at least about 80%, 81%, 82%, 83%, 84%, 85%, 86%, 87%, 88%, 89%, 90%, 91%, 92%, 93%, 94%, 95%, 96%, 97%, 98%, or 99% identity with SEQ ID NO: 12. In another embodiment, one or more polypeptide(s) encoded by one or more gene(s) or gene cassette(s) and expressed by the engineered bacteria comprise the sequence of SEQ ID NO: 12. In yet another embodiment one or more polypeptide(s) encoded by one or more gene(s) or gene cassette(s) and expressed by the engineered bacteria consist of the sequence of SEQ ID NO: 12.

In some embodiments, the bacterial cell comprises one or more heterologous gene sequence(s) encoding at least one oxalate catabolism enzyme(s) operably linked to a first promoter and one or more heterologous gene sequence(s) encoding an importer of oxalate. In some embodiments, the one or more heterologous gene sequence(s) encoding an importer of oxalate is operably linked to the first promoter. In other embodiments, the one or more heterologous gene sequence(s) encoding an importer of oxalate is operably linked to a second promoter. In one embodiment, the one or more gene sequence(s) encoding an importer of oxalate is directly operably linked to the second promoter. In another embodiment, the one or more gene sequence(s) encoding an importer of oxalate is indirectly operably linked to the second promoter.

In some embodiments, expression of one or more gene sequence(s) encoding an importer of oxalate is controlled by a different promoter than the promoter that controls expression of the gene sequence(s) encoding the one or more oxalate catabolism enzyme(s). In some embodiments, expression of the one or more gene sequence(s) encoding an importer of oxalate is controlled by the same promoter that controls expression of the one or more oxalate catabolism enzyme(s). In some embodiments, one or more gene sequence(s) encoding an importer of oxalate and the oxalate catabolism enzyme are divergently transcribed from a promoter region. In some embodiments, expression of each of genes encoding the gene sequence(s) encoding an importer of oxalate and the gene sequence(s) encoding the one or more oxalate catabolism enzyme(s) is controlled by different promoters.

In one embodiment, the promoter is not operably linked with the one or more gene sequence(s) encoding an importer of oxalate in nature. In some embodiments, the one or more gene sequence(s) encoding an importer of oxalate is controlled by its native promoter. In some embodiments, the one or more gene sequence(s) encoding an importer of oxalate is controlled by an inducible promoter. In some embodiments, the one or more gene sequence(s) encoding the importer of oxalate is controlled by a promoter that is stronger than its native promoter. In some embodiments, the one or more gene sequence(s) encoding an importer of oxalate is controlled by a constitutive promoter.

In another embodiment, the promoter is an inducible promoter. Inducible promoters are described in more detail infra.

In one embodiment, the one or more gene sequence(s) encoding an importer of oxalate is located on a plasmid in the bacterial cell. In another embodiment, the one or more gene sequence(s) encoding an importer of oxalate is located in the chromosome of the bacterial cell. In yet another embodiment, a native copy of the one or more gene sequence(s) encoding an importer of oxalate is located in the chromosome of the bacterial cell, and a copy of one or more gene sequence(s) encoding an importer of oxalate from a different species of bacteria is located on a plasmid in the bacterial cell. In yet another embodiment, a native copy of the one or more gene sequence(s) encoding an importer of oxalate is located on a plasmid in the bacterial cell, and a copy of one or more gene sequence(s) encoding an importer of oxalate from a different species of bacteria is located on a plasmid in the bacterial cell. In yet another embodiment, a native copy of the one or more gene sequence(s) encoding an importer of oxalate is located in the chromosome of the bacterial cell, and a copy of the one or more gene sequence(s) encoding an importer of oxalate from a different species of bacteria is located in the chromosome of the bacterial cell.

In some embodiments, the at least one native gene encoding the importer of oxalate in the bacterial cell is not modified, and one or more additional copies of the native importer of oxalate are inserted into the genome. In one embodiment, the one or more additional copies of the native importer that is inserted into the genome are under the control of the same inducible promoter that controls expression of the one or more gene sequence(s) encoding the oxalate catabolism enzyme, e.g., the FNR responsive promoter, or a different inducible promoter than the one that controls expression of the at least one oxalate catabolism enzyme, or a constitutive promoter. In alternate embodiments, the at least one native gene encoding the importer is not modified, and one or more additional copies of the importer from a different bacterial species is inserted into the genome of the bacterial cell. In one embodiment, the one or more additional copies of the importer inserted into the genome of the bacterial cell are under the control of the same inducible promoter that controls expression of the one or more gene sequence(s) encoding the oxalate catabolism enzyme, e.g., the FNR responsive promoter, or a different inducible promoter than the one that controls expression of the gene sequence(s) encoding the one or more oxalate catabolism enzyme(s), or a constitutive promoter.

In one embodiment, when the importer of oxalate is expressed in the recombinant bacterial cells of the invention, the bacterial cells import 10% more oxalate into the bacterial cell when the importer is expressed than unmodified bacteria of the same bacterial subtype under the same conditions. In another embodiment, when the importer of oxalate is expressed in the recombinant bacterial cells of the invention, the bacterial cells import 20%, 30%, 40%, 50%, 60%, 70%, 80%, 90% or 100% more oxalate into the bacterial cell when the importer is expressed than unmodified bacteria of the same bacterial subtype under the same conditions. In yet another embodiment, when the importer of oxalate is expressed in the recombinant bacterial cells of the invention, the bacterial cells import two-fold more oxalate into the cell when the importer is expressed than unmodified bacteria of the same bacterial subtype under the same conditions. In yet another embodiment, when the importer of oxalate is expressed in the recombinant bacterial cells of the invention, the bacterial cells import three-fold, four-fold, five-fold, six-fold, seven-fold, eight-fold, nine-fold, or ten-fold more oxalate into the cell when the importer is expressed than unmodified bacteria of the same bacterial subtype under the same conditions.

In some embodiments, the bacterial cell comprises a genetic mutation in one or more endogenous gene(s) encoding a transporter (importer) of formate, wherein the genetic mutation reduces influx of formate into the bacterial cell. Without wishing to be bound by theory, such mutations may decrease intracellular formate concentrations and increase the flux through oxalate catabolism pathways. FocA of E. coli catalyzes bidirectional formate transport and may function by a channel-type mechanism (Flake et al., Unexpected oligomeric structure of the FocA formate channel of Escherichia coli: a paradigm for the formate-nitrite transporter family of integral membrane proteins”. FEMS microbiology letters. 303 (1): 69-75). FocA may be able to switch its mode of operation from a passive export channel at high external pH to a secondary active formate/H importer at low pH. In a non-limiting example, the genetically engineered bacteria may comprise a mutation and/or deletion in FocA, rendering it non-functional.

Exporters of Formate

Formate is a major metabolite in the anaerobic fermentation of glucose by many intestinal bacteria. Several types of formate import and export proteins are known in the art. For example, formate is translocated across cellular membranes by the pentameric ion channel/transporter FocA in E coli and other Enterobacteriaceae. FocA acts as a passive exporter for formate anions generated in the cytoplasm. In the periplasm, formate is subsequently reduced by formate dehydrogenase into carbon dioxide. Another form of formate dehydrogenase and/or formate lyase also exists in the cytoplasm in E coli. A functional switch of transport mode occurs when the pH of the growth medium drops below 6.8. With ample protons available in the periplasm, the cell switches to active import of formate and again uses FocA for the task.

In another example, as mentioned above, the uptake of oxalate into the anaerobic bacterium, Oxalobacter formigenes, has been found to occur via the oxalate transporter OxlT. OxlT allows the exchange of oxalate with the intracellular formate derived from oxalate decarboxylation. The overall effect of these associated activities (exchange and decarboxylation) is generation of a proton-motive force to support membrane functions, including ATP synthesis, accumulation of growth substrates and extrusion of waste products. As such, “exporter of formate” in some embodiments also encompasses a transporter of oxalate, e.g., as in the case of OxlT, the formate:oxalate antiporter.

Formate exporters and/or formate exporters with coupled oxalate import functions may be expressed or modified in the bacteria in order to enhance formate export (and in cases when coupled to oxalate import, thereby enhance oxalate import). Specifically, in some embodiments, when the exporter of formate is expressed in the engineered bacterial cells, the bacterial cells export more formate outside of the cell when the exporter is expressed than unmodified bacteria of the same bacterial subtype under the same conditions. In one embodiment, the bacterial cell comprises one or more gene sequence(s) encoding an exporter of formate. In one embodiment, the bacterial cell comprises a heterologous gene encoding an exporter of formate and at least one heterologous gene or gene cassette encoding at least one oxalate catabolism enzyme.

Thus, in some embodiments, the disclosure provides a bacterial cell that comprises one or more gene sequence(s) encoding one or more oxalate catabolism enzyme(s) operably linked to a first promoter and one or more gene sequence(s) encoding an exporter of formate. In some embodiments, the one or more gene sequence(s) encoding an exporter of formate is operably linked to the first promoter. In another embodiment, the one or more gene sequence(s) encoding one or more oxalate catabolism enzyme(s) is operably is linked to a first promoter, and the one or more gene sequence(s) encoding an exporter of formate is operably linked to a second promoter. In one embodiment, the first promoter and the second promoter are separate copies of the same promoter. In another embodiment, the first promoter and the second promoter are different promoters.

In one embodiment, the bacterial cell comprises one or more gene sequence(s) encoding an exporter of formate from a different organism, e.g., a different species of bacteria. In one embodiment, the bacterial cell comprises at least one native gene sequence(s) encoding an exporter of formate. In some embodiments, the at least one native gene sequence(s) encoding an exporter of formate is not modified. In another embodiment, the bacterial cell comprises more than one copy of at least one gene native sequence(s) encoding an exporter of formate. In yet another embodiment, the bacterial cell comprises a copy one or more gene sequence(s) encoding a native exporter of formate, as well as at least one copy of at least one heterologous gene sequence(s) encoding an exporter of formate from a different bacterial species. In one embodiment, the bacterial cell comprises at least one, two, three, four, five, or six copies of the at least one heterologous gene sequences encoding an exporter of formate. In one embodiment, the bacterial cell comprises multiple copies of one or more heterologous gene sequence(s) encoding an exporter of formate.

In some embodiments, the exporter of formate is encoded by an exporter of formate gene derived from a bacterial genus or species, including but not limited to, Bifidobacterium, Bordetella, Bradyrhizobium, Burkholderia, Clostridium, Enterococcus, Escherichia, Eubacterium, Lactobacillus, Magnetospirillium, Mycobacterium, Neurospora, Oxalobacter, e.g., Oxalobacter formigenes, Ralstonia, Rhodopseudomonas, Shigella, Thermoplasma, and Thauera, e.g., Bifidobacterium animalis, Bifidobacterium bifidum, Bifidobacterium infantis, Bifidobacterium lactis, Bifidobacterium longum, Bordatella bronchiseptica, Bordatella parapertussis, Burkholderia fungorum, Burkholderia xenovorans, Bradyrhizobium japonicum, Clostridium acetobutylicum, Clostridium difficile, Clostridium scindens, Clostridium sporogenes, Clostridium tentani, Enterococcus faecalis, Escherichia coli, Eubacterium lentum, Lactobacillus acidophilus, Lactobacillus bulgaricus, Lactobacillus casei, Lactobacillus gasseri, Lactobacillus plantarum, Lactobacillus rhamnosus, Lactococcus lactis, Magnetospirillium magentotaticum, Mycobacterium avium, Mycobacterium intracellulare, Mycobacterium kansasii, Mycobacterium leprae, Mycobacterium smegmatis, Mycobacterium tuberculosis, Mycobacterium ulcerans, Neurospora crassa, Oxalobacter formigenes, Providencia rettgeri, Eubacterium lentum, Ralstonia eutropha, Ralstonia metallidurans, Rhodopseudomonas palustris, Shigella flexneri, Thermoplasma volcanium, and Thauera aromatica.

The present disclosure further comprises genes encoding functional fragments of an exporter of formate or functional variants of an exporter of formate. As used herein, the term “functional fragment thereof” or “functional variant thereof” of an exporter of formate relates to an element having qualitative biological activity in common with the wild-type exporter of formate from which the fragment or variant was derived. For example, a functional fragment or a functional variant of a mutated exporter of formate protein is one which retains essentially the same ability to import formate into the bacterial cell as does the exporter protein from which the functional fragment or functional variant was derived. In one embodiment, the engineered bacterial cell comprises at least one heterologous gene encoding a functional fragment of an exporter of formate. In another embodiment, the engineered bacterial cell comprises at least one heterologous gene encoding a functional variant of an exporter of formate.

Assays for testing the activity of an exporter of formate, an exporter of formate functional variant, or an exporter of formate functional fragment are well known to one of ordinary skill in the art. For example, formate export can be assessed by expressing the protein, functional variant, or fragment thereof, in an engineered bacterial cell that lacks an endogenous formate exporter and assessing formate levels in the media after expression of the protein. Methods for measuring formate export are well known to one of ordinary skill in the art (see, e.g., Wraight et al., Structure and mechanism of a pentameric formate channel Nat Struct Mol Biol. 2010 January; 17(1): 31-37).

In one embodiment the genes encoding the exporter of formate have been codon-optimized for use in the host organism. In one embodiment, the genes encoding the exporter of formate have been codon-optimized for use in Escherichia coli.

The present disclosure also encompasses genes encoding an exporter of formate comprising amino acids in its sequence that are substantially the same as an amino acid sequence described herein. Amino acid sequences that are substantially the same as the sequences described herein include sequences comprising conservative amino acid substitutions, as well as amino acid deletions and/or insertions.

In some embodiments, the at least one gene encoding an exporter of formate is mutagenized; mutants exhibiting increased formate transport are selected; and the mutagenized at least one gene encoding an exporter of formate is isolated and inserted into the bacterial cell. In a non-limiting example, increasing export of formate may also allow increased oxalate import. In some embodiments, the at least one gene encoding an exporter of formate is mutagenized; mutants exhibiting decreased formate transport are selected; and the mutagenized at least one gene encoding an exporter of formate is isolated and inserted into the bacterial cell. The exporter modifications described herein may be present on a plasmid or chromosome.

In one embodiment, the formate exporter is OxlT. In one embodiment, the OxlT gene has at least about 80% identity to SEQ ID NO: 11. Accordingly, in one embodiment, the OxlT gene has at least about 90% identity to SEQ ID NO: 11. Accordingly, in one embodiment, the OxlT gene has at least about 95% identity to SEQ ID NO: 11. Accordingly, in one embodiment, the OxlT gene has at least about 85%, 86%, 87%, 88%, 89%, 90%, 91%, 92%, 93%, 94%, 95%, 96%, 97%, 98%, or 99% identity to SEQ ID NO: 11. In another embodiment, the OxlT gene comprises the sequence of SEQ ID NO: 11. In yet another embodiment the OxlT gene consists of the sequence of SEQ ID NO: 11.

In one embodiment, the OxlT gene encodes a polypeptide which has at least about 80% identity to SEQ ID NO: 12. Accordingly, in one embodiment, the OxlT gene encodes a polypeptide which has at least about 90% identity to SEQ ID NO: 12. Accordingly, in one embodiment, the OxlT gene encodes a polypeptide which has at least about 95% identity to SEQ ID NO: 12. Accordingly, in one embodiment, the OxlT gene encodes a polypeptide which has at least about 85%, 86%, 87%, 88%, 89%, 90%, 91%, 92%, 93%, 94%, 95%, 96%, 97%, 98%, or 99% identity to SEQ ID NO: 12. In another embodiment, the OxlT gene encodes a polypeptide which comprises the sequence of SEQ ID NO: 12. In yet another embodiment the OxlT gene encodes a polypeptide which consists of the sequence of SEQ ID NO: 12.

In some embodiments, the bacterial cell comprises one or more heterologous gene sequence(s) encoding at least one oxalate catabolism enzyme operably linked to a first promoter and one or more heterologous gene sequence(s) encoding an exporter of formate. In some embodiments, the one or more heterologous gene sequence(s) encoding an exporter of formate are operably linked to the first promoter. In other embodiments, the one or more heterologous gene sequence(s) encoding an exporter of formate are operably linked to a second promoter. In one embodiment, one or more heterologous gene sequence(s) encoding an exporter of formate are directly operably linked to the second promoter. In another embodiment, the one or more heterologous gene sequence(s) encoding an exporter of formate are indirectly operably linked to the second promoter.

In some embodiments, expression one or more gene sequence(s) encoding an exporter of formate is controlled by a different promoter than the promoter that controls expression of the at least one gene encoding the at least one oxalate catabolism enzyme. In some embodiments, expression of the one or more gene sequence(s) encoding an exporter of formate is controlled by the same promoter that controls expression of the at least one oxalate catabolism enzyme. In some embodiments, the one or more gene sequence(s) encoding an exporter of formate and the oxalate catabolism enzyme are divergently transcribed from a promoter region. In some embodiments, expression of each of genes encoding the one or more gene sequence(s) encoding an exporter of formate and the one or more gene sequence(s) encoding the at least one oxalate catabolism enzyme is controlled by different promoters.

In one embodiment, the promoter is not operably linked with the one or more gene sequence(s) encoding an exporter of formate in nature. In some embodiments, the one or more gene sequence(s) encoding the exporter of formate is controlled by its native promoter. In some embodiments, the one or more gene sequence(s) encoding the exporter of formate is controlled by an inducible promoter. In some embodiments, the one or more gene sequence(s) encoding the exporter of formate is controlled by a promoter that is stronger than its native promoter. In some embodiments, the one or more gene sequence(s) encoding the exporter of formate is controlled by a constitutive promoter.

In another embodiment, the promoter is an inducible promoter. Inducible promoters are described in more detail infra.

In one embodiment, the one or more gene sequence(s) encoding an exporter of formate is located on a plasmid in the bacterial cell. In another embodiment, the one or more gene sequence(s) encoding an exporter of formate is located in the chromosome of the bacterial cell. In yet another embodiment, a native copy of the one or more gene sequence(s) encoding an exporter of formate is located in the chromosome of the bacterial cell, and a copy of at least one gene encoding an exporter of formate from a different species of bacteria is located on a plasmid in the bacterial cell. In yet another embodiment, a native copy of the one or more gene sequence(s) encoding an exporter of a formate is located on a plasmid in the bacterial cell, and a copy of at least one gene encoding an exporter of formate from a different species of bacteria is located on a plasmid in the bacterial cell. In yet another embodiment, a native copy of the one or more gene sequence(s) encoding an exporter of formate is located in the chromosome of the bacterial cell, and a copy of the one or more gene sequence(s) encoding an exporter of formate from a different species of bacteria is located in the chromosome of the bacterial cell.

In some embodiments, the at least one native gene encoding the exporter in the bacterial cell is not modified, and one or more additional copies of the native exporter are inserted into the genome. In one embodiment, the one or more additional copies of the native exporter that is inserted into the genome are under the control of the same inducible promoter that controls expression of the at least one gene encoding the oxalate catabolism enzyme, e.g., the FNR responsive promoter, or a different inducible promoter than the one that controls expression of the at least one oxalate catabolism enzyme, or a constitutive promoter. In alternate embodiments, the at least one native gene encoding the exporter is not modified, and one or more additional copies of the exporter from a different bacterial species is inserted into the genome of the bacterial cell. In one embodiment, the one or more additional copies of the exporter inserted into the genome of the bacterial cell are under the control of the same inducible promoter that controls expression of the at least one gene encoding the oxalate catabolism enzyme, e.g., the FNR responsive promoter, or a different inducible promoter than the one that controls expression of the at least one gene encoding the at least one oxalate catabolism enzyme, or a constitutive promoter.

In one embodiment, when the exporter of formate is expressed in the engineered bacterial cells, the bacterial cells export 10% more formate out of the bacterial cell when the exporter is expressed than unmodified bacteria of the same bacterial subtype under the same conditions. In another embodiment, when the exporter of formate is expressed in the engineered bacterial cells, the bacterial cells export 20%, 30%, 40%, 50%, 60%, 70%, 80%, 90% or 100% more formate out of the bacterial cell when the exporter is expressed than unmodified bacteria of the same bacterial subtype under the same conditions. In yet another embodiment, when the exporter of formate is expressed in the engineered bacterial cells, the bacterial cells export two-fold more formate out of the cell when the exporter is expressed than unmodified bacteria of the same bacterial subtype under the same conditions. In yet another embodiment, when the exporter of formate is expressed in the engineered bacterial cells, the bacterial cells export three-fold, four-fold, five-fold, six-fold, seven-fold, eight-fold, nine-fold, or ten-fold more formate out of the cell when the exporter is expressed than unmodified bacteria of the same bacterial subtype under the same conditions.

In one embodiment, the bacterial cell comprises a mutation or deletion in an exporter of oxalate, rendering the exporter less functional or non-functional. Such a mutation may prevent intracellular oxalate from being exported and increase the catabolism of oxalate.

In some embodiments, the genetically engineered bacteria further comprise a mutation or deletion in one or more endogenous formate exporters, e.g., FocA. In a non-limiting example, such genetically engineered bacteria comprising a mutation in FocA comprise one or more gene sequence(s) encoding a formate:oxalate antiporter, e.g., OxlT. In a non-limiting example one or more endogenous formate exporter(s) are mutagenized or deleted, e.g., (e.g., FocA) to reduce or prevent the export of formate without the concurrent import of oxalate through a formate: oxalate antiporter, e.g., OxlT. Such a mutation may increase the uptake and catabolism of oxalate in the bacterial cell.

In some embodiments, formate dehydrogenase and/or formate lyase is mutated or deleted, e.g. to prevent the catabolism of formate in the bacterial cell. Without wishing to be bound by theory, such mutations may increase intracellular formate concentrations, allowing an increase in the flux through a formate oxalate antiporter, and thereby allowing increased oxalate uptake.

Inducible Promoters

In some embodiments, the bacterial cell comprises a stably maintained plasmid or chromosome carrying the gene and/or gene cassette encoding one or more oxalate catabolism enzyme(s), e.g., selected from formyl-CoA:oxalate CoA-transferase, e.g., frc (from O. formigenes), oxalyl-CoA synthetase, e.g., ScAAE3 (from S. cerevisiae), oxalyl-CoA decarboxylase, e.g., selected from oxc (from O. formigenes), and/or acetyl-CoA:oxalate CoA-transferase, e.g., YfdE (from E. coli), genes. such that the oxalate catabolism enzyme(s) can be expressed in the host cell, and the host cell is capable of survival and/or growth in vitro, e.g., in medium, and/or in vivo, e.g., in the gut. In some embodiments, bacterial cell comprises two or more distinct oxalate catabolism enzymes, e.g., formyl-CoA:oxalate CoA-transferase, e.g., frc (from O. formigenes), oxalyl-CoA synthetase, e.g., ScAAE3 (from S. cerevisiae), oxalyl-CoA decarboxylase, e.g., oxc (from O. formigenes), and/or acetyl-CoA:oxalate CoA-transferase, e.g., YfdE (from E. coli) genes. In some embodiments, the genetically engineered bacteria comprise multiple copies of the same oxalate catabolism enzyme gene and/or gene cassette. In some embodiments, the genetically engineered bacteria comprise multiple copies of different oxalate catabolism enzyme genes. In some embodiments, the gene and/or gene cassette encoding one or more oxalate catabolism enzyme(s) is present on a plasmid and operably linked to a directly or indirectly inducible promoter. In some embodiments, the gene and/or gene cassette encoding one or more oxalate catabolism enzyme(s) is present on a plasmid and operably linked to a promoter that is induced under low-oxygen or anaerobic conditions. In some embodiments, the gene and/or gene cassette encoding one or more oxalate catabolism enzyme(s) is present on a chromosome and operably linked to a directly or indirectly inducible promoter. In some embodiments, the gene and/or gene cassette encoding one or more oxalate catabolism enzyme(s) is present in the chromosome and operably linked to a promoter that is induced under low-oxygen or anaerobic conditions. In some embodiments, the gene and/or gene cassette encoding one or more oxalate catabolism enzyme(s) is present on a plasmid and operably linked to a promoter that is induced by exposure to tetracycline or arabinose.

In some embodiments, the bacterial cell comprises a stably maintained plasmid or chromosome carrying the gene and/or gene cassette encoding one or more transporter(s) of oxalate, e.g., OxlT from O. formigenes, such that the transporter, e.g., OxlT from O. formigenes, can be expressed in the host cell, and the host cell is capable of survival and/or growth in vitro, e.g., in medium, and/or in vivo, e.g., in the gut. In some embodiments, bacterial cell comprises two or more distinct copies of the gene and/or gene cassette encoding one or more transporter(s) of oxalate, e.g., OxlT from O. formigenes. In some embodiments, the genetically engineered bacteria comprise multiple copies of the same gene and/or gene cassette encoding one or more transporter(s) of oxalate, e.g., OxlT from O. formigenes. In some embodiments, the at least one gene and/or gene cassette encoding one or more transporter(s) of oxalate, e.g., OxlT from O. formigenes, is present on a plasmid and operably linked to a directly or indirectly inducible promoter. In some embodiments, the gene and/or gene cassette encoding one or more transporter(s) of oxalate, e.g., OxlT from O. formigenes, is present on a plasmid and operably linked to a promoter that is induced under low-oxygen or anaerobic conditions. In some embodiments, the gene and/or gene cassette encoding one or more transporter(s) of oxalate, e.g., OxlT from O. formigenes, is present on a chromosome and operably linked to a directly or indirectly inducible promoter. In some embodiments, the gene and/or gene cassette encoding one or more transporter(s) of oxalate, e.g., OxlT from O. formigenes, is present in the chromosome and operably linked to a promoter that is induced under low-oxygen or anaerobic conditions. In some embodiments, the gene and/or gene cassette encoding one or more transporter(s) of oxalate, e.g., OxlT from O. formigenes, is present on a plasmid and operably linked to a promoter that is induced by exposure to tetracycline.

In some embodiments, the promoter that is operably linked to the gene and/or gene cassette encoding one or more oxalate catabolism enzyme(s) and the promoter that is operably linked to the gene and/or gene cassette encoding one or more transporter(s) of oxalate, e.g., OxlT from O. formigenes, is directly induced by exogenous environmental conditions. In some embodiments, the promoter that is operably linked to the gene and/or gene cassette encoding one or more oxalate catabolism enzyme(s) and the promoter that is operably linked to the gene and/or gene cassette encoding one or more transporter(s) of oxalate, e.g., OxlT from O. formigenes, is indirectly induced by exogenous environmental conditions. In some embodiments, the promoter is directly or indirectly induced by exogenous environmental conditions specific to the gut of a mammal. In some embodiments, the promoter is directly or indirectly induced by exogenous environmental conditions specific to the small intestine of a mammal. In some embodiments, the promoter is directly or indirectly induced by low-oxygen or anaerobic conditions such as the environment of the mammalian gut. In some embodiments, the promoter is directly or indirectly induced by molecules or metabolites that are specific to the gut of a mammal, e.g., propionate. In some embodiments, the promoter is directly or indirectly induced by a molecule that is co-administered with the bacterial cell.

Oxygen Dependent Regulation

In certain embodiments, the bacterial cell comprises a gene and/or gene cassette encoding one or more oxalate catabolism enzyme(s), e.g., selected from formyl-CoA:oxalate CoA-transferase, e.g., frc (from O. formigenes), oxalyl-CoA synthetase, e.g., ScAAE3 (from S. cerevisiae), oxalyl-CoA decarboxylase, e.g., oxc (from O. formigenes), and/or acetyl-CoA:oxalate CoA-transferase, e.g., YfdE (from E. coli), is expressed under the control of the fumarate and nitrate reductase regulator (FNR) promoter. In certain embodiments, the bacterial cell comprises gene and/or gene cassette encoding one or more transporter(s) of oxalate, e.g., OxlT from O. formigenes, is expressed under the control of the fumarate and nitrate reductase regulator (FNR) promoter. In E. coli, FNR is a major transcriptional activator that controls the switch from aerobic to anaerobic metabolism (Unden et al., 1997). In the anaerobic state, FNR dimerizes into an active DNA binding protein that activates hundreds of genes responsible for adapting to anaerobic growth. In the aerobic state, FNR is prevented from dimerizing by oxygen and is inactive.

FNR responsive promoters include, but are not limited to, the FNR responsive promoters listed in the chart, below. Underlined sequences are predicted ribosome binding sites, and bolded sequences are restriction sites used for cloning.

TABLE 5
FNR responsive promoters

| | |
|---|---|
| FNR Responsive Promoter | SEQ ID NO |

SEQ ID NO: 13
SEQ ID NO: 14
SEQ ID NO: 15
SEQ ID NO: 16
SEQ ID NO: 17

In one embodiment, the FNR responsive promoter comprises SEQ ID NO: 13. In one embodiment, the FNR responsive promoter comprises SEQ ID NO: 14. In another embodiment, the FNR responsive promoter comprises SEQ ID NO: 15. In another embodiment, the FNR responsive promoter comprises SEQ ID NO: 16. In another embodiment, the FNR responsive promoter comprises SEQ ID NO:17. Additional FNR responsive promoters are shown below in Table 6.

TABLE 6
FNR Promoter Sequences

| FNR-responsive regulatory | |
|---|---|
| region Sequence | SEQ ID NO |
| | SEQ ID NO: 18 |
| | SEQ ID NO: 19 |
| nirB1 | SEQ ID NO: 20 |
| nirB2 | SEQ ID NO: 21 |
| nirB3 | SEQ ID NO: 22 |
| ydfZ | SEQ ID NO: 23 |
| nirB + RBS | SEQ ID NO: 24 |
| ydfZ + RBS | SEQ ID NO: 25 |
| fnrS1 | SEQ ID NO: 26 |
| fnrS2 | SEQ ID NO: 27 |
| nirB + crp | SEQ ID NO: 28 |
| fnrS + crp | SEQ ID NO: 29 |

In one embodiment, the FNR responsive promoter comprises SEQ ID NO: 18. In one embodiment, the FNR responsive promoter comprises SEQ ID NO: 19. In another embodiment, the FNR responsive promoter comprises SEQ ID NO: 20. In another embodiment, the FNR responsive promoter comprises SEQ ID NO: 21. In another embodiment, the FNR responsive promoter comprises SEQ ID NO:22. In one embodiment, the FNR responsive promoter comprises SEQ ID NO: 23. In one embodiment, the FNR responsive promoter comprises SEQ ID NO: 24. In another embodiment, the FNR responsive promoter comprises SEQ ID NO: 25. In another embodiment, the FNR responsive promoter comprises SEQ ID NO: 26. In another embodiment, the FNR responsive promoter comprises SEQ ID NO:27. In another embodiment, the FNR responsive promoter comprises SEQ ID NO: 28. In another embodiment, the FNR responsive promoter comprises SEQ ID NO:29.

In some embodiments, multiple distinct FNR nucleic acid sequences are inserted in the genetically engineered bacteria. In alternate embodiments, the genetically engineered bacteria comprise a gene and/or gene cassette(s) encoding one or more oxalate catabolism enzyme(s), e.g., selected from formyl-CoA:oxalate CoA-transferase, e.g., Frc (from O. formigenes), oxalyl-CoA synthetase, e.g., ScAAE3 (from S. cerevisiae), oxalyl-CoA decarboxylase, e.g., Oxc (from O. formigenes), and/or acetyl-CoA:oxalate CoA-transferase, e.g., YfdE (from E. coli) or other enzyme disclosed herein, is expressed under the control of an alternate oxygen level-dependent promoter, e.g., DNR (Trunk et al., 2010) or ANR (Ray et al., 1997). In alternate embodiments, the genetically engineered bacteria comprise a gene and/or gene cassette encoding one or more transporter(s) of oxalate, e.g., OxIT from O. formigenes, which is expressed under the control of an alternate oxygen level-dependent promoter, e.g., DNR (Trunk et al., 2010) or ANR (Ray et al., 1997). In these embodiments, catabolism of oxalate and/or its metabolites, is particularly activated in a low-oxygen or anaerobic environment, such as in the gut. In some embodiments, gene expression is further optimized by methods known in the art, e.g., by optimizing ribosomal binding sites and/or increasing mRNA stability. In one embodiment, the mammalian gut is a human mammalian gut.

In some embodiments, the bacterial cell comprises an oxygen-level dependent transcriptional regulator, e.g., FNR, ANR, or DNR, and corresponding promoter from a different bacterial species. The heterologous oxygen-level dependent transcriptional regulator and promoter increase the transcription of genes operably linked to said promoter, e.g., the gene and/or gene cassette encoding one or more oxalate catabolism enzyme(s), and/or the gene and/or gene cassette encoding one or more transporter(s) of oxalate, e.g., OxlT from O. formigenes in a low-oxygen or anaerobic environment, as compared to the native gene(s) and promoter in the bacteria under the same conditions. In certain embodiments, the non-native oxygen-level dependent transcriptional regulator is an FNR protein from N gonorrhoeae (see, e.g., Isabella et al., 2011). In some embodiments, the corresponding wild-type transcriptional regulator is left intact and retains wild-type activity. In alternate embodiments, the corresponding wild-type transcriptional regulator is deleted or mutated to reduce or eliminate wild-type activity.

In some embodiments, the genetically engineered bacteria comprise a wild-type oxygen-level dependent transcriptional regulator, e.g., FNR, ANR, or DNR, and corresponding promoter that is mutated relative to the wild-type promoter from bacteria of the same subtype. The mutated promoter enhances binding to the wild-type transcriptional regulator and increases the transcription of genes operably linked to said promoter, e.g., the gene and/or gene cassette encoding one or more oxalate catabolism enzyme(s), and/or the gene and/or gene cassette encoding one or more transporter(s) of oxalate, e.g., OxlT from O. formigenes in a low-oxygen or anaerobic environment, as compared to the wild-type promoter under the same conditions. In some embodiments, the genetically engineered bacteria comprise a wild-type oxygen-level dependent promoter, e.g., FNR, ANR, or DNR promoter, and corresponding transcriptional regulator that is mutated relative to the wild-type transcriptional regulator from bacteria of the same subtype. The mutated transcriptional regulator enhances binding to the wild-type promoter and increases the transcription of genes operably linked to said promoter, e.g., the gene and/or gene cassette encoding one or more oxalate catabolism enzyme(s), and/or the gene and/or gene cassette encoding one or more transporter(s) of oxalate, e.g., OxIT from O. formigenes in a low-oxygen or anaerobic environment, as compared to the wild-type transcriptional regulator under the same conditions. In certain embodiments, the mutant oxygen-level dependent transcriptional regulator is an FNR protein comprising amino acid substitutions that enhance dimerization and FNR activity (see, e.g., Moore et al., 2006).

In some embodiments, the bacterial cells disclosed herein comprise multiple copies of the endogenous gene encoding the oxygen level-sensing transcriptional regulator, e.g., the FNR gene. In some embodiments, the gene encoding the oxygen level-sensing transcriptional regulator is present on a plasmid. In some embodiments, the gene encoding the oxygen level-sensing transcriptional regulator and the gene and/or gene cassette encoding one or more oxalate catabolism enzyme(s) are present on different plasmids. In some embodiments, the gene encoding the oxygen level-sensing transcriptional regulator and the gene and/or gene cassette encoding one or more oxalate catabolism enzyme(s) and/or the gene encoding a transporter of an oxalate are present on different plasmids. In some embodiments, the gene encoding the oxygen level-sensing transcriptional regulator and the gene and/or gene cassette encoding one or more oxalate catabolism enzyme(s) and/or the gene and/or gene cassette encoding one or more transporter(s) of oxalate are present on the same plasmid.

In some embodiments, the gene encoding the oxygen level-sensing transcriptional regulator is present on a chromosome. In some embodiments, the gene encoding the oxygen level-sensing transcriptional regulator and the gene and/or gene cassette encoding one or more oxalate catabolism enzyme(s) and/or the gene and/or gene cassette encoding one or more a transporter(s) of oxalate are present on different chromosomes. In some embodiments, the gene encoding the oxygen level-sensing transcriptional regulator and the gene and/or gene cassette encoding one or more oxalate catabolism enzyme(s) and/or the gene and/or gene cassette encoding one or more transporter(s) of oxalate are present on the same chromosome. In some instances, it may be advantageous to express the oxygen level-sensing transcriptional regulator under the control of an inducible promoter in order to enhance expression stability. In some embodiments, expression of the transcriptional regulator is controlled by a different promoter than the promoter that controls expression of the gene and/or gene cassette encoding one or more oxalate catabolism enzyme(s) and/or oxalate transporter. In some embodiments, expression of the transcriptional regulator is controlled by the same promoter that controls expression of the oxalate catabolism enzyme(s) and/or Oxalate transporter(s). In some embodiments, the transcriptional regulator and the oxalate catabolism enzyme(s) are divergently transcribed from a promoter region.

RNS-Dependent Regulation

In some embodiments, the genetically engineered bacteria comprise a gene and/or gene cassette encoding one or more oxalate catabolism enzyme(s) that is expressed under the control of an inducible promoter. In some embodiments, the genetically engineered bacterium that expresses one or more oxalate catabolism enzyme(s) and/or oxalate transporter(s) is under the control of a promoter that is activated by inflammatory conditions. In one embodiment, the gene and/or gene cassette for producing the oxalate catabolism enzyme(s) and/or oxalate transporter is expressed under the control of an inflammatory-dependent promoter that is activated in inflammatory environments, e.g., a reactive nitrogen species or RNS promoter.

As used herein, “reactive nitrogen species” and “RNS” are used interchangeably to refer to highly active molecules, ions, and/or radicals derived from molecular nitrogen. RNS can cause deleterious cellular effects such as nitrosative stress. RNS includes, but is not limited to, nitric oxide (NO.), peroxynitrite or peroxynitrite anion (ONOO—), nitrogen dioxide (.NO2), dinitrogen trioxide (N2O3), peroxynitrous acid (ONOOH), and nitroperoxycarbonate (ONOOCO2-) (unpaired electrons denoted by). Bacteria have evolved transcription factors that are capable of sensing RNS levels. Different RNS signaling pathways are triggered by different RNS levels and occur with different kinetics.

As used herein, “RNS-inducible regulatory region” refers to a nucleic acid sequence to which one or more RNS-sensing transcription factors is capable of binding, wherein the binding and/or activation of the corresponding transcription factor activates downstream gene expression; in the presence of RNS, the transcription factor binds to and/or activates the regulatory region. In some embodiments, the RNS-inducible regulatory region comprises a promoter sequence. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; in the presence of RNS, the transcription factor undergoes a conformational change, thereby activating downstream gene expression. The RNS-inducible regulatory region may be operatively linked to a gene and/or gene ore gene cassette, e.g., an oxalate catabolism enzyme gene sequence(s), e.g., any of the oxalate catabolism enzymes described herein. For example, in the presence of RNS, a transcription factor senses RNS and activates a corresponding RNS-inducible regulatory region, thereby driving expression of an operatively linked gene sequence. Thus, RNS induces expression of the gene or gene sequences.

As used herein, “RNS-derepressible regulatory region” refers to a nucleic acid sequence to which one or more RNS-sensing transcription factors is capable of binding, wherein the binding of the corresponding transcription factor represses downstream gene expression; in the presence of RNS, the transcription factor does not bind to and does not repress the regulatory region. In some embodiments, the RNS-derepressible regulatory region comprises a promoter sequence. The RNS-derepressible regulatory region may be operatively linked to a gene or gene cassette, e.g., one or more oxalate catabolism enzyme gene sequence(s) and oxalate transporter sequence(s). For example, in the presence of RNS, a transcription factor senses RNS and no longer binds to and/or represses the regulatory region, thereby derepressing an operatively linked gene sequence or gene cassette. Thus, RNS derepresses expression of the gene or gene cassette.

As used herein, “RNS-repressible regulatory region” refers to a nucleic acid sequence to which one or more RNS-sensing transcription factors is capable of binding, wherein the binding of the corresponding transcription factor represses downstream gene expression; in the presence of RNS, the transcription factor binds to and represses the regulatory region. In some embodiments, the RNS-repressible regulatory region comprises a promoter sequence. In some embodiments, the transcription factor that senses RNS is capable of binding to a regulatory region that overlaps with part of the promoter sequence. In alternate embodiments, the transcription factor that senses RNS is capable of binding to a regulatory region that is upstream or downstream of the promoter sequence. The RNS-repressible regulatory region may be operatively linked to a gene sequence or gene cassette. For example, in the presence of RNS, a transcription factor senses RNS and binds to a corresponding RNS-repressible regulatory region, thereby blocking expression of an operatively linked gene sequence or gene sequences. Thus, RNS represses expression of the gene or gene sequences.

As used herein, a “RNS-responsive regulatory region” refers to a RNS-inducible regulatory region, a RNS-repressible regulatory region, and/or a RNS-derepressible regulatory region. In some embodiments, the RNS-responsive regulatory region comprises a promoter sequence. Each regulatory region is capable of binding at least one corresponding RNS-sensing transcription factor. Examples of transcription factors that sense RNS and their corresponding RNS-responsive genes, promoters, and/or regulatory regions include, but are not limited to, those shown in Table 7.

TABLE 7
Examples of RNS-sensing transcription
factors and RNS-responsive genes

| RNS-sensing | Primarily | Examples of responsive genes, |
|---|---|---|
| transcription | capable | promoters, and/or regulatory |
| factor: | of sensing: | regions: |
| NsrR | NO | norB, aniA, nsrR hmpA, ytfE, ygbA, hcp, |
| | | hcr, nrfA, aox |
| NorR | NO | norVW, norR |
| DNR | NO | norCB, nir, nor, nos |

In some embodiments, the genetically engineered bacteria of the invention comprise a tunable regulatory region that is directly or indirectly controlled by a transcription factor that is capable of sensing at least one reactive nitrogen species. The tunable regulatory region is operatively linked to a gene and/or gene cassette capable of directly or indirectly driving the expression of one or more oxalate catabolism enzyme(s), oxalate transporter(s), thus controlling expression of the oxalate catabolism enzyme, oxalate transporter(s), relative to RNS levels. For example, the tunable regulatory region is a RNS-inducible regulatory region, and the payload is one or more oxalate catabolism enzyme(s), oxalate transporter(s), such as any of the oxalate catabolism enzymes, and/or oxalate transporter(s) provided herein; when RNS is present, e.g., in an inflamed tissue, a RNS-sensing transcription factor binds to and/or activates the regulatory region and drives expression of the oxalate catabolism enzyme and/or oxalate transporter gene or gene cassette. Subsequently, when inflammation is ameliorated, RNS levels are reduced, and production of the oxalate catabolism enzyme(s) and oxalate transporter(s) is decreased or eliminated.

In some embodiments, the tunable regulatory region is a RNS-inducible regulatory region; in the presence of RNS, a transcription factor senses RNS and activates the RNS-inducible regulatory region, thereby driving expression of an operatively linked gene or gene cassette. In some embodiments, the transcription factor senses RNS and subsequently binds to the RNS-inducible regulatory region, thereby activating downstream gene expression. In alternate embodiments, the transcription factor is bound to the RNS-inducible regulatory region in the absence of RNS; when the transcription factor senses RNS, it undergoes a conformational change, thereby inducing downstream gene expression.

In some embodiments, the tunable regulatory region is a RNS-inducible regulatory region, and the transcription factor that senses RNS is NorR. NorR “is an NO-responsive transcriptional activator that regulates expression of the norVW genes encoding flavorubredoxin and an associated flavoprotein, which reduce NO to nitrous oxide” (Spiro 2006). The genetically engineered bacteria of the invention may comprise any suitable RNS-responsive regulatory region from a gene that is activated by NorR. Genes that are capable of being activated by NorR are known in the art (see, e.g., Spiro 2006; Vine et al., 2011; Karlinsey et al., 2012; Table 1). In certain embodiments, the genetically engineered bacteria of the invention comprise a RNS-inducible regulatory region from norVW that is operatively linked to a gene and/or gene cassette, e.g., one or more oxalate catabolism enzyme(s) and/or oxalate transporter(s). In the presence of RNS, a NorR transcription factor senses RNS and activates to the norVW regulatory region, thereby driving expression of the operatively linked gene and/or gene cassette and producing the oxalate catabolism enzyme(s) and/or oxalate transporter(s).

In some embodiments, the tunable regulatory region is a RNS-inducible regulatory region, and the transcription factor that senses RNS is DNR. DNR (dissimilatory nitrate respiration regulator) “promotes the expression of the nir, the nor and the nos genes” in the presence of nitric oxide (Castiglione et al., 2009). The genetically engineered bacteria of the invention may comprise any suitable RNS-responsive regulatory region from a gene that is activated by DNR. Genes that are capable of being activated by DNR are known in the art (see, e.g., Castiglione et al., 2009; Giardina et al., 2008; Table 1). In certain embodiments, the genetically engineered bacteria of the invention comprise a RNS-inducible regulatory region from norCB that is operatively linked to a gene or gene cassette, e.g., a butyrogenic gene cassette. In the presence of RNS, a DNR transcription factor senses RNS and activates to the norCB regulatory region, thereby driving expression of the operatively linked gene or gene cassette and producing one or more oxalate catabolism enzymes. In some embodiments, the DNR is Pseudomonas aeruginosa DNR.

In some embodiments, the tunable regulatory region is a RNS-derepressible regulatory region, and binding of a corresponding transcription factor represses downstream gene expression; in the presence of RNS, the transcription factor no longer binds to the regulatory region, thereby derepressing the operatively linked gene or gene cassette.

In some embodiments, the tunable regulatory region is a RNS-derepressible regulatory region, and the transcription factor that senses RNS is NsrR. NsrR is “an Rrf2-type transcriptional repressor [that] can sense NO and control the expression of genes responsible for NO metabolism” (Isabella et al., 2009). The genetically engineered bacteria of the invention may comprise any suitable RNS-responsive regulatory region from a gene that is repressed by NsrR. In some embodiments, the NsrR is Neisseria gonorrhoeae NsrR. Genes that are capable of being repressed by NsrR are known in the art (see, e.g., Isabella et al., 2009; Dunn et al., 2010; Table 1). In certain embodiments, the genetically engineered bacteria of the invention comprise a RNS-derepressible regulatory region from norB that is operatively linked to a gene or gene cassette, e.g., an oxalate catabolism enzyme gene or gene cassette. In the presence of RNS, an NsrR transcription factor senses RNS and no longer binds to the norB regulatory region, thereby derepressing the operatively linked oxalate catabolism enzyme and/or oxalate transporter gene or gene cassette and producing the encoding an oxalate catabolism enzyme(s).

In some embodiments, it is advantageous for the genetically engineered bacteria to express a RNS-sensing transcription factor that does not regulate the expression of a significant number of native genes in the bacteria. In some embodiments, the genetically engineered bacterium of the invention expresses a RNS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the RNS-sensing transcription factor is NsrR, e.g., from is Neisseria gonorrhoeae, wherein the Escherichia coli does not comprise binding sites for said NsrR. In some embodiments, the heterologous transcription factor minimizes or eliminates off-target effects on endogenous regulatory regions and genes in the genetically engineered bacteria.

In some embodiments, the tunable regulatory region is a RNS-repressible regulatory region, and binding of a corresponding transcription factor represses downstream gene expression; in the presence of RNS, the transcription factor senses RNS and binds to the RNS-repressible regulatory region, thereby repressing expression of the operatively linked gene or gene cassette. In some embodiments, the RNS-sensing transcription factor is capable of binding to a regulatory region that overlaps with part of the promoter sequence. In alternate embodiments, the RNS-sensing transcription factor is capable of binding to a regulatory region that is upstream or downstream of the promoter sequence.

In these embodiments, the genetically engineered bacteria may comprise a two repressor activation regulatory circuit, which is used to express an oxalate catabolism enzyme. The two repressor activation regulatory circuit comprises a first RNS-sensing repressor and a second repressor, which is operatively linked to a gene or gene cassette, e.g., encoding an oxalate catabolism enzyme. In one aspect of these embodiments, the RNS-sensing repressor inhibits transcription of the second repressor, which inhibits the transcription of the gene or gene cassette. Examples of second repressors useful in these embodiments include, but are not limited to, TetR, C1, and LexA. In the absence of binding by the first repressor (which occurs in the absence of RNS), the second repressor is transcribed, which represses expression of the gene or gene cassette. In the presence of binding by the first repressor (which occurs in the presence of RNS), expression of the second repressor is repressed, and the gene(s) or gene cassette(s), e.g., one or more oxalate catabolism enzyme(s) and/or oxalate transporter(s) gene or gene cassette is expressed.

A RNS-responsive transcription factor may induce, derepress, or repress gene expression depending upon the regulatory region sequence used in the genetically engineered bacteria. One or more types of RNS-sensing transcription factors and corresponding regulatory region sequences may be present in genetically engineered bacteria. In some embodiments, the genetically engineered bacteria comprise one type of RNS-sensing transcription factor, e.g., NsrR, and one corresponding regulatory region sequence, e.g., from norB. In some embodiments, the genetically engineered bacteria comprise one type of RNS-sensing transcription factor, e.g., NsrR, and two or more different corresponding regulatory region sequences, e.g., from norB and aniA. In some embodiments, the genetically engineered bacteria comprise two or more types of RNS-sensing transcription factors, e.g., NsrR and NorR, and two or more corresponding regulatory region sequences, e.g., from norB and norR, respectively. One RNS-responsive regulatory region may be capable of binding more than one transcription factor. In some embodiments, the genetically engineered bacteria comprise two or more types of RNS-sensing transcription factors and one corresponding regulatory region sequence. Nucleic acid sequences of several RNS-regulated regulatory regions are known in the art (see, e.g., Spiro 2006; Isabella et al., 2009; Dunn et al., 2010; Vine et al., 2011; Karlinsey et al., 2012).

In some embodiments, the genetically engineered bacteria of the invention comprise a gene encoding a RNS-sensing transcription factor, e.g., the nsrR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the RNS-sensing transcription factor under the control of an inducible promoter in order to enhance expression stability. In some embodiments, expression of the RNS-sensing transcription factor is controlled by a different promoter than the promoter that controls expression of the therapeutic molecule. In some embodiments, expression of the RNS-sensing transcription factor is controlled by the same promoter that controls expression of the therapeutic molecule. In some embodiments, the RNS-sensing transcription factor and therapeutic molecule are divergently transcribed from a promoter region.

In some embodiments, the genetically engineered bacteria of the invention comprise a gene for a RNS-sensing transcription factor from a different species, strain, or substrain of bacteria. In some embodiments, the genetically engineered bacteria comprise a RNS-responsive regulatory region from a different species, strain, or substrain of bacteria. In some embodiments, the genetically engineered bacteria comprise a RNS-sensing transcription factor and corresponding RNS-responsive regulatory region from a different species, strain, or substrain of bacteria. The heterologous RNS-sensing transcription factor and regulatory region may increase the transcription of genes operatively linked to said regulatory region in the presence of RNS, as compared to the native transcription factor and regulatory region from bacteria of the same subtype under the same conditions.

In some embodiments, the genetically engineered bacteria comprise a RNS-sensing transcription factor, NsrR, and corresponding regulatory region, nsrR, from Neisseria gonorrhoeae. In some embodiments, the native RNS-sensing transcription factor, e.g., NsrR, is left intact and retains wild-type activity. In alternate embodiments, the native RNS-sensing transcription factor, e.g., NsrR, is deleted or mutated to reduce or eliminate wild-type activity.

In some embodiments, the genetically engineered bacteria of the invention comprise multiple copies of the endogenous gene encoding the RNS-sensing transcription factor, e.g., the nsrR gene. In some embodiments, the gene encoding the RNS-sensing transcription factor is present on a plasmid. In some embodiments, the gene encoding the RNS-sensing transcription factor and the gene or gene cassette for producing the therapeutic molecule are present on different plasmids. In some embodiments, the gene encoding the RNS-sensing transcription factor and the gene or gene cassette for producing the therapeutic molecule are present on the same plasmid. In some embodiments, the gene encoding the RNS-sensing transcription factor is present on a chromosome. In some embodiments, the gene encoding the RNS-sensing transcription factor and the gene or gene cassette for producing the therapeutic molecule are present on different chromosomes. In some embodiments, the gene encoding the RNS-sensing transcription factor and the gene or gene cassette for producing the therapeutic molecule are present on the same chromosome.

In some embodiments, the genetically engineered bacteria comprise a wild-type gene encoding a RNS-sensing transcription factor, e.g., the NsrR gene, and a corresponding regulatory region, e.g., a norB regulatory region, that is mutated relative to the wild-type regulatory region from bacteria of the same subtype. The mutated regulatory region increases the expression of the oxalate catabolism enzyme in the presence of RNS, as compared to the wild-type regulatory region under the same conditions. In some embodiments, the genetically engineered bacteria comprise a wild-type RNS-responsive regulatory region, e.g., the norB regulatory region, and a corresponding transcription factor, e.g., NsrR, that is mutated relative to the wild-type transcription factor from bacteria of the same subtype. The mutant transcription factor increases the expression of the oxalate catabolism enzyme(s) in the presence of RNS, as compared to the wild-type transcription factor under the same conditions. In some embodiments, both the RNS-sensing transcription factor and corresponding regulatory region are mutated relative to the wild-type sequences from bacteria of the same subtype in order to increase expression of the oxalate catabolism enzyme(s) and/or oxalate transporter(s) in the presence of RNS.

In some embodiments, the gene or gene cassette for producing the anti-inflammation and/or gut barrier function enhancer molecule is present on a plasmid and operably linked to a promoter that is induced by RNS. In some embodiments, expression is further optimized by methods known in the art, e.g., by optimizing ribosomal binding sites, manipulating transcriptional regulators, and/or increasing mRNA stability.

In some embodiments, any of the gene(s) of the present disclosure may be integrated into the bacterial chromosome at one or more integration sites. For example, one or more copies of an oxalate catabolism enzyme and/or oxalate transporter gene(s) may be integrated into the bacterial chromosome. Having multiple copies of the gene or gen(s) integrated into the chromosome allows for greater production of the oxalate catabolism enzyme(s) and also permits fine-tuning of the level of expression. Alternatively, different circuits described herein, such as any of the secretion or exporter circuits, in addition to the therapeutic gene(s) or gene cassette(s) could be integrated into the bacterial chromosome at one or more different integration sites to perform multiple different functions.

ROS-Dependent Regulation

In some embodiments, the genetically engineered bacteria comprise a gene and/or gene cassette for producing one or more oxalate catabolism enzyme(s) and/or oxalate transporter(s) that is expressed under the control of an inducible promoter. In some embodiments, the genetically engineered bacterium that expresses one or more oxalate catabolism enzyme(s) and/or oxalate transporter(s) under the control of a promoter that is activated by conditions of cellular damage. In one embodiment, the gene and/or gene cassette for producing one or more oxalate catabolism enzyme(s) is expressed under the control of a cellular damaged-dependent promoter that is activated in environments in which there is cellular or tissue damage, e.g., a reactive oxygen species or ROS promoter.

As used herein, “reactive oxygen species” and “ROS” are used interchangeably to refer to highly active molecules, ions, and/or radicals derived from molecular oxygen. ROS can be produced as byproducts of aerobic respiration or metal-catalyzed oxidation and may cause deleterious cellular effects such as oxidative damage. ROS includes, but is not limited to, hydrogen peroxide (H2O2), organic peroxide (ROOH), hydroxyl ion (OH—), hydroxyl radical (.OH), superoxide or superoxide anion (.O2-), singlet oxygen (1O2), ozone (O3), carbonate radical, peroxide or peroxyl radical (.O2-2), hypochlorous acid (HOCl), hypochlorite ion (OCl—), sodium hypochlorite (NaOCl), nitric oxide (NO.), and peroxynitrite or peroxynitrite anion (ONOO—) (unpaired electrons denoted by .). Bacteria have evolved transcription factors that are capable of sensing ROS levels. Different ROS signaling pathways are triggered by different ROS levels and occur with different kinetics (Marinho et al., 2014).

As used herein, “ROS-inducible regulatory region” refers to a nucleic acid sequence to which one or more ROS-sensing transcription factors is capable of binding, wherein the binding and/or activation of the corresponding transcription factor activates downstream gene expression; in the presence of ROS, the transcription factor binds to and/or activates the regulatory region. In some embodiments, the ROS-inducible regulatory region comprises a promoter sequence. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; in the presence of ROS, the transcription factor undergoes a conformational change, thereby activating downstream gene expression. The ROS-inducible regulatory region may be operatively linked to a gene sequence or gene sequence, e.g., a sequence or sequences encoding one or more oxalate catabolism enzyme(s). For example, in the presence of ROS, a transcription factor, e.g., OxyR, senses ROS and activates a corresponding ROS-inducible regulatory region, thereby driving expression of an operatively linked gene sequence or gene sequences. Thus, ROS induces expression of the gene or gene cassette.

As used herein, “ROS-derepressible regulatory region” refers to a nucleic acid sequence to which one or more ROS-sensing transcription factors is capable of binding, wherein the binding of the corresponding transcription factor represses downstream gene expression; in the presence of ROS, the transcription factor does not bind to and does not repress the regulatory region. In some embodiments, the ROS-derepressible regulatory region comprises a promoter sequence. The ROS-derepressible regulatory region may be operatively linked to a gene or gene cassette, e.g., one or more genes encoding one or more oxalate catabolism enzyme(s). For example, in the presence of ROS, a transcription factor, e.g., OhrR, senses ROS and no longer binds to and/or represses the regulatory region, thereby derepressing an operatively linked gene sequence or gene cassette. Thus, ROS derepresses expression of the gene or gene cassette.

As used herein, “ROS-repressible regulatory region” refers to a nucleic acid sequence to which one or more ROS-sensing transcription factors is capable of binding, wherein the binding of the corresponding transcription factor represses downstream gene expression; in the presence of ROS, the transcription factor binds to and represses the regulatory region. In some embodiments, the ROS-repressible regulatory region comprises a promoter sequence. In some embodiments, the transcription factor that senses ROS is capable of binding to a regulatory region that overlaps with part of the promoter sequence. In alternate embodiments, the transcription factor that senses ROS is capable of binding to a regulatory region that is upstream or downstream of the promoter sequence. The ROS-repressible regulatory region may be operatively linked to a gene sequence or gene sequences. For example, in the presence of ROS, a transcription factor, e.g., PerR, senses ROS and binds to a corresponding ROS-repressible regulatory region, thereby blocking expression of an operatively linked gene sequence or gene sequences. Thus, ROS represses expression of the gene or gene cassette.

As used herein, a “ROS-responsive regulatory region” refers to a ROS-inducible regulatory region, a ROS-repressible regulatory region, and/or a ROS-derepressible regulatory region. In some embodiments, the ROS-responsive regulatory region comprises a promoter sequence. Each regulatory region is capable of binding at least one corresponding ROS-sensing transcription factor. Examples of transcription factors that sense ROS and their corresponding ROS-responsive genes, promoters, and/or regulatory regions include, but are not limited to, those shown in Table 8.

TABLE 8
Examples of ROS-sensing transcription
factors and ROS-responsive genes

| ROS-sensing | Primarily | Examples of responsive genes, |
|---|---|---|
| transcription | capable | promoters, and/or regulatory |
| factor: | of sensing: | regions: |
| OxyR | H₂O₂ | ahpC; ahpF; dps; dsbG; fhuF; flu; |
| | | fur; gor; grxA; hemH; katG; oxyS; |
| | | sufA; sufB; sufC; sufD; sufE; sufS; |
| | | trxC; uxuA; yaaA; yaeH; yaiA; ybjM; |
| | | ydcH; ydeN; ygaQ; yljA; ytfK |
| PerR | H₂O₂ | katA; ahpCF; mrgA; zoaA; fur; |
| | | hemAXCDBL; srfA |
| OhrR | Organic peroxides | ohrA |
| | NaOCl | |
| SoxR | •O₂⁻ | soxS |
| | NO• | |
| | (also capable of | |
| | sensing H₂O₂) | |
| RosR | H₂O₂ | rbtT; tnp16a; rluC1; tnp5a; mscL; |
| | | tnp2d; phoD; tnp15b; pstA; tnp5b; |
| | | xylC; gabD1; rluC2; cgtS9; |
| | | azlC; narKGHJI; rosR |

In some embodiments, the genetically engineered bacteria comprise a tunable regulatory region that is directly or indirectly controlled by a transcription factor that is capable of sensing at least one reactive oxygen species. The tunable regulatory region is operatively linked to a gene or gene cassette capable of directly or indirectly driving the expression of an oxalate catabolism enzyme, thus controlling expression of the oxalate catabolism enzyme(s) relative to ROS levels. For example, the tunable regulatory region is a ROS-inducible regulatory region, and the molecule is an oxalate catabolism enzyme; when ROS is present, e.g., in an inflamed tissue, a ROS-sensing transcription factor binds to and/or activates the regulatory region and drives expression of the gene sequence and/or gene cassette sequence for one or more the oxalate catabolism enzyme(s) and/or oxalate transporter(s) thereby producing the oxalate catabolism enzyme(s) and/or oxalate transporter(s). Subsequently, when inflammation is ameliorated, ROS levels are reduced, and production of the oxalate catabolism enzyme(s) and/or oxalate transporter(s) is decreased or eliminated.

In some embodiments, the tunable regulatory region is a ROS-inducible regulatory region; in the presence of ROS, a transcription factor senses ROS and activates the ROS-inducible regulatory region, thereby driving expression of an operatively linked gene or gene cassette. In some embodiments, the transcription factor senses ROS and subsequently binds to the ROS-inducible regulatory region, thereby activating downstream gene expression. In alternate embodiments, the transcription factor is bound to the ROS-inducible regulatory region in the absence of ROS; when the transcription factor senses ROS, it undergoes a conformational change, thereby inducing downstream gene expression.

In some embodiments, the tunable regulatory region is a ROS-inducible regulatory region, and the transcription factor that senses ROS is OxyR. OxyR “functions primarily as a global regulator of the peroxide stress response” and is capable of regulating dozens of genes, e.g., “genes involved in H₂O₂ detoxification (katE, ahpCF), heme biosynthesis (hemH), reductant supply (grxA, gor, trxC), thiol-disulfide isomerization (dsbG), Fe—S center repair (sufA-E, sufS), iron binding (yaaA), repression of iron import systems (fur)” and “OxyS, a small regulatory RNA” (Dubbs et al., 2012). The genetically engineered bacteria may comprise any suitable ROS-responsive regulatory region from a gene that is activated by OxyR. Genes that are capable of being activated by OxyR are known in the art (see, e.g., Zheng et al., 2001; Dubbs et al., 2012; Table 1). In certain embodiments, the genetically engineered bacteria of the invention comprise a ROS-inducible regulatory region from oxyS that is operatively linked to a gene and/or gene cassette, e.g., one or more oxalate catabolism enzyme(s) and/or oxalate transporter genes. In the presence of ROS, e.g., H2O2, an OxyR transcription factor senses ROS and activates to the oxyS regulatory region, thereby driving expression of the operatively linked oxalate catabolism enzyme and/or oxalate transporter gene and or gene cassette and producing the oxalate catabolism enzyme(s) and/or oxalate transporter(s). In some embodiments, OxyR is encoded by an E. coli oxyR gene. In some embodiments, the oxyS regulatory region is an E. coli oxyS regulatory region. In some embodiments, the ROS-inducible regulatory region is selected from the regulatory region of katG, dps, and ahpC.

In alternate embodiments, the tunable regulatory region is a ROS-inducible regulatory region, and the corresponding transcription factor that senses ROS is SoxR. When SoxR is “activated by oxidation of its [2Fe-2S] cluster, it increases the synthesis of SoxS, which then activates its target gene expression” (Koo et al., 2003). “SoxR is known to respond primarily to superoxide and nitric oxide” (Koo et al., 2003), and is also capable of responding to H2O2. The genetically engineered bacteria of the invention may comprise any suitable ROS-responsive regulatory region from a gene that is activated by SoxR. Genes that are capable of being activated by SoxR are known in the art (see, e.g., Koo et al., 2003; Table 1). In certain embodiments, the genetically engineered bacteria of the invention comprise a ROS-inducible regulatory region from soxS that is operatively linked to a gene and/or gene cassette, e.g., an oxalate catabolism enzyme. In the presence of ROS, the SoxR transcription factor senses ROS and activates the soxS regulatory region, thereby driving expression of the operatively linked oxalate catabolism enzyme and/or oxalate transporter gene or gene cassette and producing one or more oxalate catabolism enzyme(s) and/or oxalate transporter(s).

In some embodiments, the tunable regulatory region is a ROS-derepressible regulatory region, and binding of a corresponding transcription factor represses downstream gene expression; in the presence of ROS, the transcription factor no longer binds to the regulatory region, thereby derepressing the operatively linked gene or gene cassette.

In some embodiments, the tunable regulatory region is a ROS-derepressible regulatory region, and the transcription factor that senses ROS is OhrR. OhrR “binds to a pair of inverted repeat DNA sequences overlapping the ohrA promoter site and thereby represses the transcription event,” but oxidized OhrR is “unable to bind its DNA target” (Duarte et al., 2010). OhrR is a “transcriptional repressor [that] . . . senses both organic peroxides and NaOCl” (Dubbs et al., 2012) and is “weakly activated by H₂O₂ but it shows much higher reactivity for organic hydroperoxides” (Duarte et al., 2010). The genetically engineered bacteria of the invention may comprise any suitable ROS-responsive regulatory region from a gene that is repressed by OhrR. Genes that are capable of being repressed by OhrR are known in the art (see, e.g., Dubbs et al., 2012; Table 1). In certain embodiments, the genetically engineered bacteria of the invention comprise a ROS-derepressible regulatory region from ohrA that is operatively linked to a gene or gene cassette, e.g., an oxalate catabolism enzyme and/or oxalate transporter gene. In the presence of ROS, e.g., NaOCl, an OhrR transcription factor senses ROS and no longer binds to the ohrA regulatory region, thereby derepressing the operatively linked oxalate catabolism enzyme and/or oxalate transporter gene and producing the oxalate catabolism enzyme and/or oxalate transporter.

OhrR is a member of the MarR family of ROS-responsive regulators. “Most members of the MarR family are transcriptional repressors and often bind to the −10 or −35 region in the promoter causing a steric inhibition of RNA polymerase binding” (Bussmann et al., 2010). Other members of this family are known in the art and include, but are not limited to, OspR, MgrA, RosR, and SarZ. In some embodiments, the transcription factor that senses ROS is OspR, MgRA, RosR, and/or SarZ, and the genetically engineered bacteria of the invention comprises one or more corresponding regulatory region sequences from a gene that is repressed by OspR, MgRA, RosR, and/or SarZ. Genes that are capable of being repressed by OspR, MgRA, RosR, and/or SarZ are known in the art (see, e.g., Dubbs et al., 2012).

In some embodiments, the tunable regulatory region is a ROS-derepressible regulatory region, and the corresponding transcription factor that senses ROS is RosR. RosR is “a MarR-type transcriptional regulator” that binds to an “18-bp inverted repeat with the consensus sequence TTGTTGAYRYRTCAACWA (SEQ ID NO: 64)” and is “reversibly inhibited by the oxidant H2O2” (Bussmann et al., 2010). RosR is capable of repressing numerous genes and putative genes, including but not limited to “a putative polyisoprenoid-binding protein (cg1322, gene upstream of and divergent from rosR), a sensory histidine kinase (cgtS9), a putative transcriptional regulator of the Crp/FNR family (cg3291), a protein of the glutathione S-transferase family (cg1426), two putative FMN reductases (cg1150 and cg1850), and four putative monooxygenases (cg0823, cg1848, cg2329, and cg3084)” (Bussmann et al., 2010). The genetically engineered bacteria of the invention may comprise any suitable ROS-responsive regulatory region from a gene that is repressed by RosR. Genes that are capable of being repressed by RosR are known in the art (see, e.g., Bussmann et al., 2010; Table 1). In certain embodiments, the genetically engineered bacteria of the invention comprise a ROS-derepressible regulatory region from cgtS9 that is operatively linked to a gene or gene cassette, e.g., an enzyme and/or oxalate transporter. In the presence of ROS, e.g., H2O2, a RosR transcription factor senses ROS and no longer binds to the cgtS9 regulatory region, thereby derepressing the operatively linked oxalate catabolism enzyme and/or oxalate transporter gene and producing the oxalate catabolism enzyme and/or oxalate transporter.

In some embodiments, it is advantageous for the genetically engineered bacteria to express a ROS-sensing transcription factor that does not regulate the expression of a significant number of native genes in the bacteria. In some embodiments, the genetically engineered bacterium of the invention expresses a ROS-sensing transcription factor from a different species, strain, or substrain of bacteria, wherein the transcription factor does not bind to regulatory sequences in the genetically engineered bacterium of the invention. In some embodiments, the genetically engineered bacterium of the invention is Escherichia coli, and the ROS-sensing transcription factor is RosR, e.g., from Corynebacterium glutamicum, wherein the Escherichia coli does not comprise binding sites for said RosR. In some embodiments, the heterologous transcription factor minimizes or eliminates off-target effects on endogenous regulatory regions and genes in the genetically engineered bacteria.

In some embodiments, the tunable regulatory region is a ROS-repressible regulatory region, and binding of a corresponding transcription factor represses downstream gene expression; in the presence of ROS, the transcription factor senses ROS and binds to the ROS-repressible regulatory region, thereby repressing expression of the operatively linked gene or gene cassette. In some embodiments, the ROS-sensing transcription factor is capable of binding to a regulatory region that overlaps with part of the promoter sequence. In alternate embodiments, the ROS-sensing transcription factor is capable of binding to a regulatory region that is upstream or downstream of the promoter sequence.

In some embodiments, the tunable regulatory region is a ROS-repressible regulatory region, and the transcription factor that senses ROS is PerR. In Bacillus subtilis, PerR “when bound to DNA, represses the genes coding for proteins involved in the oxidative stress response (katA, ahpC, and mrgA), metal homeostasis (hemAXCDBL, fur, and zoaA) and its own synthesis (perR)” (Marinho et al., 2014). PerR is a “global regulator that responds primarily to H2O2” (Dubbs et al., 2012) and “interacts with DNA at the per box, a specific palindromic consensus sequence (TTATAATNATTATAA (SEQ ID NO: 65)) residing within and near the promoter sequences of PerR-controlled genes” (Marinho et al., 2014). PerR is capable of binding a regulatory region that “overlaps part of the promoter or is immediately downstream from it” (Dubbs et at, 2012). The genetically engineered bacteria of the invention may comprise any suitable ROS-responsive regulatory region from a gene that is repressed by PerR. Genes that are capable of being repressed by PerR are known in the art (see, e.g., Dubbs et al., 2012; Table 1).

In these embodiments, the genetically engineered bacteria may comprise a two repressor activation regulatory circuit, which is used to express an oxalate catabolism enzyme. The two repressor activation regulatory circuit comprises a first ROS-sensing repressor, e.g., PerR, and a second repressor, e.g., TetR, which is operatively linked to a gene or gene cassette, e.g., an oxalate catabolism enzyme. In one aspect of these embodiments, the ROS-sensing repressor inhibits transcription of the second repressor, which inhibits the transcription of the gene or gene cassette. Examples of second repressors useful in these embodiments include, but are not limited to, TetR, C1, and LexA. In some embodiments, the ROS-sensing repressor is PerR. In some embodiments, the second repressor is TetR. In this embodiment, a PerR-repressible regulatory region drives expression of TetR, and a TetR-repressible regulatory region drives expression of the gene or gene cassette, e.g., an oxalate catabolism enzyme. In the absence of PerR binding (which occurs in the absence of ROS), tetR is transcribed, and TetR represses expression of the gene or gene cassette, e.g., an oxalate catabolism enzyme. In the presence of PerR binding (which occurs in the presence of ROS), tetR expression is repressed, and the gene or gene cassette, e.g., an oxalate catabolism enzyme and/or oxalate transporter is expressed.

A ROS-responsive transcription factor may induce, derepress, or repress gene expression depending upon the regulatory region sequence used in the genetically engineered bacteria. For example, although “OxyR is primarily thought of as a transcriptional activator under oxidizing conditions . . . OxyR can function as either a repressor or activator under both oxidizing and reducing conditions” (Dubbs et al., 2012), and OxyR “has been shown to be a repressor of its own expression as well as that of fhuF (encoding a ferric ion reductase) and flu (encoding the antigen 43 outer membrane protein)” (Zheng et al., 2001). The genetically engineered bacteria of the invention may comprise any suitable ROS-responsive regulatory region from a gene that is repressed by OxyR. In some embodiments, OxyR is used in a two repressor activation regulatory circuit, as described above. Genes that are capable of being repressed by OxyR are known in the art (see, e.g., Zheng et al., 2001; Table 1). Or, for example, although RosR is capable of repressing a number of genes, it is also capable of activating certain genes, e.g., the narKGHJI operon. In some embodiments, the genetically engineered bacteria comprise any suitable ROS-responsive regulatory region from a gene that is activated by RosR. In addition, “PerR-mediated positive regulation has also been observed . . . and appears to involve PerR binding to distant upstream sites” (Dubbs et al., 2012). In some embodiments, the genetically engineered bacteria comprise any suitable ROS-responsive regulatory region from a gene that is activated by PerR.

One or more types of ROS-sensing transcription factors and corresponding regulatory region sequences may be present in genetically engineered bacteria. For example, “OhrR is found in both Gram-positive and Gram-negative bacteria and can co-reside with either OxyR or PerR or both” (Dubbs et al., 2012). In some embodiments, the genetically engineered bacteria comprise one type of ROS-sensing transcription factor, e.g., OxyR, and one corresponding regulatory region sequence, e.g., from oxyS. In some embodiments, the genetically engineered bacteria comprise one type of ROS-sensing transcription factor, e.g., OxyR, and two or more different corresponding regulatory region sequences, e.g., from oxyS and katG. In some embodiments, the genetically engineered bacteria comprise two or more types of ROS-sensing transcription factors, e.g., OxyR and PerR, and two or more corresponding regulatory region sequences, e.g., from oxyS and katA, respectively. One ROS-responsive regulatory region may be capable of binding more than one transcription factor. In some embodiments, the genetically engineered bacteria comprise two or more types of ROS-sensing transcription factors and one corresponding regulatory region sequence.

Nucleic acid sequences of several exemplary OxyR-regulated regulatory regions are shown in Table 5. OxyR binding sites are underlined and bolded. In some embodiments, genetically engineered bacteria comprise a nucleic acid sequence that is at least about 80%, at least about 85%, at least about 90%, at least about 95%, or at least about 99% homologous to the DNA sequence of SEQ ID NO: 46, 47, 48, or 49, or a functional fragment thereof.

TABLE 9
Nucleotide sequences of exemplary
OxyR-regulated regulatory regions

| Regulatory sequence | SEQ ID NO |
|---|---|
| katG | SEQ ID NO: 30 |
| dps | SEQ ID NO: 31 |
| ahpC | SEQ ID NO: 32 |
| oxyS | SEQ ID NO: 33 |

In some embodiments, the genetically engineered bacteria of the invention comprise a gene encoding a ROS-sensing transcription factor, e.g., the oxyR gene, that is controlled by its native promoter, an inducible promoter, a promoter that is stronger than the native promoter, e.g., the GlnRS promoter or the P(Bla) promoter, or a constitutive promoter. In some instances, it may be advantageous to express the ROS-sensing transcription factor under the control of an inducible promoter in order to enhance expression stability. In some embodiments, expression of the ROS-sensing transcription factor is controlled by a different promoter than the promoter that controls expression of the therapeutic molecule. In some embodiments, expression of the ROS-sensing transcription factor is controlled by the same promoter that controls expression of the therapeutic molecule. In some embodiments, the ROS-sensing transcription factor and therapeutic molecule are divergently transcribed from a promoter region.

In some embodiments, the genetically engineered bacteria of the invention comprise a gene for a ROS-sensing transcription factor from a different species, strain, or substrain of bacteria. In some embodiments, the genetically engineered bacteria comprise a ROS-responsive regulatory region from a different species, strain, or substrain of bacteria. In some embodiments, the genetically engineered bacteria comprise a ROS-sensing transcription factor and corresponding ROS-responsive regulatory region from a different species, strain, or substrain of bacteria. The heterologous ROS-sensing transcription factor and regulatory region may increase the transcription of genes operatively linked to said regulatory region in the presence of ROS, as compared to the native transcription factor and regulatory region from bacteria of the same subtype under the same conditions.

In some embodiments, the genetically engineered bacteria comprise a ROS-sensing transcription factor, OxyR, and corresponding regulatory region, oxyS, from Escherichia coli. In some embodiments, the native ROS-sensing transcription factor, e.g., OxyR, is left intact and retains wild-type activity. In alternate embodiments, the native ROS-sensing transcription factor, e.g., OxyR, is deleted or mutated to reduce or eliminate wild-type activity.

In some embodiments, the genetically engineered bacteria of the invention comprise multiple copies of the endogenous gene encoding the ROS-sensing transcription factor, e.g., the oxyR gene. In some embodiments, the gene encoding the ROS-sensing transcription factor is present on a plasmid. In some embodiments, the gene encoding the ROS-sensing transcription factor and the gene or gene cassette for producing the therapeutic molecule are present on different plasmids. In some embodiments, the gene encoding the ROS-sensing transcription factor and the gene or gene cassette for producing the therapeutic molecule are present on the same. In some embodiments, the gene encoding the ROS-sensing transcription factor is present on a chromosome. In some embodiments, the gene encoding the ROS-sensing transcription factor and the gene or gene cassette for producing the therapeutic molecule are present on different chromosomes. In some embodiments, the gene encoding the ROS-sensing transcription factor and the gene or gene cassette for producing the therapeutic molecule are present on the same chromosome.

In some embodiments, the genetically engineered bacteria comprise a wild-type gene encoding a ROS-sensing transcription factor, e.g., the soxR gene, and a corresponding regulatory region, e.g., a soxS regulatory region, that is mutated relative to the wild-type regulatory region from bacteria of the same subtype. The mutated regulatory region increases the expression of the oxalate catabolism enzyme and/or oxalate transporter in the presence of ROS, as compared to the wild-type regulatory region under the same conditions. In some embodiments, the genetically engineered bacteria comprise a wild-type ROS-responsive regulatory region, e.g., the oxyS regulatory region, and a corresponding transcription factor, e.g., OxyR, that is mutated relative to the wild-type transcription factor from bacteria of the same subtype. The mutant transcription factor increases the expression of the oxalate catabolism enzyme and/or oxalate transporter in the presence of ROS, as compared to the wild-type transcription factor under the same conditions. In some embodiments, both the ROS-sensing transcription factor and corresponding regulatory region are mutated relative to the wild-type sequences from bacteria of the same subtype in order to increase expression of the oxalate catabolism enzyme(s) in the presence of ROS.

In some embodiments, the gene or gene cassette for producing the oxalate catabolism enzyme(s) is present on a plasmid and operably linked to a promoter that is induced by ROS. In some embodiments, the gene or gene cassette for producing the oxalate catabolism enzyme(s) is present in the chromosome and operably linked to a promoter that is induced by ROS. In some embodiments, the gene or gene cassette for producing the oxalate catabolism enzyme(s) is present on a chromosome and operably linked to a promoter that is induced by exposure to tetracycline. In some embodiments, the gene or gene cassette for producing the oxalate catabolism enzyme and/or oxalate transporter is present on a plasmid and operably linked to a promoter that is induced by exposure to tetracycline. In some embodiments, expression is further optimized by methods known in the art, e.g., by optimizing ribosomal binding sites, manipulating transcriptional regulators, and/or increasing mRNA stability.

In some embodiments, the genetically engineered bacteria may comprise multiple copies of the gene(s) capable of producing an oxalate catabolism enzyme(s) and/or oxalate transporter(s). In some embodiments, the gene(s) capable of producing an oxalate catabolism enzyme(s) and/or oxalate transporter(s) is present on a plasmid and operatively linked to a ROS-responsive regulatory region. In some embodiments, the gene(s) capable of producing an oxalate catabolism enzyme and/or oxalate transporter is present in a chromosome and operatively linked to a ROS-responsive regulatory region.

Thus, in some embodiments, the genetically engineered bacteria or genetically engineered virus produce one or more oxalate catabolism enzymes under the control of an oxygen level-dependent promoter, a reactive oxygen species (ROS)-dependent promoter, or a reactive nitrogen species (RNS)-dependent promoter, and a corresponding transcription factor.

In some embodiments, the genetically engineered bacteria comprise a stably maintained plasmid or chromosome carrying a gene for producing an oxalate catabolism enzyme and/or oxalate transporter such that the oxalate catabolism enzyme and/or oxalate transporter can be expressed in the host cell, and the host cell is capable of survival and/or growth in vitro, e.g., in medium, and/or in vivo. In some embodiments, a bacterium may comprise multiple copies of the gene and/or gene cassette encoding one or more oxalate catabolism enzyme(s) and/or oxalate transporter(s). In some embodiments, the gene and/or gene cassette encoding one or more oxalate catabolism enzyme(s) and/or oxalate transporter(s) is expressed on a low-copy plasmid. In some embodiments, the low-copy plasmid may be useful for increasing stability of expression. In some embodiments, the low-copy plasmid may be useful for decreasing leaky expression under non-inducing conditions. In some embodiments, the gene and/or gene cassette encoding one or more oxalate catabolism enzyme(s) and/or oxalate transporter(s) is expressed on a high-copy plasmid. In some embodiments, the high-copy plasmid may be useful for increasing expression of the oxalate catabolism enzyme(s) and/or oxalate transporter(s). In some embodiments, the gene and/or gene cassette encoding one or more oxalate catabolism enzyme(s) and/or oxalate transporter(s) is expressed on a chromosome.

In some embodiments, the bacteria are genetically engineered to include multiple mechanisms of action (MOAs), e.g., circuits producing multiple copies of the same product (e.g., to enhance copy number) or circuits performing multiple different functions. For example, the genetically engineered bacteria may include four copies of the gene and/or gene cassette encoding one or more particular oxalate catabolism enzyme(s) and/or oxalate transporter(s) inserted at four different insertion sites. Alternatively, the genetically engineered bacteria may include three copies of the gene encoding a particular oxalate catabolism enzyme and/or oxalate transporter inserted at three different insertion sites and three copies of the gene encoding a different oxalate catabolism enzyme and/or oxalate transporter inserted at three different insertion sites.

In some embodiments, under conditions where the oxalate catabolism enzyme(s) and/or oxalate transporter is expressed, the genetically engineered bacteria of the disclosure produce at least about 1.5-fold, at least about 2-fold, at least about 10-fold, at least about 15-fold, at least about 20-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, at least about 1,000-fold, or at least about 1,500-fold more of the oxalate catabolism enzyme(s) and/or oxalate transporter(s) and/or transcript of the gene(s) in the operon as compared to unmodified bacteria of the same subtype under the same conditions.

In some embodiments, quantitative PCR (qPCR) is used to amplify, detect, and/or quantify mRNA expression levels of the oxalate catabolism enzyme(s) and/or oxalate transporter(s) gene(s). Primers specific for oxalate catabolism enzyme and/or oxalate transporter gene(s) may be designed and used to detect mRNA in a sample according to methods known in the art. In some embodiments, a fluorophore is added to a sample reaction mixture that may contain oxalate catabolism enzyme mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the oxalate catabolism enzyme and/or oxalate transporter gene(s).

In some embodiments, quantitative PCR (qPCR) is used to amplify, detect, and/or quantify mRNA expression levels of the oxalate catabolism enzyme(s) and/or oxalate transporter gene(s). Primers specific for oxalate catabolism enzyme and/or oxalate transporter gene(s) may be designed and used to detect mRNA in a sample according to methods known in the art. In some embodiments, a fluorophore is added to a sample reaction mixture that may contain oxalate catabolism enzyme and/or oxalate transporter mRNA, and a thermal cycler is used to illuminate the sample reaction mixture with a specific wavelength of light and detect the subsequent emission by the fluorophore. The reaction mixture is heated and cooled to predetermined temperatures for predetermined time periods. In certain embodiments, the heating and cooling is repeated for a predetermined number of cycles. In some embodiments, the reaction mixture is heated and cooled to 90-100° C., 60-70° C., and 30-50° C. for a predetermined number of cycles. In a certain embodiment, the reaction mixture is heated and cooled to 93-97° C., 55-65° C., and 35-45° C. for a predetermined number of cycles. In some embodiments, the accumulating amplicon is quantified after each cycle of the qPCR. The number of cycles at which fluorescence exceeds the threshold is the threshold cycle (CT). At least one CT result for each sample is generated, and the CT result(s) may be used to determine mRNA expression levels of the oxalate catabolism enzyme and/or oxalate transporter gene(s).

In other embodiments, the inducible promoter is a propionate responsive promoter. For example, the prpR promoter is a propionate responsive promoter. In one embodiment, the propionate responsive promoter comprises SEQ ID NO: 58.

Essential Genes and Auxotrophs

As used herein, the term “essential gene” refers to a gene which is necessary to for cell growth and/or survival. Bacterial essential genes are well known to one of ordinary skill in the art, and can be identified by directed deletion of genes and/or random mutagenesis and screening (see, for example, Zhang and Lin, 2009, DEG 5.0, a database of essential genes in both prokaryotes and eukaryotes, Nucl. Acids Res., 37:D455-D458 and Gerdes et al., Essential genes on metabolic maps, Curr. Opin. Biotechnol., 17(5):448-456, the entire contents of each of which are expressly incorporated herein by reference).

An “essential gene” may be dependent on the circumstances and environment in which an organism lives. For example, a mutation of, modification of, or excision of an essential gene may result in the recombinant bacteria of the disclosure becoming an auxotroph. An auxotrophic modification is intended to cause bacteria to die in the absence of an exogenously added nutrient essential for survival or growth because they lack the gene(s) necessary to produce that essential nutrient.

An auxotrophic modification is intended to cause bacteria to die in the absence of an exogenously added nutrient essential for survival or growth because they lack the gene(s) necessary to produce that essential nutrient. In some embodiments, any of the genetically engineered bacteria described herein also comprise a deletion or mutation in one or more gene(s) required for cell survival and/or growth.

In some embodiments, the bacterial cell comprises a genetic mutation in one or more endogenous gene(s) encoding an oxalate biosynthesis gene, wherein the genetic mutation reduces biosynthesis of oxalate in the bacterial cell.

In one embodiment, the essential gene is an oligonucleotide synthesis gene, for example, thyA. In another embodiment, the essential gene is a cell wall synthesis gene, for example, dapA. In yet another embodiment, the essential gene is an amino acid gene, for example, serA or MetA. Any gene required for cell survival and/or growth may be targeted, including but not limited to, cysE, glnA, ilvD, leuB, lysA, serA, metA, glyA, hisB, ilvA, pheA, proA, thrC, trpC, tyrA, thyA, uraA, dapA, dapB, dapD, dapE, dapF, flhD, metB, metC, proAB, and thi1, as long as the corresponding wild-type gene product is not produced in the bacteria.

Table 10 lists depicts exemplary bacterial genes which may be disrupted or deleted to produce an auxotrophic strain. These include, but are not limited to, genes required for oligonucleotide synthesis, amino acid synthesis, and cell wall synthesis.

TABLE 10
Non-limiting Examples of Bacterial Genes
Useful for Generation of an Auxotroph

| Amino Acid | Oligonucleotide | Cell Wall |
|---|---|---|
| cysE | thyA | dapA |
| glnA | uraA | dapB |
| ilvD | | dapD |
| leuB | | dapE |
| lysA | | dapF |
| serA | | |
| metA | | |
| glyA | | |
| hisB | | |
| ilvA | | |
| pheA | | |
| proA | | |
| thrC | | |
| trpC | | |
| tyrA | | |

Table 11 shows the survival of various amino acid auxotrophs in the mouse gut, as detected 24 hrs and 48 hrs post-gavage. These auxotrophs were generated using BW25113, a non-Nissle strain of E. coli.

TABLE 11
Survival of amino acid auxotrophs in the mouse gut

| Gene | AA Auxotroph | Pre-Gavage | 24 hours | 48 hours |
|---|---|---|---|---|
| argA | Arginine | Present | Present | Absent |
| cysE | Cysteine | Present | Present | Absent |
| glnA | Glutamine | Present | Present | Absent |
| glyA | Glycine | Present | Present | Absent |
| hisB | Histidine | Present | Present | Present |
| ilvA | Isoleucine | Present | Present | Absent |
| leuB | Leucine | Present | Present | Absent |
| lysA | Lysine | Present | Present | Absent |
| metA | Methionine | Present | Present | Present |
| pheA | Phenylalanine | Present | Present | Present |
| proA | Proline | Present | Present | Absent |
| sera | Serine | Present | Present | Present |
| thrC | Threonine | Present | Present | Present |
| trpC | Tryptophan | Present | Present | Present |
| tyrA | Tyrosine | Present | Present | Present |
| ilvD | Valine/Isoleucine/ | Present | Present | Absent |
| | Leucine | | | |
| thyA | Thiamine | Present | Absent | Absent |
| uraA | Uracil | Present | Absent | Absent |
| flhD | FlhD | Present | Present | Present |

For example, thymine is a nucleic acid that is required for bacterial cell growth; in its absence, bacteria undergo cell death. The thyA gene encodes thymidylate synthetase, an enzyme that catalyzes the first step in thymine synthesis by converting dUMP to dTMP (Sat et al., 2003). In some embodiments, the bacterial cell of the disclosure is a thyA auxotroph in which the thyA gene is deleted and/or replaced with an unrelated gene. A thyA auxotroph can grow only when sufficient amounts of thymine are present, e.g., by adding thymine to growth media in vitro, or in the presence of high thymine levels found naturally in the human gut in vivo. In some embodiments, the bacterial cell of the disclosure is auxotrophic in a gene that is complemented when the bacterium is present in the mammalian gut. Without sufficient amounts of thymine, the thyA auxotroph dies. In some embodiments, the auxotrophic modification is used to ensure that the bacterial cell does not survive in the absence of the auxotrophic gene product (e.g., outside of the gut).

Diaminopimelic acid (DAP) is an amino acid synthetized within the lysine biosynthetic pathway and is required for bacterial cell wall growth (Meadow et al., 1959; Clarkson et al., 1971). In some embodiments, any of the genetically engineered bacteria described herein is a dapD auxotroph in which dapD is deleted and/or replaced with an unrelated gene. A dapD auxotroph can grow only when sufficient amounts of DAP are present, e.g., by adding DAP to growth media in vitro. Without sufficient amounts of DAP, the dapD auxotroph dies. In some embodiments, the auxotrophic modification is used to ensure that the bacterial cell does not survive in the absence of the auxotrophic gene product (e.g., outside of the gut).

In other embodiments, the genetically engineered bacterium of the present disclosure is a uraA auxotroph in which uraA is deleted and/or replaced with an unrelated gene. The uraA gene codes for UraA, a membrane-bound transporter that facilitates the uptake and subsequent metabolism of the pyrimidine uracil (Andersen et al., 1995). A uraA auxotroph can grow only when sufficient amounts of uracil are present, e.g., by adding uracil to growth media in vitro. Without sufficient amounts of uracil, the uraA auxotroph dies. In some embodiments, auxotrophic modifications are used to ensure that the bacteria do not survive in the absence of the auxotrophic gene product (e.g., outside of the gut).

In complex communities, it is possible for bacteria to share DNA. In very rare circumstances, an auxotrophic bacterial strain may receive DNA from a non-auxotrophic strain, which repairs the genomic deletion and permanently rescues the auxotroph. Therefore, engineering a bacterial strain with more than one auxotroph may greatly decrease the probability that DNA transfer will occur enough times to rescue the auxotrophy. In some embodiments, the genetically engineered bacteria comprise a deletion or mutation in two or more genes required for cell survival and/or growth.

Other examples of essential genes include, but are not limited to yhbV, yagG, hemB, secD, secF, ribD, ribE, thiL, dxs, ispA, dnaX, adk, hemH, lpxH, cysS, fold, rplT, infC, thrS, nadE, gapA, yeaZ, aspS, argS, pgsA, yefM, metG, folE, yejM, gyrA, nrdA, nrdB, folC, accD, fabB, gltX, ligA, zipA, dapE, dapA, der, hisS, ispG, suhB, tadA, acpS, era, rnc, ftsB, eno, pyrG, chpR, lgt, fbaA, pgk, yqgD, metK, yqgF, plsC, ygiT, pare, ribB, cca, ygjD, tdcF, yraL, yihA, ftsN, murl, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, dnaC, ribF, lspA, ispH, dapB, folA, imp, yabQ, ftsL, ftsl, murE, murF, mraY, murD, ftsW, murG, murC, ftsQ, ftsA, ftsZ, lpxC, secM, secA, can, folK, hemL, yadR, dapD, map, rpsB, infB, nusA, ftsH, obgE, rpmA, rplU, ispB, murA, yrbB, yrbK, yhbN, rpsl, rplM, degS, mreD, mreC, mreB, accB, accC, yrdC, def, fmt, rplQ, rpoA, rpsD, rpsK, rpsM, entD, mrdB, mrdA, nadD, hlepB, rpoE, pssA, yfiO, rplS, trmD, rpsP, ffh, grpE, yfiB, csrA, ispF, ispD, rplW, rplD, rplC, rpsJ, fusA, rpsG, rpsL, trpS, yrjF, asd, rpoH, ftsX, ftsE, ftsY, frr, dxr, ispU, rfaK, kdtA, coaD, rpmB, dfp, dut, gmk, spot, gyrB, dnaN, dnaA, rpmH, rnpA, yidC, tnaB, glmS, glmU, wzyE, hemD, hemC, yigP, ubiB, ubiD, hemG, secY, rplO, rpmD, rpsE, rplR, rplF, rpsH, rpsN, rplE, rplX, rplN, rpsQ, rpmC, rplP, rpsC, rplV, rpsS, rplB, cdsA, yaeL, yaeT, lpxD, fabZ, lpxA, lpxB, dnaE, accA, tilS, proS, yajF, tsf, pyrH, olA, rlpB, leuS, lnt, glnS, fldA, cydA, infA, cydC, ftsK, lolA, serS, rpsA, msbA, lpxK, kdsB, mukF, mukE, mukB, asnS, fabA, mviN, me, yceQ, fabD, fabG, acpP, tmk, holB, lolC, lolD, lolE, purB, ymjK, minE, mind, pth, rsA, ispE, lolB, hemA, prfA, prmC, kdsA, topA, ribA, fabl, racR, dicA, ydfB, tyrS, ribC, ydiL, pheT, pheS, yhhQ, bcsB, glyQ, yibJ, and gpsA. Other essential genes are known to those of ordinary skill in the art.

In some embodiments, the genetically engineered bacterium of the present disclosure is a synthetic ligand-dependent essential gene (SLiDE) bacterial cell. SLiDE bacterial cells are synthetic auxotrophs with a mutation in one or more essential genes that only grow in the presence of a particular ligand (see Lopez and Anderson “Synthetic Auxotrophs with Ligand-Dependent Essential Genes for a BL21 (DE3 Biosafety Strain,” ACS Synthetic Biology (2015) DOI: 10.1021/acssynbio.5b00085, the entire contents of which are expressly incorporated herein by reference).

In some embodiments, the SLiDE bacterial cell comprises a mutation in an essential gene. In some embodiments, the essential gene is selected from the group consisting of pheS, dnaN, tyrS, metG and adk. In some embodiments, the essential gene is dnaN comprising one or more of the following mutations: H191N, R240C, I317S, F319V, L340T, V347I, and S345C. In some embodiments, the essential gene is dnaN comprising the mutations H191N, R240C, I317S, F319V, L340T, V347I, and S345C. In some embodiments, the essential gene is pheS comprising one or more of the following mutations: F125G, P183T, P184A, R186A, and I188L. In some embodiments, the essential gene is pheS comprising the mutations F125G, P183T, P184A, R186A, and I188L. In some embodiments, the essential gene is tyrS comprising one or more of the following mutations: L36V, C38A and F40G. In some embodiments, the essential gene is tyrS comprising the mutations L36V, C38A and F40G. In some embodiments, the essential gene is metG comprising one or more of the following mutations: E45Q, N47R, I49G, and A51C. In some embodiments, the essential gene is metG comprising the mutations E45Q, N47R, I49G, and A51C. In some embodiments, the essential gene is adk comprising one or more of the following mutations: I4L, L5I and L6G. In some embodiments, the essential gene is adk comprising the mutations I4L, L5I and L6G.

In some embodiments, the genetically engineered bacterium is complemented by a ligand. In some embodiments, the ligand is selected from the group consisting of benzothiazole, indole, 2-aminobenzothiazole, indole-3-butyric acid, indole-3-acetic acid, and L-histidine methyl ester. For example, bacterial cells comprising mutations in metG (E45Q, N47R, I49G, and A51C) are complemented by benzothiazole, indole, 2-aminobenzothiazole, indole-3-butyric acid, indole-3-acetic acid or L-histidine methyl ester. Bacterial cells comprising mutations in dnaN (H191N, R240C, I317S, F319V, L340T, V347I, and S345C) are complemented by benzothiazole, indole or 2-aminobenzothiazole. Bacterial cells comprising mutations in pheS (F125G, P183T, P184A, R186A, and I188L) are complemented by benzothiazole or 2-aminobenzothiazole. Bacterial cells comprising mutations in tyrS (L36V, C38A, and F40G) are complemented by benzothiazole or 2-aminobenzothiazole. Bacterial cells comprising mutations in adk (I4L, L5I and L6G) are complemented by benzothiazole or indole.

In some embodiments, the genetically engineered bacterium comprises more than one mutant essential gene that renders it auxotrophic to a ligand. In some embodiments, the bacterial cell comprises mutations in two essential genes. For example, in some embodiments, the bacterial cell comprises mutations in tyrS (L36V, C38A, and F40G) and metG (E45Q, N47R, I49G, and A51C). In other embodiments, the bacterial cell comprises mutations in three essential genes. For example, in some embodiments, the bacterial cell comprises mutations in tyrS (L36V, C38A, and F40G), metG (E45Q, N47R, I49G, and A51C), and pheS (F125G, P183T, P184A, R186A, and I188L).

In some embodiments, the genetically engineered bacterium is a conditional auxotroph whose essential gene(s) is replaced using the arabinose system described herein.

In some embodiments, the genetically engineered bacterium of the disclosure is an auxotroph and also comprises kill-switch circuitry, such as any of the kill-switch components and systems described herein. For example, the recombinant bacteria may comprise a deletion or mutation in an essential gene required for cell survival and/or growth, for example, in a DNA synthesis gene, for example, thyA, cell wall synthesis gene, for example, dapA and/or an amino acid gene, for example, serA or MetA or ilvC, and may also comprise a toxin gene that is regulated by one or more transcriptional activators that are expressed in response to an environmental condition(s) and/or signal(s) (such as the described arabinose system) or regulated by one or more recombinases that are expressed upon sensing an exogenous environmental condition(s) and/or signal(s) (such as the recombinase systems described herein). Other embodiments are described in Wright et al., “GeneGuard: A Modular Plasmid System Designed for Biosafety,” ACS Synthetic Biology (2015) 4: 307-16, the entire contents of which are expressly incorporated herein by reference). In some embodiments, the genetically engineered bacterium of the disclosure is an auxotroph and also comprises kill-switch circuitry, such as any of the kill-switch components and systems described herein, as well as another biosecurity system, such a conditional origin of replication (see Wright et al., supra).

Genetic Regulatory Circuits

In some embodiments, the genetically engineered bacteria comprise multi-layered genetic regulatory circuits for expressing the constructs described herein (see, e.g., U.S. Provisional Application No. 62/184,811, incorporated herein by reference in its entirety). The genetic regulatory circuits are useful to screen for mutant bacteria that produce one or more oxalate catabolism enzyme(s) and/or oxalate transporter(s) or rescue an auxotroph. In certain embodiments, the invention provides methods for selecting genetically engineered bacteria that produce one or more genes of interest.

In some embodiments, the invention provides genetically engineered bacteria comprising a gene or gene cassette for producing a payload and a T7 polymerase-regulated genetic regulatory circuit. For example, the genetically engineered bacteria comprise a first gene encoding a T7 polymerase, wherein the first gene is operably linked to a fumarate and nitrate reductase regulator (FNR)-responsive promoter; a second gene or gene cassette for producing a payload, wherein the second gene or gene cassette is operably linked to a T7 promoter that is induced by the T7 polymerase; and a third gene encoding an inhibitory factor, lysY, that is capable of inhibiting the T7 polymerase. In the presence of oxygen, FNR does not bind the FNR-responsive promoter, and the payload is not expressed. LysY is expressed constitutively (P-lac constitutive) and further inhibits T7 polymerase. In the absence of oxygen, FNR dimerizes and binds to the FNR-responsive promoter, T7 polymerase is expressed at a level sufficient to overcome lysY inhibition, and the payload is expressed. In some embodiments, the lysY gene is operably linked to an additional FNR binding site. In the absence of oxygen, FNR dimerizes to activate T7 polymerase expression as described above, and also inhibits lysY expression.

In some embodiments, the invention provides genetically engineered bacteria comprising a gene or gene cassette for producing a payload and a protease-regulated genetic regulatory circuit. For example, the genetically engineered bacteria comprise a first gene encoding an mf-lon protease, wherein the first gene is operably linked to a FNR-responsive promoter; a second gene or gene cassette for producing a payload operably linked to a Tet regulatory region (tetO); and a third gene encoding an mf-lon degradation signal linked to a Tet repressor (tetR), wherein the tetR is capable of binding to the Tet regulatory region and repressing expression of the second gene or gene cassette. The mf-lon protease is capable of recognizing the mf-lon degradation signal and degrading the tetR. In the presence of oxygen, FNR does not bind the FNR-responsive promoter, the repressor is not degraded, and the payload is not expressed. In the absence of oxygen, FNR dimerizes and binds the FNR-responsive promoter, thereby inducing expression of mf-lon protease. The mf-lon protease recognizes the mf-lon degradation signal and degrades the tetR, and the payload is expressed.

In some embodiments, the invention provides genetically engineered bacteria comprising a gene or gene cassette for producing a payload and a repressor-regulated genetic regulatory circuit. For example, the genetically engineered bacteria comprise a first gene encoding a first repressor, wherein the first gene is operably linked to a FNR-responsive promoter; a second gene or gene cassette for producing a payload operably linked to a first regulatory region comprising a constitutive promoter; and a third gene encoding a second repressor, wherein the second repressor is capable of binding to the first regulatory region and repressing expression of the second gene or gene cassette. The third gene is operably linked to a second regulatory region comprising a constitutive promoter, wherein the first repressor is capable of binding to the second regulatory region and inhibiting expression of the second repressor. In the presence of oxygen, FNR does not bind the FNR-responsive promoter, the first repressor is not expressed, the second repressor is expressed, and the payload is not expressed. In the absence of oxygen, FNR dimerizes and binds the FNR-responsive promoter, the first repressor is expressed, the second repressor is not expressed, and the payload is expressed.

Examples of repressors useful in these embodiments include, but are not limited to, ArgR, TetR, ArsR, AscG, Lad, CscR, DeoR, DgoR, FruR, GalR, GatR, CI, LexA, RafR, QacR, and PtxS (US20030166191).

In some embodiments, the invention provides genetically engineered bacteria comprising a gene or gene cassette for producing a payload and a regulatory RNA-regulated genetic regulatory circuit. For example, the genetically engineered bacteria comprise a first gene encoding a regulatory RNA, wherein the first gene is operably linked to a FNR-responsive promoter, and a second gene or gene cassette for producing a payload. The second gene or gene cassette is operably linked to a constitutive promoter and further linked to a nucleotide sequence capable of producing an mRNA hairpin that inhibits translation of the payload. The regulatory RNA is capable of eliminating the mRNA hairpin and inducing payload translation via the ribosomal binding site. In the presence of oxygen, FNR does not bind the FNR-responsive promoter, the regulatory RNA is not expressed, and the mRNA hairpin prevents the payload from being translated. In the absence of oxygen, FNR dimerizes and binds the FNR-responsive promoter, the regulatory RNA is expressed, the mRNA hairpin is eliminated, and the payload is expressed.

In some embodiments, the invention provides genetically engineered bacteria comprising a gene or gene cassette for producing a payload and a CRISPR-regulated genetic regulatory circuit. For example, the genetically engineered bacteria comprise a Cas9 protein; a first gene encoding a CRISPR guide RNA, wherein the first gene is operably linked to a FNR-responsive promoter; a second gene or gene cassette for producing a payload, wherein the second gene or gene cassette is operably linked to a regulatory region comprising a constitutive promoter; and a third gene encoding a repressor operably linked to a constitutive promoter, wherein the repressor is capable of binding to the regulatory region and repressing expression of the second gene or gene cassette. The third gene is further linked to a CRISPR target sequence that is capable of binding to the CRISPR guide RNA, wherein said binding to the CRISPR guide RNA induces cleavage by the Cas9 protein and inhibits expression of the repressor. In the presence of oxygen, FNR does not bind the FNR-responsive promoter, the guide RNA is not expressed, the repressor is expressed, and the payload is not expressed. In the absence of oxygen, FNR dimerizes and binds the FNR-responsive promoter, the guide RNA is expressed, the repressor is not expressed, and the payload is expressed.

In some embodiments, the invention provides genetically engineered bacteria comprising a gene or gene cassette for producing a payload and a recombinase-regulated genetic regulatory circuit. For example, the genetically engineered bacteria comprise a first gene encoding a recombinase, wherein the first gene is operably linked to a FNR-responsive promoter, and a second gene or gene cassette for producing a payload operably linked to a constitutive promoter. The second gene or gene cassette is inverted in orientation (3′ to 5′) and flanked by recombinase binding sites, and the recombinase is capable of binding to the recombinase binding sites to induce expression of the second gene or gene cassette by reverting its orientation (5′ to 3′). In the presence of oxygen, FNR does not bind the FNR-responsive promoter, the recombinase is not expressed, the payload remains in the 3′ to 5′ orientation, and no functional payload is produced. In the absence of oxygen, FNR dimerizes and binds the FNR-responsive promoter, the recombinase is expressed, the payload is reverted to the 5′ to 3′ orientation, and functional payload is produced.

In some embodiments, the invention provides genetically engineered bacteria comprising a gene or gene cassette for producing a payload and a polymerase- and recombinase-regulated genetic regulatory circuit. For example, the genetically engineered bacteria comprise a first gene encoding a recombinase, wherein the first gene is operably linked to a FNR-responsive promoter; a second gene or gene cassette for producing a payload operably linked to a T7 promoter; a third gene encoding a T7 polymerase, wherein the T7 polymerase is capable of binding to the T7 promoter and inducing expression of the payload. The third gene encoding the T7 polymerase is inverted in orientation (3′ to 5′) and flanked by recombinase binding sites, and the recombinase is capable of binding to the recombinase binding sites to induce expression of the T7 polymerase gene by reverting its orientation (5′ to 3′). In the presence of oxygen, FNR does not bind the FNR-responsive promoter, the recombinase is not expressed, the T7 polymerase gene remains in the 3′ to 5′ orientation, and the payload is not expressed. In the absence of oxygen, FNR dimerizes and binds the FNR-responsive promoter, the recombinase is expressed, the T7 polymerase gene is reverted to the 5′ to 3′ orientation, and the payload is expressed.

Kill Switches

In some embodiments, the genetically engineered bacteria also comprise a kill switch (see, e.g., U.S. Provisional Application Nos. 62/183,935 and 62/263,329, each of which are expressly incorporated herein by reference in their entireties). The kill switch is intended to actively kill engineered microbes in response to external stimuli. As opposed to an auxotrophic mutation where bacteria die because they lack an essential nutrient for survival, the kill switch is triggered by a particular factor in the environment that induces the production of toxic molecules within the microbe that cause cell death.

Bacteria engineered with kill switches have been engineered for in vitro research purposes, e.g., to limit the spread of a biofuel-producing microorganism outside of a laboratory environment. Bacteria engineered for in vivo administration to treat a disease or disorder may also be programmed to die at a specific time after the expression and delivery of a heterologous gene or gene cassette, for example, a therapeutic gene(s) or after the subject has experienced the therapeutic effect. For example, in some embodiments, the kill switch is activated to kill the bacteria after a period of time following expression of an oxalate catabolism enzyme. In some embodiments, the kill switch is activated in a delayed fashion following expression of the oxalate catabolism gene, for example, after the production of the oxalate catabolism enzyme. Alternatively, the bacteria may be engineered to die after the bacteria has spread outside of a disease site. Specifically, it may be useful to prevent long-term colonization of subjects by the microorganism, spread of the microorganism outside the area of interest (for example, outside the gut) within the subject, or spread of the microorganism outside of the subject into the environment (for example, spread to the environment through the stool of the subject).

Examples of such toxins that can be used in kill-switches include, but are not limited to, bacteriocins, lysins, and other molecules that cause cell death by lysing cell membranes, degrading cellular DNA, or other mechanisms. Such toxins can be used individually or in combination. The switches that control their production can be based on, for example, transcriptional activation (toggle switches; see, e.g., Gardner et al., 2000), translation (riboregulators), or DNA recombination (recombinase-based switches), and can sense environmental stimuli such as anaerobiosis or reactive oxygen species. These switches can be activated by a single environmental factor or may require several activators in AND, OR, NAND and NOR logic configurations to induce cell death. For example, an AND riboregulator switch is activated by tetracycline, isopropyl β-D-1-thiogalactopyranoside (IPTG), and arabinose to induce the expression of lysins, which permeabilize the cell membrane and kill the cell. IPTG induces the expression of the endolysin and holin mRNAs, which are then derepressed by the addition of arabinose and tetracycline. All three inducers must be present to cause cell death. Examples of kill switches are known in the art (Callura et al., 2010). In some embodiments, the kill switch is activated to kill the bacteria after a period of time following oxygen level-dependent expression of an oxalate catabolism enzyme. In some embodiments, the kill switch is activated in a delayed fashion following oxygen level-dependent expression of an oxalate catabolism enzyme.

Kill-switches can be designed such that a toxin is produced in response to an environmental condition or external signal (e.g., the bacteria are killed in response to an external cue; i.e., an activation-based kill switch) or, alternatively designed such that a toxin is produced once an environmental condition no longer exists or an external signal is ceased (i.e., a repression-based kill switch).

Thus, in some embodiments, the genetically engineered bacteria of the disclosure are further programmed to die after sensing an exogenous environmental signal, for example, in a low oxygen environment. In some embodiments, the genetically engineered bacteria of the present disclosure, e.g., bacteria expressing an oxalate catabolism enzyme, comprise one or more genes encoding one or more recombinase(s), whose expression is induced in response to an environmental condition or signal and causes one or more recombination events that ultimately leads to the expression of a toxin which kills the cell. In some embodiments, the at least one recombination event is the flipping of an inverted heterologous gene encoding a bacterial toxin which is then constitutively expressed after it is flipped by the first recombinase. In one embodiment, constitutive expression of the bacterial toxin kills the genetically engineered bacterium. In these types of kill-switch systems once the engineered bacterial cell senses the exogenous environmental condition and expresses the heterologous gene of interest, the recombinant bacterial cell is no longer viable.

In another embodiment in which the genetically engineered bacteria of the present disclosure, e.g., bacteria expressing an oxalate catabolism enzyme, express one or more recombinase(s) in response to an environmental condition or signal causing at least one recombination event, the genetically engineered bacterium further expresses a heterologous gene encoding an anti-toxin in response to an exogenous environmental condition or signal. In one embodiment, the at least one recombination event is flipping of an inverted heterologous gene encoding a bacterial toxin by a first recombinase. In one embodiment, the inverted heterologous gene encoding the bacterial toxin is located between a first forward recombinase recognition sequence and a first reverse recombinase recognition sequence. In one embodiment, the heterologous gene encoding the bacterial toxin is constitutively expressed after it is flipped by the first recombinase. In one embodiment, the anti-toxin inhibits the activity of the toxin, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In another embodiment, the at least one recombination event is flipping of an inverted heterologous gene encoding a second recombinase by a first recombinase, followed by the flipping of an inverted heterologous gene encoding a bacterial toxin by the second recombinase. In one embodiment, the inverted heterologous gene encoding the second recombinase is located between a first forward recombinase recognition sequence and a first reverse recombinase recognition sequence. In one embodiment, the inverted heterologous gene encoding the bacterial toxin is located between a second forward recombinase recognition sequence and a second reverse recombinase recognition sequence. In one embodiment, the heterologous gene encoding the second recombinase is constitutively expressed after it is flipped by the first recombinase. In one embodiment, the heterologous gene encoding the bacterial toxin is constitutively expressed after it is flipped by the second recombinase. In one embodiment, the genetically engineered bacterium is killed by the bacterial toxin. In one embodiment, the genetically engineered bacterium further expresses a heterologous gene encoding an anti-toxin in response to the exogenous environmental condition. In one embodiment, the anti-toxin inhibits the activity of the toxin when the exogenous environmental condition is present, thereby delaying death of the genetically engineered bacterium. In one embodiment, the genetically engineered bacterium is killed by the bacterial toxin when the heterologous gene encoding the anti-toxin is no longer expressed when the exogenous environmental condition is no longer present.

In one embodiment, the at least one recombination event is flipping of an inverted heterologous gene encoding a second recombinase by a first recombinase, followed by flipping of an inverted heterologous gene encoding a third recombinase by the second recombinase, followed by flipping of an inverted heterologous gene encoding a bacterial toxin by the third recombinase. Accordingly, in one embodiment, the disclosure provides at least 1, 2, 3, 4, 5, 6, 7, 8, 9, 10, 11, 12, 13, 14, 15, 16, 17, 18, 19, or 20 recombinases that can be used serially.

In one embodiment, the at least one recombination event is flipping of an inverted heterologous gene encoding a first excision enzyme by a first recombinase. In one embodiment, the inverted heterologous gene encoding the first excision enzyme is located between a first forward recombinase recognition sequence and a first reverse recombinase recognition sequence. In one embodiment, the heterologous gene encoding the first excision enzyme is constitutively expressed after it is flipped by the first recombinase. In one embodiment, the first excision enzyme excises a first essential gene. In one embodiment, the programmed recombinant bacterial cell is not viable after the first essential gene is excised.

In one embodiment, the first recombinase further flips an inverted heterologous gene encoding a second excision enzyme. In one embodiment, the wherein the inverted heterologous gene encoding the second excision enzyme is located between a second forward recombinase recognition sequence and a second reverse recombinase recognition sequence. In one embodiment, the heterologous gene encoding the second excision enzyme is constitutively expressed after it is flipped by the first recombinase. In one embodiment, the genetically engineered bacterium dies or is no longer viable when the first essential gene and the second essential gene are both excised. In one embodiment, the genetically engineered bacterium dies or is no longer viable when either the first essential gene is excised or the second essential gene is excised by the first recombinase.

In one embodiment, the first excision enzyme is Xis1. In one embodiment, the first excision enzyme is Xis2. In one embodiment, the first excision enzyme is Xis1, and the second excision enzyme is Xis2.

In one embodiment, the genetically engineered bacterium dies after the at least one recombination event occurs. In another embodiment, the genetically engineered bacterium is no longer viable after the at least one recombination event occurs.

In any of these embodiment, the recombinase can be a recombinase selected from the group consisting of: Bxb1, PhiC31, TP901, Bxb1, PhiC31, TP901, HK022, HP1, R4, Int1, Int2, Int3, Int4, Int5, Int6, Int1, Int8, Int9, Int10, Int1l, Int12, Int13, Int14, Int15, Int16, Int17, Int18, Int19, Int20, Int21, Int22, Int23, Int24, Int25, Int26, Int27, Int28, Int29, Int30, Int31, Int32, Int33, and Int34, or a biologically active fragment thereof.

In the above-described kill-switch circuits, a toxin is produced in the presence of an environmental factor or signal. In another aspect of kill-switch circuitry, a toxin may be repressed in the presence of an environmental factor (not produced) and then produced once the environmental condition or external signal is no longer present. Such kill switches are called repression-based kill switches and represent systems in which the bacterial cells are viable only in the presence of an external factor or signal, such as arabinose or other sugar. Exemplary kill switch designs in which the toxin is repressed in the presence of an external factor or signal (and activated once the external signal is removed) are shown in the figures of PCT Publication Number WO 2017/040719 (Attorney Reference No.: 126046-00520). The disclosure provides recombinant bacterial cells which express one or more heterologous gene(s) upon sensing arabinose or other sugar in the exogenous environment. In this aspect, the recombinant bacterial cells contain the araC gene, which encodes the AraC transcription factor, as well as one or more genes under the control of the araBAD promoter. In the absence of arabinose, the AraC transcription factor adopts a conformation that represses transcription of genes under the control of the araBAD promoter. In the presence of arabinose, the AraC transcription factor undergoes a conformational change that allows it to bind to and activate the araBAD promoter, which induces expression of the desired gene, for example tetR, which represses expression of a toxin gene. In this embodiment, the toxin gene is repressed in the presence of arabinose or other sugar. In an environment where arabinose is not present, the tetR gene is not activated and the toxin is expressed, thereby killing the bacteria. The arabinose system can also be used to express an essential gene, in which the essential gene is only expressed in the presence of arabinose or other sugar and is not expressed when arabinose or other sugar is absent from the environment.

Thus, in some embodiments in which one or more heterologous gene(s) are expressed upon sensing arabinose in the exogenous environment, the one or more heterologous genes are directly or indirectly under the control of the araBAD promoter. In some embodiments, the expressed heterologous gene is selected from one or more of the following: a heterologous therapeutic gene, a heterologous gene encoding an antitoxin, a heterologous gene encoding a repressor protein or polypeptide, for example, a TetR repressor, a heterologous gene encoding an essential protein not found in the bacterial cell, and/or a heterologous encoding a regulatory protein or polypeptide.

Arabinose inducible promoters are known in the art, including Pₐᵣₐ, P_{araB}, P_{araC}, and P_{araBAD}. In one embodiment, the arabinose inducible promoter is from E. coli. In some embodiments, the P_{araC} promoter and the P_{araBAD} promoter operate as a bidirectional promoter, with the P_{araBAD} promoter controlling expression of a heterologous gene(s) in one direction, and the P_{araC} (in close proximity to, and on the opposite strand from the P_{araBAD} promoter), controlling expression of a heterologous gene(s) in the other direction. In the presence of arabinose, transcription of both heterologous genes from both promoters is induced. However, in the absence of arabinose, transcription of both heterologous genes from both promoters is not induced.

In one exemplary embodiment of the disclosure, the engineered bacteria of the present disclosure contains a kill-switch having at least the following sequences: a P_{araBAD} promoter operably linked to a heterologous gene encoding a Tetracycline Repressor Protein (TetR), a P_{araC} promoter operably linked to a heterologous gene encoding AraC transcription factor, and a heterologous gene encoding a bacterial toxin operably linked to a promoter which is repressed by the Tetracycline Repressor Protein (P_{TetR}). In the presence of arabinose, the AraC transcription factor activates the P_{araBAD} promoter, which activates transcription of the TetR protein which, in turn, represses transcription of the toxin. In the absence of arabinose, however, AraC suppresses transcription from the P_{araBAD} promoter and no TetR protein is expressed. In this case, expression of the heterologous toxin gene is activated, and the toxin is expressed. The toxin builds up in the recombinant bacterial cell, and the recombinant bacterial cell is killed. In one embodiment, the araC gene encoding the AraC transcription factor is under the control of a constitutive promoter and is therefore constitutively expressed.

In one embodiment of the disclosure, the recombinant bacterial cell further comprises an antitoxin under the control of a constitutive promoter. In this situation, in the presence of arabinose, the toxin is not expressed due to repression by TetR protein, and the antitoxin protein builds-up in the cell. However, in the absence of arabinose, TetR protein is not expressed, and expression of the toxin is induced. The toxin begins to build-up within the recombinant bacterial cell. The recombinant bacterial cell is no longer viable once the toxin protein is present at either equal or greater amounts than that of the anti-toxin protein in the cell, and the recombinant bacterial cell will be killed by the toxin.

In another embodiment of the disclosure, the recombinant bacterial cell further comprises an antitoxin under the control of the P_{araBAD} promoter. In this situation, in the presence of arabinose, TetR and the anti-toxin are expressed, the anti-toxin builds up in the cell, and the toxin is not expressed due to repression by TetR protein. However, in the absence of arabinose, both the TetR protein and the anti-toxin are not expressed, and expression of the toxin is induced. The toxin begins to build-up within the recombinant bacterial cell. The recombinant bacterial cell is no longer viable once the toxin protein is expressed, and the recombinant bacterial cell will be killed by the toxin.

In another exemplary embodiment of the disclosure, the engineered bacteria of the present disclosure contain a kill-switch having at least the following sequences: A P_{araBAD} promoter operably linked to a heterologous gene encoding an essential polypeptide not found in the recombinant bacterial cell (and required for survival), and a P_{araC} promoter operably linked to a heterologous gene encoding AraC transcription factor. In the presence of arabinose, the AraC transcription factor activates the P_{araBAD} promoter, which activates transcription of the heterologous gene encoding the essential polypeptide, allowing the recombinant bacterial cell to survive. In the absence of arabinose, however, AraC suppresses transcription from the P_{araBAD} promoter and the essential protein required for survival is not expressed. In this case, the recombinant bacterial cell dies in the absence of arabinose. In some embodiments, the sequence of P_{araBAD} promoter operably linked to a heterologous gene encoding an essential polypeptide not found in the recombinant bacterial cell can be present in the bacterial cell in conjunction with the TetR/toxin kill-switch system described directly above. In some embodiments, the sequence of P_{araBAD} promoter operably linked to a heterologous gene encoding an essential polypeptide not found in the recombinant bacterial cell can be present in the bacterial cell in conjunction with the TetR/toxin/anti-toxin kill-switch system described directly above.

In yet other embodiments, the bacteria may comprise a plasmid stability system with a plasmid that produces both a short-lived anti-toxin and a long-lived toxin. In this system, the bacterial cell produces equal amounts of toxin and anti-toxin to neutralize the toxin. However, if/when the cell loses the plasmid, the short-lived anti-toxin begins to decay. When the anti-toxin decays completely the cell dies as a result of the longer-lived toxin killing it.

In some embodiments, the engineered bacteria of the present disclosure, for example, bacteria expressing an oxalate catabolism enzyme further comprise the gene(s) encoding the components of any of the above-described kill-switch circuits.

In any of the above-described embodiments, the bacterial toxin is selected from the group consisting of a lysin, Hok, Fst, TisB, LdrD, Kid, SymE, MazF, FlmA, Ibs, XCV2162, dinJ, CcdB, MazF, ParE, YafO, Zeta, hicB, relB, yhaV, yoeB, chpBK, hipA, microcin B, microcin B17, microcin C, microcin C7-C51, microcin J25, microcin ColV, microcin 24, microcin L, microcin D93, microcin L, microcin E492, microcin H47, microcin I47, microcin M, colicin A, colicin E1, colicin K, colicin N, colicin U, colicin B, colicin Ia, colicin Ib, colicin 5, colicin10, colicin S4, colicin Y, colicin E2, colicin E7, colicin E8, colicin E9, colicin E3, colicin E4, colicin E6; colicin E5, colicin D, colicin M, and cloacin DF13, or a biologically active fragment thereof.

In any of the above-described embodiments, the anti-toxin is selected from the group consisting of an anti-lysin, Sok, RNAII, IstR, RdlD, Kis, SymR, MazE, FlmB, Sib, ptaRNA1, yafQ, CcdA, MazE, ParD, yafN, Epsilon, HicA, relE, prlF, yefM, chpBI, hipB, MccE, MccE^{CTD}, MccF, Cai, ImmE1, Cki, Cni, Cui, Cbi, Iia, Imm, Cfi, Im10, Csi, Cyi, Im2, Im7, Im8, Im9, Im3, Im4, ImmE6, cloacin immunity protein (Cim), ImmE5, ImmD, and Cmi, or a biologically active fragment thereof.

In one embodiment, the bacterial toxin is bactericidal to the genetically engineered bacterium. In one embodiment, the bacterial toxin is bacteriostatic to the genetically engineered bacterium.

In one embodiment, the method further comprises administering a second recombinant bacterial cell to the subject, wherein the second recombinant bacterial cell comprises a heterologous reporter gene operably linked to an inducible promoter that is directly or indirectly induced by an exogenous environmental condition. In one embodiment, the heterologous reporter gene is a fluorescence gene. In one embodiment, the fluorescence gene encodes a green fluorescence protein (GFP). In another embodiment, the method further comprises administering a second recombinant bacterial cell to the subject, wherein the second recombinant bacterial cell expresses a lacZ reporter construct that cleaves a substrate to produce a small molecule that can be detected in urine (see, for example, Danio et al., Science Translational Medicine, 7(289):1-12, 2015, the entire contents of which are expressly incorporated herein by reference).

Isolated Plasmids

In other embodiments, the disclosure provides an isolated plasmid comprising a first nucleic acid encoding a first payload operably linked to a first inducible promoter, and a second nucleic acid encoding a second payload operably linked to a second inducible promoter. In other embodiments, the disclosure provides an isolated plasmid further comprising a third nucleic acid encoding a third payload operably linked to a third inducible promoter. In other embodiments, the disclosure provides a plasmid comprising four, five, six, or more nucleic acids encoding four, five, six, or more payloads operably linked to inducible promoters. In any of the embodiments described here, the first, second, third, fourth, fifth, sixth, etc. “payload(s)” can be an oxalate catabolism enzyme, a transporter of oxalate, or other sequence described herein. In one embodiment, the nucleic acid encoding the first payload and the nucleic acid encoding the second payload are operably linked to the first inducible promoter. In one embodiment, the nucleic acid encoding the first payload is operably linked to a first inducible promoter and the nucleic acid encoding the second payload is operably linked to a second inducible promoter. In one embodiment, the first inducible promoter and the second inducible promoter are separate copies of the same inducible promoter. In another embodiment, the first inducible promoter and the second inducible promoter are different inducible promoters. In other embodiments comprising a third nucleic acid, the nucleic acid encoding the third payload and the nucleic acid encoding the first and second payloads are all operably linked to the same inducible promoter. In other embodiments, the nucleic acid encoding the first payload is operably linked to a first inducible promoter, the nucleic acid encoding the second payload is operably linked to a second inducible promoter, and the nucleic acid encoding the third payload is operably linked to a third inducible promoter. In some embodiments, the first, second, and third inducible promoters are separate copies of the same inducible promoter. In other embodiments, the first inducible promoter, the second inducible promoter, and the third inducible promoter are different inducible promoters. In some embodiments, the first promoter, the second promoter, and the optional third promoter, or the first promoter and the second promoter and the optional third promoter, are each directly or indirectly induced by low-oxygen or anaerobic conditions. In other embodiments, the first promoter, the second promoter, and the optional third promoter, or the first promoter and the second promoter and the optional third promoter, are each a fumarate and nitrate reduction regulator (FNR) responsive promoter. In other embodiments, the first promoter, the second promoter, and the optional third promoter, or the first promoter and the second promoter and the optional third promoter are each a ROS-inducible regulatory region. In other embodiments, the first promoter, the second promoter, and the optional third promoter, or the first promoter and the second promoter and the optional third promoter are each a RNS-inducible regulatory region.

In some embodiments, the heterologous gene and/or gene cassette encoding one or more oxalate catabolism enzyme(s) is operably linked to a constitutive promoter. In one embodiment, the constitutive promoter is a lac promoter. In another embodiment, the constitutive promoter is a Tet promoter. In another embodiment, the constitutive promoter is a constitutive Escherichia coli σ³² promoter. In another embodiment, the constitutive promoter is a constitutive Escherichia coli σ⁷⁰ promoter. In another embodiment, the constitutive promoter is a constitutive Bacillus subtilis σ^{A} promoter. In another embodiment, the constitutive promoter is a constitutive Bacillus subtilis σ^{B} promoter. In another embodiment, the constitutive promoter is a Salmonella promoter. In other embodiments, the constitutive promoter is a bacteriophage T7 promoter. In other embodiments, the constitutive promoter is and a bacteriophage SP6 promoter. In any of the above-described embodiments, the plasmid further comprises a heterologous gene encoding a transporter oxalate, and/or a kill switch construct, which may be operably linked to a constitutive promoter or an inducible promoter.

In some embodiments, the isolated plasmid comprises at least one heterologous oxalate catabolism enzyme gene operably linked to a first inducible promoter; a heterologous gene encoding a TetR protein operably linked to a P_{araBAD} promoter, a heterologous gene encoding AraC operably linked to a P_{araC} promoter, a heterologous gene encoding an antitoxin operably linked to a constitutive promoter, and a heterologous gene encoding a toxin operably linked to a P_{TetR} promoter. In another embodiment, the isolated plasmid comprises at least one heterologous gene and/or gene cassette encoding one or more oxalate catabolism enzyme(s) operably linked to a first inducible promoter; a heterologous gene encoding a TetR protein and an anti-toxin operably linked to a P_{araBAD} promoter, a heterologous gene encoding AraC operably linked to a P_{araC} promoter, and a heterologous gene encoding a toxin operably linked to a P_{TetR} promoter.

In some embodiments, a first nucleic acid encoding one or more oxalate catabolism enzyme(s) comprises a Formyl CoA: oxalate CoA transferase (e.g., frc) gene. In one embodiment, the frc gene is from O. formigenes. In one embodiment, the frc gene has at least about 90% identity to SEQ ID NO: 1. In another embodiment, the frc gene comprises SEQ ID NO: 1. In other embodiments, a first nucleic acid encoding one or more oxalate catabolism enzyme(s) comprises an Oxalate-CoA ligase (e.g., ScAAE3) gene. In one embodiment, the ScAAE3 gene is from S. cerevisiae. In one embodiment, the ScAAE3 gene has at least about 90% identity to SEQ ID NO: 3. In another embodiment, the ScAAE3 gene comprises SEQ ID NO: 3. In other embodiments, a first nucleic acid encoding one or more oxalate catabolism enzyme(s) comprises an acetyl-CoA: oxalate CoA-transferase (e.g., YfdE) gene. In one embodiment, the YfdE gene is from E. coli. In one embodiment, the YfdE gene has at least about 90% identity to SEQ ID NO: 4. In another embodiment, the YfdE gene comprises SEQ ID NO: 4.

In some embodiments, a first nucleic acid encoding one or more oxalate catabolism enzyme(s) comprises a Oxalyl-CoA Decarboxylase (e.g., oxc) gene. In some embodiments, the frc and/or ScAAE3 and/or YfdE gene(s) are co-expressed with a Oxalyl-CoA Decarboxylase (e.g., oxc) gene. In one embodiment, the oxc gene is from O. formigenes. In one embodiment, the oxc gene has at least about 90% identity to SEQ ID NO: 2. In another embodiment, the oxc gene comprises SEQ ID NO: 2.

In some embodiments, a second nucleic acid encoding a transporter of oxalate comprises OxlT. In one embodiment, the OxlT transporter is from O. formigenes. In another embodiment, the OxlT transporter has at least about 90% identity to SEQ ID NO: 11. In another embodiment, the OxlT transporter comprises SEQ ID NO: 11.

In one embodiment, the plasmid is a high-copy plasmid. In another embodiment, the plasmid is a low-copy plasmid.

In another aspect, the disclosure provides a recombinant bacterial cell comprising an isolated plasmid described herein. In another embodiment, the disclosure provides a pharmaceutical composition comprising the recombinant bacterial cell.

In one embodiment, the bacterial cell further comprises a genetic mutation in an endogenous gene encoding an exporter of oxalate, wherein the genetic mutation reduces export of oxalate from the bacterial cell.

In one embodiment, the bacterial cell further comprises a genetic mutation in an endogenous gene encoding an oxalate biosynthesis gene, wherein the genetic mutation reduces biosynthesis of oxalate in the bacterial cell.

Combinations

In some embodiments, the bacteria are genetically engineered to include multiple mechanisms of action (MOAs), e.g., circuits producing multiple copies of the same product (e.g., to enhance copy number) or circuits performing multiple different functions. Examples of insertion sites include, but are not limited to, malE/K, insB/I, araC/BAD, lacZ, dapA, and cea. For example, the genetically engineered bacteria may include four copies of an oxalate catabolism gene or oxalate catabolism gene cassette, or four copies of an oxalate catabolism gene inserted at four different insertion sites, e.g., malE/K, insB/I, araC/BAD, and lacZ. Alternatively, the genetically engineered bacteria may include one or more copies of an oxalate catabolism gene or gene cassette inserted at one or more different insertion sites, e.g., malE/K, insB/I, and lacZ, one or more copies of an oxalate catabolism gene or gene cassette inserted at one or more different insertion sites, e.g., dapA, cea, and araC/BAD and/or one or more copies of an oxalate catabolism gene or gene cassette inserted at one or more different insertion sites.

In some embodiments, the genetically engineered bacteria comprise one or more of: (1) one or more gene(s) and/or gene cassettes encoding one or more oxalate catabolism enzyme(s) described herein, in wild type or in a mutated form (for increased stability or metabolic activity); (2) one or more gene(s) and/or gene cassette(s) encoding one or more transporter(s) for uptake of oxalate, in wild type or in mutated form (for increased stability or metabolic activity), which in some embodiments are coupled to formate export; (3) one or more gene(s) and/or gene cassette(s) encoding one or more exporters(s) for export of formate, in wild type or in mutated form (for increased stability or metabolic activity), which in some embodiments are be coupled to oxalate import; (4) one or more gene(s) and/or gene cassette(s) encoding one or more oxalate:formate antiporters, in wild type or in mutated form (for increased stability or metabolic activity), which in some embodiments are be coupled to oxalate import; (5) one or more gene(s) or gene cassette(s) encoding one or more oxalate catabolism enzyme(s) described herein for secretion and extracellular degradation of oxalate (6) one or more gene(s) or gene cassette(s) encoding one or more components of secretion machinery, as described herein (7) one or more auxotrophies, e.g., deltaThyA; (8) one or more gene(s) or gene cassette(s) encoding one or more antibiotic resistance(s), including but not limited to, kanamycin or chloramphenicol resistance; (9) one or more modifications that increase oxalate import into the bacterial cell (10) one or more modifications that increase formate export from the bacterial cell (11) one or modifications that reduce formate import into the bacterial cell; (12) one or modifications that reduce oxalate export from the bacterial cell; (13) mutations/deletions in genes of the endogenous oxalate synthesis pathway; (14) mutations and/or deletions in formate exporters, which result in increased flux through a oxalate:formate antiporter and increased oxalate import.

In some embodiments, the genetically engineered bacteria comprise two or more different pathway cassettes or operons comprising oxalate catabolism enzymes. In some embodiments, the genetically engineered bacteria comprise one or more oxalate catabolism gene(s) or gene cassette(s) encoding one or more oxalate catabolism enzymes. In some embodiments, the genetically engineered bacteria comprise gene sequence(s) encoding one or more oxalate catabolism enzymes selected formyl-CoA:oxalate CoA-transferase, including but not limited to frc from O. formigenes, oxalyl-CoA synthetase, including but not limited to ScAAE3 from S. cerevisiae, oxalyl-CoA decarboxylase, including but not limited to oxc from O. formigenes, and/or acetyl-CoA:oxalate CoA-transferase, including but not limited to YfdE from E. coli.

In some embodiments, the genetically engineered bacteria comprise one or more oxalate catabolism gene(s) or gene cassette(s) encoding formyl-CoA:oxalate CoA-transferase, including but not limited to frc from O. formigenes. In some embodiments, the genetically engineered bacteria comprise one or more oxalate catabolism gene(s) or gene cassette(s) encoding oxalyl-CoA synthetase, including but not limited to ScAAE3 from S. cerevisiae. In some embodiments, the genetically engineered bacteria comprise one or more oxalate catabolism gene(s) or gene cassette(s) encoding oxalyl-CoA decarboxylase, including but not limited to oxc from O. formigenes. In some embodiments, the genetically engineered bacteria comprise one or more oxalate catabolism gene(s) or gene cassette(s) encoding acetyl-CoA:oxalate CoA-transferase, including but not limited to YfdE from E. coli. In some embodiments, the genetically engineered bacteria comprise one or more oxalate catabolism gene(s) or gene cassette(s) encoding formyl-CoA:oxalate CoA-transferase, including but not limited to frc from O. formigenes, and oxalyl-CoA synthetase, including but not limited to ScAAE3 from S. cerevisiae. In some embodiments, the genetically engineered bacteria comprise one or more oxalate catabolism gene(s) or gene cassette(s) encoding formyl-CoA:oxalate CoA-transferase, including but not limited to frc from O. formigenes, and oxalyl-CoA decarboxylase, including but not limited to oxc from O. formigenes. In some embodiments, the genetically engineered bacteria comprise one or more oxalate catabolism gene(s) or gene cassette(s) encoding formyl-CoA:oxalate CoA-transferase, including but not limited to frc from O. formigenes, and acetyl-CoA:oxalate CoA-transferase, including but not limited to YfdE from E. coli. In some embodiments, the genetically engineered bacteria comprise one or more oxalate catabolism gene(s) or gene cassette(s) encoding oxalyl-CoA synthetase, including but not limited to ScAAE3 from S. cerevisiae, and oxalyl-CoA decarboxylase, including but not limited to oxc from O. formigenes. In some embodiments, the genetically engineered bacteria comprise one or more oxalate catabolism gene(s) or gene cassette(s) encoding oxalyl-CoA synthetase, including but not limited to ScAAE3 from S. cerevisiae, and acetyl-CoA:oxalate CoA-transferase, including but not limited to YfdE from E. coli. In some embodiments, the genetically engineered bacteria comprise one or more oxalate catabolism gene(s) or gene cassette(s) encoding oxalyl-CoA decarboxylase, including but not limited to oxc from O. formigenes, and acetyl-CoA:oxalate CoA-transferase, including but not limited to YfdE from E. coli. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, and oxalyl-CoA decarboxylase, including but not limited to oxc from O. formigenes. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, and acetyl-CoA:oxalate CoA-transferase, including but not limited to YfdE from E. coli. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA decarboxylase, including but not limited to oxc from O. formigenes, and acetyl-CoA:oxalate CoA-transferase, including but not limited to YfdE from E. coli. In some embodiments, the genetically engineered bacteria comprise one or more oxalate catabolism gene(s) or gene cassette(s) encoding oxalyl-CoA synthetase, including but not limited to ScAAE3 from S. cerevisiae, oxalyl-CoA decarboxylase, including but not limited to oxc from O. formigenes, and acetyl-CoA:oxalate CoA-transferase, including but not limited to YfdE from E. coli. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, oxalyl-CoA decarboxylase, including but not limited to oxc from O. formigenes, and acetyl-CoA:oxalate CoA-transferase, including but not limited to YfdE from E. coli.

In some embodiments, the genetically engineered bacteria further comprise one or more oxalate transporters and/or antiporters, including but not limited to OxIT from O. formigenenes. In some embodiments, the genetically engineered bacteria comprise one or more oxalate catabolism gene(s) or gene cassette(s) encoding an oxalate transporter, e.g, the oxalate:formate antiporter OxlT from O. formigenes. In some embodiments, the genetically engineered bacteria comprise one or more oxalate catabolism gene(s) or gene cassette(s) encoding formyl-CoA:oxalate CoA-transferase, including but not limited to frc from O. formigenes, and an oxalate transporter, e.g., the oxalate:formate antiporter OxlT from O. formigenes. In some embodiments, the genetically engineered bacteria comprise one or more oxalate catabolism gene(s) or gene cassette(s) encoding oxalyl-CoA synthetase, including but not limited to ScAAE3 from S. cerevisiae, and an oxalate transporter, e.g., the oxalate:formate antiporter OxIT from O. formigenes. In some embodiments, the genetically engineered bacteria comprise one or more oxalate catabolism gene(s) or gene cassette(s) encoding oxalyl-CoA decarboxylase, including but not limited to oxc from O. formigenes, and an oxalate transporter, e.g., the oxalate:formate antiporter OxlT from O. formigenes. In some embodiments, the genetically engineered bacteria comprise one or more oxalate catabolism gene(s) or gene cassette(s) encoding acetyl-CoA:oxalate CoA-transferase, including but not limited to YfdE from E. coli, and an oxalate transporter, e.g., the oxalate:formate antiporter OxlT from O. formigenes. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, and an oxalate transporter, e.g., the oxalate:formate antiporter OxlT from O. formigenes. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA decarboxylase, including but not limited to oxc from O. formigenes, and an oxalate transporter, e.g., the oxalate:formate antiporter OxlT from O. formigenes. In some embodiments, the genetically engineered bacteria comprise one or more oxalate catabolism gene(s) or gene cassette(s) encoding formyl-CoA:oxalate CoA-transferase, including but not limited to frc from O. formigenes, acetyl-CoA:oxalate CoA-transferase, including but not limited to YfdE from E. coli, and an oxalate transporter, e.g., the oxalate:formate antiporter OxlT from O. formigenes. In some embodiments, the genetically engineered bacteria comprise one or more oxalate catabolism gene(s) or gene cassette(s) encoding oxalyl-CoA synthetase, including but not limited to ScAAE3 from S. cerevisiae, oxalyl-CoA decarboxylase, including but not limited to oxc from O. formigenes, and an oxalate transporter, e.g., the oxalate:formate antiporter OxlT from O. formigenes. In some embodiments, the genetically engineered bacteria comprise one or more oxalate catabolism gene(s) or gene cassette(s) encoding oxalyl-CoA synthetase, including but not limited to ScAAE3 from S. cerevisiae, acetyl-CoA:oxalate CoA-transferase, including but not limited to YfdE from E. coli, and an oxalate transporter, e.g., the oxalate:formate antiporter OxlT from O. formigenes. In some embodiments, the genetically engineered bacteria comprise one or more oxalate catabolism gene(s) or gene cassette(s) encoding oxalyl-CoA decarboxylase, including but not limited to oxc from O. formigenes, acetyl-CoA:oxalate CoA-transferase, including but not limited to YfdE from E. coli, and an oxalate transporter, e.g., the oxalate:formate antiporter OxlT from O. formigenes. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, oxalyl-CoA decarboxylase, including but not limited to oxc from O. formigenes, and an oxalate transporter, e.g., the oxalate:formate antiporter OxIT from O. formigenes. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, acetyl-CoA:oxalate CoA-transferase, including but not limited to YfdE from E. coli, and an oxalate transporter, e.g., the oxalate:formate antiporter OxlT from O. formigenes. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA decarboxylase, including but not limited to oxc from O. formigenes, acetyl-CoA:oxalate CoA-transferase, including but not limited to YfdE from E. coli, and an oxalate transporter, e.g., the oxalate:formate antiporter OxlT from O. formigenes. In some embodiments, the genetically engineered bacteria comprise one or more oxalate catabolism gene(s) or gene cassette(s) encoding oxalyl-CoA synthetase, including but not limited to ScAAE3 from S. cerevisiae, oxalyl-CoA decarboxylase, including but not limited to oxc from O. formigenes, acetyl-CoA:oxalate CoA-transferase, including but not limited to YfdE from E. coli, and an oxalate transporter, e.g., the oxalate:formate antiporter OxlT from O. formigenes. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, oxalyl-CoA decarboxylase, including but not limited to oxc from O. formigenes, acetyl-CoA:oxalate CoA-transferase, including but not limited to YfdE from E. coli, and an oxalate transporter, e.g., the oxalate:formate antiporter OxlT from O. formigenes.

In some embodiments, the genetically engineered bacteria, which comprise one or more oxalate catabolism gene(s) or gene cassette(s) and one or more oxalate transporter(s), encode a formate exporter, e.g., as described herein. In some embodiments, the genetically engineered bacteria comprise one or more oxalate catabolism gene(s) or gene cassette(s) and one or more oxalate transporter(s), which also function(s) as an formate exporter, e.g., OxlT, described herein. In some embodiments, formate export and oxalate import are separate. In some embodiments, the genetically engineered bacteria which comprise one or more oxalate catabolism gene(s) or gene cassette(s), comprise a formate exporter. In some embodiments, the genetically engineered bacteria comprise one or more oxalate catabolism gene(s) or gene cassette(s) encoding formyl-CoA:oxalate CoA-transferase, including but not limited to frc from O. formigenes, and a formate exporter, e.g., described herein. In some embodiments, the genetically engineered bacteria comprise one or more oxalate catabolism gene(s) or gene cassette(s) encoding oxalyl-CoA synthetase, including but not limited to ScAAE3 from S. cerevisiae, and a formate exporter, e.g., described herein. In some embodiments, the genetically engineered bacteria comprise one or more oxalate catabolism gene(s) or gene cassette(s) encoding oxalyl-CoA decarboxylase, including but not limited to oxc from O. formigenes, and a formate exporter, e.g., described herein. In some embodiments, the genetically engineered bacteria comprise one or more oxalate catabolism gene(s) or gene cassette(s) encoding acetyl-CoA:oxalate CoA-transferase, including but not limited to YfdE from E. coli, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA decarboxylase, including but not limited to oxc from O. formigenes, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, acetyl-CoA:oxalate CoA-transferase, including but not limited to YfdE from E. coli, and a formate exporter, e.g., described herein. In some embodiments, the genetically engineered bacteria comprise one or more oxalate catabolism gene(s) or gene cassette(s) encoding oxalyl-CoA synthetase, including but not limited to ScAAE3 from S. cerevisiae, oxalyl-CoA decarboxylase, including but not limited to oxc from O. formigenes, and a formate exporter, e.g., described herein. In some embodiments, the genetically engineered bacteria comprise one or more oxalate catabolism gene(s) or gene cassette(s) encoding oxalyl-CoA synthetase, including but not limited to ScAAE3 from S. cerevisiae, acetyl-CoA:oxalate CoA-transferase, including but not limited to YfdE from E. coli, and a formate exporter, e.g., described herein. In some embodiments, the genetically engineered bacteria comprise one or more oxalate catabolism gene(s) or gene cassette(s) encoding oxalyl-CoA decarboxylase, including but not limited to oxc from O. formigenes, acetyl-CoA:oxalate CoA-transferase, including but not limited to YfdE from E. coli, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, oxalyl-CoA decarboxylase, including but not limited to oxc from O. formigenes, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, acetyl-CoA:oxalate CoA-transferase, including but not limited to YfdE from E. coli, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA decarboxylase, including but not limited to oxc from O. formigenes, acetyl-CoA:oxalate CoA-transferase, including but not limited to YfdE from E. coli, and a formate exporter, e.g., described herein. In some embodiments, the genetically engineered bacteria comprise one or more oxalate catabolism gene(s) or gene cassette(s) encoding oxalyl-CoA synthetase, including but not limited to ScAAE3 from S. cerevisiae, oxalyl-CoA decarboxylase, including but not limited to oxc from O. formigenes, acetyl-CoA:oxalate CoA-transferase, including but not limited to YfdE from E. coli, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, oxalyl-CoA decarboxylase, including but not limited to oxc from O. formigenes, acetyl-CoA:oxalate CoA-transferase, including but not limited to YfdE from E. coli, and a formate exporter, e.g., described herein. In some embodiments, the genetically engineered bacteria comprise one or more oxalate catabolism gene(s) or gene cassette(s) encoding. In some embodiments, the genetically engineered bacteria comprise one or more oxalate catabolism gene(s) or gene cassette(s) encoding an oxalate transporter, e.g., the oxalate:formate antiporter OxlT from O. formigenes, a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, an oxalate transporter, e.g., the oxalate:formate antiporter OxlT from O. formigenes, and a formate exporter, e.g., described herein. In some embodiments, the genetically engineered bacteria comprise one or more oxalate catabolism gene(s) or gene cassette(s) encoding oxalyl-CoA synthetase, including but not limited to ScAAE3 from S. cerevisiae, an oxalate transporter, e.g, the oxalate:formate antiporter OxlT from O. formigenes, and a formate exporter, e.g., described herein. In some embodiments, the genetically engineered bacteria comprise one or more oxalate catabolism gene(s) or gene cassette(s) encoding oxalyl-CoA decarboxylase, including but not limited to oxc from O. formigenes, an oxalate transporter, e.g., the oxalate:formate antiporter OxlT from O. formigenes, and a formate exporter, e.g., described herein. In some embodiments, the genetically engineered bacteria comprise one or more oxalate catabolism gene(s) or gene cassette(s) encoding acetyl-CoA:oxalate CoA-transferase, including but not limited to YfdE from E. coli, an oxalate transporter, e.g., the oxalate:formate antiporter OxlT from O. formigenes, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, an oxalate transporter, e.g., the oxalate:formate antiporter OxIT from O. formigenes, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA decarboxylase, including but not limited to oxc from O. formigenes, an oxalate transporter, e.g., the oxalate:formate antiporter OxlT from O. formigenes, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, acetyl-CoA:oxalate CoA-transferase, including but not limited to YfdE from E. coli, an oxalate transporter, e.g., the oxalate:formate antiporter OxlT from O. formigenes, and a formate exporter, e.g., described herein. In some embodiments, the genetically engineered bacteria comprise one or more oxalate catabolism gene(s) or gene cassette(s) encoding oxalyl-CoA synthetase, including but not limited to ScAAE3 from S. cerevisiae, oxalyl-CoA decarboxylase, including but not limited to oxc from O. formigenes, an oxalate transporter, e.g., the oxalate:formate antiporter OxlT from O. formigenes, and a formate exporter, e.g., described herein. In some embodiments, the genetically engineered bacteria comprise one or more oxalate catabolism gene(s) or gene cassette(s) encoding oxalyl-CoA synthetase, including but not limited to ScAAE3 from S. cerevisiae, acetyl-CoA:oxalate CoA-transferase, including but not limited to YfdE from E. coli, an oxalate transporter, e.g., the oxalate:formate antiporter OxlT from O. formigenes, and a formate exporter, e.g., described herein. In some embodiments, the genetically engineered bacteria comprise one or more oxalate catabolism gene(s) or gene cassette(s) encoding oxalyl-CoA decarboxylase, including but not limited to oxc from O. formigenes, acetyl-CoA:oxalate CoA-transferase, including but not limited to YfdE from E. coli, an oxalate transporter, e.g., the oxalate:formate antiporter OxlT from O. formigenes, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, oxalyl-CoA decarboxylase, including but not limited to oxc from O. formigenes, an oxalate transporter, e.g., the oxalate:formate antiporter OxlT from O. formigenes, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, acetyl-CoA:oxalate CoA-transferase, including but not limited to YfdE from E. coli, an oxalate transporter, e.g, the oxalate:formate antiporter OxlT from O. formigenes, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA decarboxylase, including but not limited to oxc from O. formigenes, acetyl-CoA:oxalate CoA-transferase, including but not limited to YfdE from E. coli, an oxalate transporter, e.g., the oxalate:formate antiporter OxlT from O. formigenes, and a formate exporter, e.g., described herein. In some embodiments, the genetically engineered bacteria comprise one or more oxalate catabolism gene(s) or gene cassette(s) encoding oxalyl-CoA synthetase, including but not limited to ScAAE3 from S. cerevisiae, oxalyl-CoA decarboxylase, including but not limited to oxc from O. formigenes, acetyl-CoA:oxalate CoA-transferase, including but not limited to YfdE from E. coli, an oxalate transporter, e.g., the oxalate:formate antiporter OxIT from O. formigenes, and a formate exporter, e.g., described herein. In some embodiments, the genetically engineered bacteria comprise one or more oxalate catabolism gene(s) or gene cassette(s) encoding formyl-CoA:oxalate CoA-transferase, including but not limited to frc from O. formigenes, oxalyl-CoA synthetase, including but not limited to ScAAE3 from S. cerevisiae, oxalyl-CoA decarboxylase, including but not limited to oxc from O. formigenes, acetyl-CoA:oxalate CoA-transferase, including but not limited to YfdE from E. coli, an oxalate transporter, e.g., the oxalate:formate antiporter OxlT from O. formigenes, and a formate exporter, e.g., described herein.

In some embodiments, the genetically engineered bacteria further comprise one or more of auxotrophies, e.g., deltaThyA; one or more gene(s) or gene cassette(s) encoding one or more antibiotic resistance(s), including but not limited to, kanamycin or chloramphenicol resistance; one or more modifications that increase formate export from the bacterial cell; one or modifications that reduce formate import into the bacterial cell; mutations/deletions in genes, as described herein, e.g., oxalate exporters; mutations/deletions in genes of the endogenous oxalate synthesis pathway. In some embodiments, the genetically engineered bacteria comprise two or more different pathway cassettes or operons comprising oxalate catabolism enzymes. In one embodiment, the gene sequence(s) encoding the one or more oxalate catabolism enzyme(s) is directly operably linked to a first promoter. In another embodiment, the gene sequence(s) encoding the one or more oxalate catabolism enzyme(s) is indirectly operably linked to a first promoter. In one embodiment, the promoter is not operably linked with the at least one gene encoding the oxalate catabolism enzyme in nature.

In some embodiments, the gene sequence(s) encoding the one or more oxalate catabolism enzyme(s) is expressed under the control of a constitutive promoter. In another embodiment, the gene sequence(s) encoding the one or more oxalate catabolism enzyme(s) is expressed under the control of an inducible promoter. In some embodiments, the gene sequence(s) encoding the one or more oxalate catabolism enzyme(s) is expressed under the control of a promoter that is directly or indirectly induced by exogenous environmental conditions. In one embodiment, the gene sequence(s) encoding the one or more oxalate catabolism enzyme(s) is expressed under the control of a promoter that is directly or indirectly induced by low-oxygen or anaerobic conditions, wherein expression of the gene sequence(s) encoding the one or more oxalate catabolism enzyme(s) is activated under low-oxygen or anaerobic environments, such as the environment of the mammalian gut. In some embodiments, the gene sequence(s) encoding the one or more oxalate catabolism enzyme(s) is expressed under the control of a promoter that is directly or indirectly induced by inflammatory conditions. Exemplary inducible promoters described herein include oxygen level-dependent promoters (e.g., FNR-inducible promoter), promoters induced by inflammation or an inflammatory response (RNS, ROS promoters), and promoters induced by a metabolite that may or may not be naturally present (e.g., can be exogenously added) in the gut, e.g., arabinose and tetracycline. Examples of inducible promoters include, but are not limited to, an FNR responsive promoter, a P_{araC} promoter, a P_{araBAD} promoter, a P_{TetR} promoter, and a P_{LacI} promoter, each of which are described in more detail herein. Inducible promoters are described in more detail infra.

The at least one gene encoding the at least one oxalate catabolism enzyme may be present on a plasmid or chromosome in the bacterial cell. In one embodiment, the gene sequence(s) encoding the one or more oxalate catabolism enzyme(s) is located on a plasmid in the bacterial cell. In another embodiment, the gene sequence(s) encoding the one or more oxalate catabolism enzyme(s) is located in the chromosome of the bacterial cell. In yet another embodiment, a native copy of the gene sequence(s) encoding the one or more oxalate catabolism enzyme(s) is located in the chromosome of the bacterial cell, and at least one gene encoding at least one oxalate catabolism enzyme from a different species of bacteria is located on a plasmid in the bacterial cell. In yet another embodiment, a native copy of the gene sequence(s) encoding the one or more oxalate catabolism enzyme(s) is located on a plasmid in the bacterial cell, and at least one gene encoding the at least one oxalate catabolism enzyme from a different species of bacteria is located on a plasmid in the bacterial cell. In yet another embodiment, a native copy of the gene sequence(s) encoding the one or more oxalate catabolism enzyme(s) is located in the chromosome of the bacterial cell, and at least one gene encoding the at least one oxalate catabolism enzyme from a different species of bacteria is located in the chromosome of the bacterial cell.

In some embodiments, the gene sequence(s) encoding the one or more oxalate catabolism enzyme(s) is expressed on a low-copy plasmid. In some embodiments, the gene sequence(s) encoding the one or more oxalate catabolism enzyme(s) is expressed on a high-copy plasmid. In some embodiments, the high-copy plasmid may be useful for increasing expression of the at least one oxalate catabolism enzyme, thereby increasing the catabolism of oxalate, oxalic acid, and/or oxalyl-CoA.

In some embodiments, a recombinant bacterial cell of the invention comprising at least one gene encoding at least one oxalate catabolism enzyme expressed on a high-copy plasmid does not increase oxalate catabolism or decrease oxalate and/or oxalic acid levels as compared to a recombinant bacterial cell comprising the same gene expressed on a low-copy plasmid in the absence of a heterologous importer of oxalate and additional copies of a native importer of oxalate. Furthermore, in some embodiments that incorporate a transporter (importer) of oxalate into the recombinant bacterial cell, there may be additional advantages to using a low-copy plasmid comprising the gene sequence(s) encoding the one or more oxalate catabolism enzyme(s) in conjunction in order to enhance the stability of expression of the oxalate catabolism enzyme, while maintaining high oxalate catabolism and to reduce negative selection pressure on the transformed bacterium. In alternate embodiments, the importer of oxalate is used in conjunction with a high-copy plasmid.

In some embodiments, the genetically engineered bacteria described above further comprise one or more of the modifications, mutations, and/or deletions in endogenous genes described herein.

Host-Plasmid Mutual Dependency

In some embodiments, the genetically engineered bacteria also comprise a plasmid that has been modified to create a host-plasmid mutual dependency. In certain embodiments, the mutually dependent host-plasmid platform is GeneGuard (Wright et al., 2015). In some embodiments, the GeneGuard plasmid comprises (i) a conditional origin of replication, in which the requisite replication initiator protein is provided in trans; (ii) an auxotrophic modification that is rescued by the host via genomic translocation and is also compatible for use in rich media; and/or (iii) a nucleic acid sequence which encodes a broad-spectrum toxin. The toxin gene may be used to select against plasmid spread by making the plasmid DNA itself disadvantageous for strains not expressing the anti-toxin (e.g., a wild-type bacterium). In some embodiments, the GeneGuard plasmid is stable for at least 100 generations without antibiotic selection. In some embodiments, the GeneGuard plasmid does not disrupt growth of the host. The GeneGuard plasmid is used to greatly reduce unintentional plasmid propagation in the genetically engineered bacteria described herein.

The mutually dependent host-plasmid platform may be used alone or in combination with other biosafety mechanisms, such as those described herein (e.g., kill switches, auxotrophies). In some embodiments, the genetically engineered bacteria comprise a GeneGuard plasmid. In other embodiments, the genetically engineered bacteria comprise a GeneGuard plasmid and/or one or more kill switches. In other embodiments, the genetically engineered bacteria comprise a GeneGuard plasmid and/or one or more auxotrophies. In still other embodiments, the genetically engineered bacteria comprise a GeneGuard plasmid, one or more kill switches, and/or one or more auxotrophies.

In some embodiments, the vector comprises a conditional origin of replication. In some embodiments, the conditional origin of replication is a R6K or ColE2-P9. In embodiments where the plasmid comprises the conditional origin of replication R6K, the host cell expresses the replication initiator protein 7c. In embodiments where the plasmid comprises the conditional origin or replication ColE2, the host cell expresses the replication initiator protein RepA. It is understood by those of skill in the art that the expression of the replication initiator protein may be regulated so that a desired expression level of the protein is achieved in the host cell to thereby control the replication of the plasmid. For example, in some embodiments, the expression of the gene encoding the replication initiator protein may be placed under the control of a strong, moderate, or weak promoter to regulate the expression of the protein.

In some embodiments, the vector comprises a gene encoding a protein required for complementation of a host cell auxotrophy, preferably a rich-media compatible auxotrophy. In some embodiments, the host cell is auxotrophic for thymidine (ΔthyA), and the vector comprises the thymidylate synthase (thyA) gene. In some embodiments, the host cell is auxotrophic for diaminopimelic acid (ΔdapA) and the vector comprises the 4-hydroxy-tetrahydrodipicolinate synthase (dapA) gene. It is understood by those of skill in the art that the expression of the gene encoding a protein required for complementation of the host cell auxotrophy may be regulated so that a desired expression level of the protein is achieved in the host cell.

In some embodiments, the vector comprises a toxin gene. In some embodiments, the host cell comprises an anti-toxin gene encoding and/or required for the expression of an anti-toxin. In some embodiments, the toxin is Zeta and the anti-toxin is Epsilon. In some embodiments, the toxin is Kid, and the anti-toxin is Kis. In preferred embodiments, the toxin is bacteriostatic. Any of the toxin/antitoxin pairs described herein may be used in the vector systems of the present disclosure. It is understood by those of skill in the art that the expression of the gene encoding the toxin may be regulated using art known methods to prevent the expression levels of the toxin from being deleterious to a host cell that expresses the anti-toxin. For example, in some embodiments, the gene encoding the toxin may be regulated by a moderate promoter. In other embodiments, the gene encoding the toxin may be cloned adjacent to ribosomal binding site of interest to regulate the expression of the gene at desired levels (see, e.g., Wright et al. (2015)).

Integration

In some embodiments, any of the gene(s) or gene cassette(s) of the present disclosure may be integrated into the bacterial chromosome at one or more integration sites. One or more copies of the gene (for example, an oxalate catabolism gene, oxalate transporter gene, and/or oxalate binding protein gene) or gene cassette (for example, a gene cassette comprising an oxalate catabolism gene and/or an oxalate transporter gene may be integrated into the bacterial chromosome. Having multiple copies of the gene or gene cassette integrated into the chromosome allows for greater production of the payload, e.g., one or more oxalate catabolism enzyme(s) and/or oxalate transporter gene(s) and other enzymes of a gene cassette, and also permits fine-tuning of the level of expression. Alternatively, different circuits described herein, such as any of the kill-switch circuits, in addition to the therapeutic gene(s) or gene cassette(s) could be integrated into the bacterial chromosome at one or more different integration sites to perform multiple different functions.

Secretion

In some embodiments, the genetically engineered bacteria further comprise a native secretion mechanism (e.g., gram positive bacteria) or non-native secretion mechanism (e.g., gram negative bacteria) that is capable of secreting the oxalate catabolism enzyme from the bacterial cytoplasm. Many bacteria have evolved sophisticated secretion systems to transport substrates across the bacterial cell envelope. Substrates, such as small molecules, proteins, and DNA, may be released into the extracellular space or periplasm (such as the gut lumen or other space), injected into a target cell, or associated with the bacterial membrane.

In Gram-negative bacteria, secretion machineries may span one or both of the inner and outer membranes. In some embodiments, the genetically engineered bacteria further comprise a non-native double membrane-spanning secretion system. Double membrane-spanning secretion systems include, but are not limited to, the type I secretion system (T1SS), the type II secretion system (T2SS), the type III secretion system (T3SS), the type W secretion system (T4SS), the type VI secretion system (T6SS), and the resistance-nodulation-division (RND) family of multi-drug efflux pumps (Pugsley 1993; Gerlach et al., 2007; Collinson et al., 2015; Costa et al., 2015; Reeves et al., 2015; WO2014138324A1, incorporated herein by reference). Mycobacteria, which have a Gram-negative-like cell envelope, may also encode a type VII secretion system (T7SS) (Stanley et al., 2003). With the exception of the T2SS, double membrane-spanning secretions generally transport substrates from the bacterial cytoplasm directly into the extracellular space or into the target cell. In contrast, the T2SS and secretion systems that span only the outer membrane may use a two-step mechanism, wherein substrates are first translocated to the periplasm by inner membrane-spanning transporters, and then transferred to the outer membrane or secreted into the extracellular space. Outer membrane-spanning secretion systems include, but are not limited to, the type V secretion or autotransporter system (T5SS), the curli secretion system, and the chaperone-usher pathway for pili assembly (Saier, 2006; Costa et al., 2015).

In some embodiments, the genetically engineered bacteria of the invention further comprise a type III or a type III-like secretion system (T3SS) from Shigella, Salmonella, E. coli, Bivrio, Burkholderia, Yersinia, Chlamydia, or Pseudomonas. The T3SS is capable of transporting a protein from the bacterial cytoplasm to the host cytoplasm through a needle complex. The T3SS may be modified to secrete the molecule from the bacterial cytoplasm, but not inject the molecule into the host cytoplasm. Thus, the molecule is secreted into the gut lumen or other extracellular space. In some embodiments, the genetically engineered bacteria comprise said modified T3SS and are capable of secreting the oxalate catabolism enzyme from the bacterial cytoplasm. In some embodiments, the secreted molecule, such as a heterologous protein or peptide, e.g., an oxalate catabolism enzyme, comprises a type III secretion sequence that allows the oxalate catabolism enzyme to be secreted from the bacteria.

In some embodiments, a flagellar type III secretion pathway is used to secrete the molecule of interest, e.g., an oxalate catabolism enzyme. In some embodiments, an incomplete flagellum is used to secrete a therapeutic peptide of interest by recombinantly fusing the peptide to an N-terminal flagellar secretion signal of a native flagellar component. In this manner, the intracellularly expressed chimeric peptide can be mobilized across the inner and outer membranes into the surrounding host environment.

In some embodiments, a Type V Autotransporter Secretion System is used to secrete the molecule of interest, e.g., therapeutic peptide. Due to the simplicity of the machinery and capacity to handle relatively large protein fluxes, the Type V secretion system is attractive for the extracellular production of recombinant proteins. In some embodiments, a therapeutic peptide (star) can be fused to an N-terminal secretion signal, a linker, and the beta-domain of an autotransporter. The N-terminal signal sequence directs the protein to the SecA-YEG machinery which moves the protein across the inner membrane into the periplasm, followed by subsequent cleavage of the signal sequence. The Beta-domain is recruited to the Bam complex (‘Beta-barrel assembly machinery’) where the beta-domain is folded and inserted into the outer membrane as a beta-barrel structure. The therapeutic peptide is thread through the hollow pore of the beta-barrel structure ahead of the linker sequence. Once exposed to the extracellular environment, the therapeutic peptide can be freed from the linker system by an autocatalytic cleavage (left side of Bam complex) or by targeting of a membrane-associated peptidase (black scissors; right side of Bam complex) to a complimentary protease cut site in the linker. Thus, in some embodiments, the secreted molecule, such as a heterologous protein or peptide, e.g., aanoxalate catabolism enzyme, comprises an N-terminal secretion signal, a linker, and beta-domain of an autotransporter so as to allow the molecule to be secreted from the bacteria.

In some embodiments, a Hemolysin-based Secretion System is used to secrete the molecule of interest, e.g., therapeutic peptide. Type I Secretion systems offer the advantage of translocating their passenger peptide directly from the cytoplasm to the extracellular space, obviating the two-step process of other secretion types. Alpha-hemolysin (HlyA) of uropathogenic Escherichia coli is secreted by HlyB, an ATP-binding cassette transporter; HlyD, a membrane fusion protein; and TolC, an outer membrane protein. The assembly of these three proteins forms a channel through both the inner and outer membranes. Natively, this channel is used to secrete HlyA, however, to secrete the therapeutic peptide of the present disclosure, the secretion signal-containing C-terminal portion of HlyA is fused to the C-terminal portion of a therapeutic peptide (star) to mediate secretion of this peptide.

In alternate embodiments, the genetically engineered bacteria further comprise a non-native single membrane-spanning secretion system. Single membrane-spanning transporters may act as a component of a secretion system, or may export substrates independently. Such transporters include, but are not limited to, ATP-binding cassette translocases, flagellum/virulence-related translocases, conjugation-related translocases, the general secretory system (e.g., the SecYEG complex in E. coli), the accessory secretory system in mycobacteria and several types of Gram-positive bacteria (e.g., Bacillus anthracis, Lactobacillus johnsonii, Corynebacterium glutamicum, Streptococcus gordonii, Staphylococcus aureus), and the twin-arginine translocation (TAT) system (Saier, 2006; Rigel and Braunstein, 2008; Albiniak et al., 2013). It is known that the general secretory and TAT systems can both export substrates with cleavable N-terminal signal peptides into the periplasm, and have been explored in the context of biopharmaceutical production. The TAT system may offer particular advantages, however, in that it is able to transport folded substrates, thus eliminating the potential for premature or incorrect folding. In certain embodiments, the genetically engineered bacteria comprise a TAT or a TAT-like system and are capable of secreting the oxalate catabolism enzyme from the bacterial cytoplasm. One of ordinary skill in the art would appreciate that the secretion systems disclosed herein may be modified to act in different species, strains, and subtypes of bacteria, and/or adapted to deliver different payloads.

In order to translocate a protein, e.g., therapeutic polypeptide, to the extracellular space, the polypeptide must first be translated intracellularly, mobilized across the inner membrane and finally mobilized across the outer membrane. Many effector proteins (e.g., therapeutic polypeptides)—particularly those of eukaryotic origin—contain disulphide bonds to stabilize the tertiary and quaternary structures. While these bonds are capable of correctly forming in the oxidizing periplasmic compartment with the help of periplasmic chaperones, in order to translocate the polypeptide across the outer membrane the disulphide bonds must be reduced and the protein unfolded again.

One way to secrete properly folded proteins in gram-negative bacteria—particularly those requiring disulphide bonds—is to target the periplasm in a bacterium with a destabilized outer membrane. In this manner the protein is mobilized into the oxidizing environment and allowed to fold properly. In contrast to orchestrated extracellular secretion systems, the protein is then able to escape the periplasmic space in a correctly folded form by membrane leakage. These “leaky” gram-negative mutants are therefore capable of secreting bioactive, properly disulphide-bonded polypeptides. In some embodiments, the genetically engineered bacteria have a “leaky” or de-stabilized outer membrane. Destabilizing the bacterial outer membrane to induce leakiness can be accomplished by deleting or mutagenizing genes responsible for tethering the outer membrane to the rigid peptidoglycan skeleton, including for example, lpp, ompC, ompA, ompF, tolA, tolB, pal, degS, degP, and nlpl. Lpp is the most abundant polypeptide in the bacterial cell existing at 500,000 copies per cell and functions as the primary ‘staple’ of the bacterial cell wall to the peptidoglycan. (Silhavy, T. J., Kahne, D. & Walker, S. The bacterial cell envelope. Cold Spring Harb Perspect Biol 2, a000414 (2010)). TolA-PAL and OmpA complexes function similarly to Lpp and are other deletion targets to generate a leaky phenotype. Additionally, leaky phenotypes have been observed when periplasmic proteases are deactivated. The periplasm is very densely packed with protein and therefore encode several periplasmic proteins to facilitate protein turnover. Removal of periplasmic proteases such as degS, degP or nlpl can induce leaky phenotypes by promoting an excessive build-up of periplasmic protein. Mutation of the proteases can also preserve the effector polypeptide by preventing targeted degradation by these proteases. Moreover, a combination of these mutations may synergistically enhance the leaky phenotype of the cell without major sacrifices in cell viability. Thus, in some embodiments, the engineered bacteria have one or more deleted or mutated membrane genes. In some embodiments, the engineered bacteria have a deleted or mutated lpp gene. In some embodiments, the engineered bacteria have one or more deleted or mutated gene(s), selected from ompA, ompA, and ompF genes. In some embodiments, the engineered bacteria have one or more deleted or mutated gene(s), selected from tolA, tolB, and pal genes. in some embodiments, the engineered bacteria have one or more deleted or mutated periplasmic protease genes. In some embodiments, the engineered bacteria have one or more deleted or mutated periplasmic protease genes selected from degS, degP, and nlpl. In some embodiments, the engineered bacteria have one or more deleted or mutated gene(s), selected from lpp, ompA, ompA, ompF, tolA, tolB, pal, degS, degP, and nlpl genes.

To minimize disturbances to cell viability, the leaky phenotype can be made inducible by placing one or more membrane or periplasmic protease genes, e.g., selected from lpp, ompA, ompA, ompF, tolA, tolB, pal, degS, degP, and nlpl, under the control of an inducible promoter. For example, expression of lpp or other cell wall stability protein or periplasmic protease can be repressed in conditions where the therapeutic polypeptide needs to be delivered (secreted). For instance, under inducing conditions a transcriptional repressor protein or a designed antisense RNA can be expressed which reduces transcription or translation of a target membrane or periplasmic protease gene. Conversely, overexpression of certain peptides can result in a destabilized phenotype, e.g., overexpression of colicins or the third topological domain of TolA, which peptide overexpression can be induced in conditions in which the therapeutic polypeptide needs to be delivered (secreted). These sorts of strategies would decouple the fragile, leaky phenotypes from biomass production. Thus, in some embodiments, the engineered bacteria have one or more membrane and/or periplasmic protease genes under the control of an inducible promoter.

The tables below list secretion systems for Gram positive bacteria and Gram negative bacteria.

TABLE 12
Secretion systems for gram positive bacteria

| Bacterial Strain | Relevant Secretion System |
|---|---|
| C. novyi-NT (Gram+) | Sec pathway |
| | Twin- arginine (TAT) pathway |
| C. butryicum (Gram+) | Sec pathway |
| | Twin- arginine (TAT) pathway |
| Listeria monocytogenes (Gram+) | Sec pathway |
| | Twin- arginine (TAT) pathway |

TABLE 13
Secretion Systems for Gram negative bacteria
Protein secretary pathways (SP) in gram-negative bacteria and their descendants

| | | | | | | | |
|---|---|---|---|---|---|---|---|
| Type | | | | | | # Proteins/ | Energy |
| (Abbreviation) | Name | TC#² | Bacteria | Archaea | Eukarya | System | Source |

IMPS-Gram-negative bacterial inner membrane channel-orming translocases

| | | | | | | | |
|---|---|---|---|---|---|---|---|
| ABC (SIP) | ATP binding | 3.A.1 | + | + | + | 3-4 | ATP |
| | cassette | | | | | | |
| | translocase | | | | | | |
| SEC (IISP) | General | 3.A.5 | + | + | + | ~12 | GTP |
| | secretory | | | | | | OR |
| | translocase | | | | | | ATP + |
| | | | | | | | PMF |
| Fla/Path | Flagellum/ | 3.A.6 | + | − | − | >10 | ATP |
| (IIISP) | virulence-related | | | | | | |
| | translocase | | | | | | |
| Conj | Conjugation- | 3.A.7 | + | − | − | >10 | ATP |
| (IVSP) | related | | | | | | |
| | translocase | | | | | | |
| Tat (IISP) | Twin-arginine | 2.A.64 | + | + | + | 2-4 | PMF |
| | targeting | | | | (chloroplasts) | | |
| | translocase | | | | | | |
| Oxal | Cytochrome | 2.A.9 | + | + | + | 1 | None or |
| (YidC) | oxidase | | | | (mitochondria) | | PMF |
| | biogenesis | | | | | | |
| | family | | | | chloroplasts) | | |
| MscL | Large | 1.A.22 | + | + | + | 1 | None |
| | conductance | | | | | | |
| | mechanosensit | | | | | | |
| | ive channel | | | | | | |
| | family | | | | | | |
| Holins | Holin | 1.E.1• | + | − | − | 1 | None |
| | functional | 21 | | | | | |
| | superfamily | | | | | | |

Eukaryotic Organelles

| | | | | | | | |
|---|---|---|---|---|---|---|---|
| MPT | Mitochondrial | 3.A.B | − | − | + | >20 | ATP |
| | protein | | | | (mitochondrial) | | |
| | translocase | | | | | | |
| CEPT | Chloroplast | 3.A.9 | (+) | − | + | ≥3 | GTP |
| | envelope | | | | (chloroplasts) | | |
| | protein | | | | | | |
| | translocase | | | | | | |
| Bcl-2 | Eukaryotic | 1.A.21 | − | − | + | 1? | None |
| | Bcl-2 family | | | | | | |
| | (programmed | | | | | | |
| | cell death) | | | | | | |

Gram-negative bacterial outer membrane channel-forming translocases

| | | | | | | | |
|---|---|---|---|---|---|---|---|
| MTB | Main terminal | 3.A.15 | +^{b} | − | − | ~14 | ATP; |
| (IISP) | branch of the | | | | | | PMF |
| | general | | | | | | |
| | secretory | | | | | | |
| | translocase | | | | | | |
| FUP AT-1 | Fimbrial usher | 1.B.11 | +^{b} | − | − | 1 | None |
| | protein | 1.B.12 | +^{b} | − | − | 1 | None |
| | Autotransporter-1 | | | | | | |
| AT-2 OMF | Autotransporter-2 | 1.B.40 | +^{b} | − | − | 1 | None |
| (ISP) | | 1.B.17 | +^{b} | | +(?) | 1 | None |
| TPS | | 1.B.20 | + | − | + | 1 | None |
| Secretin | | 1.B.22 | +^{b} | | − | 1 | None |
| (IISP and IISP) | | | | | | | |
| OmpIP | Outer | 1.B.33 | + | − | + | ≥4 | None? |
| | membrane | | | | (mitochondria; | | |
| | insertion porin | | | | chloroplasts) | | |

The above tables for gram positive and gram negative bacteria list secretion systems that can be used to secret oxalate catabolism enzyme(s) and other polypeptides from the engineered bacteria, which are reviewed in Milton H. Saier, Jr. Microbe/Volume 1, Number 9, 2006 “Protein Secretion Systems in Gram-Negative Bacteria Gram-negative bacteria possess many protein secretion-membrane insertion systems that apparently evolved independently”, the contents of which is herein incorporated by reference in its entirety.

In some embodiments, one or more oxalate catabolic enzymes described herein are secreted. In some embodiments, the one or more oxalate catabolic enzymes described herein are further modified to improve secretion efficiency, decreased susceptibility to proteases, stability, and/or half-life. In some embodiments, formyl-CoA:oxalate CoA-transferase, e.g., Frc (from O. formigenes) is secreted, alone or in combination other oxalate catabolic enzymes, e.g., oxalyl-CoA synthetase, e.g., ScAAE3 (from S. cerevisiae) and/or oxalyl-CoA decarboxylase, e.g., Oxc (from O. formigenes), and/or acetyl-CoA:oxalate CoA-transferase, e.g., YfdE (from E. coli). In some embodiments, oxalyl-CoA synthetase, e.g., ScAAE3 (from S. cerevisiae) is secreted, alone or in combination other oxalate catabolic enzymes, e.g., formyl-CoA:oxalate CoA-transferase, e.g., Frc (from O. formigenes) and/or oxalyl-CoA decarboxylase, e.g., Oxc (from O. formigenes), and/or acetyl-CoA:oxalate CoA-transferase, e.g., YfdE (from E. coli). In some embodiments, oxalyl-CoA decarboxylase, e.g., Oxc (from O. formigenes) is secreted, alone or in combination other oxalate catabolic enzymes, e.g., oxalyl-CoA synthetase, e.g., ScAAE3 (from S. cerevisiae) and/or formyl-CoA:oxalate CoA-transferase, e.g., Frc (from O. formigenes) and/or, and/or acetyl-CoA:oxalate CoA-transferase, e.g., YfdE (from E. coli). In some embodiments, acetyl-CoA:oxalate CoA-transferase, e.g., YfdE (from E. coli) is secreted, alone or in combination other oxalate catabolic enzymes, e.g., oxalyl-CoA decarboxylase, e.g., Oxc (from O. formigenes) and/or oxalyl-CoA synthetase, e.g., ScAAE3 (from S. cerevisiae) and/or formyl-CoA:oxalate CoA-transferase, e.g., Frc (from O. formigenes).

In some embodiments, one or more oxalate catabolism enzyme(s) selected from alanine glyoxalate aminotransferase (AGT, encoded by the AGXT gene, e.g. the human form) and/or glyoxylate/hydroxypyruvate reductase (GRHPR; an enzyme having glyoxylate reductase (GR), hydroxypyruvate reductase (HPR), and D-glycerate dehydrogenase (DGDH) activities, e.g., the human form), and/or 4-hydroxy 2-oxoglutarate aldolase (encoded by the HOGA1 gene, e.g. in humans, is secreted alone or in various combinations, or in combination with other oxalate catabolism enzymes described herein.

Pharmaceutical Compositions and Formulations

Pharmaceutical compositions comprising the genetically engineered microorganisms of the invention may be used to treat, manage, ameliorate, and/or prevent diseases or disorders in which oxalate is detrimental in a subject. In another embodiment, the disorder in which oxalate is detrimental is a disorder that results in daily urinary oxalate excretion over 40 mg per 24 hours. Pharmaceutical compositions of the invention comprising one or more genetically engineered bacteria, and/or one or more genetically engineered virus, alone or in combination with prophylactic agents, therapeutic agents, and/or pharmaceutically acceptable carriers are provided.

In certain embodiments, the pharmaceutical composition comprises one species, strain, or subtype of bacteria that are engineered to comprise one or more of the genetic modifications described herein, e.g., selected from expression of at least one oxalate catabolism enzyme, oxalate importer/transporter and/or formate exporter and/or oxalate:formate antiporter, auxotrophy, kill-switch, knock-out, etc. In alternate embodiments, the pharmaceutical composition comprises two or more species, strains, and/or subtypes of bacteria that are each engineered to comprise the genetic modifications described herein, e.g., one oxalate catabolism enzyme, oxalate importer/transporter and/or formate exporter and/or oxalate:formate antiporter, auxotrophy, kill-switch, knock-out, etc.

The pharmaceutical compositions of the disclosure may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The genetically engineered microorganisms may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, immediate-release, pulsatile-release, delayed-release, or sustained release). Suitable dosage amounts for the genetically engineered bacteria may range from about 10⁴ to 10¹² bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In on embodiment, the pharmaceutical composition is administered after the subject eats a meal

The genetically engineered bacteria or genetically engineered virus may be formulated into pharmaceutical compositions comprising one or more pharmaceutically acceptable carriers, thickeners, diluents, buffers, buffering agents, surface active agents, neutral or cationic lipids, lipid complexes, liposomes, penetration enhancers, carrier compounds, and other pharmaceutically acceptable carriers or agents. For example, the pharmaceutical composition may include, but is not limited to, the addition of calcium bicarbonate, sodium bicarbonate, calcium phosphate, various sugars and types of starch, cellulose derivatives, gelatin, vegetable oils, polyethylene glycols, and surfactants, including, for example, polysorbate 20. In some embodiments, the genetically engineered bacteria of the invention may be formulated in a solution of sodium bicarbonate, e.g., 1 molar solution of sodium bicarbonate (to buffer an acidic cellular environment, such as the stomach, for example). The genetically engineered bacteria may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The genetically engineered microorganisms may be administered intravenously, e.g., by infusion or injection.

The genetically engineered microorganisms of the disclosure may be administered intrathecally. In some embodiments, the genetically engineered microorganisms of the invention may be administered orally. The genetically engineered microorganisms disclosed herein may be administered topically and formulated in the form of an ointment, cream, transdermal patch, lotion, gel, shampoo, spray, aerosol, solution, emulsion, or other form well known to one of skill in the art. See, e.g., “Remington's Pharmaceutical Sciences,” Mack Publishing Co., Easton, Pa. In an embodiment, for non-sprayable topical dosage forms, viscous to semi-solid or solid forms comprising a carrier or one or more excipients compatible with topical application and having a dynamic viscosity greater than water are employed. Suitable formulations include, but are not limited to, solutions, suspensions, emulsions, creams, ointments, powders, liniments, salves, etc., which may be sterilized or mixed with auxiliary agents (e.g., preservatives, stabilizers, wetting agents, buffers, or salts) for influencing various properties, e.g., osmotic pressure. Other suitable topical dosage forms include sprayable aerosol preparations wherein the active ingredient in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms. Examples of such additional ingredients are well known in the art. In one embodiment, the pharmaceutical composition comprising the recombinant bacteria of the invention may be formulated as a hygiene product. For example, the hygiene product may be an antibacterial formulation, or a fermentation product such as a fermentation broth. Hygiene products may be, for example, shampoos, conditioners, creams, pastes, lotions, and lip balms.

The genetically engineered microorganisms disclosed herein may be administered orally and formulated as tablets, pills, dragees, capsules, liquids, gels, syrups, slurries, suspensions, etc. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the genetically engineered microorganisms are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

In some embodiments, enteric coating materials may be used, in one or more coating layers (e.g., outer, inner and/o intermediate coating layers). Enteric coated polymers remain unionised at low pH, and therefore remain insoluble. But as the pH increases in the gastrointestinal tract, the acidic functional groups are capable of ionisation, and the polymer swells or becomes soluble in the intestinal fluid.

Materials used for enteric coatings include Cellulose acetate phthalate (CAP), Poly(methacrylic acid-co-methyl methacrylate), Cellulose acetate trimellitate (CAT), Poly(vinyl acetate phthalate) (PVAP) and Hydroxypropyl methylcellulose phthalate (HPMCP), fatty acids, waxes, Shellac (esters of aleurtic acid), plastics and plant fibers. Additionally, Zein, Aqua-Zein (an aqueous zein formulation containing no alcohol), amylose starch and starch derivatives, and dextrins (e.g., maltodextrin) are also used. Other known enteric coatings include ethylcellulose, methylcellulose, hydroxypropyl methylcellulose, amylose acetate phthalate, cellulose acetate phthalate, hydroxyl propyl methyl cellulose phthalate, an ethylacrylate, and a methylmethacrylate.

Coating polymers also may comprise one or more of, phthalate derivatives, CAT, HPMCAS, polyacrylic acid derivatives, copolymers comprising acrylic acid and at least one acrylic acid ester, Eudragit™ S (poly(methacrylic acid, methyl methacrylate)1:2); Eudragit L100™ S (poly(methacrylic acid, methyl methacrylate)1:1); Eudragit L30D™, (poly(methacrylic acid, ethyl acrylate)1:1); and (Eudragit L100-55) (poly(methacrylic acid, ethyl acrylate)1:1) (Eudragit™ L is an anionic polymer synthesized from methacrylic acid and methacrylic acid methyl ester), polymethyl methacrylate blended with acrylic acid and acrylic ester copolymers, alginic acid, ammonia alginate, sodium, potassium, magnesium or calcium alginate, vinyl acetate copolymers, polyvinyl acetate 30D (30% dispersion in water), a neutral methacrylic ester comprising poly(dimethylaminoethylacrylate) (“Eudragit E™), a copolymer of methylmethacrylate and ethylacrylate with trimethylammonioethyl methacrylate chloride, a copolymer of methylmethacrylate and ethylacrylate, Zein, shellac, gums, or polysaccharides, or a combination thereof.

Coating layers may also include polymers which contain Hydroxypropylmethylcellulose (HPMC), Hydroxypropylethylcellulose (HPEC), Hydroxypropylcellulose (HPC), hydroxypropylethylcellulose (HPEC), hydroxymethylpropylcellulose (HMPC), ethylhydroxyethylcellulose (EHEC) (Ethulose), hydroxyethylmethylcellulose (HEMC), hydroxymethylethylcellulose (HMEC), propylhydroxyethylcellulose (PHEC), methylhydroxyethylcellulose (MHEC), hydrophobically modified hydroxyethylcellulose (NEXTON), carboxymethyl hydroxyethylcellulose (CMHEC), Methylcellulose, Ethylcellulose, water soluble vinyl acetate copolymers, gums, polysaccharides such as alginic acid and alginates such as ammonia alginate, sodium alginate, potassium alginate, acid phthalate of carbohydrates, amylose acetate phthalate, cellulose acetate phthalate (CAP), cellulose ester phthalates, cellulose ether phthalates, hydroxypropylcellulose phthalate (HPCP), hydroxypropylethylcellulose phthalate (HPECP), hydroxyproplymethylcellulose phthalate (HPMCP), hydroxyproplymethylcellulose acetate succinate (HPMCAS).

Liquid preparations for oral administration may take the form of solutions, syrups, suspensions, or a dry product for constitution with water or other suitable vehicle before use. Such liquid preparations may be prepared by conventional means with pharmaceutically acceptable agents such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of the genetically engineered microorganisms described herein.

In one embodiment, the genetically engineered microorganisms of the disclosure may be formulated in a composition suitable for administration to pediatric subjects. As is well known in the art, children differ from adults in many aspects, including different rates of gastric emptying, pH, gastrointestinal permeability, etc. (Ivanovska et al., Pediatrics, 134(2):361-372, 2014). Moreover, pediatric formulation acceptability and preferences, such as route of administration and taste attributes, are critical for achieving acceptable pediatric compliance. Thus, in one embodiment, the composition suitable for administration to pediatric subjects may include easy-to-swallow or dissolvable dosage forms, or more palatable compositions, such as compositions with added flavors, sweeteners, or taste blockers. In one embodiment, a composition suitable for administration to pediatric subjects may also be suitable for administration to adults.

In one embodiment, the composition suitable for administration to pediatric subjects may include a solution, syrup, suspension, elixir, powder for reconstitution as suspension or solution, dispersible/effervescent tablet, chewable tablet, gummy candy, lollipop, freezer pop, troche, chewing gum, oral thin strip, orally disintegrating tablet, sachet, soft gelatin capsule, sprinkle oral powder, or granules. In one embodiment, the composition is a gummy candy, which is made from a gelatin base, giving the candy elasticity, desired chewy consistency, and longer shelf-life. In some embodiments, the gummy candy may also comprise sweeteners or flavors.

In one embodiment, the composition suitable for administration to pediatric subjects may include a flavor. As used herein, “flavor” is a substance (liquid or solid) that provides a distinct taste and aroma to the formulation. Flavors also help to improve the palatability of the formulation. Flavors include, but are not limited to, strawberry, vanilla, lemon, grape, bubble gum, and cherry.

In certain embodiments, the genetically engineered microorganisms may be orally administered, for example, with an inert diluent or an assimilable edible carrier. The compound may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation.

In another embodiment, the pharmaceutical composition comprising the recombinant bacteria of the invention may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the recombinant bacteria of the invention are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the recombinant bacteria of the invention are well known in the art. For example, see U.S. 2015/0359894 and US 2015/0238545, the entire contents of each of which are expressly incorporated herein by reference. In yet another embodiment, the pharmaceutical composition of the invention is injected into, sprayed onto, or sprinkled onto a food product, such as bread, yogurt, or cheese.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The genetically engineered microorganisms described herein may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The genetically engineered microorganisms may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

In some embodiments, the invention provides pharmaceutically acceptable compositions that are not in the form of or incorporated into a food or edible product.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

Dosage regimens may be adjusted to provide a therapeutic response. Dosing can depend on several factors, including severity and responsiveness of the disease, route of administration, time course of treatment (days to months to years), and time to amelioration of the disease. For example, a single bolus may be administered at one time, several divided doses may be administered over a predetermined period of time, or the dose may be reduced or increased as indicated by the therapeutic situation. The specification for the dosage is dictated by the unique characteristics of the active compound and the particular therapeutic effect to be achieved. Dosage values may vary with the type and severity of the condition to be alleviated. For any particular subject, specific dosage regimens may be adjusted over time according to the individual need and the professional judgment of the treating clinician. Toxicity and therapeutic efficacy of compounds provided herein can be determined by standard pharmaceutical procedures in cell culture or animal models. For example, LD50, ED50, EC50, and IC50 may be determined, and the dose ratio between toxic and therapeutic effects (LD50/ED50) may be calculated as the therapeutic index. Compositions that exhibit toxic side effects may be used, with careful modifications to minimize potential damage to reduce side effects. Dosing may be estimated initially from cell culture assays and animal models. The data obtained from in vitro and in vivo assays and animal studies can be used in formulating a range of dosage for use in humans.

In one embodiment, the recombinant bacteria is administered at a dose of about 1×10¹¹ live recombinant bacteria, about 2×10¹¹ live recombinant bacteria, about 3×10¹¹ live recombinant bacteria, about 4×10¹¹ live recombinant bacteria, about 5×10¹¹ live recombinant bacteria, about 6×10¹¹ live recombinant bacteria, about 1×10¹² live recombinant bacteria, or about 2×10¹² live recombinant bacteria. In one embodiment, the recombinant bacteria is administered at a dose of about 6×10¹¹ live recombinant bacteria. In one embodiment, the recombinant bacteria is administered at a dose of about 1×10¹¹ live recombinant bacteria. In one embodiment, the administering is about 5×10¹¹ live recombinant bacteria. In one embodiment, the recombinant bacteria is administered at a dose of about 1×10¹² live recombinant bacteria. In one embodiment, the recombinant bacteria is administered at a dose of about 2×10¹² live recombinant bacteria. In one embodiment, the administering is about 5×10¹¹ live recombinant bacteria with meals three times per day. In one embodiment, the recombinant bacteria is administered at a dose of about 6×10¹¹ live recombinant bacteria with meals three times per day. In one embodiment, the recombinant bacteria is administered at a dose of about 1×10¹¹ live recombinant bacteria with meals three times per day. In one embodiment, the recombinant bacteria is administered at a dose of about 1×10¹² live recombinant bacteria with meals three times per day. In one embodiment, the recombinant bacteria is administered at a dose of about 2×10¹² live recombinant bacteria with meals three times per day.

The ingredients are supplied either separately or mixed together in unit dosage form, for example, as a dry lyophilized powder or water-free concentrate in a hermetically sealed container such as an ampoule or sachet indicating the quantity of active agent. If the mode of administration is by injection, an ampoule of sterile water for injection or saline can be provided so that the ingredients may be mixed prior to administration.

The pharmaceutical compositions may be packaged in a hermetically sealed container such as an ampoule or sachet indicating the quantity of the agent. In one embodiment, one or more of the pharmaceutical compositions is supplied as a dry sterilized lyophilized powder or water-free concentrate in a hermetically sealed container and can be reconstituted (e.g., with water or saline) to the appropriate concentration for administration to a subject. In an embodiment, one or more of the prophylactic or therapeutic agents or pharmaceutical compositions is supplied as a dry sterile lyophilized powder in a hermetically sealed container stored between 2° C. and 8° C. and administered within 1 hour, within 3 hours, within 5 hours, within 6 hours, within 12 hours, within 24 hours, within 48 hours, within 72 hours, or within one week after being reconstituted. Cryoprotectants can be included for a lyophilized dosage form, principally 0-10% sucrose (optimally 0.5-1.0%). Other suitable cryoprotectants include trehalose and lactose. Other suitable bulking agents include glycine and arginine, either of which can be included at a concentration of 0-0.05%, and polysorbate-80 (optimally included at a concentration of 0.005-0.01%). Additional surfactants include but are not limited to polysorbate 20 and BRIJ surfactants. The pharmaceutical composition may be prepared as an injectable solution and can further comprise an agent useful as an adjuvant, such as those used to increase absorption or dispersion, e.g., hyaluronidase.

In some embodiments, the genetically engineered viruses are prepared for delivery, taking into consideration the need for efficient delivery and for overcoming the host antiviral immune response. Approaches to evade antiviral response include the administration of different viral serotypes as par of the treatment regimen (serotype switching), formulation, such as polymer coating to mask the virus from antibody recognition and the use of cells as delivery vehicles.

In another embodiment, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The genetically engineered bacteria of the invention may be administered and formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In Vivo Methods

The recombinant bacteria of the invention may be evaluated in vivo, e.g., in an animal model. Any suitable animal model of a disease or condition in which oxalate is detrimental may be used. For example, an alanine glyoxylate aminotransferase-deficient (agxt −/−) mouse model of PHI as described by Salido et al. can be used (see, e.g., Salido et al., Proc. Natl. Acad. Sci. 103: 18249-54 (2006)). A glyoxylate reductase/hydroxypyruvate reductase knock-out (GRHPR −/−) mouse model of PHII can also be used (see, e.g., Knight et al., Am. J. Physiol. Renal. Physiol. 302: F688-93 (2012)). Mice deficient in the oxalate transporter protein SLC26A6 (Slc26a6-null mice) which develop hyperoxaluria can also be used (see, e.g., Jiang et al. Nature Gen. 38: 474-8 (2006)).

Alternatively, a rat model may be used. For example, Canales et al describe a rat model of Roux-en-Y gastric bypass (RYGB) surgery, in which high fat feeding results in steatorrhea, hyperoxaluria, and low urine pH. RYGB animals on normal fat and no oxalate diets excreted twice as much oxalate as age-matched, sham controls; hyperoxaluria was partially reversible by lowering dietary fat and oxalate content (Canales et al., Steatorrhea And Hyperoxaluria Occur After Gastric Bypass Surgery In Obese Rats Regardless Of Dietary Fat Or Oxalate; J Urol. 2013 September; 190(3): 1102-1109).

The recombinant bacterial cells of the invention may be administered to the animal, e.g., by oral gavage, and treatment efficacy is determined, e.g., by measuring urine levels of oxalic acid before and after treatment. The animal may be sacrificed, and tissue samples may be collected and analyzed.

The following Table 14 includes additional rat models which can be used to assess in vivo activity of the genetically engineered bacteria.

TABLE 14
Rat Models of Calcium Oxalate Nephrolithiasis

| Induction | | Renal changes and | |
|---|---|---|---|
| Technique | Crystal Deposition | urinary changes | Strengths |
| Ethylene glycol in | Intraluminal in renal | Necrotic and apoptotic | Easy to induce |
| drinking water | tubules of both | renal injury, interstitial | consistent |
| | cortex and medulla, | inflammation, increased | hyperoxaluria, |
| | crystals deposit in | synthesis and urinary | crystalluria, and CaOx |
| | association with | excretion of OPN, | nephrolithiasis |
| | cellular degradation | Bikunin, MCP-1, alpha-1 - | |
| | products, plaques and | microglobulin, | |
| | stones at papillary | hyperoxaluria, enzymuria, | |
| | tips. | membranuria, and CaOx | |
| | | crystaluria | |
| Hydroxy-L-proline | No crystals in the | Kidneys appear normal, | Simple, more |
| in drinking water | renal fomices and | hyperoxaluria, enzymuria, | physiological than the |
| | pelvis | and CaOx crystalluria, | administration of |
| | | increased synthesis of | ethylene glycol or |
| | | OPN by papillary surface | some other oxalate |
| | | epithelial cells | precursors |
| Hydroxy-L-proline | Intraluminal in renal | Hyperoxaluria, CaOx | Simple, more |
| mixed with food | tubules of both | crystalluria, signs of renal | physiological than the |
| | cortex and medulla, | injury and inflammation in | administration of |
| | plaques and stones at | association with the | ethylene glycol or |
| | papillary tips | crystals | some other oxalate |
| | | | precursors |
| Implantation of | Intraluminal in renal | Hyperoxaluria, CaOx | Reliable and consistent |
| osmotic mini- | tubules of both | crystalluria, upregulation | hyperoxaluria and |
| pumps filled with | cortex and medulla | of TNF receptor kidney | CaOx nephrolithiasis |
| oxalate | | injury marker and OPN | |
| Vitamin B-6- | CaOx crystals | Hyperoxaluria, | |
| deficient diet | intraluminal in | hypercalciuria, enzymuria, | |
| | tubules of medulla, | hypocitraturia, CaOx | |
| | plaques at papillary | crystalluria | |
| | tips, stones in renal | | |
| | fornices, pelvis, | | |
| | ureters, and bladder | | |
| Glycolic acid in | CaOx crystals in | Hyperoxaluria | |
| diet | tubules of renal | | |
| | cortex and medulla, | | |
| | stones in renal pelvis | | |
| Ileal resection and | CaOx crystals mixed | Tubular obstruction and | Models nephrolithiasis |
| feeding of oxalate | with CaP and Ca | interstitial inflammation, | after ileal resection or |
| | carbonate crystals, | hyperoxaluria, | bypass surgery |
| | intraluminal in both | hypocitraturia | |
| | cortex and medulla, | | |
| | interstitial in the | | |
| | papilla, plaques on | | |
| | papillary surface | | |

Methods of Screening

In some embodiments of the invention, the efficacy or activity of any of the importers, exporters, antiporters, and oxalate catabolism enzymes can be improved through mutations in any of these genes. Methods for directed mutation and screening are known in the art.

Generation of Bacterial Strains with Enhance Ability to Transport Metabolites of Interest

Due to their ease of culture, short generation times, very high population densities and small genomes, microbes can be evolved to unique phenotypes in abbreviated timescales. Adaptive laboratory evolution (ALE) is the process of passaging microbes under selective pressure to evolve a strain with a preferred phenotype. Most commonly, this is applied to increase utilization of carbon/energy sources or adapting a strain to environmental stresses (e.g., temperature, pH), whereby mutant strains more capable of growth on the carbon substrate or under stress will outcompete the less adapted strains in the population and will eventually come to dominate the population.

This same process can be extended to any essential metabolite by creating an auxotroph. An auxotroph is a strain incapable of synthesizing an essential metabolite and must therefore have the metabolite provided in the media to grow. In this scenario, by making an auxotroph and passaging it on decreasing amounts of the metabolite, the resulting dominant strains should be more capable of obtaining and incorporating this essential metabolite.

For example, if the biosynthetic pathway for producing a metabolite of interest is disrupted a strain capable of high-affinity capture of the metabolite of interest can be evolved via ALE. First, the strain is grown in varying concentrations of the auxotrophic metabolite of interest, until a minimum concentration to support growth is established. The strain is then passaged at that concentration, and diluted into lowering concentrations of the metabolite of interest at regular intervals. Over time, cells that are most competitive for the metabolite of interest—at growth-limiting concentrations—will come to dominate the population. These strains will likely have mutations in their metabolite of interest-transporters resulting in increased ability to import the essential and limiting metabolite of interest.

Similarly, by using an auxotroph that cannot use an upstream metabolite to form the metabolite of interest, a strain can be evolved that not only can more efficiently import the upstream metabolite, but also convert the metabolite into the essential downstream metabolite of interest. These strains will also evolve mutations to increase import of the upstream metabolite, but may also contain mutations which increase expression or reaction kinetics of downstream enzymes, or that reduce competitive substrate utilization pathways.

A metabolite innate to the microbe can be made essential via mutational auxotrophy and selection applied with growth-limiting supplementation of the endogenous metabolite. However, phenotypes capable of consuming non-native compounds can be evolved by tying their consumption to the production of an essential compound. For example, if a gene from a different organism is isolated which can produce an essential compound or a precursor to an essential compound this gene can be recombinantly introduced and expressed in the heterologous host. This new host strain will now have the ability to synthesize an essential nutrient from a previously non-metabolizable substrate.

Hereby, a similar ALE process can be applied by creating an auxotroph incapable of converting an immediately downstream metabolite and selecting in growth-limiting amounts of the non-native compound with concurrent expression of the recombinant enzyme. This will result in mutations in the transport of the non-native substrate, expression and activity of the heterologous enzyme and expression and activity of downstream native enzymes. It should be emphasized that the key requirement in this process is the ability to tether the consumption of the non-native metabolite to the production of a metabolite essential to growth.

Once the basis of the selection mechanism is established and minimum levels of supplementation have been established, the actual ALE experimentation can proceed. Throughout this process several parameters must be vigilantly monitored. It is important that the cultures are maintained in an exponential growth phase and not allowed to reach saturation/stationary phase. This means that growth rates must be check during each passaging and subsequent dilutions adjusted accordingly. If growth rate improves to such a degree that dilutions become large, then the concentration of auxotrophic supplementation should be decreased such that growth rate is slowed, selection pressure is increased and dilutions are not so severe as to heavily bias subpopulations during passaging. In addition, at regular intervals cells should be diluted, grown on solid media and individual clones tested to confirm growth rate phenotypes observed in the ALE cultures.

Predicting when to halt the stop the ALE experiment also requires vigilance. As the success of directing evolution is tied directly to the number of mutations “screened” throughout the experiment and mutations are generally a function of errors during DNA replication, the cumulative cell divisions (CCD) acts as a proxy for total mutants which have been screened. Previous studies have shown that beneficial phenotypes for growth on different carbon sources can be isolated in about 10^{11.2} CCD¹. This rate can be accelerated by the addition of chemical mutagens to the cultures—such as N-methyl-N-nitro-N-nitrosoguanidine (NTG)—which causes increased DNA replication errors. However, when continued passaging leads to marginal or no improvement in growth rate the population has converged to some fitness maximum and the ALE experiment can be halted.

At the conclusion of the ALE experiment, the cells should be diluted, isolated on solid media and assayed for growth phenotypes matching that of the culture flask. Best performers from those selected are then prepped for genomic DNA and sent for whole genome sequencing. Sequencing with reveal mutations occurring around the genome capable of providing improved phenotypes, but will also contain silent mutations (those which provide no benefit but do not detract from desired phenotype). In cultures evolved in the presence of NTG or other chemical mutagen, there will be significantly more silent, background mutations. If satisfied with the best performing strain in its current state, the user can proceed to application with that strain. Otherwise the contributing mutations can be deconvoluted from the evolved strain by reintroducing the mutations to the parent strain by genome engineering techniques. See Lee, D.-H., Feist, A. M., Barrett, C. L. & Palsson, B. Ø. Cumulative Number of Cell Divisions as a Meaningful Timescale for Adaptive Laboratory Evolution of Escherichia coli. PLoS ONE 6, e26172 (2011).

Similar methods can be used to generate E. coli Nissle mutants that consume or import oxalate.

Methods of Treatment

Another aspect of the invention provides methods of treating a disorder in which oxalate is detrimental in a subject, or symptom(s) associated with the disorder in which oxalate is detrimental in a subject. In one embodiment, the disorder in which oxalate is detrimental is a disorder associated with increased levels of oxalate. In one embodiment, a disorder associated with increased levels of oxalate is a disorder in which daily urinary oxalate excretion is 40 mg or higher per 24 hours. Disorders associated with increased levels of oxalate include PHI, PHII, PHIII, secondary hyperoxaluria, enteric hyperoxaluria, dietary hyperoxaluria, idiopathic hyperoxaluria, syndrome of bacterial overgrowth, Crohn's disease, inflammatory bowel disease, hyperoxaluria following renal transplantation, hyperoxaluria after a jejunoileal bypass for obesity, hyperoxaluria after gastric ulcer surgery, chronic mesenteric ischemia, gastric bypass, cystic fibrosis, short bowel syndrome, biliary/pancreatic diseases (e.g., chronic pancreatitis), hyperoxaluria with recurrent kidney stones with relatively preserved renal function, and hyperoxaluria with recurrent kidney stones with severe renal dysfunction (e.g., including patients on hemodialysis). In one embodiment, the disorder in which oxalate is detrimental is PHI. In one embodiment, the disorder in which oxalate is detrimental is PHII. In another embodiment, the disorder in which oxalate is detrimental is PHIII. In one embodiment, the disorder in which oxalate is detrimental is secondary hyperoxaluria. In another embodiment, the disorder in which oxalate is detrimental is dietary hyperoxaluria. In one embodiment, the disorder in which oxalate is detrimental is idiopathic hyperoxaluria. In another embodiment, the disorder in which oxalate is detrimental is enteric hyperoxaluria. In one embodiment, the disorder in which oxalate is detrimental is the syndrome of bacterial overgrowth. In another embodiment, the disorder in which oxalate is detrimental is Crohn's disease. In one embodiment, the disorder in which oxalate is detrimental is inflammatory bowel disease. In another embodiment, the disorder in which oxalate is detrimental is hyperoxaluria following renal transplantation. In one embodiment, the disorder in which oxalate is detrimental is hyperoxaluria after a jejunoileal bypass for obesity. In another embodiment, the disorder in which oxalate is detrimental is hyperoxaluria after gastric ulcer surgery. In one embodiment, the disorder in which oxalate is detrimental is chronic mesenteric ischemia. In another embodiment, the disorder in which oxalate is detrimental is gastric bypass. In another embodiment, the disorder in which oxalate is detrimental is cystic fibrosis. In another embodiment, the disorder in which oxalate is detrimental is short bowel syndrome. In another embodiment, the disorder in which oxalate is detrimental are biliary/pancreatic diseases. In another embodiment, the disorder in which oxalate is detrimental is chronic pancreatitis. In another embodiment, the disorder in which oxalate is detrimental is hyperoxaluria with recurrent kidney stones with relatively preserved renal function. In another embodiment, the disorder in which oxalate is detrimental is hyperoxaluria with recurrent kidney stones with severe renal dysfunction (e.g., including patients on hemodialysis).

The present disclosure surprisingly demonstrates that pharmaceutical compositions comprising the recombinant bacterial cells disclosed herein may be used to treat disorders in which oxalate is detrimental, such as PHI and PHI.

In one embodiment, the subject having PHI has a mutation in a AGXT gene. In another embodiment, the subject having PHII has a mutation in a GRHPR gene. In one embodiment, the subject having PHIII has a mutation in a HOGA1 gene. In another aspect, the invention provides methods for decreasing the plasma level of oxalate and/or oxalic acid in a subject by administering a pharmaceutical composition comprising a bacterial cell of the invention to the subject, thereby decreasing the plasma level of the oxalate and/or oxalic acid in the subject. In one embodiment, the subject has a disease or disorder in which oxalate is detrimental. In one embodiment, the disorder in which oxalate is detrimental is PHI.

In one embodiment, the disorder in which oxalate is detrimental is PHII. In another embodiment, the disorder in which oxalate is detrimental is PHIII. In one embodiment, the disorder in which oxalate is detrimental is secondary hyperoxaluria. In another embodiment, the disorder in which oxalate is detrimental is dietary hyperoxaluria. In one embodiment, the disorder in which oxalate is detrimental is idiopathic hyperoxaluria. In another embodiment, the disorder in which oxalate is detrimental is enteric hyperoxaluria. In one embodiment, the disorder in which oxalate is detrimental is the syndrome of bacterial overgrowth. In another embodiment, the disorder in which oxalate is detrimental is Crohn's disease. In one embodiment, the disorder in which oxalate is detrimental is inflammatory bowel disease. In another embodiment, the disorder in which oxalate is detrimental is hyperoxaluria following renal transplantation. In one embodiment, the disorder in which oxalate is detrimental is hyperoxaluria after a jejunoileal bypass for obesity. In another embodiment, the disorder in which oxalate is detrimental is hyperoxaluria after gastric ulcer surgery. In one embodiment, the disorder in which oxalate is detrimental is chronic mesenteric ischemia. In another embodiment, the disorder in which oxalate is detrimental is gastric bypass. In another embodiment, the disorder in which oxalate is detrimental is cystic fibrosis. In another embodiment, the disorder in which oxalate is detrimental is short bowel syndrome. In another embodiment, the disorder in which oxalate is detrimental are biliary/pancreatic diseases. In another embodiment, the disorder in which oxalate is detrimental is chronic pancreatitis.

In some embodiments, the disclosure provides methods for reducing, ameliorating, or eliminating one or more symptom(s) associated with these diseases, including but not limited to fever, vomiting, nausea, diarrhea, kidney stones, oxalosis, bone disease, erythropoietin refractory anemia, skin ulcers, digital gangrene, cardiac arrhythmias, and cardiomyopathy. In some embodiments, the disease is secondary to other conditions, e.g., liver disease.

In certain embodiments, the bacterial cells disclosed herein are capable of catabolizing oxalate and/or oxalic acid in a subject in order to treat a disorder in which oxalate is detrimental. In these embodiments, a patient suffering from a disorder in which oxalate is detrimental, e.g., PHI or PHII, may be able to resume a substantially normal diet, or a diet that is less restrictive than an oxalate-free or a very low-oxalate diet. In some embodiments, the bacterial cells may be capable of catabolizing oxalate and/or oxalic acid, from additional sources, e.g., the blood, in order to treat a disorder in which oxalate is detrimental.

In some embodiments, dietary uptake of oxalate is suppressed by providing the genetically engineered bacteria described herein. In some embodiments, oxalate generated through metabolic pathways, e.g., in a mammal is reduced.

The method may comprise preparing a pharmaceutical composition with at least one genetically engineered species, strain, or subtype of bacteria described herein, and administering the pharmaceutical composition to a subject in a therapeutically effective amount. In some embodiments, the method of treating a disease or disorder associated with elevated oxalate comprises administering to a subject in need thereof an engineered bacterium comprising gene sequence(s) encoding one or more oxalate catabolism enzyme(s) or a pharmaceutical composition thereof. In some embodiments, the method of treating a disease or disorder associated with elevated oxalate comprises administering to a subject in need thereof an engineered bacterium comprising gene sequence(s) encoding one or more oxalate transporter(s) or a pharmaceutical composition thereof. In some embodiments, the method of treating a disease or disorder associated with elevated oxalate comprises administering to a subject in need thereof an engineered bacterium comprising gene sequence(s) encoding one or more formate importers(s) or a pharmaceutical composition thereof. In some embodiments, the method of treating a disease or disorder associated with elevated oxalate comprises administering to a subject in need thereof an engineered bacterium comprising gene sequence(s) encoding one or more oxalate:formate antiporter(s) or a pharmaceutical composition thereof. In some embodiments, the method of treating a disease or disorder associated with elevated oxalate comprises administering to a subject in need thereof an engineered bacterium comprising gene sequence(s) encoding one or more oxalate catabolism enzyme(s) and gene sequence(s) encoding one or more of the following: (i) one or more oxalate transporter(s); (ii) one or more formate exporter(s); (iii) one or more oxalate:formate antiporter(s); and (iv) combinations thereof or a pharmaceutical composition thereof. In some embodiments, the bacterial cells disclosed herein are administered orally, e.g., in a liquid suspension. In some embodiments, the bacterial cells disclosed herein are lyophilized in a gel cap and administered orally. In some embodiments, the bacterial cells disclosed herein are administered via a feeding tube or gastric shunt. In some embodiments, the bacterial cells disclosed herein are administered rectally, e.g., by enema. In some embodiments, the genetically engineered bacteria are administered topically, intraintestinally, intrajejunally, intraduodenally, intraileally, and/or intracolically.

In certain embodiments, the administering the pharmaceutical composition described herein is administered to reduce oxalate and/or oxalic acid levels in a subject. In some embodiments, the methods of the present disclosure reduce the oxalate and/or oxalic acid levels in a subject by at least about 10%, 20%, 25%, 30%, 40%, 50%, 60%, 70%, 75%, 80%, 85%, 90%, 95%, or more. In another embodiment, the methods of the present invention reduce the oxalate and/or oxalic acid levels in a subject by at least two-fold, three-fold, four-fold, five-fold, six-fold, seven-fold, eight-fold, nine-fold, or ten-fold. In another embodiment, the methods of the present invention reduce the daily urinary oxalate excretion of a subject to less than 40 mg per 24 hours. In some embodiments, reduction is measured by comparing the oxalate and/or oxalic acid level in a subject before and after administration of the pharmaceutical composition. In one embodiment, the oxalate and/or oxalic acid level is reduced in the gut of the subject. In one embodiment, the oxalate and/or oxalic acid level is reduced in the urine of the subject. In another embodiment, the oxalate and/or oxalic acid level is reduced in the blood of the subject. In another embodiment, the oxalate and/or oxalic acid level is reduced in the plasma of the subject. In another embodiment, the oxalate and/or oxalic acid level is reduced in the fecal matter of the subject. In another embodiment, the oxalate and/or oxalic acid level is reduced in the brain of the subject.

In one embodiment, the pharmaceutical composition described herein is administered to reduce oxalate and/or oxalic acid levels in a subject to normal levels. In another embodiment, the pharmaceutical composition described herein is administered to reduce oxalate and/or oxalic acid levels in a subject to below a normal level. In another embodiment, the pharmaceutical composition described herein is administered to reduce the daily urinary oxalate excretion of a subject to less than 40 mg per 24 hours.

In certain embodiments, the pharmaceutical composition described herein is administered to reduce oxalate levels in a subject. In some embodiments, the methods of the present disclosure reduce the oxalate levels, in a subject by at least about 10%, 20%, 25%, 30%, 40%, 50%, 60%, 70%, 75%, 80%, 85%, 90%, 95%, or more as compared to levels in an untreated or control subject. In another embodiment, the methods of the present disclosure reduce the oxalate levels, in a subject by at least two-fold, three-fold, four-fold, five-fold, six-fold, seven-fold, eight-fold, nine-fold, or ten-fold. In some embodiments, reduction is measured by comparing the oxalate levels in a subject before and after administration of the pharmaceutical composition. In one embodiment, the oxalate level is reduced in the gut of the subject. In another embodiment, the oxalate level is reduced in the blood of the subject. In another embodiment, the oxalate level is reduced in the plasma of the subject. In another embodiment, the oxalate level is reduced in the liver of the subject. In another embodiment, the oxalate level is reduced in the kidney of the subject.

In one embodiment, the pharmaceutical composition described herein is administered to reduce oxalate in a subject to a normal level.

In some embodiments, the method of treating the disorder in which oxalate is detrimental, e.g., PHI or PHII, allows one or more symptoms of the condition or disorder to improve by at least about 10%, 20%, 30%, 40%, 50%, 60%, 70%, 80%, 90%, 95%, or more. In some embodiments, the method of treating the disorder in which oxalate is detrimental, e.g., PHI or PHII, allows one or more symptoms of the condition or disorder to improve by at least about two-fold, three-fold, four-fold, five-fold, six-fold, seven-fold, eight-fold, nine-fold, or ten-fold.

Before, during, and after the administration of the pharmaceutical composition, oxalate and/or oxalic acid levels in the subject may be measured in a biological sample, such as blood, serum, plasma, urine, peritoneal fluid, cerebrospinal fluid, fecal matter, intestinal mucosal scrapings, a sample collected from a tissue, and/or a sample collected from the contents of one or more of the following: the stomach, kidney, liver, duodenum, jejunum, ileum, cecum, colon, rectum, and anal canal. In some embodiments, the methods may include administration of the compositions disclosed herein to reduce levels of the oxalate and/or oxalic acid. In some embodiments, the methods may include administration of the compositions of the invention to reduce the oxalate and/or oxalic acid to undetectable levels in a subject. In some embodiments, the methods may include administration of the compositions of the invention to reduce the oxalate and/or oxalic acid concentrations to undetectable levels, or to less than about 1%, 2%, 5%, 10%, 20%, 25%, 30%, 40%, 50%, 60%, 70%, 75%, 80%, 85%, 90%, or 95% of the subject's oxalate and/or oxalic acid levels prior to treatment.

In some embodiments, the recombinant bacterial cells disclosed herein produce an oxalate catabolism enzyme under exogenous environmental conditions, such as the low-oxygen environment of the mammalian gut, to reduce levels of oxalate and/or oxalic acid in the urine, blood or plasma by at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold as compared to unmodified bacteria of the same subtype under the same conditions.

In one embodiment, the bacteria disclosed herein reduce plasma levels of oxalate will be reduced to less than 4 mg/dL. In one embodiment, the bacteria disclosed herein reduce plasma levels of oxalate will be reduced to less than 3.9 mg/dL. In one embodiment, the bacteria disclosed herein reduce plasma levels of oxalate, to less than 3.8 mg/dL, 3.7 mg/dL, 3.6 mg/dL, 3.5 mg/dL, 3.4 mg/dL, 3.3 mg/dL, 3.2 mg/dL, 3.1 mg/dL, 3.0 mg/dL, 2.9 mg/dL, 2.8 mg/dL, 2.7 mg/dL, 2.6 mg/dL, 2.5 mg/dL, 2.0 mg/dL, 1.75 mg/dL, 1.5 mg/dL, 1.0 mg/dL, or 0.5 mg/dL.

In one embodiment, the subject has plasma levels of at least 4 mg/dL oxalate prior to administration of the pharmaceutical composition disclosed herein. In another embodiment, the subject has plasma levels of at least 4.1 mg/dL, 4.2 mg/dL, 4.3 mg/dL, 4.4 mg/dL, 4.5 mg/dL, 4.75 mg/dL, 5.0 mg/dL, 5.5 mg/dL, 6 mg/dL, 7 mg/dL, 8 mg/dL, 9 mg/dL, or 10 mg/dL prior to administration of the pharmaceutical composition disclosed herein.

Certain unmodified bacteria will not have appreciable levels of oxalate or oxalyl-CoA processing. In embodiments using genetically modified forms of these bacteria, processing of oxalate and/or oxalyl-CoA will be appreciable under exogenous environmental conditions.

Oxalate and/or oxalic acid levels may be measured by methods known in the art. For example, plasma oxalate levels can be measured using the spectrophotometric plasma oxalate assay described by Ladwig et al. (Ladwig et. al., Clin. Chem. 51: 2377-80 (2005)). Further, urine oxalate levels can be measured for example, by using a oxalate oxidase colorimetric enzymatic assay (Kasidas and Rose, Ann. Clin. Biochem. 22: 412-9 (1985)). In some embodiments, oxalate catabolism enzyme, e.g., Frc, expression is measured by methods known in the art. In another embodiment, oxalate catabolism enzyme activity is measured by methods known in the art to assess Frc activity (see oxalate catabolism enzyme sections, supra).

In certain embodiments, the recombinant bacteria are E. coli Nissle. The recombinant bacteria may be destroyed, e.g., by defense factors in the gut or blood serum (Sonnenborn et al., 2009) or by activation of a kill switch, several hours or days after administration. Thus, the pharmaceutical composition comprising the recombinant bacteria may be re-administered at a therapeutically effective dose and frequency. In alternate embodiments, the recombinant bacteria are not destroyed within hours or days after administration and may propagate and colonize the gut.

In one embodiments, the bacterial cells disclosed herein are administered to a subject once daily. In another embodiment, the bacterial cells disclosed herein are administered to a subject twice daily. In another embodiment, the bacterial cells disclosed herein are administered to a subject in combination with a meal. In another embodiment, the bacterial cells disclosed herein are administered to a subject prior to a meal. In another embodiment, the bacterial cells disclosed herein are administered to a subject after a meal. In another embodiment, the bacterial cells of the invention are not administered in the form of a food or edible product or incorporated into a food or edible product. The dosage of the pharmaceutical composition and the frequency of administration may be selected based on the severity of the symptoms and the progression of the disease. The appropriate therapeutically effective dose and/or frequency of administration can be selected by a treating clinician.

The methods disclosed herein may comprise administration of a composition disclosed herein alone or in combination with one or more additional therapies, e.g., pyridoxine, citrate, orthophosphate, and magnesium, oral calcium supplementation, and bile acid sequestrants, or a low fat and/or low oxalate diet. An important consideration in the selection of the one or more additional therapeutic agents is that the agent(s) should be compatible with the bacteria disclosed herein, e.g., the agent(s) must not interfere with or kill the bacteria. In some embodiments, the genetically engineered bacteria are administered in combination with a low fat and/or low oxalate diet. In some embodiments, administration of the genetically engineered bacteria provides increased tolerance, so that the patient can consume more oxalate and/or fat.

The methods disclosed herein may further comprise isolating a plasma sample from the subject prior to administration of a composition disclosed herein and determining the level of the oxalate and/or oxalic acid in the sample. In some embodiments, the methods disclosed herein may further comprise isolating a plasma sample from the subject after to administration of a composition disclosed herein and determining the level of oxalate and/or oxalic acid in the sample.

The methods of the invention may further comprise isolating a urine sample from the subject prior to administration of a composition of the invention and determining the level of the oxalate and/or oxalic acid in the sample. In some embodiments, the methods of the invention may further comprise isolating a urine sample from the subject after to administration of a composition of the invention and determining the level of oxalate and/or oxalic acid in the sample.

In one embodiment, the methods disclosed herein further comprise comparing the level of the oxalate and/or oxalic acid in the plasma sample from the subject after administration of a composition disclosed herein to the subject to the plasma sample from the subject before administration of a composition disclosed herein to the subject. In one embodiment, a reduced level of the oxalate and/or oxalic acid in the plasma sample from the subject after administration of a composition disclosed herein indicates that the plasma levels of the oxalate and/or oxalic acid are decreased, thereby treating the disorder in which oxalate is detrimental in the subject. In one embodiment, the plasma level of oxalate and/or oxalic acid is decreased at least 10%, 20%, 30%, 40%, 50%, 60%, 70%, 80%, 90%, or 100% in the sample after administration of the pharmaceutical composition as compared to the plasma level in the sample before administration of the pharmaceutical composition. In another embodiment, the plasma level of the oxalate and/or oxalic acid is decreased at least two-fold, three-fold, four-fold, or five-fold in the sample after administration of the pharmaceutical composition as compared to the plasma level in the sample before administration of the pharmaceutical composition.

In one embodiment, the methods of the invention further comprise comparing the level of the oxalate and/or oxalic acid in the urine sample from the subject after administration of a composition of the invention to the subject to the urine sample from the subject before administration of a composition of the invention to the subject. In one embodiment, a reduced level of the oxalate and/or oxalic acid in the urine sample from the subject after administration of a composition of the invention indicates that the urine levels of the oxalate and/or oxalic acid are decreased, thereby treating the disorder in which oxalate is detrimental in the subject. In one embodiment, the urine level of oxalate and/or oxalic acid is decreased at least 10%, 20%, 30%, 40%, 50%, 60%, 70%, 80%, 90%, or 100% in the sample after administration of the pharmaceutical composition as compared to the plasma level in the sample before administration of the pharmaceutical composition. In another embodiment, the urine level of the oxalate and/or oxalic acid is decreased at least two-fold, three-fold, four-fold, or five-fold in the sample after administration of the pharmaceutical composition as compared to the urine level in the sample before administration of the pharmaceutical composition.

In one embodiment, the methods disclosed herein further comprise comparing the level of the oxalate/oxalic acid in the plasma sample from the subject after administration of a composition disclosed herein to a control level of oxalate and/or oxalic acid.

In another embodiment, the methods of the invention further comprise comparing the level of the oxalate/oxalic acid in the urine sample from the subject after administration of a composition of the invention to a control level of oxalate and/or oxalic acid.

### EXAMPLES

The present invention is further illustrated by the following examples which should not be construed as limiting in any way. The contents of all cited references, including literature references, issued patents, and published patent applications, as cited throughout this application are hereby expressly incorporated herein by reference. It should further be understood that the contents of all the figures and tables attached hereto are also expressly incorporated herein by reference.

### Example 1. Genetically Engineered E. coli Nissle Bacterial Strains Decrease Oxalate Concentration Over Time

Both in vitro and in vivo experiments were conducted which demonstrate that E. coli Nissle bacterial strains decrease oxalate concentration over time.

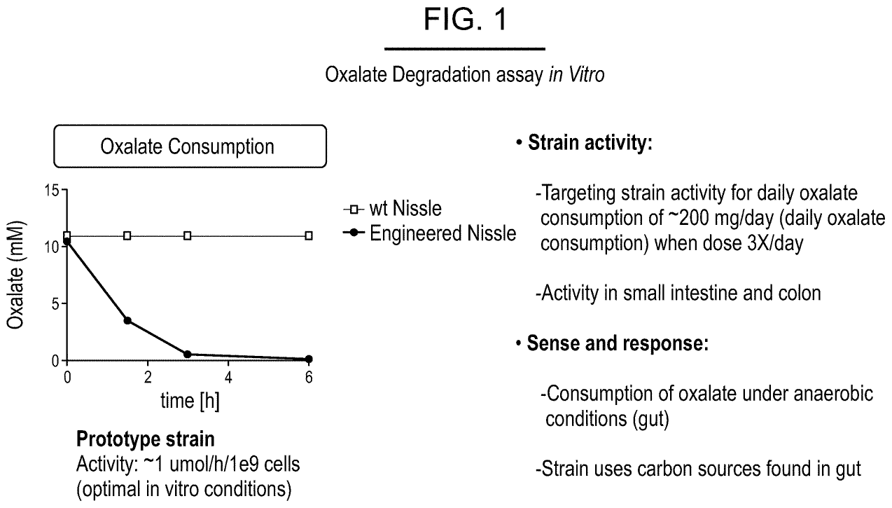

Specifically, a functional in vitro assay was conducted as indicated in FIG. 1. The results from this assay demonstrate that the genetically Engineered E. coli Nissle bacterial strain decreases oxalate concentration over time as compared to the wild type E. coli Nissle bacterial strain (see FIG. 1).

In vivo experiments in animals were also conducted. On day 0, mice were weighed, marked, and randomized into 4 groups. Starting at Day 1, the following protocol was used.

Day 1:

T0:
PO dose with 1004 (100 m) of 13C-Oxalate
PO dose with 2004 of Treatment

T1:
PO dose with 3004 of Treatment

T6: collect urine, feces

The animals were dosed a high dose at 3.12e10 CFU, a mid dose at 1.04e10 CFU and a low dose at 3.46e9 CFU (total CFUs).

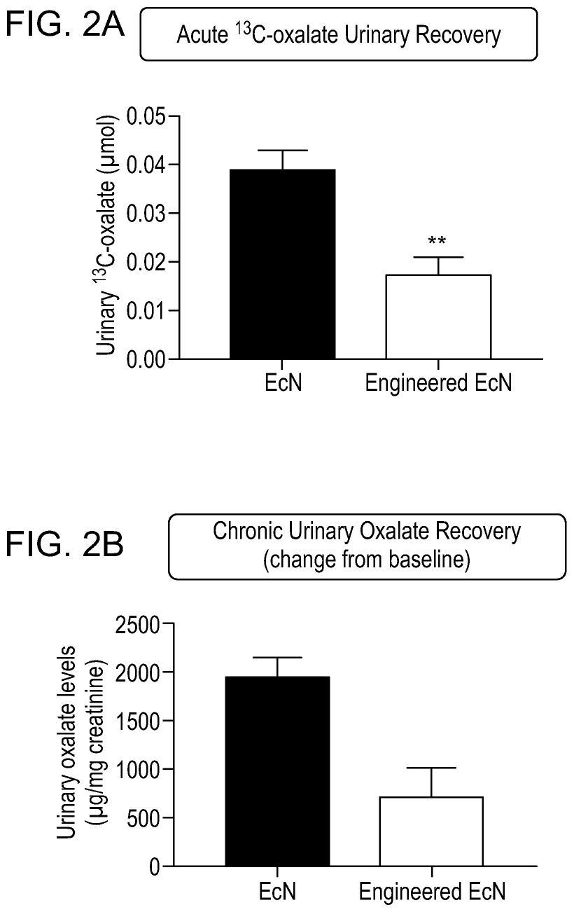

The results depicted in FIG. 2A demonstrate that the genetically Engineered E. coli Nissle bacterial strain reduces acute levels of ¹³C-oxalate, detected in the urine of the treated mice, as compared to the wild type strain.

The results depicted in FIG. 2B demonstrate that the genetically Engineered E. coli Nissle bacterial strain reduces chronic levels of oxalate, detected in the urine of the treated mice, as compared to the wild type strain.

TABLE 15
Construct comprising oxalate catabolism cassette
driven by Tet responsive promoter

| Description | SEQ ID NO |
|---|---|
| Construct comprising TetR in reverse orientation, | SEQ ID NO: 34 |
| TetR/TetA promoter, and oxalate catabolism cassette | |
| comprising ScAAE3 (oxalate-CoA ligase from | |
| S. cerevisiae), oxy, oxylyl-coA decarboxylase from | |
| O. formigenes, and formyl-coA transferase from | |
| O. formigenes, separated by ribosome binding sites | |
| Construct comprising TetR/TetA promoter, and oxalate | SEQ ID NO: 35 |
| catabolism cassette comprising ScAAE3 (oxalate-CoA | |
| ligase from S. cerevisiae), oxc, oxylyl-coA | |
| decarboxylase from O. formigenes, and frc, formyl- | |
| coA transferase from O. formigenes, separated by | |
| ribosome binding sites | |
| Construct comprising oxalate catabolism cassette | SEQ ID NO: 36 |
| comprising ScAAE3 (oxalate-CoA ligase from | |
| S. cerevisiae), oxy, oxylyl-coA decarboxylase from | |
| O. formigenes, and formyl-coA transferase from | |
| O. formigenes, separated by ribosome binding sites | |

Table 16 lists the construct for the chromosomally integrated OxlT at the lacZ locus.

TABLE 16
Construct comprising OxlT (oxalate: formate
antiporter) driven by tet-inducible promoter

| Description | SEQ ID NO |
|---|---|
| Construct comprising TetR in reverse orientation, | SEQ ID NO: 37 |
| TetR/TetA promoter, driving OxlT (oxalate: formate | |
| antiporter from O. formigenes) | |
| Construct comprising TetR/TetA promoter, driving | SEQ ID NO: 38 |
| OxlT (oxalate: formate antiporter from | |
| O. formigenes) | |
| Construct comprising RBS and leader region | SEQ ID NO: 39 |
| driving OxlT (oxalate: formate antiporter from | |
| O. formigenes) | |

TABLE 17
Construct comprising oxalate catabolism cassette (pFNRS-
ScAAE3- oxc-frc) under control of a FNR promoter

| Description | SEQ ID NO |
|---|---|
| Construct comprising promoter with FNR binding | SEQ ID NO: 40 |
| site driving expression of cassette comprising | |
| ScAAE3 (oxalate-CoA ligase from S. cerevisiae), | |
| oxy, oxylyl-coA decarboxylase from O. formigenes, | |
| and formyl-coA transferase from O. formigenes, | |
| separated by ribosome binding sites | |
| FNR promoter with RBS and leader region | SEQ ID NO: 41 |
| FNR promoter without RBS and leader region | SEQ ID NO: 66 |

TABLE 18
Construct comprising OxlT (oxalate: formate
antiporter) under control of a FNR promoter

| Description | SEQ ID NO |
|---|---|
| Construct comprising FNR promoter, | SEQ ID NO: 42 |
| driving OxlT (oxalate: formate antiporter | |
| from O. formigenes) | |

TABLE 19
Oxalate catabolism cassette (oxc-frc) driven by FNRS promoter

| Description | SEQ ID NO |
|---|---|
| Construct comprising FNR promoter, driving a | SEQ ID NO: 43 |
| oxalate catabolism cassette comprising oxc-frc. | |
| Construct comprising oxalate catabolism cassette | SEQ ID NO: 44 |
| comprising oxc-frc with RBS and leader region | |

TABLE 20
Oxalate catabolism cassette (yfdE-
oxc-frc) driven by FNRS promoter

| Description | SEQ ID NO |
|---|---|
| Construct comprising FNR promoter, driving a oxalate | SEQ ID NO: 45 |
| catabolism cassette comprising yfdE-oxc-frc. | |
| Construct comprising oxalate catabolism cassette | SEQ ID NO: 46 |
| comprising yfdE-oxc-frc with RBS and leader region | |

Sample Preparation

Oxalic acid stock 10 mg/mL was prepared in water and aliquoted in 1.5 mL microcentrifuge tubes (100 μL), and stored at −20° C. Standards (1000, 500, 250, 100, 20, 4, and 0.8 μg/mL) are prepared in water. On ice, 20 μL of sample (and standards) were mixed with 180 μL of H₂O containing 10 μg/mL of oxalic acid-d2 in the final solution in a V-bottom 96-well plate. The plate was heat-sealed with a ClearASeal sheet and mix well.

LC-MS/MS Method

Oxalate was measured by liquid chromatography coupled to tandem mass spectrometry (LC-MS/MS) using a Thermo TSQ Quantum Max triple quadrupole mass spectrometer. Table 21, Table 22 and Table 23 provide the summary of the LC-MS/MS method.

TABLE 21

| | |
|---|---|
| Column | Synergi Hydro column, 4 μm (75 × 4.6 mm) |
| Mobile Phase A | 5 mM Ammonium acetate |
| Mobile Phase B | Methanol |
| Injection volume | 10 uL |

TABLE 22
HPLC Method:

| | Flow Rate | | |
|---|---|---|---|
| Time (min) | (μL/min) | A % | B % |
| 0.0 | 500 | 100 | 0 |
| 0.5 | 500 | 100 | 0 |
| 1.0 | 500 | 5 | 95 |
| 2.5 | 500 | 5 | 95 |
| 2.51 | 500 | 100 | 0 |
| 2.75 | 500 | 100 | 0 |

TABLE 23
Tandem Mass Spectrometry

| | |
|---|---|
| Ion Source | HESI-II |
| Polarity | Negative |
| SRM transitions | Oxalate: 90.5/61.2 |
| SRM transitions | Oxalate-d2: 92.5/62.2 |

TABLE 24
Primer Sequences

| Name | Description | SEQ ID NO |
|---|---|---|
| SR36 | Round 1: binds on pKD3 | SEQ ID NO 47 |
| SR38 | Round 1: binds on pKD3 | SEQ ID NO 48 |
| SR33 | Round 2: binds to round 1 PCR product | SEQ ID NO 49 |
| SR34 | Round 2: binds to round 1 PCR product | SEQ ID NO 50 |
| SR43 | Round 3: binds to round 2 PCR product | SEQ ID NO 51 |
| SR44 | Round 3: binds to round 2 PCR product | SEQ ID NO 52 |

TABLE 25
Pfnr1-lacZ construct Sequences

| Description | SEQ ID NO |
|---|---|
| Nucleotide sequences of | SEQ ID NO: 53 |
| Pfnr1-lacZ construct, low-copy | |

TABLE 26
Pfnr2-lacZ construct sequences

| Description | SEQ ID NO |
|---|---|
| Nucleotide sequences | SEQ ID NO: 54 |
| of Pfnr2-lacZ construct, low-copy | |

TABLE 27
Pfnr3-lacZ construct Sequences

| Description | SEQ ID NO |
|---|---|
| Nucleotide sequences of | SEQ ID NO: 55 |
| Pfnr3-lacZ construct, low-copy | |

TABLE 28
Pfnr4-lacZ construct Sequences

| Description | SEQ ID NO |
|---|---|
| Nucleotide sequences of | SEQ ID NO: 56 |
| Pfnr4-lacZ construct, low-copy | |

TABLE 29
Pfnrs-lacZ construct Sequences

| Description | SEQ ID NO |
|---|---|
| Nucleotide sequences of | SEQ ID NO: 57 |
| Pfnrs-lacZ construct, low-copy | |

### Example 2. Other Sequences of Interest

TABLE 30
prpR Propionate-Responsive Promoter Sequence

| Description | SEQ ID NO |
|---|---|
| Prp promoter | SEQ ID NO: 58 |

TABLE 31
Wild-type clbA and clbA knock-out

| Description | SEQ ID NO |
|---|---|
| Wild-type clbA | SEQ ID NO: 59 |
| clbA knock-out | SEQ ID NO: 60 |

### Example 3. Reduction of Oxalate Concentrations in Acute Mice Models and Healthy Monkeys

Enteric hyperoxaluria occurs when there is excess absorption of oxalate in the gastrointestinal (GI) tract, which results in an accumulation of oxalate in kidneys, and may lead to recurrent kidney stones and kidney failure. It has been shown that reduction of oxalate in GI track is clinically beneficial for patients. However, there is currently no available therapy, and there are more than 80,0000 severe patients in the United States alone. These patients can have recurrent kidney stones and risk for kidney failure.

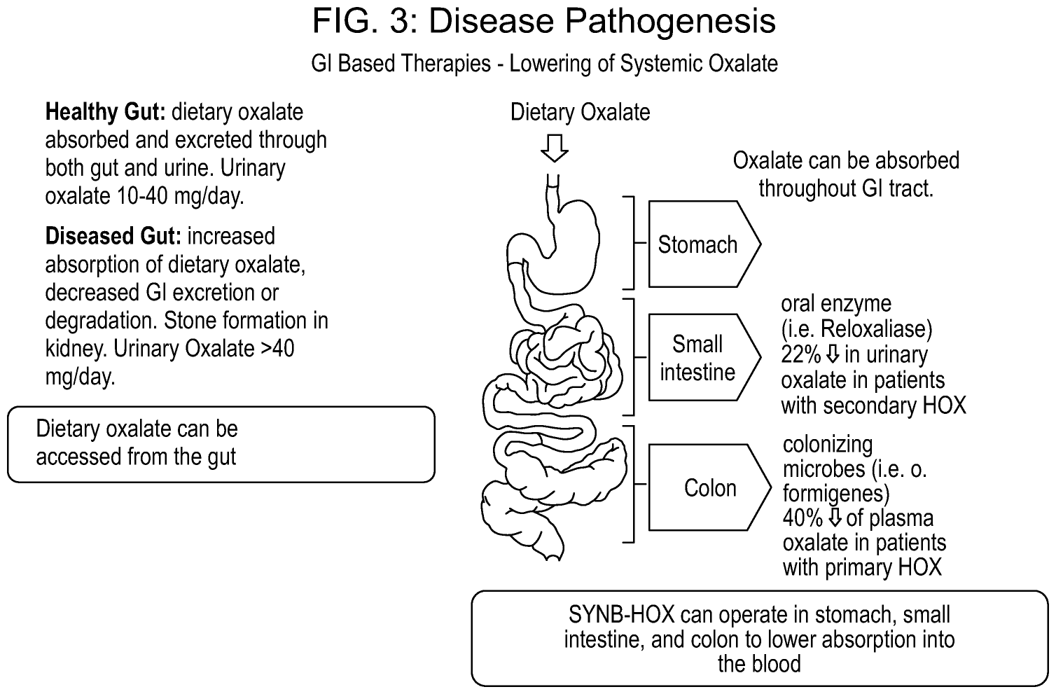

As demonstrated herein, the engineered bacterial strains have a great potential to operate in stomach, small intestine, and colon to lower absorption of oxalate into the blood (FIG. 3).

In vivo experiments were conducted in both acute mouse models and healthy monkeys which demonstrate that E. coli Nissle bacterial strains decrease oxalate concentration over time. Specifically, three engineered E. coli Nissle strains (SYN5752, SYN7169, and SYNB8802) had been constructed. SYN7169 is derived from SYN5752, the only difference is the thyA and phage 3 ko (sequences included in Table 33). Genotypes were shown below.

SYN5752: HA910::FNR_oxlT, HA12::FNR_scaaE3-oxcd-frc

SYN7169: HA910::FNR_oxlT, HA12::FNR_scaaE3-oxcd-frc, ThyA::KanR, phage 3:: CamR

SYN7169 and SYNB8802 have identical genetic modifications, but SYN7169 also has a chloramphenicol and kanamycin resistance cassette to aid in isolation on selective mediate. Relevant sequences were included in Tables shown above and Table 33.

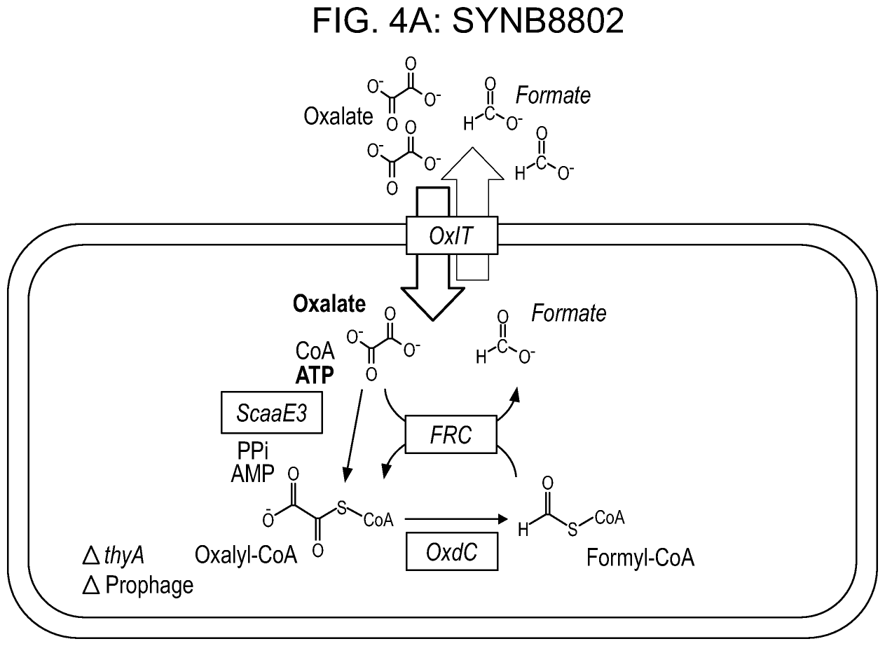

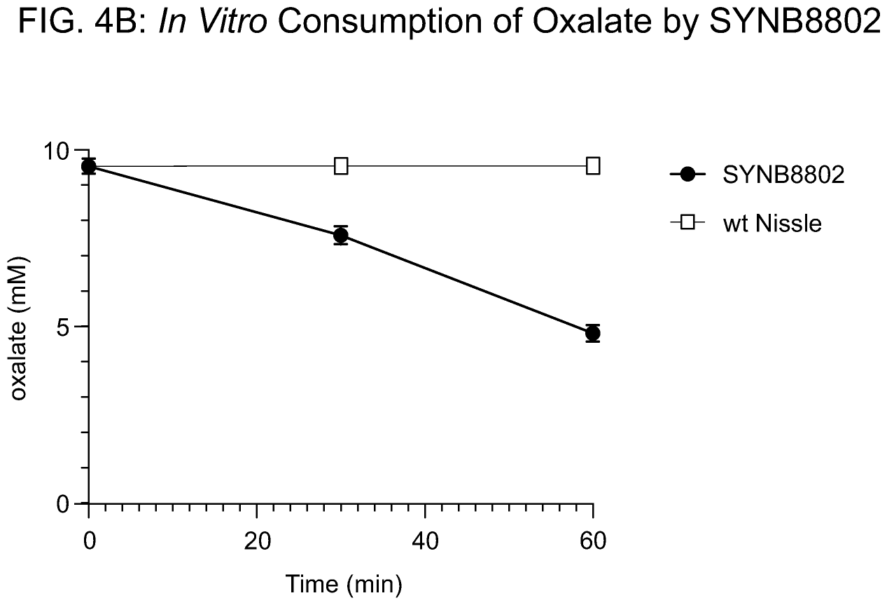

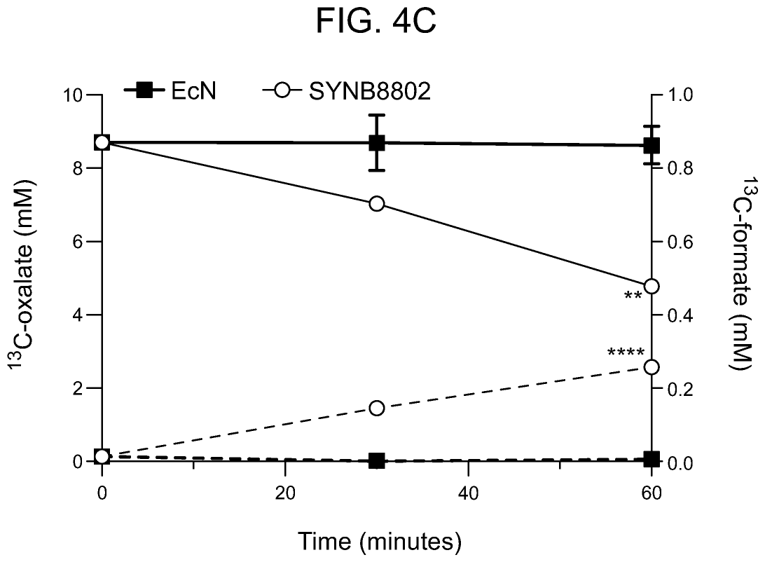

SYNB8802 (FIG. 4A) includes an insertion of one gene encoding an oxalate antiporter (OxlT) derived from Oxalobacter formigenes under the regulatory control of an anaerobic-inducible promoter (pFnrs) and the anaerobic-responsive transcriptional activator FNR, and insertion of one operon, encoding three genes under the regulatory control of an anaerobic-inducible promoter (pFnrs) and the anaerobic-responsive transcriptional activator FNR (oxalyl-CoA synthetase (scaae3) derived from Saccharomyces cerevisiae, oxalate decarboxylase (oxdc) derived from Oxalobacter formigenes, and formyl-CoA transferase derived from Oxalobacter formigenes, a deletion of the thyA gene that encodes thymidylate synthase to create a thymidine auxotroph, and the endogenous Nissle prophage has been inactivated. Relevant sequences are included in the Tables herein.

In vitro experiments were conducted as described above. The results from this assay demonstrate that the genetically Engineered E. coli Nissle bacterial strain (SYNB8802) decreases oxalate concentration over time as compared to the wild type E. coli Nissle bacterial strain (see FIG. 4B).

Additionally, the genetically engineered E. coli Nissle bacterial strain (SYNB8802) increases formate concentration over time as compared to wild type E. coli Nissle bacterial strain (FIG. 4C). E. coli Nissle (control) and SYNB8802 were grown in shake flasks and subsequently activated in an anaerobic chamber, followed by concentration and freezing at ≤−65° C. in glycerol-based formulation buffer. In assay media containing 10 mM ¹³C-oxalate, optical density=5 activated cells were incubated statically at 37° C. Supernatant samples were removed at 30 and 60 minutes to determine the concentrations of ¹³C-oxalate and ¹³C-formate. The concentrations of ¹³C-oxalate and ¹³C-formate were determined by liquid chromatography-tandem mass spectrometry (LC-MS/MS).

### In Vitro Gastrointestial Simulation (IVS)

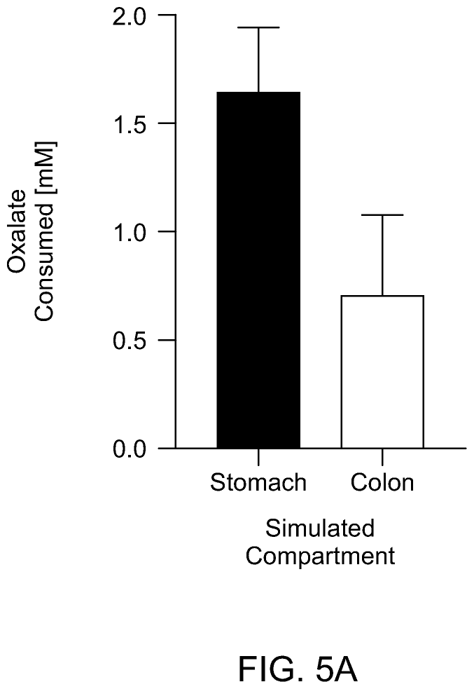

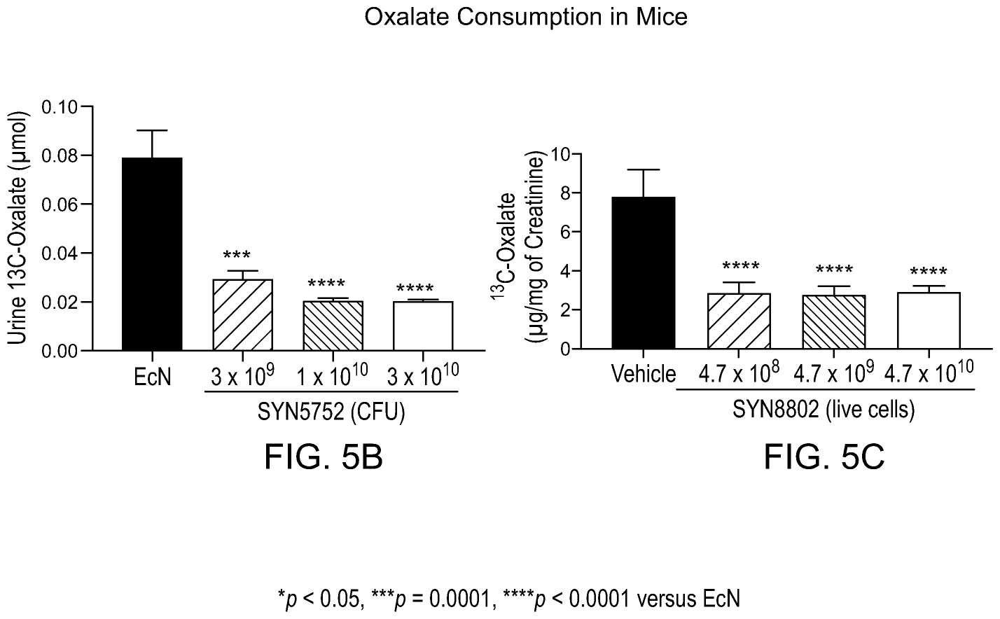

Both stomach and colon simulated gut fluids were capable of activating SYNHOX in simulated in vitro (FIG. 5A). Simulated stomach fluid activated SYNHOX (SYN5752) oxalate consummation activity more than twice as much when compared to oxalate consumption in simulated colon fluid (FIG. 5A).

To characterize the viability and metabolic activity of engineered bacterial strains, and to predict their function in vivo, an in vitro gastrointestinal simulation (IVS) model was designed to simulate key aspects of the human gastrointestinal tract, including oxygen concentration, gastric and pancreatic enzymes, and bile. The IVS model is comprised of a series of incubations in 96-well microplate format designed to simulate stomach, small intestine, and colonic conditions. The stomach, small intestinal, colon portions of the IVS model were adapted from Minekus et al., 2014.

Briefly, frozen aliquots of bacterial cells were first thawed at room temperature and resuspended in 0.077 M sodium bicarbonate buffer at 5.0×10⁹ live cells/mL. This solution was then mixed with equal parts of simulated gastric fluid (SGF; Minekus et al., 2014) containing 10 mM oxalate and incubated for 2 hours at 37° C. with shaking in a Coy microaerobic chamber. The atmosphere within the microaerobic chamber was initially calibrated to 7% oxygen and gradually decreased to 2% oxygen over 2 hours. The cell density in SGF is 2.5×10⁹ live cells/mL. After 2 hours cells were then mixed in volumes of 1:1 with simulated intestinal fluid (SIF; Minekus et al., 2014) and incubated for an additional 2 hours at 37° C. with shaking in a Coy microaerobic chamber. Cell density in SIF is 1.25×10⁹ live cells/mL. After 2 hours, cells were moved into the anaerobic chamber and mixed 1:6 with colon simulated media based off of SGF; Minekus et al., 2014 (CSM). CSM had an additional 10 mM Oxalate and were incubated for 3 hours at 37° C. Cell density in CSM is 2.08×10⁶ live cells/mL.

To determine strain activity over time, aliquots were collected periodically and centrifuged at 4000 rpm for 5 mins using a tabletop centrifuge. Cell free supernatants were collected and stored at −80° C. prior to mass spectrometry analysis of oxalate concentration.

In Vivo Mouse Model Studies

For in vivo mice studies, on day 0, mice were weighed, marked, and randomized into 4 groups. Starting at Day 1, the following protocol was used.

Day 1:

T0:
PO dose with 1004 (100 μg) of ¹³C-Oxalate
PO dose with 2004 of Treatment

T1:
PO dose with 3004 of Treatment

T6: collect urine, feces

The animals were dosed a high dose at 3e10 CFU, a mid dose at 1e10 CFU and a low dose at 3e9 CFU (total CFUs).

As shown in FIG. 5B, SYN-5752 strain in acute, isotope model demonstrated urinary oxalate consumption in gut. ¹³C-Oxalate consumption had been measured in multiple acute mouse studies and the efficacy of the strains ranged between 50-75% (FIG. 5B). SYN7169 behaved similarly to SYN5752 in this model.

In a different experiment, C57BL/6J male mice were group housed and orally administered a dose of ¹³C-oxalate (100 μg) followed by a dose of vehicle (13.8% w/v Trehalose, 68 mM Tris, 55 mM HCl, lx PBS) or SYNB8802. Mice were immediately placed into metabolic cages (n=3/cage) and received another dose of vehicle or SYNB8802 1 hour post first dose, for a daily total of 4.7×10⁸, 4.7×10⁹ or 4.7×10¹⁰ live cells. Urine was collected 6 hours following dose 1 and ¹³C-oxalate and creatinine levels were quantitated by liquid chromatography/tandem mass spectrometry (LC-MS/MS). Two studies were conducted, and the results were combined (FIG. 5C).

In Vivo Monkey Model Studies

For in vivo monkey studies, animals were randomly distributed in 2 groups, vehicle (formulation buffer) and SYN7169 (Sell cells). N=6 in each group. After overnight fasting, each animal received same amount of spinach smoothie, ¹³C-2 labeled oxalate, sodium bicarbonate (1M) and either vehicle or strain (see Table 32). The spinach smoothie was prepared by blending baby spinach leaves in tap water until smooth at a spinach:water ratio of 60 gm:40 mL.

Specifically, treatments were administered to the appropriate animals by oral gavage on Day 1. Capped bacteria tube was inverted 3 times before each dose administration. Dose formulations were administered by oral gavage using a disposable catheter attached to a plastic syringe. Following dosing, the gavage tube were rinsed with 5 mL of the animal drinking water, into the animal's stomach. Each animal was dosed with a clean gavage tube. The first day of dosing was designated as Day 1.

TABLE 32
Experimental Design

| | | | Dose | |
|---|---|---|---|---|
| | No. | | Volume | Dose |
| Group | Animals | Treatment | (mL) | Regimen |
| 1 | 6 | Spinach smoothie | 25.7 | PO |
| | | Sodium bicarbonate | 1.8 | |
| | | ¹³C2 oxalate (20 mg/ml) | 2.5 | |
| | | Formulation buffer | 5.0 | |
| 2 | 6 | Spinach smoothie | 25.7 | |
| | | Sodium bicarbonate | 1.8 | |
| | | ¹³C2 oxalate (20mg/ml) | 2.5 | |
| | | Bacteria (SYN7169021920B-001) | 5.0 | |

Urine was collected after 6 hrs of PO dosing. Animals were separated and a clean collection pan was inserted prior to dose to assist in urine collection at room temperature. At conclusion of 6 hours post dose, the total amount of urine were measured and recorded. One aliquot of 1 mL samples was collected in uniquely labeled clear polypropylene tube and immediately frozen on dry ice. A second aliquot of approximately 100 uL was collected in a 96-deep well plate and immediately frozen on dry ice.

Oxalate, ¹³C2-oxalate, and creatinine were measured in monkey or non-human primates (NHP) urine by liquid chromatography (LC) tandem mass spectrometry (MS/MS) with selected reaction monitoring (SRM) of analyte specific fragmentation products using a Thermo Vanquish-TSQ Altis LC-MS/MS system. Urine was diluted tenfold with 10 mM ammonium acetate containing creatinine-d5, 2 uL injected and separated using a Waters Acquity HSS T3 column (2.1×100 mm) at 0.4 uL/min and 50° C. from 0 to 95% B over two minutes (A: 10 mM ammonium acetate; B: acetonitrile). SRM ion transitions were as follows in electrospray negative mode: oxalate 89>61, ¹³C2-oxalate 91>62; electrospray positive mode: creatinine 114>44, creatinine-d5 199>49. Sample concentrations of oxalate and ¹³C2-oxalate were calculated using absolute peak areas and a matrix based standard curve constructed in NHP urine. Creatinine concentrations were calculated using creatinine/creatinine-d5 peak area ratios and a water based standard curve.

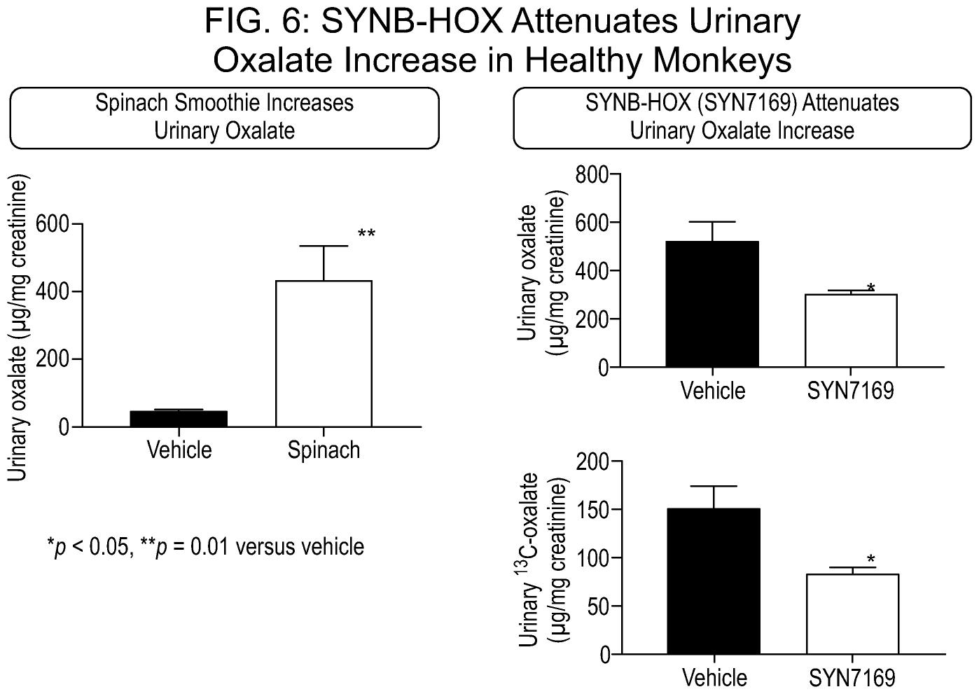

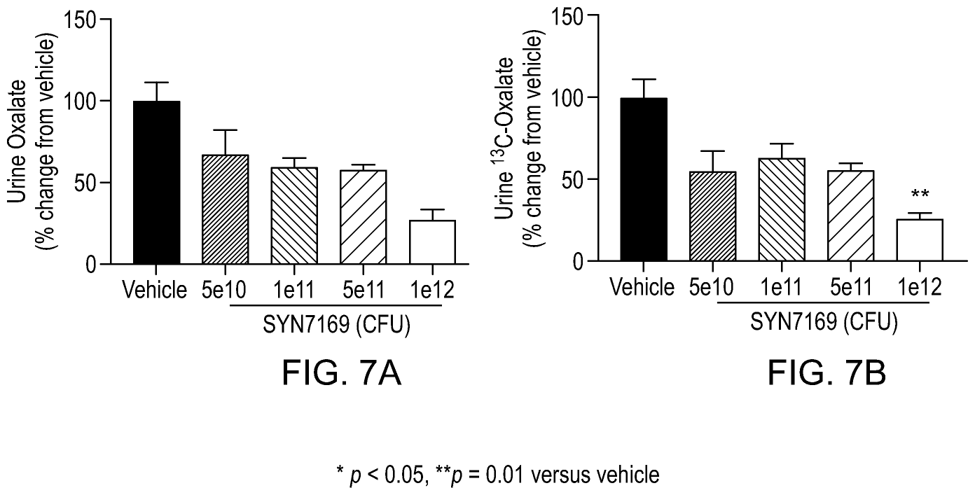

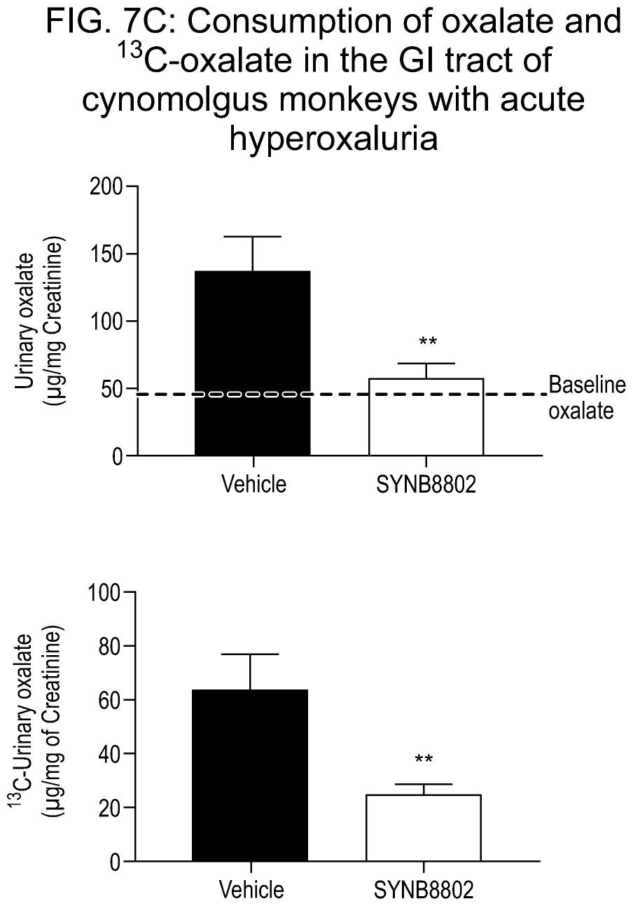

As shown in FIG. 6, while spinach smoothie increased urinary oxalate levels in the treated monkeys, engineering EcNs (SYN7169) attenuated these increase in urinary oxalate significantly. SYN7169 dose-dependently lowered the recovery of urinary oxalate by 45%, 37%, 45% and 75% at 5×10¹⁰, 1×10¹¹, 5×10¹¹ or 1×10¹² CFU as compared to vehicle, respectively (see FIG. 7A). The effects of SYN7169 on ¹³C-oxalate followed the same trends (see FIG. 7B). In conclusion, these studies indicate that SYN7169 was capable of consuming oxalate in monkeys with acute hyperoxaluria.

In a second study with 12 male cynomolgus monkeys receiving both vehicle and SYNB8802. Animals were fasted the night prior to the study for approximately 16-18 hours. On the morning of the experiment, each monkey was removed from its cage and administered vehicle (water) or spinach suspension (39 g), sodium bicarbonate (1.8 mmol), ¹³C-oxalate (50 mg), and vehicle (13.8% w/v Trehalose, 68 mM Tris, 55 mM HCl, lx PBS) or SYNB8802 (1×10¹² live cells). Animals were then returned to their cages and a clean urine collection pan was placed at the bottom of each cage. Urine was collected at 6 hours postdosing, and the levels of oxalate, ¹³C-oxalate and creatinine were quantitated by liquid chromatography/tandem mass spectrometry (LC-MS/MS) (FIG. 7C).

Viable SYNHOX from Fecal Samples

Viable SYNHOX was recovered in feces 6 and 24 hours after oral doses were administered to both mice and non human primates (NHP). A significant number of viable SYNHOX (SYN7169) and SYNB8802 were recovered at both time points.

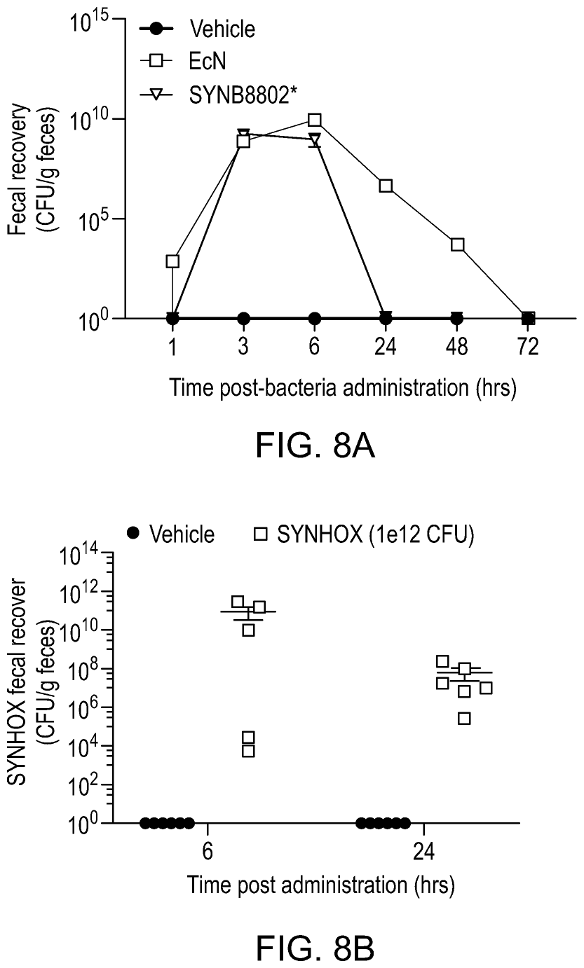

Viable SYNB8802 and wild type E. coli were recovered from mouse feces and were viable at multiple time points over 72 hours after ingestion (FIG. 8A). SYNB8802 cleared from feces after 24 hours while the wild type strain cleared at 72 hours after ingestion. Briefly, C57BL/6J mice were group housed and assigned to groups (n=16) based on average cage body weight. Mice received a single oral dose of the treatment (1.3×10¹⁰ CFU) and feces were collected fresh by free catch and placed into pre-weighed BeadBug tubes containing 500 mL of PBS, weighed, and then processed for serial dilution plating to determine viable colony-forming units (CFUs) immediately after collection. Data presented as mean bacterial strain fecal recovery±standard error of the mean. CFU=colony forming unit, SYNB8802*=antibiotic-resistant SYNB8802.

In a separate experiment, twelve male monkeys were fasted the night prior to the study. On the morning of the study, each monkey was removed from its cage and administered a spinach suspension, sodium bicarbonate, ¹³C-oxalate, and formulation buffer or bacteria. Feces were collected 6 and 24 hours postdosing, and the total weight was recorded. Fecal samples were homogenized with phosphate-buffered saline (PBS; 10 times sample weight), and the final volume of buffer added was recorded. Fecal sample suspensions were serially diluted in PBS and plated on selective LB agar media to enumerate SYN7169 or SYNB8802 colony-forming units (CFU) (FIG. 8B).

### Example 4: Formulations and Human Treatment

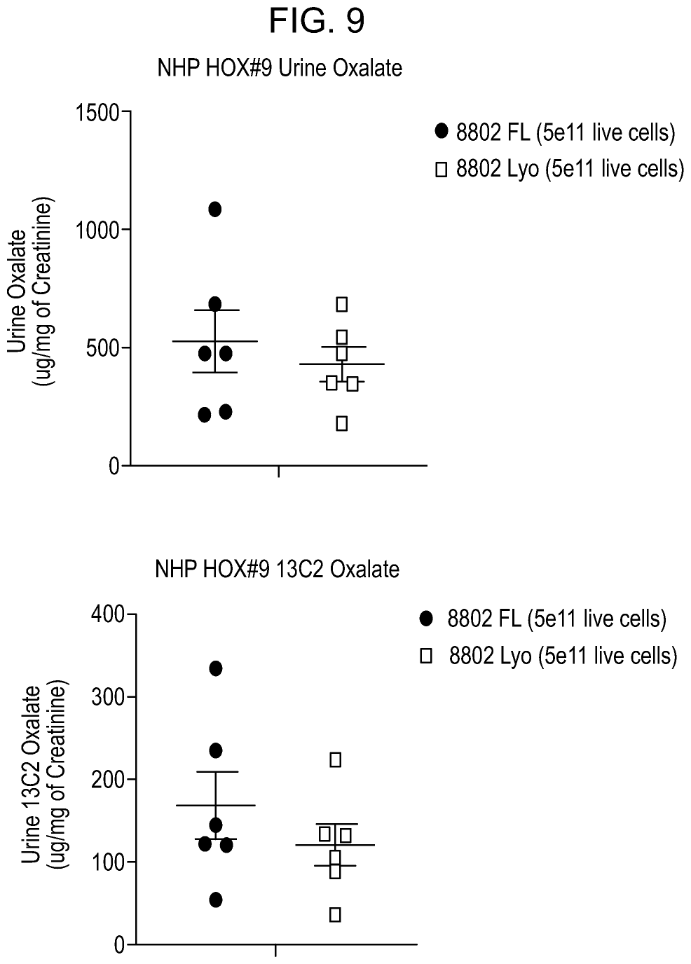

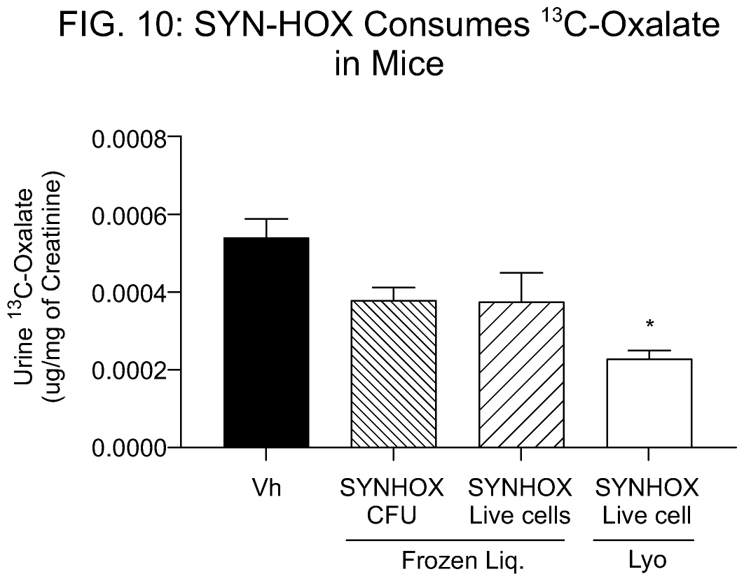

As shown in FIGS. 9-10, oxalate consumption with SYBNB8802 and SYN7169, respectively, lyophilized formulations versus frozen liquid formulations was tested. Male cynomolgus monkeys (approximately 2-5 years of age and average weight of 3.3 kg) were fasted overnight. The cynomolgus monkeys (n=12) received a spinach preparation containing approximately 400 mg of oxalate (including labeled ¹³C-oxalate). One group received 5×10¹¹ live cells of SYNB8802 frozen liquid (n=6) and the other group received 5×10¹¹ live cells of SYNB8802 lyophilized material (n=6). Urine was collected for 6 hours and cumulative urinary oxalate and creatinine levels were measured via liquid chromatography/mass spectrometry. Strains and amounts tested in the NHP “spinach smoothie” model disclosed above are disclosed in Table 33, below. Live cell determination was calculated as described at least in PCT International Application No. PCT/US2020/030468, entitled “Enumeration of Genetically Engineered Microorganisms by Live Cell Counting Techniques,” the entire contents of which are expressly incorporated herein by reference.

TABLE 33

| Study # | Description | Strain Info and Amount Used |
|---|---|---|
| NHP HOX#7 | Dose response of 7169 FL, | SYN7169021920B-001 |
| (20247591) | 1e12 cells (CFU) | 1.01e11 cfu/mL |
| | | Total volume: 60 ml |
| NHP HOX#8 | Dose response of 7169 FL, | SYN7169021920B-001 |
| (20248321) | 5e10 cells (CFU) | 1.01e11 cfu/mL |
| | | Total volume: 30 ml |
| NHP HOX#9 | Efficacy of 8802 FL and Lyo, | SYN7182032620C-003 |
| (20249257) | 5e11 live cells | (1.33 g vial) |
| | | 1.2e+11 live cells/ml |
| | | Total amount: 6 vials |
| | | SYN7182032420H-001 |
| | | L41e+11 live cells/ml |
| | | Total volume: 30 ml |
| NHPHOX#10 | Dose response of 8802 FL, | SYN7182032420C-001 |
| (20250016) | 1e12 cells (CFU) | 1.3e+11 cfu/ml |
| | | Total volume: 60 ml |
| NHPHOX#11 | Dose response of 8802 FL, | SYN7182032420C-001 |
| (20250590) | 1e11 cells (CFU) | 1.3e+11 cfu/ml |
| | | Total volume: 10 ml |
| NHPHOX#12 | Dose response of 8802 Lyo, | SYN7182032620C-003 |
| | 1e12 live cells | (1.33g vial) |
| | | 1.2e+11 live cells/ml |
| | | Total amount: 10 vials |

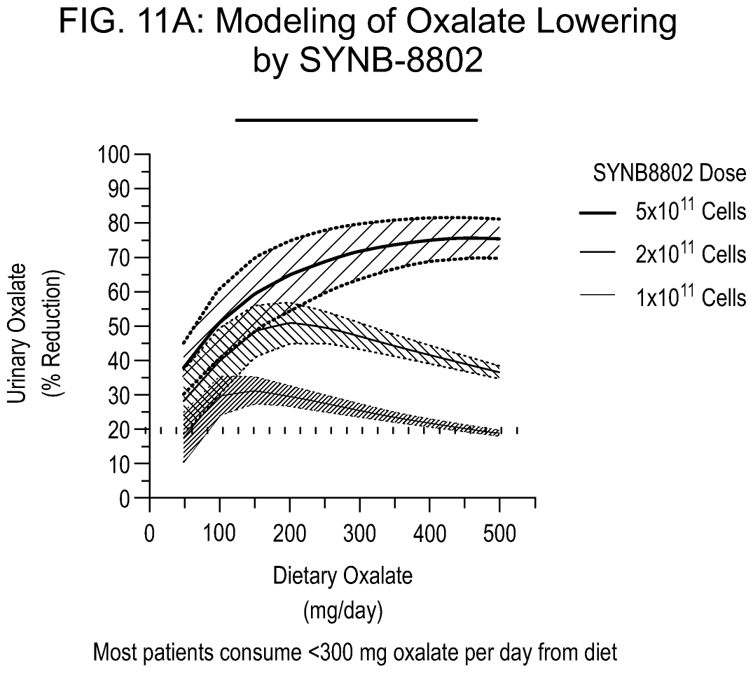

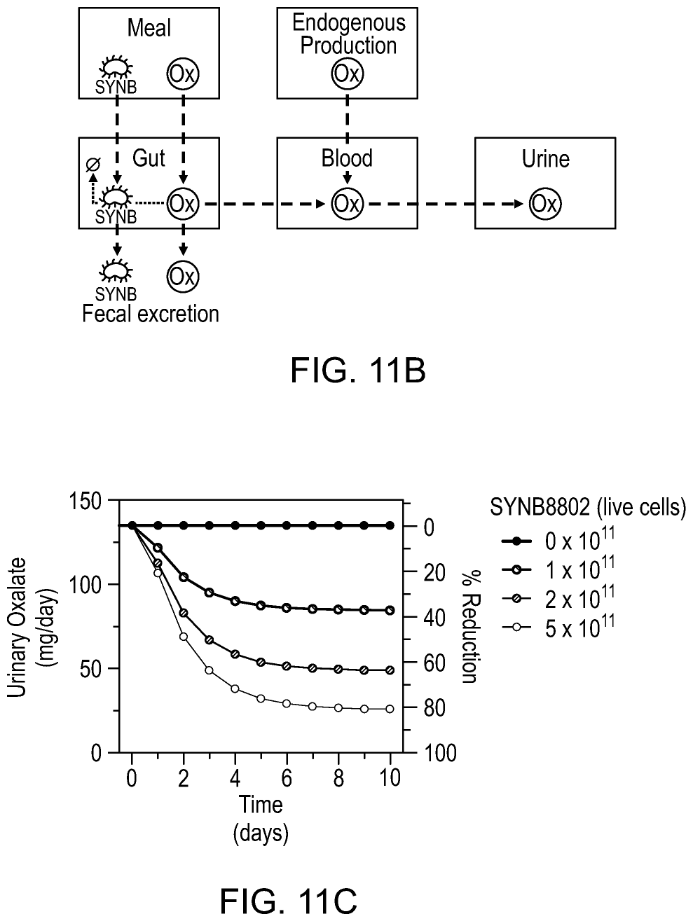

As shown in FIG. 11A, modeling predicts SYNB8802 has potential to achieve 20%-50% urinary oxalate lowering at target dose ranges. Modeling incorporates strain activity assessments in simulated conditions within different gut compartments, known levels of dietary oxalate consumption, oxalate absorption levels with the GI tract, and urinary oxalate excretion. Accordingly, in one embodiment, the dosage of SYNB8802 is 5×10¹¹ cells. In one embodiment, the dosage of SYNB8802 is 2×10¹¹ cells. In one embodiment, the dosage of SYNB8802 is 1×10¹¹ cells.

In silico stimulation (ISS) connects in vitro strain activity knowledge to host and disease biology. The strain-side model simulates the consumption of oxalate by SYNB8802 within the gastrointestinal physiology (FIG. 11B). The host-side model (overall schematic) simulates the impact of consumption by SYNB8802 on the distribution of oxalate throughout the body (FIG. 11B). The model assumes SYNB8802 is dosed with a meal and predicts consumption of gut oxalate and reduction of its absorption into the blood. ISS predicts that SYNB8802 has the potential to achieve greater than 20% urinary oxalate lowering in patients at doses greater than 1×10¹¹ cells.

ISS predicts a dose-dependent lowering of urinary oxalate (FIG. 11C). Data presented as baseline assumption of increased dietary oxalate absorption in HOX patients (4× healthy absorption); bounded region represents the range of the assumption (3×-5× healthy absorption).

### Example 5: Clinical Trials

Clinical trial, Part I, is designed to test SYNB8802 in an inpatient, double-blind, randomized, placebo-controlled, multiple ascending dose (MAD) study in healthy volunteers (HV). SYNB8802 will be administered at multiple ascending doses, for example 1×10¹¹ cells, 3×10¹¹ cells, and 1×10¹² cells, preferably at doses of 6×10¹¹ cells and 2×10¹² cells, or placebo. Dose of SYNB8802 will preferably not exceed 2×10¹² cells. Doses will be administered three times daily (TID) for 5 days. Four days prior, multiple ascending doses will be administered to reach the final dose concentration. Optional cohorts in Part I will include healthy volunteers receiving SYNB8802 at a dose to be determined based on the data from the first cohorts tested administered three times a day for 5 days.

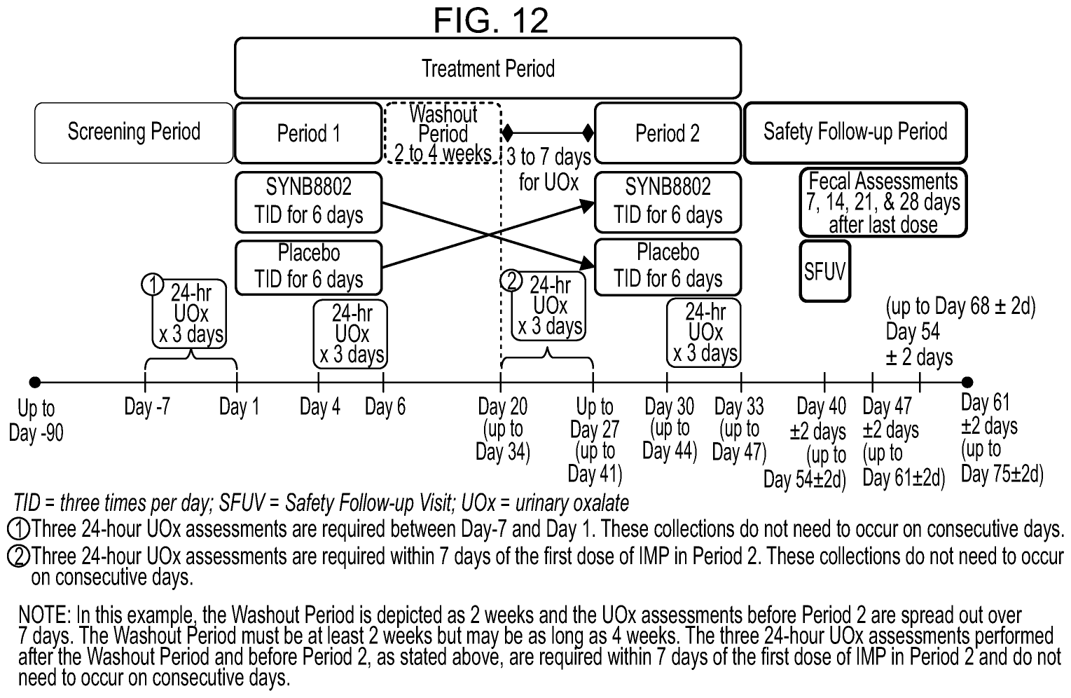

Clinical trial, part II, is designed to test SYNB8802 is an outpatient, double-blind (sponsor-open), randomized, placebo-controlled, crossover study in patients with enteric hyperoxaluria. In order to determine baseline UOx levels for Period 1, 24-hour urine samples will be collected for 3 days, within 7 days of the first dose of investigational medicinal product (IMP) (FIG. 12). IMP at doses 1×10¹¹, 3×10¹¹, or 1×10¹² and not to exceed 2×10¹² will be administered TID. Subjects will take a proton pump inhibitor (PPI; esomeprazole) once a day, 60-90 minutes before the meal of their choosing, starting four days prior to the first IMP dose of each period through the last IMP dose of each period. Subjects will be randomized on Day 1 to receive SYNB8802 at or below the maximum tolerated dose (MTD) determined in Part 1 or placebo and will then be dosed three times daily (TID) with meals on Days 1-6. Four days prior, multiple ascending doses will be administered to reach the final dose concentration. Urine samples for determination of 24-hour oxalate levels will be collected on Days 4-6. After a washout period of at least 2 weeks and no more than 4 weeks, subjects will crossover and begin the second period. In order to determine baseline UOx levels for Period 2.24 hour urine samples will be collected for 3 days, within 7 days of the first dose of Period 2 IMP. Subjects will then crossover to dosing with SYNB8802 or placebo for 6 days. Urine samples for 24-hour oxalate levels will again be collected on the fourth, fifth, and sixth days of Period 2. A Safety Follow-up Visit (or telemedicine) will occur 7 days after last dose of IMP. Subjects will collect weekly fecal samples for 4 weeks after last dose of IMP.

| Arm | Intervention/Treatment |
|---|---|
| Experimental: MAD HV: SYNB8802 | Drug: SYNB8802 |
| live cells) | SYNB8802 is formulated as a |
| HV subjects receive SYNB8802 (1 x 10^{A}l l live | non-sterile solution intended |
| cells) TID for 5 days in the MAD study (Part 1). | for oral administration |
| Experimental: MAD HV: SYNB8802 | Drug: SYNB8802 |
| (3 × 10\hat{ }11 live cells) | SYNB8802 is formulated as a |
| HV subjects receive SYNB8802 (3 × 10\hat{ }11 live | nonsterile solution intended |
| cells) TID for 5 days in the MAD study (Part 1). | for oral administration |
| Experimental: MAD HV: SYNB8802 | Drug: SYNB8802 |
| (1 × 10\hat{ }12 live cells) | SYNB8802 is formulated as a |
| HV subjects receive SYNB8802 (1 × 10\hat{ }12 live | nonsterile solution intended |
| cells) TID for 5 days in the MAD study (Part 1). | for oral administration |
| Experimental: MADHV: SYNB8802 (optional | Drug: SYNB8802 |
| cohort 1) | SYNB8802 is formulated as a |
| HV subjects receive SYNB8802 (at a dose to be | nonsterile solution intended |
| determined based on the data from the first 3 | for oral administration |
| cohorts) | |
| TID for 5 days in the MAD study (Part 1). | |
| Experimental: MADHV: SYNB8802 (optional | Drug: SYNB8802 |
| cohort 2) | SYNB8802 is formulated as a |
| HV subjects receive SYNB8802 (at a dose to be | nonsterile solution intended |
| determined based on the data from the first 3 | for oral administration |
| cohorts) | |
| TID for 5 days in the MAD study (Part 1). | |
| Placebo Comparator: MAD HV: Placebo | Drug: Placebo |
| HV subjects receive placebo TID for 5 days in | In order to maintain study blinding, |
| the MAD study (Part 1). | matching placebo in identical |
| | packaging will be manufactured |
| | using an inactive powder |
| Crossover Arm 1: SYNB8802 crossover to | Drug: SYNB8802 |
| Placebo | SYNB8802 is formulated as a |
| in Part 2 subjects will be randomized (1:1) to | nonsterile solution intended |
| | for oral administration |
| receive SYNB8802 TID for 6 days and then, | Drug: Placebo |
| following a | In order to maintain study blinding, |
| washout period, receive Placebo TID for 6 days. | matching placebo in identical |
| | packaging will be manufactured |
| | using an inactive powder |

Primary outcome measure will be number of subjects with treatment-emergent adverse events. Toxicity will be graded in accordance with National Cancer Institute Common Terminology for Adverse Events (CTCAE0, version 5.0. Acverse events (AEs) are reported based on clinical laboratory tests, vital signs, physical examination, electrocardiograms, and other medically indicated assessments from the time informed consent is singed through the end of the safety follow-up period. AEs are considered to be treatment emergent (TEAE) if they occur or worsen in severity after the first dose of study treatment. TEAEs are considered treatment-related if relationship to study drug is possibly related, probably related, or definitely related.

### Eligibility Criteria

Part I: Inclusion Criteria

1. Age ≥18 to ≤64 years.

2. Body mass index (BMI) 18.5 to 28 kg/m2.

3. Able and willing to voluntarily complete the informed consent process.

4. Available for and agree to all study procedures, including feces, urine, and blood collection and adherence to diet control, inpatient monitoring, follow-up visits, and compliance with all study procedures.

5. Male subjects who are sexually abstinent or surgically sterilized (vasectomy), or those who are sexually active with a female partner(s) and agree to use an acceptable method of contraception (such as a condom with spermicide) combined with an acceptable method of contraception for their non-pregnant female partner(s) (as defined in Inclusion Criterion #6) after informed consent, throughout the study, and for a minimum of 3 months after the last dose of IMP, and who do not intend to donate sperm in the period from Screening until 3 months following administration of the investigational medical product.

6. Female subjects who meet 1 of the following:

a. Woman of childbearing potential (WOCBP) must have a negative pregnancy test (human chorionic gonadotropin) at Screening and at baseline prior to the start of IMP and must agree to use

acceptable method(s) of contraception, combined with an acceptable method of contraception for their male partner(s) (as defined in Inclusion Criterion #5) after informed consent, throughout the study and for a minimum of 3 months after the last dose of IMP. Acceptable methods of contraception include hormonal contraception, hormonal or non-hormonal intrauterine device, bilateral tubal occlusion, complete abstinence, vasectomized partner with documented azoospermia 3 months after procedure, diaphragm with spermicide, cervical cap with spermicide, vaginal sponge with spermicide, or male or female condom with or without spermicide.

b. Premenopausal woman with at least 1 of the following:

i. Documented hysterectomy ii. Documented bilateral salpingectomy Documented bilateral oophorectomy iv. Documented tubal ligation/occlusion v. Sexual abstinence is preferred or usual lifestyle of the subject

c. Postmenopausal women (12 months or more amenorrhea verified by follicle-stimulating hormone [FSH] assessment and over 45 years of age in the absence of other biological or physiological causes).

Part I: Exclusion Criteria

1. Acute or chronic medical (including COVID-19 infection), surgical, psychiatric, or social condition or laboratory abnormality that may increase subject risk associated with study participation, compromise adherence to study procedures and requirements, or may confound interpretation of study safety or PD results and, in the judgment of the investigator, would make the subject inappropriate for enrollment.

2. Body mass index (BMI)<18.5 or >28 kg/m2.

3. Oxalobacter formigenes carrier.

4. Pregnant (self or partner), or lactating.

5. Unable or unwilling to discontinue vitamin C supplementation for the study duration.

6. History of or current immunodeficiency disorder including autoimmune disorders and human immunodeficiency virus (HW) antibody positivity.

7. Hepatitis B surface antigen positivity (subjects with hepatitis B surface antibody positivity and hepatitis B core antibody positivity are not excluded, provided that the hepatitis B surface antigen is negative).

8. Hepatitis C antibody positivity, unless a hepatitis C virus ribonucleic acid test is performed, and the result is negative.

9. History of febrile illness, confirmed bacteremia, or other active infection deemed clinically significant by the investigator within 30 days prior to the anticipated first dose of IMP.

10. History of (within the past month) passage of 3 or more loose stools per day; where ‘loose stool’ is defined as a Type 6 or Type 7 on the Bristol Stool Chart (see Appendix 1: Bristol Stool Chart).

11. History of kidney stones, renal or pancreatic disease.

12. GI disorder (including inflammatory or irritable bowel disorder of any grade and surgical removal of bowel sections) that could be associated with increased UOx levels.

13. Active or past history of GI bleeding within 60 days prior to the Screening Visit as confirmed by hospitalization-related event(s) or medical history of hematemesis or hematochezia.

14. Intolerance of or allergic reaction to EcN, esomeprazole or PPIs in general, or any of the ingredients in SYNB8802 or placebo formulations.

15. Any condition (e.g., celiac disease, gastrectomy, bypass surgery, ileostomy), prescription medication, or over-the-counter product that may possibly affect absorption of medications or nutrients.

16. Currently taking or plans to take any type of systemic (e.g., oral or intravenous) antibiotic within 30 days prior to Day 1 through the final day of inpatient monitoring. Exception: topical antibiotics are allowed.

17. Major surgery (an operation upon an organ within the cranium, chest, abdomen, or pelvic cavity) or inpatient hospital stay within the past 3 months prior to Screening.

18. Planned surgery, hospitalizations, dental work, or interventional studies between Screening and last anticipated visit that might require antibiotics.

19. Taking or planning to take probiotic supplements (enriched foods excluded) within 30 days prior to Day −1 and for the duration of participation and follow-up.

20. Dependence on alcohol or drugs of abuse.

21. Administration or ingestion of an investigational drug within 30 days or 5 half-lives, whichever is longer, prior to Screening Visit; or current enrollment in an investigational study.

22. Screening laboratory parameters (e.g., chemistry panel, hematology, coagulation) and ECG outside of the normal limits based on standard ranges, or as defined in the table below, or as judged to be

clinically significant by the investigator. A single repeat evaluation is acceptable.

Part II: Inclusion Criteria

1. Age ≥18 to ≤74 years.

2. Able and willing to voluntarily complete the informed consent process.

3. Available for and agree to all study procedures, including feces, urine, and blood collection and adherence to diet control, follow-up visits, and compliance with all study procedures.

4. Enteric hyperoxaluria secondary to Roux-en-Y bariatric surgery (at least 12 months post-surgery).

5. Urinary oxalate ≥70 mg/24 hours (mean of at least 2 urine collections during Screening).

6. Male subjects who are sexually abstinent or surgically sterilized (vasectomy), or those who are sexually active with a female partner(s) and agree to use an acceptable method of contraception (such as condom with spermicide) combined with an acceptable method of contraception for their non-pregnant female partner(s) (as defined in Inclusion Criterion #7) after informed consent, throughout the study, and for a minimum of 3 months after the last dose of IMP, and who do not intend to donate sperm in the period from Screening until 3 months following administration of the investigational medical product.

7. Female subjects who meet 1 of the following:

a. Woman of childbearing potential (WOCBP) must have a negative pregnancy test (human chorionic gonadotropin) at Screening and at baseline prior to the start of IMP and must agree to use acceptable method(s) of contraception, combined with an acceptable method of contraception for their male partner(s) (as defined in Inclusion Criterion #6) after informed consent, throughout the study and for a minimum of 3 months after the last dose of IMP. Acceptable methods of contraception include hormonal contraception, hormonal or non-hormonal intrauterine device, bilateral tubal occlusion, complete abstinence, vasectomized partner with documented azoospermia 3 months after procedure, diaphragm with spermicide, cervical cap with spermicide, vaginal sponge with spermicide, or male or female condom with or without spermicide.

b. Premenopausal woman with at least 1 of the following:

i. Documented hysterectomy ii. Documented bilateral salpingectomy Documented bilateral oophorectomy iv. Documented tubal ligation/occlusion v. Sexual abstinence is preferred or usual lifestyle of the subject

c. Postmenopausal women (12 months or more amenorrhea verified by FSH assessment and over 45 years of age in the absence of other biological or physiological causes).

8. Screening laboratory evaluations (e.g., chemistry panel, complete blood count with differential, prothrombin time [PT]/activated partial thromboplastin time [aPTT], urinalysis) and electrocardiogram (ECG) must be within normal limits or judged to be not clinically significant by the investigator.

Part II: Exclusion Criteria

1. Acute or chronic medical (including COVID-19 infection), surgical, psychiatric, or social condition or laboratory abnormality that may increase subject risk associated with study participation, compromise adherence to study procedures and requirements, or may confound interpretation of study safety or PD results and, in the judgment of the investigator, would make the subject inappropriate for enrollment.

2. Acute renal failure or eGFR <45 mL/min/1.73 m2. A single repeat evaluation is acceptable.

3. Unable or unwilling to discontinue vitamin C supplementation for the study duration.

4. Diagnosis of primary hyperoxaluria or any other cause of hyperoxaluria.

5. Oxalobacter formigenes carrier.

6. Pregnant (self or partner), or lactating.

7. Currently taking or plans to take any type of systemic (e.g., oral or intravenous) antibiotic within 30 days prior to Day 1 through the final safety assessment. Exception: topical antibiotics are allowed.

8. Major surgery (an operation upon an organ within the cranium, chest, abdomen, or pelvic cavity) or inpatient hospital stay within the past 3 months prior to Screening.

9. Planned surgery, hospitalizations, dental work, or interventional studies between Screening and last anticipated visit.

10. Taking or planning to take probiotic supplements (enriched foods excluded) within 30 days prior to Day −1 and for the duration of participation.

11. Intolerance of or allergic reaction to EcN, esomeprazole or PPIs in general, or any of the ingredients in SYNB8802 or placebo formulations.

12. Dependence on alcohol or drugs of abuse.

13. History of or current immunodeficiency disorder including autoimmune disorders and HIV antibody positivity.

14. Hepatitis B surface antigen positivity (subjects with hepatitis B surface antibody positivity and hepatitis B core antibody positivity are not excluded, provided that the hepatitis B surface antigen is negative).

15. Hepatitis C antibody positivity, unless a hepatitis C virus ribonucleic acid test is performed, and the result is negative.

16. Administration or ingestion of an investigational drug within 30 days or 5 half-lives, whichever is longer, prior to Screening Visit; or current enrollment in an investigational study.

17. History of bacteremia within 30 days prior to the anticipated first dose of IMP.

18. History of inflammatory bowel disease.

TABLE 34
Other Sequences Related to SYN7169 and SYNB8802

| Description | Sequence ID NO |
|---|---|
| >gn1\|ECOLI\|THYMIDYLATESYN-MONOMER | SEQ ID NO: 61 |
| thymidylate synthase (complement2964361 . . . | |
| 2965155)) Escherichia coli K-12 substr. MG1655 | |
| >gn1\|ECOLI\|EG11002 thyA THYMIDYLATES- | SEQ ID NO: 62 |
| MONOMER (complement(2964361 . . . 2965155)) | |
| Escherichia coli K-12 substr. MG1655 | |
| phage 3 ko sequence | SEQ ID NO: 63 |

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A method for reducing the levels of oxalate in a subject, the method comprising administering to the subject a pharmaceutical composition comprising a recombinant bacterium comprising one or more gene sequences encoding one or more oxalate catabolism enzymes operably linked to a directly or indirectly first promoter that is not associated with the oxalate catabolism enzyme gene in nature, thereby reducing the levels of oxalate in the subject.

2. The method of claim 1, wherein the recombinant bacterium has an oxalate consumption activity of at least about 1 μmol/1×10⁹ cell.

3. The method of claim 1 or claim 2, wherein the recombinant bacterium has an oxalate consumption activity of about 50 to about 600 mg/day under anaerobic conditions.

4. The method of claim 3, wherein the recombinant bacterium has an oxalate consumption activity of about 211 mg/day under anaerobic conditions.

5. The method of any one of the previous claims, wherein the recombinant bacterium has an oxalate consumption activity of about 211 mg/day under anaerobic conditions when administered to the subject three times per day.

6. The method of any one of claims 3-5, wherein the anaerobic conditions are conditions in the intestine and/or colon of the subject.

7. The method of any one of the previous claims, wherein the method reduces acute levels of oxalate in the subject by about two fold.

8. The method of any one of claims 1-6, wherein the method reduces acute levels of oxalate in the subject by about three fold.

9. The method of any one of claims 1-6, wherein the method reduces chronic levels of oxalate in the subject by about two fold.

10. The method of any one of claims 1-6, wherein the method reduces chronic levels of oxalate in the subject by about three fold.

11. The method of any one of claims 1-6, wherein the method reduces acute levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day.

12. The method of any one of claims 1-6, wherein the method reduces chronic levels of oxalate in the subject to about 25 mg/day, about 30 mg/day, about 40 mg/day, about 50 mg/day, about 60 mg/day, about 70 mg/day, about 80 mg/day, about 90 mg/day, or about 100 mg/day.

13. The method of any one of the previous claims, wherein the recombinant bacterium is of the genus Escherichia.

14. The method of claim 13, wherein the recombinant bacterium is of the species Escherichia coli strain Nissle.

15. The method of any one of the previous claims, wherein the pharmaceutical composition is administered orally.

16. The method of any one of the previous claims, wherein the subject is fed a meal within one hour of administering the pharmaceutical composition.

17. The method of any one of claims 1-15, wherein the subject is fed a meal concurrently with administering the pharmaceutical composition.

18. The method of any one of the previous claims, wherein the subject is a human subject.

19. The method of any one of the previous claims, wherein the one or more gene sequences comprise a scaaE3 gene, an frc gene, and an oxdC gene.

20. The method of claim 19, wherein the scaaE3 gene comprises a sequence having at least 90%, 91%, 92%, 93%, 94%, 95%, 96%, 97%, 98%, or 99% identity to, comprises, or consists of SEQ ID NO:3.

21. The method of claim 19 or claim 20, wherein the frc gene comprises a sequence having at least 90%, 91%, 92%, 93%, 94%, 95%, 96%, 97%, 98%, or 99% identity to, comprises, or consists of SEQ ID NO:1.

22. The method of any one of claims 19-21, wherein the oxdC gene comprises a sequence having at least 90%, 91%, 92%, 93%, 94%, 95%, 96%, 97%, 98%, or 99% identity to, comprises, or consists of SEQ ID NO:2.

23. The method of any one of the previous claims, wherein the recombinant bacterium further comprises a gene encoding an oxalate importer.

24. The method of claim 23, wherein the gene encoding the oxalate importer is an oxlT gene.

25. The method of claim 24, wherein the oxlT gene comprises a sequence having at least 90%, 91%, 92%, 93%, 94%, 95%, 96%, 97%, 98%, or 99% identity to, comprises, or consists of SEQ ID NO: 11.

26. The method of any one of the previous claims, wherein the recombinant bacterium further comprises an auxotrophy.

27. The method of claim 26, wherein the auxotrophy is a thyA auxotrophy.

28. The method of any one of the previous claims, wherein the recombinant bacterium further comprises a deletion in an endogenous phage.

29. The method of claim 26, wherein the endogenous phage comprises a sequence having at least 90%, 91%, 92%, 93%, 94%, 95%, 96%, 97%, 98%, or 99% identity to, comprises, or consists of SEQ ID NO: 63.

30. The method of any one of the previous claims, wherein the recombinant bacterium does not comprise a gene encoding for antibiotic resistance.

31. The method of any one of the previous claims, wherein the first promoter is an inducible promoter, optionally when the inducible promoter is a FNR promoter, optionally wherein the FNR promoter comprises a sequence having at least 90%, 91%, 92%, 93%, 94%, 95%, 96%, 97%, 98%, or 99% identity to, comprises, or consists of any one of SEQ ID NOs:13-29.

32. The method of any one of claims 1-17, wherein the recombinant bacterium is SYN5752, SYN7169, or SYNB8802.

33. The method of any one of the previous claims, wherein the subject has hyperoxaluria.

34. The method of claim 33, wherein the hyperoxaluria is primary hyperoxaluria, dietary hyperoxaluria, or enteric hyperoxaluria.

35. The method of claim 33 or claim 34, wherein the subject has short bowel syndrome, chronic pancreatitis, inflammatory bowel disease (IBD), cystic fibrosis, kidney disease, and/or Roux-en-Y gastric bypass.

36. The method of any one of the previous claims, wherein the subject has urinary oxalate (Uox) levels of at least 70 mg/day prior to the administering.

37. The method of any one of the previous claims, wherein the subject exhibits a decrease in Uox levels of at least 10%, 15%, 20%, 25%, 30%, 35%, 40%, 45%, 50%, 55%, 60%, 65%, 70%, 75%, or 80% after the administering.

38. The method of any one of the previous claims, wherein the subject has eGFR <30 mL/min/1.73 m², requires hemodialysis, or has systemic oxalosis prior to the administering.

39. The method of any one of the previous claims, wherein the recombinant bacteria is administered at a dose of about 1×10¹¹ live recombinant bacteria, about 3×10¹¹ live recombinant bacteria, about 6×10¹¹ live recombinant bacteria, about 1×10¹² live recombinant bacteria, or about 2×10¹² live recombinant bacteria.

40. The method of any one of claims 1-38, wherein the recombinant bacteria is administered at a dose of about 6×10¹¹ live recombinant bacteria.

41. The method of any one of claims 1-38, wherein the recombinant bacteria is administered at a dose of about 2×10¹² live recombinant bacteria.

42. The method of any one of claims 1-38, wherein the recombinant bacteria is administered at a dose of about 1×10¹² live recombinant bacteria.

43. The method of any one of claims 1-38, wherein the recombinant bacteria is administered at a dose of about 1×10¹¹ live recombinant bacteria.

44. The method of any one of claims 1-38, wherein the recombinant bacteria is administered at a dose of about 3×10¹¹ live recombinant bacteria.

45. The method of any one of the previous claims, wherein the administering is about 5×10¹¹ live recombinant bacteria with meals three times per day.

46. The method of any one of the previous claims, further comprising administering a proton pump inhibitor (PPI) to the subject.

47. The method of claim 46, wherein the PPI is esomeprazole.

48. The method of claim 46 or 47, wherein the administering of the PPI is once a day.

49. A recombinant bacterium comprising one or more gene sequences encoding one or more oxalate catabolism enzymes operably linked to a directly or indirectly first promoter that is not associated with the oxalate catabolism enzyme gene in nature.

50. The recombinant bacterium of claim 49, wherein the recombinant bacterium has an oxalate consumption activity of at least about 1 μmol/1×10⁹ cell.

51. The recombinant bacterium of claim 49 or claim 50, wherein the recombinant bacterium has an oxalate consumption activity of about 50-600 mg/day under anaerobic conditions.

52. The recombinant bacterium of any one of claims 45-50, wherein the recombinant bacterium is SYNB8802, SYN7169, or SYN5752.

53. The recombinant bacterium of claim 52, wherein the recombinant bacterium is SYNB8802.


SEQUENCE LISTING (66 sequences)

SEQ ID NO: 1 - DNA, 1287 bp, Oxalobacter formigenes
atgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 2 - DNA, 1707 bp, Oxalobacter formigenes
atgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataa

SEQ ID NO: 3 - DNA, 1632 bp, Saccharomyces cerevisiae
atgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaa

SEQ ID NO: 4 - DNA, 1146 bp, Escherichia coli
atgacaaataatgaaagcaaagggccgtttgaaggcttattagttatcgatatgacacatgtccttaatggacctttcggaactcaacttctttgtaatatgggcgcaagggtaattaaagttgagccgccgggtcatggtgatgatacccgcacatttggtccctatgtggatggacagtcactctattacagttttattaatcatggcaaagagagtgtggttcttgatttaaagaatgatcacgataaaagtatatttataaatatgctcaaacaagctgatgtattagctgagaattttcgcccaggtacaatggaaaaactggggttttcatgggaaacgcttcaagaaatcaacccgcgcctcatatatgcttcatcgtcaggtttcggacataccggtccgctaaaagatgctcctgcctacgataccatcattcaggcaatgagcgggataatgatggaaacaggatatcctgatgctccgccagtgcgcgttggtacatctcttgcggatctatgcggcggtgtctatttattcagcggaatagtgagtgcactttatggccgcgaaaagagccagagaggggcgcatgtcgatatagcgatgtttgatgccacgctgagttttctggagcatggtctgatggcatatatcgcaactgggaagtcaccacaacgtctgggaaatcgccatccctacatggcaccttttgatgttttcaatactcaggataagccgattacgatttgttgtggtaatgacaagcttttttctgcgttatgccaggcactggagcttacggaactggttaatgatccccgatttagcagcaatattttacgcgtacaaaaccaggctattcttaaacaatatattgagcggacgttaaaaacgcaggcagctgaagtttggttagccagaatacatgaagttggtgtacccgtcgcgccgttattaagtgtggctgaggccattaaattgccacaaactcaggcgagaaatatgttgattgaagccgggggaataatgatgccgggtaatccgataaaaatcagcggctgcgcggacccgcatgttatgccgggagcggcaacgctcgaccagcatggggaacaaattcgccaggagttctcatcataa

SEQ ID NO: 5 - protein, 428 aa, Oxalobacter formigenes
MTKPLDGINVLDFTHVQAGPACTQMMGFLGANVIKIERRGSGDMTRGWLQDKPNVDSLYFTMFNCNKRSIELDMKTPEGKELLEQMIKKADVMVENFGPGALDRMGFTWEYIQELNPRVILASVKGYAEGHANEHLKVYENVAQCSGGAAATTGFWDGPPTVSGAALGDSNSGMHLMIGILAALEMRHKTGRGQKVAVAMQDAVLNLVRIKLRDQQRLERTGILAEYPQAQPNFAFDRDGNPLSFDNITSVPRGGNAGGGGQPGWMLKCKGWETDADSYVYFTIAANMWPQICDMIDKPEWKDDPAYNTFEGRVDKLMDIFSFIETKFADKDKFEVTEWAAQYGIPCGPVMSMKELAHDPSLQKVGTVVEVVDEIRGNHLTVGAPFKFSGFQPEITRAPLLGEHTDEVLKELGLDDAKIKELHAKQVV

SEQ ID NO: 6 - protein, 568 aa, Oxalobacter formigenes
MSNDDNVELTDGFHVLIDALKMNDIDTMYGVVGIPITNLARMWQDDGQRFYSFRHEQHAGYAASIAGYIEGKPGVCLTVSAPGFLNGVTSLAHATTNCFPMILLSGSSEREIVDLQQGDYEEMDQMNVARPHCKASFRINSIKDIPIGIARAVRTAVSGRPGGVYVDLPAKLFGQTISVEEANKLLFKPIDPAPAQIPAEDAIARAADLIKNAKRPVIMLGKGAAYAQCDDEIRALVEETGIPFLPMGMAKGLLPDNHPQSAAATRAFALAQCDVCVLIGARLNWLMQHGKGKTWGDELKKYVQIDIQANEMDSNQPIAAPVVGDIKSAVSLLRKALKGAPKADAEWTGALKAKVDGNKAKLAGKMTAETPSGMMNYSNSLGVVRDFMLANPDISLVNEGANALDNTRMIVDMLKPRKRLDSGTWGVMGIGMGYCVAAAAVTGKPVIAVEGDSAFGFSGMELETICRYNLPVTVIIMNNGGIYKGNEADPQPGVISCTRLTRGRYDMMMEAFGGKGYVANTPAELKAALEEAVASGKPCLINAMIDPDAGVESGRIKSLNVVSKVGKK

SEQ ID NO: 7 - protein, 543 aa, Saccharomyces cerevisiae
MTSAATVTASFNDTFSVSDNVAVIVPETDTQVTYRDLSHMVGHFQTMFTNPNSPLYGAVFRQDTVAISMRNGLEFIVAFLGATMDAKIGAPLNPNYKEKEFNFYLNDLKSKAICVPKGTTKLQSSEILKSASTFGCFIVELAFDATRFRVEYDIYSPEDNYKRVIYRSLNNAKFVNTNPVKFPGFARSSDVALILHTSGTTSTPKTVPLLHLNIVRSTLNIANTYKLTPLDRSYVVMPLFHVHGLIGVLLSTFRTQGSVVVPDGFHPKLFWDQFVKYNCNWFSCVPTISMIMLNMPKPNPFPHIRFIRSCSSALAPATFHKLEKEFNAPVLEAYAMTEASHQMTSNNLPPGKRKPGTVGQPQGVTVVILDDNDNVLPPGKVGEVSIRGENVTLGYANNPKANKENFTKRENYFRTGDQGYFDPEGFLVLTGRIKELINRGGEKISPIELDGIMLSHPKIDEAVAFGVPDDMYGQVVQAAIVLKKGEKMTYEELVNFLKKHLASFKIPTKVYFVDKLPKTATGKIQRRVIAETFAKSSRNKSKL

SEQ ID NO: 8 - protein, 381 aa, Escherichia coli
MTNNESKGPFEGLLVIDMTHVLNGPFGTQLLCNMGARVIKVEPPGHGDDTRTFGPYVDGQSLYYSFINHGKESVVLDLKNDHDKSIFINMLKQADVLAENFRPGTMEKLGFSWETLQEINPRLIYASSSGFGHTGPLKDAPAYDTIIQAMSGIMMETGYPDAPPVRVGTSLADLCGGVYLFSGIVSALYGREKSQRGAHVDIAMFDATLSFLEHGLMAYIATGKSPQRLGNRHPYMAPFDVFNTQDKPITICCGNDKLFSALCQALELTELVNDPRFSSNILRVQNQAILKQYIERTLKTQAAEVWLARIHEVGVPVAPLLSVAEAIKLPQTQARNMLIEAGGIMMPGNPIKISGCADPHVMPGAATLDQHGEQIRQEFSS

SEQ ID NO: 9 - protein, 437 aa, Escherichia coli
SYYHHHHHHLESTSLYKKAGLMSTPLQGIKVLDFTGVQSGPSCTQMLAWFGADVIKIERPGVGDVTRHQLRDIPDIDALYFTMLNSNKRSIELNTKTAEGKEVMEKLIREADILVENFHPGAIDHMGFTWEHIQEINPRLIFGSIKGFDECSPYVNVKAYENVAQAAGGAASTTGFWDGPPLVSAAALGDSNTGMHLLIGLLAALLHREKTGRGQRVTMSMQDAVLNLCRVKLRDQQRLDKLGYLEEYPQYPNGTFGDAVPRGGNAGGGGQPGWILKCKGWETDPNAYIYFTIQEQNWENTCKAIGKPEWITDPAYSTAHARQPHIFDIFAEIEKYTVTIDKHEAVAYLTQFDIPCAPVLSMKEISLDPSLRQSGSVVEVEQPLRGKYLTVGCPMKFSAFTPDIKAAPLLGEHTAAVLQELGYSDDEIAAMKQNHAI

SEQ ID NO: 10 - protein, 564 aa, Escherichia coli
MSDQLQMTDGMHIIVEALKQNNIDTIYGVVGIPVTDMARHAQAEGIRYIGFRHEQSAGYAAAASGFLTQKPGICLTVSAPGFLNGLTALANATVNGFPMIMISGSSDRAIVDLQQGDYEELDQMNAAKPYAKAAFRVNQPQDLGIALARAIRVSVSGRPGGVYLDLPANVLAATMEKDEALTTIVKVENPSPALLPCPKSVTSAISLLAKAERPLIILGKGAAYSQADEQLREFIESAQIPFLPMSMAKGILEDTHPLSAAAARSFALANADVVMLVGARLNWLLAHGKKGWAADTQFIQLDIEPQEIDSNRPIAVPVVGDIASSMQGMLAELKQNTFTTPLVWRDILNIHKQQNAQKMHEKLSTDTQPLNYFNALSAVRDVLRENQDIYLVNEGANTLDNARNIIDMYKPRRRLDCGTWGVMGIGMGYAIGASVTSGSPVVAIEGDSAFGFSGMEIETICRYNLPVTIVIFNNGGIYRGDGVDLSGAGAPSPTDLLHHARYDKLMDAFRGVGYNVTTTDELRHALTTGIQSRKPTIINVVIDPAAGTESGHITKLNPKQVAGN

SEQ ID NO: 11 - DNA, 1257 bp, Oxalobacter formigenes
atgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaa

SEQ ID NO: 12 - protein, 418 aa, Oxalobacter formigenes
MNNPQTGQSTGLLGNRWFYLVLAVLLMCMISGVQYSWTLYANPVKDNLGVSLAAVQTAFTLSQVIQAGSQPGGGYFVDKFGPRIPLMFGGAMVLAGWTFMGMVDSVPALYALYTLAGAGVGIVYGIAMNTANRWFPDKRGLASGFTAAGYGLGVLPFLPLISSVLKVEGVGAAFMYTGLIMGILIILIAFVIRFPGQQGAKKQIVVTDKDFNSGEMLRTPQFWVLWTAFFSVNFGGLLLVANSVPYGRSLGLAAGVLTIGVSIQNLFNGGCRPFWGFVSDKIGRYKTMSVVFGINAVVLALFPTIAALGDVAFIAMLAIAFFTWGGSYALFPSTNSDIFGTAYSARNYGFFWAAKATASIFGGGLGAAIATNFGWNTAFLITAITSFIAFALATFVIPRMGRPVKKMVKLSPEEKAVH

SEQ ID NO: 13 - DNA, 290 bp, Unknown
  Description of Unknown FNR responsive promoter sequence
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaa

SEQ ID NO: 14 - DNA, 173 bp, Unknown
  Description of Unknown FNR responsive promoter sequence
atttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacct

SEQ ID NO: 15 - DNA, 305 bp, Unknown
  Description of Unknown FNR responsive promoter sequence
gtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacat

SEQ ID NO: 16 - DNA, 180 bp, Unknown
  Description of Unknown FNR responsive promoter sequence
catttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacat

SEQ ID NO: 17 - DNA, 199 bp, Unknown
  Description of Unknown FNR responsive promoter sequence
agttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacat

SEQ ID NO: 18 - DNA, 117 bp, Unknown
  Description of Unknown FNR promoter sequence
atccccatcactcttgatggagatcaattccccaagctgctagagcgttaccttgcccttaaacattagcaatgtcgatttatcagagggccgacaggctcccacaggagaaaaccg

SEQ ID NO: 19 - DNA, 108 bp, Unknown
  Description of Unknown FNR promoter sequence
ctcttgatcgttatcaattcccacgctgtttcagagcgttaccttgcccttaaacattagcaatgtcgatttatcagagggccgacaggctcccacaggagaaaaccg

SEQ ID NO: 20 - DNA, 290 bp, Unknown; identical to SEQ ID NO: 13
  Description of Unknown FNR promoter sequence

SEQ ID NO: 21 - DNA, 433 bp, Unknown
  Description of Unknown FNR promoter sequence
cggcccgatcgttgaacatagcggtccgcaggcggcactgcttacagcaaacggtctgtacgctgtcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaaatgtttgtttaactttaagaaggagatatacat

SEQ ID NO: 22 - DNA, 290 bp, Unknown
  Description of Unknown FNR promoter sequence
gtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtaggcggtaatagaaaagaaatcgaggcaaaa

SEQ ID NO: 23 - DNA, 173 bp, Unknown; identical to SEQ ID NO: 14
  Description of Unknown FNR promoter sequence

SEQ ID NO: 24 - DNA, 305 bp, Artificial Sequence; identical to SEQ ID NO: 15
  Description of Artificial Sequence Synthetic polynucleotide

SEQ ID NO: 25 - DNA, 180 bp, Artificial Sequence; identical to SEQ ID NO: 16
  Description of Artificial Sequence Synthetic polynucleotide

SEQ ID NO: 26 - DNA, 199 bp, Unknown; identical to SEQ ID NO: 17
  Description of Unknown FNR promoter sequence

SEQ ID NO: 27 - DNA, 207 bp, Unknown
  Description of Unknown FNR promoter sequence
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcttggatccaaagtgaactctagaaataattttgtttaactttaagaaggagatatacat

SEQ ID NO: 28 - DNA, 390 bp, Artificial Sequence
  Description of Artificial Sequence Synthetic polynucleotide
tcgtctttgtgatgtgcttcctgttaggtttcgtcagccgtcaccgtcagcataacaccctgacctctcattaattgctcatgccggacggcactatcgtcgtccggccttttcctctcttcccccgctacgtgcatctatttctataaacccgctcattttgtctattttttgcacaaacatgaaatatcagacaattccgtgacttaagaaaatttatacaaatcagcaatatacccattaaggagtatataaaggtgaatttgatttacatcaataagcggggttgctgaatcgttaaggtagaaatgtgatctagttcacatttgcggtaatagaaaagaaatcgaggcaaaaatgtttgtttaactttaagaaggagatatacat

SEQ ID NO: 29 - DNA, 200 bp, Artificial Sequence
  Description of Artificial Sequence Synthetic polynucleotide
agttgttcttattggtggtgttgctttatggttgcatcgtagtaaatggttgtaacaaaagcaatttttccggctgtctgtatacaaaaacgccgcaaagtttgagcgaagtcaataaactctctacccattcagggcaatatctctcaaatgtgatctagttcacattttttgtttaactttaagaaggagatatacat

SEQ ID NO: 30 - DNA, 355 bp, Unknown
  Description of Unknown OxyR-regulated regulatory region sequence
tgtggcttttatgaaaatcacacagtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattgatgcgaattcattaaagaggagaaaggtacc

SEQ ID NO: 31 - DNA, 228 bp, Unknown
  Description of Unknown OxyR-regulated regulatory region sequence
ttccgaaaattcctggcgagcagataaataagaattgttcttatcaatatatctaactcattgaatctttattagttttgtttttcacgcttgttaccactattagtgtgataggaacagccagaatagcggaacacatagccggtgctatacttaatctcgttaattactgggacataacatcaagaggatatgaaattcgaattcattaaagaggagaaaggtacc

SEQ ID NO: 32 - DNA, 334 bp, Unknown
  Description of Unknown OxyR-regulated regulatory region sequence
gcttagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatagatgcgaattcattaaagaggagaaaggtacc

SEQ ID NO: 33 - DNA, 134 bp, Unknown
  Description of Unknown OxyR-regulated regulatory region sequence
ctcgagttcattatccatcctccatcgccacgatagttcatggcgataggtagaatagcaatgaacgattatccctatcaagcattctgactgataattgctcacacgaattcattaaagaggagaaaggtacc

SEQ ID NO: 34 - DNA, 5409 bp, Artificial Sequence
  Description of Artificial Sequence Synthetic polynucleotide
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaaaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 35 - DNA, 4785 bp, Artificial Sequence
  Description of Artificial Sequence Synthetic polynucleotide
cattaattcctaatttttgttgacactctatcattgatagagttattttaccactccctatcagtgatagagaaaagtgaactctagaaataattttgtttaactttaagaaggagatatacatatgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaaaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 36 - DNA, 4661 bp, Artificial Sequence
  Description of Artificial Sequence Synthetic polynucleotide
atgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaaaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 37 - DNA, 2005 bp, Artificial Sequence
  Description of Artificial Sequence Synthetic polynucleotide
ttaagacccactttcacatttaagttgtttttctaatccgcatatgatcaattcaaggccgaataagaaggctggctctgcaccttggtgatcaaataattcgatagcttgtcgtaataatggcggcatactatcagtagtaggtgtttccctttcttctttagcgacttgatgctcttgatcttccaatacgcaacctaaagtaaaatgccccacagcgctgagtgcatataatgcattctctagtgaaaaaccttgttggcataaaaaggctaattgattttcgagagtttcatactgtttttctgtaggccgtgtacctaaatgtacttttgctccatcgcgatgacttagtaaagcacatctaaaacttttagcgttattacgtaaaaaatcttgccagctttccccttctaaagggcaaaagtgagtatggtgcctatctaacatctcaatggctaaggcgtcgagcaaagcccgcttattttttacatgccaatacaatgtaggctgctctacacctagcttctgggcgagtttacgggttgttaaaccttcgattccgacctcattaagcagctctaatgcgctgttaatcactttacttttatctaatctagacatcattaattcctaatttttgttgacactctatcattgatagagttattttaccactccctatcagtgatagagaaaagtgaactctagaaataattttgtttaactttaagaaggagatatacatatgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaa

SEQ ID NO: 38 - DNA, 1381 bp, Artificial Sequence
  Description of Artificial Sequence Synthetic polynucleotide
cattaattcctaatttttgttgacactctatcattgatagagttattttaccactccctatcagtgatagagaaaagtgaactctagaaataattttgtttaactttaagaaggagatatacatatgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaa

SEQ ID NO: 39 - DNA, 1300 bp, Artificial Sequence
  Description of Artificial Sequence Synthetic polynucleotide
ctctagaaataattttgtttaactttaagaaggagatatacatatgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaa

SEQ ID NO: 40 - DNA, 4874 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgaccagtgcagctacggtgaccgcgagctttaatgacactttttctgtgagcgataatgtcgcggtaatcgtaccggaaaccgatacgcaggtcacctaccgtgatctttcccacatggtaggacactttcaaacaatgttcacgaacccgaatagtcctctgtacggggcggtctttcgtcaagacacggtagcgattagcatgcgtaacggccttgaatttattgtggctttccttggagccacgatggatgcgaaaattggtgcgccactgaatcccaattataaagagaaggagtttaatttttacctgaatgacttaaagtccaaagccatctgcgtgccgaaaggcaccaccaaactgcaaagttcagaaattcttaagagtgcgtccacgttcgggtgctttattgtggaactggcgtttgacgccacccgttttcgtgttgaatatgacatttactccccggaggacaattataaacgtgtgatctaccgcagccttaacaatgctaagtttgtcaacacaaaccctgtcaagttcccgggtttcgcccgcagctcggatgttgcacttattttgcatacctcaggcaccactagtaccccaaagaccgtacccctcttgcatctgaatattgtccgttcaaccctgaatatcgccaacacttacaaacttaccccgctggatcgctcctatgttgtaatgccgctgtttcatgtacatggattaatcggcgtcttactgagtacgttccgcacccagggcagtgtagtcgtcccggacggctttcatccgaagctcttctgggatcagtttgttaaatataactgcaattggtttagttgcgtcccaacgatctctatgattatgttgaatatgcccaaaccgaatccgtttccgcacattcgctttatccgctcatgtagcagcgcgctggcgccagcaacgtttcacaagctggaaaaagaatttaatgccccagttctggaagcgtacgcgatgacagaagcatctcatcagatgaccagtaacaatctgcctcccggtaaacgtaaaccggggaccgtgggccaacctcaaggtgtaaccgtagtaatcctggatgacaacgataacgttctgccgcccggcaaagttggcgaggtgtcgatccgtggggagaacgtcaccctgggctacgctaataacccgaaagctaacaaagaaaacttcactaaacgtgaaaactatttccgtaccggggatcagggctacttcgacccggagggctttctcgtgctgaccggccgcattaaagaattgatcaatcgcggtggtgaaaaaattagtcctattgaactggacggaatcatgctctcgcatcctaaaatcgacgaggcggtggcgttcggcgttccagatgatatgtatggccaagtcgttcaggcggcaatcgtgttgaaaaagggggaaaagatgacctatgaagaattagtgaatttcctgaaaaagcatttagcaagctttaaaatcccaaccaaagtctactttgtggataagctgcctaaaacggccaccgggaagattcaacgtcgcgtaatcgccgaaaccttcgcgaaatctagtcgcaacaaaagcaaactttaaaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 41 - DNA, 213 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacat

SEQ ID NO: 42 - DNA, 1470 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgaataatccacaaacaggacaatcaacaggcctcttgggcaatcgttggttctacttggtattagcagttttgctgatgtgtatgatctcgggtgtccaatattcctggacactgtacgctaacccggttaaagacaaccttggcgtttctttggctgcggttcagacggctttcacactctctcaggtcattcaagctggttctcagcctggtggtggttacttcgttgataaattcggtccaagaattccattgatgttcggtggtgcgatggttctcgctggctggaccttcatgggtatggttgacagtgttcctgctctgtatgctctttatactctggccggtgcaggtgttggtatcgtttacggtatcgcgatgaacacggctaacagatggttcccggacaaacgcggtctggcttccggtttcaccgctgccggttacggtctgggtgttctgccgttcctgccactgatcagctccgttctgaaagttgaaggtgttggcgcagcattcatgtacaccggtttgatcatgggtatcctgattatcctgatcgctttcgttatccgtttccctggccagcaaggcgccaaaaaacaaatcgttgttaccgacaaggatttcaattctggcgaaatgctgagaacaccacaattctgggttctgtggaccgcattcttttccgttaactttggtggtttgctgctggttgccaacagcgtcccttacggtcgcagcctcggtcttgccgcaggtgtgctgacgatcggtgtttcgatccagaacctgttcaatggtggttgccgtcctttctggggtttcgtttccgataaaatcggccgttacaaaaccatgtccgtcgttttcggtatcaatgctgttgttctcgcacttttcccgacgattgctgccttgggcgatgtagcctttatcgccatgttggcaatcgcattcttcacatggggtggtagctacgctctgttcccatcgaccaacagcgatattttcggtacggcatactctgccagaaactatggtttcttctgggctgcaaaagcaactgcctcgatcttcggtggtggtctgggtgctgcaattgcaaccaacttcggatggaataccgctttcctgattactgcgattacttctttcatcgcatttgctctggctaccttcgttattccaagaatgggccgtccagtcaagaaaatggtcaaattgtctccagaagaaaaagctgtacattaa

SEQ ID NO: 43 - DNA, 3225 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 44 - DNA, 3055 bp, Artificial Sequence
  Description of Artificial Sequence Synthetic polynucleotide
ctctagaaataattttgtttaactttaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 45 - DNA, 4388 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaactctagaaataattttgtttaactttaagaaggagatatacatatgacaaataatgaaagcaaagggccgtttgaaggcttattagttatcgatatgacacatgtccttaatggacctttcggaactcaacttctttgtaatatgggcgcaagggtaattaaagttgagccgccgggtcatggtgatgatacccgcacatttggtccctatgtggatggacagtcactctattacagttttattaatcatggcaaagagagtgtggttcttgatttaaagaatgatcacgataaaagtatatttataaatatgctcaaacaagctgatgtattagctgagaattttcgcccaggtacaatggaaaaactggggttttcatgggaaacgcttcaagaaatcaacccgcgcctcatatatgcttcatcgtcaggtttcggacataccggtccgctaaaagatgctcctgcctacgataccatcattcaggcaatgagcgggataatgatggaaacaggatatcctgatgctccgccagtgcgcgttggtacatctcttgcggatctatgcggcggtgtctatttattcagcggaatagtgagtgcactttatggccgcgaaaagagccagagaggggcgcatgtcgatatagcgatgtttgatgccacgctgagttttctggagcatggtctgatggcatatatcgcaactgggaagtcaccacaacgtctgggaaatcgccatccctacatggcaccttttgatgttttcaatactcaggataagccgattacgatttgttgtggtaatgacaagcttttttctgcgttatgccaggcactggagcttacggaactggttaatgatccccgatttagcagcaatattttacgcgtacaaaaccaggctattcttaaacaatatattgagcggacgttaaaaacgcaggcagctgaagtttggttagccagaatacatgaagttggtgtacccgtcgcgccgttattaagtgtggctgaggccattaaattgccacaaactcaggcgagaaatatgttgattgaagccgggggaataatgatgccgggtaatccgataaaaatcagcggctgcgcggacccgcatgttatgccgggagcggcaacgctcgaccagcatggggaacaaattcgccaggagttctcatcataaaagaaggagatatacatatgagtaacgacgacaatgtagagttgactgatggctttcatgttttgatcgatgccctgaaaatgaatgacatcgataccatgtatggtgttgtcggcattcctatcacgaacctggctcgtatgtggcaagatgacggtcagcgtttttacagcttccgtcacgaacaacacgcaggttatgcagcttctatcgccggttacatcgaaggaaaacctggcgtttgcttgaccgtttccgcccctggcttcctgaacggcgtgacttccctggctcatgcaaccaccaactgcttcccaatgatcctgttgagcggttccagtgaacgtgaaatcgtcgatttgcaacagggcgattacgaagaaatggatcagatgaatgttgcacgtccacactgcaaagcttctttccgtatcaacagcatcaaagacattccaatcggtatcgctcgtgcagttcgcaccgctgtatccggacgtccaggtggtgtttacgttgacttgccagcaaaactgttcggtcagaccatttctgtagaagaagctaacaaactgctcttcaaaccaatcgatccagctccggcacagattcctgctgaagacgctatcgctcgcgctgctgacctgatcaagaacgccaaacgtccagttatcatgctgggtaaaggcgctgcatacgcacaatgcgacgacgaaatccgcgcactggttgaagaaaccggcatcccattcctgccaatgggtatggctaaaggcctgctgcctgacaaccatccacaatccgctgctgcaacccgtgctttcgcactggcacagtgtgacgtttgcgtactgatcggcgctcgtctgaactggctgatgcagcacggtaaaggcaaaacctggggcgacgaactgaagaaatacgttcagatcgacatccaggctaacgaaatggacagcaaccagcctatcgctgcaccagttgttggtgacatcaagtccgccgtttccctgctccgcaaagcactgaaaggcgctccaaaagctgacgctgaatggaccggcgctctgaaagccaaagttgacggcaacaaagccaaactggctggcaagatgactgccgaaaccccatccggaatgatgaactactccaattccctgggcgttgttcgtgacttcatgctggcaaatccggatatttccctggttaacgaaggcgctaatgcactcgacaacactcgtatgattgttgacatgctgaaaccacgcaaacgtcttgactccggtacctggggtgttatgggtattggtatgggctactgcgttgctgcagctgctgttaccggcaaaccggttatcgctgttgaaggcgatagcgcattcggtttctccggtatggaactggaaaccatctgccgttacaacctgccagttaccgttatcatcatgaacaatggtggtatctataaaggtaacgaagcagatccacaaccaggcgttatctcctgtacccgtctgacccgtggtcgttacgacatgatgatggaagcatttggcggtaaaggttatgttgccaatactccagcagaactgaaagctgctctggaagaagctgttgcttccggcaaaccatgcctgatcaacgcgatgatcgatccagacgctggtgtcgaatctggccgtatcaagagcctgaacgttgtaagtaaagttggcaagaaataataagaaggagatatacatatgactaaaccattagatggaattaatgtgcttgactttacccacgtccaggcaggtcctgcctgtacacagatgatgggtttcttgggcgcaaacgtcatcaagattgaaagacgtggttccggagatatgactcgtggatggctgcaggacaaaccaaatgttgattccctgtatttcacgatgttcaactgtaacaaacgttcgattgaactggacatgaaaaccccggaaggcaaagagcttctggaacagatgatcaagaaagccgacgtcatggtcgaaaacttcggaccaggcgcactggaccgtatgggctttacttgggaatacattcaggaactgaatccacgcgtcattctggcttccgttaaaggctatgcagaaggccacgccaacgaacacctgaaagtttatgaaaacgttgcacagtgttccggcggtgctgcagctaccaccggtttctgggatggtcctccaaccgtttccggcgctgctctgggtgactccaactccggtatgcacctgatgatcggtattctggccgctctggaaatgcgtcacaaaaccggccgtggtcagaaagttgccgtcgctatgcaggacgctgttctgaatctggttcgtatcaaactgcgtgaccagcaacgtctggaaagaaccggcattctggctgaatacccacaggctcagcctaactttgccttcgacagagacggtaacccactgtccttcgacaacatcacttccgttccacgtggtggtaacgcaggtggcggcggccagccaggctggatgctgaaatgtaaaggttgggaaaccgatgcggactcctacgtttacttcaccatcgctgcaaacatgtggccacagatctgcgacatgatcgacaagccagaatggaaagacgacccagcctacaacacattcgaaggtcgtgttgacaagctgatggacatcttctccttcatcgaaaccaagttcgctgacaaggacaaattcgaagttaccgaatgggctgcccagtacggcattccttgcggtccggtcatgtccatgaaagaactggctcacgatccttccctgcagaaagttggtaccgtcgttgaagttgtcgacgaaattcgtggtaaccacctgaccgttggcgcaccgttcaaattctccggattccagccggaaattacccgtgctccgctgttgggcgaacataccgacgaagttctgaaagaactgggtcttgacgatgccaagatcaaggaactgcatgcaaaacaggtagtttga

SEQ ID NO: 46 - DNA, 4388 bp, Artificial Sequence; identical to SEQ ID NO: 45
  Description of Artificial Sequence Synthetic polynucleotide

SEQ ID NO: 47 - DNA, 60 bp, Artificial Sequence
  Description of Artificial Sequence Synthetic primer
tagaactgatgcaaaaagtgctcgacgaaggcacacagatgtgtaggctggagctgcttc

SEQ ID NO: 48 - DNA, 60 bp, Artificial Sequence
  Description of Artificial Sequence Synthetic primer
gtttcgtaattagatagccaccggcgctttaatgcccggacatatgaatatcctccttag

SEQ ID NO: 49 - DNA, 52 bp, Artificial Sequence
  Description of Artificial Sequence Synthetic primer
caacacgtttcctgaggaaccatgaaacagtatttagaactgatgcaaaaag

SEQ ID NO: 50 - DNA, 46 bp, Artificial Sequence
  Description of Artificial Sequence Synthetic primer
cgcacactggcgtcggctctggcaggatgtttcgtaattagatagc

SEQ ID NO: 51 - DNA, 36 bp, Artificial Sequence
  Description of Artificial Sequence Synthetic primer
atatcgtcgcagcccacagcaacacgtttcctgagg

SEQ ID NO: 52 - DNA, 47 bp, Artificial Sequence
  Description of Artificial Sequence Synthetic primer
aagaatttaacggagggcaaaaaaaaccgacgcacactggcgtcggc

SEQ ID NO: 53 - DNA, 3383 bp, Artificial Sequence
  Description of Artificial Sequence Synthetic polynucleotide
ggtaccgtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggtaggcggtaatagaaaagaaatcgaggcaaaaatgagcaaagtcagactcgcaattatggatcctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 54 - DNA, 3258 bp, Artificial Sequence
  Description of Artificial Sequence Synthetic polynucleotide
ggtacccatttcctctcatcccatccggggtgagagtcttttcccccgacttatggctcatgcatgcatcaaaaaagatgtgagcttgatcaaaaacaaaaaatatttcactcgacaggagtatttatattgcgcccgttacgtgggcttcgactgtaaatcagaaaggagaaaacacctatgacgacctacgatcgggatcctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 55 - DNA, 3386 bp, Artificial Sequence
  Description of Artificial Sequence Synthetic polynucleotide
ggtaccgtcagcataacaccctgacctctcattaattgttcatgccgggcggcactatcgtcgtccggccttttcctctcttactctgctacgtacatctatttctataaatccgttcaatttgtctgttttttgcacaaacatgaaatatcagacaattccgtgacttaagaaaatttatacaaatcagcaatataccccttaaggagtatataaaggtgaatttgatttacatcaataagcggggttgctgaatcgttaaggatccctctagaaataattttgtttaactttaagaaggagatatacatatgactatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 56 - DNA, 3261 bp, Artificial Sequence
  Description of Artificial Sequence Synthetic polynucleotide
ggtacccatttcctctcatcccatccggggtgagagtcttttcccccgacttatggctcatgcatgcatcaaaaaagatgtgagcttgatcaaaaacaaaaaatatttcactcgacaggagtatttatattgcgcccggatccctctagaaataattttgtttaactttaagaaggagatatacatatgactatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 57 - DNA, 3279 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccctctagaaataattttgtttaactttaagaaggagatatacatatgctatgattacggattctctggccgtcgtattacaacgtcgtgactgggaaaaccctggcgttacccaacttaatcgccttgcggcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgacgccgatactgtcgtcgtcccctcaaactggcagatgcacggttacgatgcgcctatctacaccaacgtgacctatcccattacggtcaatccgccgtttgttcccgcggagaatccgacaggttgttactcgctcacatttaatattgatgaaagctggctacaggaaggccagacgcgaattatttttgatggcgttaactcggcgtttcatctgtggtgcaacgggcgctgggtcggttacggccaggacagccgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgaccacgcaaatcagcgatttccaagttaccactctctttaatgatgatttcagccgcgcggtactggaggcagaagttcagatgtacggcgagctgcgcgatgaactgcgggtgacggtttctttgtggcagggtgaaacgcaggtcgccagcggcaccgcgcctttcggcggtgaaattatcgatgagcgtggcggttatgccgatcgcgtcacactacgcctgaacgttgaaaatccggaactgtggagcgccgaaatcccgaatctctatcgtgcagtggttgaactgcacaccgccgacggcacgctgattgaagcagaagcctgcgacgtcggtttccgcgaggtgcggattgaaaatggtctgctgctgctgaacggcaagccgttgctgattcgcggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctacccgcgatgagcgaacgcgtaacgcggatggtgcagcgcgatcgtaatcacccgagtgtgatcatctggtcgctggggaatgaatcaggccacggcgctaatcacgacgcgctgtatcgctggatcaaatctgtcgatccttcccgcccggtacagtatgaaggcggcggagccgacaccacggccaccgatattatttgcccgatgtacgcgcgcgtggatgaagaccagcccttcccggcggtgccgaaatggtccatcaaaaaatggctttcgctgcctggagaaatgcgcccgctgatcctttgcgaatatgcccacgcgatgggtaacagtcttggcggcttcgctaaatactggcaggcgtttcgtcagtacccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccggcgctgacggaagcaaaacaccaacagcagtatttccagttccgtttatccgggcgaaccatcgaagtgaccagcgaatacctgttccgtcatagcgataacgagttcctgcactggatggtggcactggatggcaagccgctggcaagcggtgaagtgcctctggatgttggcccgcaaggtaagcagttgattgaactgcctgaactgccgcagccggagagcgccggacaactctggctaacggtacgcgtagtgcaaccaaacgcgaccgcatggtcagaagccggacacatcagcgcctggcagcaatggcgtctggcggaaaacctcagcgtgacactcccctccgcgtcccacgccatccctcaactgaccaccagcggaacggatttttgcatcgagctgggtaataagcgttggcaatttaaccgccagtcaggctttctttcacagatgtggattggcgatgaaaaacaactgctgaccccgctgcgcgatcagttcacccgtgcgccgctggataacgacattggcgtaagtgaagcgacccgcattgaccctaacgcctgggtcgaacgctggaaggcggcgggccattaccaggccgaagcggcgttgttgcagtgcacggcagatacacttgccgacgcggtgctgattacaaccgcccacgcgtggcagcatcaggggaaaaccttatttatcagccggaaaacctaccggattgatgggcacggtgagatggtcatcaatgtggatgttgcggtggcaagcgatacaccgcatccggcgcggattggcctgacctgccagctggcgcaggtctcagagcgggtaaactggctcggcctggggccgcaagaaaactatcccgaccgccttactgcagcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagccaacaacaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 58 - DNA, 1921 bp, Artificial Sequence
  Description of Artificial Sequence Synthetic polynucleotide
ttacccgtctggattttcagtacgcgcttttaaacgacgccacagcgtggtacggctgatccccaaataacgtgcggcggcgcgcttatcgccattaaagcgtgcgagcacctcctgcaatggaagcgcttctgctgacgagggcgtgatttctgctgtggtccccaccagttcaggtaataattgccgcataaattgtctgtccagtgttggtgcgggatcgacgcttaaaaaaagcgccaggcgttccatcatattccgcagttcgcgaatattaccgggccaatgatagttcagtagaagcggctgacactgcgtcagcccatgacgcaccgattcggtaaaagggatctccatcgcggccagcgattgttttaaaaagttttccgccagaggcagaatatcaggctgtcgctcgcgcaaggggggaagcggcagacgcagaatgctcaaacggtaaaacagatcggtacgaaaacgtccttgcgttatctcccgatccagatcgcaatgcgtggcgctgatcacccggacatctaccgggatcggctgatgcccgccaacgcgggtgacggctttttcctccagtacgcgtagaaggcgggtttgtaacggcagcggcatttcgccaatttcgtcaagaaacagcgtgccgccgtgggcgacctcaaacagccccgcacgtccacctcgtcttgagccggtaaacgctccctcctcatagccaaacagttcagcctccagcaacgactcggtaatcgcgccgcaattaacggcgacaaagggcggagaaggcttgttctgacggtggggctgacggttaaacaacgcctgatgaatcgcttgcgccgccagctctttcccggtccctgtttccccctgaatcagcactgccgcgcgggaacgggcatagagtgtaatcgtatggcgaacctgctccatttgtggtgaatcgccgaggatatcgctcagcgcataacgggtctgtaatcccttgctggaggtatgctggctatactgacgccgtgtcaggcgggtcatatccagcgcatcatggaaagcctgacgtacggtggccgctgaataaataaagatggcggtcattcctgcctcttccgccaggtcggtaattagtcctgccccaattacagcctcaatgccgttagctttgagctcgttaatttgcccgcgagcatcctcttcagtgatatagcttcgctgttcaagacggaggtgaaacgttttctgaaaggcgaccagagccggaatggtctcctgataggtcacgattcccattgaggaagtcagctttcccgcttttgccagagcctgtaatacatcgaatccgctgggtttgatgaggatgacaggtaccgacagtcggctttttaaataagcgccgttggaacctgccgcgataatcgcgtcgcagcgttcggttgccagttttttgcgaatgtaggctactgccttttcaaaaccgagctgaataggcgtgatcgtcgccagatgatcaaactccaggctgatatcccgaaatagttcgaacaggcgcgttaccgagaccgtccagatcaccggtttatcgctattatcgcgcgaagcgctatgcacagtaaccatcgtcgtagattcatgtttaaggaacgaattcttgttttatagatgtttcgttaatgttgcaatgaaacacaggcctccgtttcatgaaacgttagctgactcgtttttcttgtgactcgtctgtcagtattaaaaaagatttttcatttaactgattgtttttaaattgaattttatttaatggtttctcggtttttgggtctggcatatcccttgctttaatgagtgcatcttaattaacaattcaataacaagagggctgaatagtaatttcaacaaaataacgagcattcgaatg

SEQ ID NO: 59 - DNA, 967 bp, Unknown
  Description of Unknown clbA sequence
caaatatcacataatcttaacatatcaataaacacagtaaagtttcatgtgaaaaacatcaaacataaaatacaagctcggaatacgaatcacgctatacacattgctaacaggaatgagattatctaaatgaggattgatatattaattggacatactagtttttttcatcaaaccagtagagataacttccttcactatctcaatgaggaagaaataaaacgctatgatcagtttcattttgtgagtgataaagaactctatattttaagccgtatcctgctcaaaacagcactaaaaagatatcaacctgatgtctcattacaatcatggcaatttagtacgtgcaaatatggcaaaccatttatagtttttcctcagttggcaaaaaagattttttttaacctttcccatactatagatacagtagccgttgctattagttctcactgcgagcttggtgtcgatattgaacaaataagagatttagacaactcttatctgaatatcagtcagcatttttttactccacaggaagctactaacatagtttcacttcctcgttatgaaggtcaattacttttttggaaaatgtggacgctcaaagaagcttacatcaaatatcgaggtaaaggcctatctttaggactggattgtattgaatttcatttaacaaataaaaaactaacttcaaaatatagaggttcacctgtttatttctctcaatggaaaatatgtaactcatttctcgcattagcctctccactcatcacccctaaaataactattgagctatttcctatgcagtcccaactttatcaccacgactatcagctaattcattcgtcaaatgggcagaattgaatcgccacggataatctagacacttctgagccgtcgataatattgattttcatattccgtcggtggtgtaagtatcccgcataatcgtgccattcacatttag

SEQ ID NO: 60 - DNA, 424 bp, Unknown
  Description of Unknown clbA knock-out sequence
ggatggggggaaacatggataagttcaaagaaaaaaacccgttatctctgcgtgaaagacaagtattgcgcatgctggcacaaggtgatgagtactctcaaatatcacataatcttaacatatcaataaacacagtaaagtttcatgtgaaaaacatcaaacataaaatacaagctcggaatacgaatcacgctatacacattgctaacaggaatgagattatctaaatgaggattgatgtgtaggctggagctgcttcgaagttcctatactttctagagaataggaacttcggaataggaacttcggaataggaactaaggaggatattcatatgtcgtcaaatgggcagaattgaatcgccacggataatctagacacttctgagccgtcgataatattgattttcatattccgtcggtgg

SEQ ID NO: 61 - protein, 264 aa, Escherichia coli
MKQYLELMQKVLDEGTQKNDRTGTGTLSIFGHQMRFNLQDGFPLVTTKRCHLRSIIHELLWFLQGDTNIAYLHENNVTIWDEWADENGDLGPVYGKQWRAWPTPDGRHIDQITTVLNQLKNDPDSRRIIVSAWNVGELDKMALAPCHAFFQFYVADGKLSCQLYQRSCDVFLGLPFNIASYALLVHMMAQQCDLEVGDFVWTGGDTHLYSNHMDQTHLQLSREPRPLPKLIIKRKPESIFDYRFEDFEIEGYDPHPGIKAPVAI

SEQ ID NO: 62 - DNA, 795 bp, Escherichia coli
atgaaacagtatttagaactgatgcaaaaagtgctcgacgaaggcacacagaaaaacgaccgtaccggaaccggaacgctttccatttttggtcatcagatgcgttttaacctgcaagatggattcccgctggtgacaactaaacgttgccacctgcgttccatcatccatgaactgctgtggtttctgcagggcgacactaacattgcttatctacacgaaaacaatgtcaccatctgggacgaatgggccgatgaaaacggcgacctcgggccagtgtatggtaaacagtggcgcgcctggccaacgccagatggtcgtcatattgaccagatcactacggtactgaaccagctgaaaaacgacccggattcgcgccgcattattgtttcagcgtggaacgtaggcgaactggataaaatggcgctggcaccgtgccatgcattcttccagttctatgtggcagacggcaaactctcttgccagctttatcagcgctcctgtgacgtcttcctcggcctgccgttcaacattgccagctacgcgttattggtgcatatgatggcgcagcagtgcgatctggaagtgggtgattttgtctggaccggtggcgacacgcatctgtacagcaaccatatggatcaaactcatctgcaattaagccgcgaaccgcgtccgctgccgaagttgattatcaaacgtaaacccgaatccatcttcgactaccgtttcgaagactttgagattgaaggctacgatccgcatccgggcattaaagcgccggtggctatctaa

SEQ ID NO: 63 - DNA, 10859 bp, Artificial Sequence
  Description of Artificial Sequence Synthetic polynucleotide
ttacgctgcagttacggtaactttgcagaccgcaacgaaattaccgtcgctggtcataacaataacgtcagcggtgcctgccgccacgccggtgacggtgatcgcattaccgctaacggtgaccgttgcttttgccccgtctgaggttgccacacggaacgaggtatctgaggcactggctgggttaaccgtcacattgagcgttgtggttgcgccgacggccacgcttgccgtggctttatcgagcgtaacgcctgtcacggggatattcggggtcccgctttcttctgccagttccggcttgccggtattggtaattttcgctgtacgggttatgacctcttttgccggaatggctttacccaggctgctgcaccagccgcggaaaacgtcgacggtaccgttcgggtatttgattttgtaatagcgtactgagccatcaataaaccatgcgacaaggtctttttgcccttcttcgcccggcttccaggcgagggtgaacgaggtatcgccagcagattttgccccctgggccgtcgcgttccagtcggcatcctcgtcgtcgaggtaagtgtcgtcatacgattcggcggtcatttcgcccggcgtcagctctttaattttcgccaggcggttccagtcgatatccgagagtgggttagcgaaagcgttgcccgttccggtgtaaagccagagggtggtaccggcacctttcacaggggccagcgggtttggagtaggcataagtacctcttaaattgaataggtgattaagtacgtgaaatcgactgaaccccaggtggccatttcatcatcccgctgatagtcataaccctgcggggtgaacgtctcgaccagttcggtcagacctgggatgaaggccattgccggatacactttctcttccatccaggaatcaagcgcgctgtcggggctggaggctttaagaaatacctcgatgtgaacaaccgcctgccacgaatcttcgtcaagcgaatcgccggtgtactccgcgtcagaaaggtatacagccacggcagggagatcctgctcttcaagaaaaacagggcgcccgtcaaaccaggtgaccgtgtcggtgatctcggctttcagtttggccagaatggctgcacgaattgcgctgtgtctgttcatcgcttcaggtggatcctcagttggtttttcagggctgcggaaagttctttgggcatatcgctttcaataaggcgctttgaaatagcggtgaaggccacggtgagcggtgtctcaagaggaactttgaccacatcaatcggataacgggcctgacctacgcgccgcatgacctgccagcgcccgttcgcaagctgttggataaaagcgttacgaaaggtatagggcccgattttaaggacgctgcccgctccgtttctggccccttttttacgcgagagcctgacgcgcgccgtgccgagctttatcgcaggaagattaccgcggttgatttttatcgacgcgaccgggcgatcgtgacgggccttgcgcagacgggaacgctggcggaccagacgaaccggaagcccctttttccggttatcatcaactgttgcttctttcgctacagctttgctcccctggcttatcgttcttctggccaccctgttaagtgcttttgcggttgcctcaggaacgattaaccggctgaggctgttcaggttctgaatagccctttccagtcctttcacagacatagcgcctcctcattcgagatggatgcggggttttccgttgaacatgtcatagcgggtaacgatcaggttcttaccgtcgtagtcgacgctgtcgtttcggcgtggctggtaaagctcagagaaaaccaccagcgaagtacctgttcccgacaatggccccatttcctcgagttgctcggcgggaacaacgtcatagctgctgccattgatgatcgctgtctttcccatcttttttatagtggccgcgtccatgcgcgccgccatccggtcaaaggagttaggcattgatcttaacttcaacaacggtggtgtttgcccctgcatcttcccaggcgatgcccgcggcaacggcgtccgtttcttcgatcgtgattttgccgtccttcagatacacctgcgccccggcagtaaccgcatctgcggatacttttggcaggaggaaaacaccctcagtaaaaccgtccccggtatcgccagccgggatatcggtaattgccaccgcgataagttttccaacaacaaccgggtcgccgctgtgaacatcggttgcaccactgtttaccagagggatcgttttcccgtcctgcgcatagttcttagccataacttctccattcagcccctttcgaggctggtttcaggtataaaaaaagcccttacgggcgtctgtttgtcaggactgttttttactgaccagaggatttggtcatgccgcgatagtccagcggcgccacgccagcatcaatacgcactttcgtggcgataccatcagtggtgaagccttcctgctgatcgatgtatggcgtgtcgacgccgttgagataagcgacctcaatggtgtcggtgcccttcgcggcagccagataccaggctttcgcatcagcttcatccagacgtggttcggcaatgacttctgcaaagttctggatagggttaacgatcccggcattgatgtctgcacctttaacactggccgacttgatggtctgatttgccagagtttccagggcgacgggcaccagcatgtaggccggacggatattcagggttcgctccccctccttctgcagacgcatcagcttgcgcgattcgtccaggctggccacagaaattgcacccgagctcaggttcttgtgatcggcatggaacagcgcctttccgtctgagagtttcgggtttttggtcagaatggcgtaaaccagatcgccaatcgttgctttcgccgcgcgccccatcttcatcggtacgtcggtaagctggttcagatcgtcgttgatgatcgcctggcgagttactgagaagatttcaccatacgtggcaagcgcgatggtttcgcctttgtcactggtagtgatgtacttgtactcagccccttcgcgaacctgtcgcagagaagggaacccacccataccgacacgatgcgccgttttgaagtccgacagctggccttttttggtccactgctcgaaggtttcctgcgcctcgtcccagccctgaatcagcgctttgttcgcaacatcaagcagaatgttgccaaagtcagaggtgctgtgggtcagcgccaggccaaccatctgcatcgggttgtagctggccacgccgatacctttttctgtcagggccatacgcgcatactcgcgcagcgtcataccgttataaacgttatcccgctcctgaccttcgaacccggcacgcgccatcagtgcctggcgaataccatccgcgacgaagttaccgttgcccgcatgaatatgcggctgagtggttttattggacggcgtggccgttttaccgagttctgccagcagcaaatctttcgccttatcgacggagcaatcagggtcggccacacactgattctgcagttccatgtgcttattaccgaacatggcaaagagatcgccgatagcgttaacacgggttttctgctcagccaacacctgcgcgcggatcgcattttcatccggtgccgggtctgtttttgcctgcggtgcctgaggctgggtaataaccgggtcacgctgggtagtgttgcgcggcggggtgatcatgttgcgaatgctttttggcattttttcaaattcctcaatacgttttgaatgaatacaggccatagcctgaagggatggtgtcacctggtcggcaaaacccagttcaaggcactcgctgccgttcatccaggtttcgtcctccagcattaccgcaatttcttcggtggattttccggttttctgtgcataagccgggataagaacggattcaaccttgtcgagaagatccgcatagtcgcgcatatcgctcgcgtcaccaccagcaaacccccagggcttatggatcatcatcatcgtgttttcaggcatgatgaccggattgcctaccatcgcaatcaccgaggccatggaggccgccagaccgtcgatatgtacggtaatcgccgcgccgtggtgcttcagcgcgttataaatagcaattccgtcgaagacatcaccaccgggcgagttgatataaaggttgatgtgggtgacgtccccaagtgcccggagatcattgacgaactgtttcgccgttacgccccagtacccgatttcgtcataaataaaaatgtcggcctcactgttattgctggcctgcatgcggaaccacgaattactttttgcgctggctttcggacggtggcgcgcccggttctttggcttcggcactggtgcctcctttatcattggcggggtcggtgtcaaacaccaggccctgttcacggttctcgtcaacctcagctttacggcgtgacttaacatcatccgggttgcgaccgctggcacgtatccagtcggattcagtagcagcaccgccgcggatctgcgttttccaggcattcgcttctttaacgggatcaatccacggcataacgggccccgaataaaccgcgttataaagcgagtccatatcaatgcctctcggcagcttgatttctccggcagcaatagccatcttgagccaggctcggtacatgggccgggtcactgaaccgatgaaccagtcctgaagaatcagatagccgtcggttgactcgacaagctcctgccgctgggcactgtacgttccgttgtagtttctggatgtgctggaaaagctgaggcgactgccggcggacacggcacgcagctgtccgttacgaaaagattcgaggttagggttcgggcgatcggatttaatcatcccgatttcttccccggcctgcagttcgtcatagagcataccgggctgaatcatcagctcgcggtcatcgctgcttgaatcagaatcgaagctctgtccgtcgccttttttgatatacatgccgagtgccgcagcaattctggcagcagtaagctccgagtcctcgtattctttcagcgcgctcagacgcatcagaacaccagacaaaagagacgttccgcgggtctggtgcaggcgtcgggtgaatttgagatgcagcatgttctctgcatctatctctttggtatcaaactgacgcccggatactggcaggcttttatagacctgatattttttcgggcgtccccagttatcgacaaaaacgccctgattgagctgggtggcagcatcgctgttcatcggcacaaagtccggctccagcgcttccagccagaacggcacgccagcaaccggctgaagaccatttccggtaccgcgaaccagctgagcaaatacctcaccgtcccggagccacgttcgcagcatcagccgctccagcattgggcgggtaaactgggttgtgacatctggccttacggaccattcgccccactttcggcggatatcagtggccagctttttagcgatcttcccgttactcagcatcggatgcggttcaactatgatgcccttcgcacccaccaccctttcttccagcttgtcgaaaacgccgatcaccagatcgtggttgttatccagccagcgcgcctgctgcctcagcgaaaccgcccccatctggctgagctgatcggctgaacgattttccttctgggctttgtgggtacgcgtttgctttaccgcctcatacgctttaataactgcgcgggcacgcaggcgtgaggctttccagcctggtgaaaacaggccaatcgcatcatctaaaaaactcatccaaacctcgccagcctgtagccgggtcgcccacggcgtttgttattgagcgttgccagtcgtcgctcccattcctgacggccttttctgatttccgacaggttttcgagcgtcatctgctgcccgttgaaagtgattgatttcccctccagaacagacagctcggctgcagcatagcggtcgatcatgttttgaatatctgctggattcacacccaacctcctgacgaagaccacggattagcctgctcggttacgggcttctcacgttttggttttggtttagatttcggcgcaggcggcggggatggcatttcgccagcttccgtctgcgtgtcctcgatccacgtttcccgccgtgcccactcaggagctgacggccatttgattttttcgtaaccactaaggatggcgagcgcgtcggcataaacgagcaggtcaaatgcttcgtttgcgccccggccgggcttactccatttcccttcattcgagcgttcctcatacgtcagttcgtcatagaaccagctgcccagccaggcggggaaatgcacatagccagggccgggtgaatcacgccacagcgcattattcacccggtctttaagggcatcggtctggagaagataaagaggcacatcaccagtcgcctgtgcgcggcgcgttgatctgcccgtgttgtcgggaaacgttcgctggataagtttgctgcgcctgacgctgtcccctttgaagagatagatacgcttacccagcccctcacggcgacatctgcgccagaacttgtaggcattatccgtcacgccatcttcgccccctgagtccacggccatcgacatcagccgcatgccctttgatgggtcagctgcgagcggccacgttttatcaaagacgtcagtgagtaaaagatcccagtcctccggatagctcgccggatccacctgaatgctttcaccgttgccgtcgcagcgcagcgaatgccggatgttgtaacggtcaactatccagcgctcacccatacttccataacccgtaatctgcacaacaaagcgccggttgcgcccggcctgcacgtccacggtcgcagtgagaaactgcacgccgttcggtaccgaacgttttgggacgtcttcggcacgctgctcgagcaattcacttttacgctgctccatgctggctcgcggcaaatagggcctgccgaaatcggtgttgatcaccgtcttcagggtttcttcgctgcgcgtggattcatattcctgctcggcggtcagaaacttataaataagctgcgcccaggtctggtaagcagctgccggaccttccatccagaaggaggcaatacgggaacgacggccatcaccgctaaccaggcctttcctgtcgatggtttgcccgtcccggagccagacacatttcatgttaagcgcacgcttcatgtccggtgtgatcctgcctttacaggcagggcactgaagaaacgccgcttcgctggcaagcacaggatcgctgctgtcgcggtatccggtcatattgtccatttccggctggaaatattcgccgcaatgcgggcatggccagtaaagacgacggcggtcaccacggttatagagcgataaaattccggtggtcggaggggcttcatggggcgtggagcgccgccattttgtgtctctgatatccctcccgggcgagctctcaaccagcgtcatcccggaggacatgaatgtcgtggttcgtttcgatgccagtgaaaaagcatccccctccccgtcgatatcttccggaaagcggtcataatccgtcagcgccacacttttatagtccgaggacgacatgatattgacggatggccagcccagcttcagatagttaccggcgcggaatgtacggtcgtagacgttgttatcgttacgtcttgggcttagccgggttttaacttcagggctacagcgaaaagtacggtccaggcgttttttggaatgctcgcgcgctttttcctcagatacctgaattacaagcatatctgccggatcgcagacaatgttataaacgatccagccgtcaatcagcccgatggttttacccgttcgcgctgggcccacaaacacaaccgcatcgtattcacgcgatgccagacagttcatcggctcaatcacatagggtgccagatccggatcccacggaactgagtttcccgcccccattggcacgcgcatataagtactgaccgcatcggccaccggcatacgacgcggggctcgtaaaataccggaaacatcgcggcggatgtccctggcggatgcccgctttgccatcagtcctcctcaggctgctcctcctcttttccagcgtcctgcaccttctccgccatctggtcgcgcagatcatcgataacgctttgcacacgaactaccgcagcaggcgttaaagcacagtcgcgctcgagcacatccgggagggtttcaagtaccatgacgacggctttcgccatcaatgagaattctcgcgccacttcatctgcgggtattaactgccccgtatcctgttcgaacttcagcctctcgttctctgctttccagtgggacagcctgtcagaagggggcatatcgtcgatgttggccgaaacggtagggatcatcagttcggtcagaatgtcggtcaccagatagagctttaacttgctattgctgcctggagcaggttcaacatttttcagtctcgcggcaaccgtctgacggtgtacgccggttatccctgccagctggttgatattgagttttaaagtggcaatttcctggtccatgatggtgaacactttttgaacgattcgacatgttgcgaaaatggcctctaattaaatcaaagacctgcgcacatgatgatgatgaccctggatccgaaaaactagccgtttcccgcgagcacgccgccccgtggcagggtccccctccgggagtaccttttgataataattatcaattgcacactatcgacggcactgctgccagataacaccaccggggaaacattccatcatgatggccgtgcggacataggaagccagttcatccatcgctttcttgtctgctgccatttgctttgtgacatccagcgccgcacattcagcagcgtttttcagcgcgttttcgatcaacgtttcaatgttggtatcaacaccaggtttaactttgaacttatcggcactgacggttaccttgttctgcgctggctcatcacgctggataccaaggctgatgttgtagatattggtcaccggctgaggtgtttcgattgccgctgcgtggatagcaccatttgcgatagcggcgtccttgatgaatgacactccattgcgaataagttcgaaggagacggtgtcacgaatgcgctggtccagctcgtcgattgccttttgtgcagcagaggtatcaatctcaacgccaagcgtcatcgaagcgcaatattgctgctcaccaaaacgcgtattgaccaggtgttcaacggcaaatttctgcccttctgatgtcagaaaggtaaagtgattttctttctggtattcagttgctgtgtgtctggtttcagcaaaaccaagctcgcgcaattcggctgtgccagatttagaaggcagatcaccagacagcaacgcgccacggaaaaacagcgcataaagcacttcattagcagcgccagatagcgtaatgattttgttactcatggaatatttccttttaggcgtgagcctgtcgcacggcaatgccgcccgagaggtaaacgcaacctaacggcatcacccaggctcactactgaaagactctctttgatgtgcgcgtgcgatgcgcgtagaagactgatttatcaacctgtctttatatcaggattcattacctgactatttgtgggtaaagttcgtagtgcgctgatcgtgcaaaatgattttagttgggaacagttcgcaactctgtcccataaaaatcagcatattcccatctatcccatatccagcgcattgaccatcgggatactgaagggagattccatcatctcttagaaagatcaccatctcttttgtttcaatttgcatatagctacctggaggatttatgaatgcaaggattttcatggactattaccatgagattgattttccatctttattcgcgagagcagtggaaagcgatgacgatgtgggtactacattgcgcattcacctactttgtgagcgcatggtcgaagcatggatatgcgcatgctgtgactgccaagatctctttggaagagataaaaacaaacttttaatcgaatgtaatactaaaatatccatggcgggaaacctgggaatccccccggaacttatgaaatcacttaaaaccatcaactcaatgcgtaatgaccttgcacacaatccatcaatacaaagcattgctgattcaaggatccagagcctgaaggatactctgactgaatactttaaacagcatccaacggaacccagcatggaagaatcaaaactgggtatttttaacgccgagaatcaattaaccgaagaagtttccttagatagtgacagttcaaaaaacagacttaagttaatcttgctgttcagcaagttaatgcaggcgttaatgcaattagttgcagctaatcataatgggcgctgggataaccaatttagccaattcgtttaccatgtgaccatgaacgcaacaaagagataaatccaagcccgttttgtacgggctgttgcattatcacaggcactcagtgaatgcctgctgtaatgccgctagtcgtcgagttgcaacacaccgtgatccagtgattctgaataggcgataagtccggtataaccggggataatctcaccattatcagcttcaaattcaggaattgtgccggtggtgatggtgtattgaggctggccatcttccttcgcgaaggctgccaggtcttcaatctgcttagctgtaagaactactgtcatgctcat

SEQ ID NO: 64 - DNA, 18 bp, Artificial Sequence
  Description of Artificial Sequence Synthetic oligonucleotide
ttgttgayryrtcaacwa

SEQ ID NO: 65 - DNA, 15 bp, Artificial Sequence
  Description of Artificial Sequence Synthetic oligonucleotide
  modified_base (8)..(8): a, c, t, g, unknown or other
ttataatnattataa

SEQ ID NO: 66 - DNA, 170 bp, Artificial Sequence
  Description of Artificial Sequence Synthetic polynucleotide
ggtaccagttgttcttattggtggtgttgctttatggttgcatcgtagtaaatggttgtaacaaaagcaatttttccggctgtctgtatacaaaaacgccgtaaagtttgagcgaagtcaataaactctctacccattcagggcaatatctctcttggatccaaagtgaa